(19) United States
(12) Patent Application Publication
(10) Pub. No.: US 2023/0357424 A1
(43) Pub. Date: Nov. 9, 2023

(54) CD45 BINDING MOLECULES AND METHODS OF USE

(71) Applicant: Synthekine, Inc., Menlo Park, CA (US)

(72) Inventors: Patrick J. LUPARDUS, Redwood City, CA (US); Deepti ROKKAM, Sunnyvale, CA (US)

(21) Appl. No.: 18/040,260

(22) PCT Filed: Aug. 16, 2021
(86) PCT No.: PCT/US2021/046140

§ 371 Date: Feb. 1, 2023

Related U.S. Application Data
(60) Provisional application No. 63/068,747, filed on Aug. 21, 2020.

(51) Int. Cl. C07K 16/28

(52) U.S. Cl. CPC C07K 16/289; C07K 2317/34; C07K 2317/569; C07K 2317/35; C07K 2317/24; C07K 2317/92

(57) ABSTRACT

The present disclosure relates to biologically active molecules comprising a single domain antibody that that selectively binds to the extracellular domain of human CD45, compositions comprising such antibodies, methods of use thereof.

### CROSS-REFERENCE TO RELATED PATENT APPLICATIONS

The present application is a U.S. National phase application of PCT Application PCT/US2021/046140, filed Aug. 16, 2021, which claims benefit of priority to U.S. Provisional Patent Application No. 63/068,747, filed Aug. 21, 2020, which is incorporated by reference for all purposes.

### SEQUENCE LISTING

The instant application contains a Sequence Listing which has been submitted electronically in ASCII format and is hereby incorporated by reference in its entirety. Said ASCII copy, created on Feb. 1, 2023, is named 106249-1362159-001520US SL.TXT and is 335,753 bytes in size.

### FIELD OF THE INVENTION

The present disclosure relates to biologically active molecules comprising a single domain antibody that selectively binds to the extracellular domain of the CD45RO isoform of human CD45, compositions comprising such antibodies, and methods of use thereof.

### BACKGROUND

CD45 is an evolutionarily conserved receptor protein tyrosine phosphatase (PTRPC) and plays a central role in immune cell activation. hCD45 is a type 1 transmembrane protein, isoforms of which are observed on all differentiated hematopoietic cells except mature erythrocytes and platelets. CD45 is also referred to in the scientific literature as leukocyte common antigen (“LCA”), protein tyrosine phosphatase receptor type C (“PTPRC”), LY5, B220, L-CA, T200, CD45 receptor (“CD45R”) and GP180. Suggestive of a role in cell function, CD45 homologues are found in a variety mammalian and non-mammalian species including insects.

All CD45 isoforms contain an extracellular domain, a single transmembrane segment and two tandem intracellular catalytic domains and are classified as a receptor type PTP. Human CD45 (hCD45, UniProtKB-P08575) is encoded by the PTPRC gene (NCBI Gene ID: 5788) which contains 34 exons. The extracellular domain of CD45 contains an N-terminal mucin-like domain encoded by exons 1, 2, and 3 (corresponding to domains A, B and C of the extracellular domain of CD45), which are alternatively spliced to generate eight proteins featuring zero, one, two or all three exons as illustrated in FIG. 1. CD45R (also referred to as CD45RABC) contains all three possible exons. Although there are a significant number of possible combinations of the various ECD domains of CD45 isoforms, only six isoforms (CD45RA, CD45RO, CD45FB, CD45RAB, CD45RBC and CD45RABC) have been identified in humans. The CD45RO isoform is primarily expressed on activated and memory T cells, some B cell subsets, activated monocytes-/macrophages, and granulocytes. CD45RO enhances both T cell receptor and B cell receptor signaling mediated activation. The CD45RA and RB isoforms are associated with na\check{i}ve T cells. The CD45RABC form is associated with B cells. CD45RABC is the longest CD45 isoform and migrates at 200 kDa when isolated from T cells. B cells may also express a CD45R isoform (CD45B220), which is more extensively glycosylated and has a molecular weight of approximately 220 kDa. CD45 B220 expression is not restricted to B cells and may also be found on activated T cells, certain dendritic cells, and on other antigen-presenting cells.

CD45 is a large transmembrane glycoprotein that constitutes approximately 5-10% of the total glycoprotein on the surface of mammalian T and B cells. The extracellular domain of CD45 is unusually large (approximately 550 amino acids) and further highly glycosylated. The extracellular domain of CD45 is comprised of an N-terminal mucin-like domain, which can be alternatively spliced to a 180 kDa CD45 isoform containing only the core RO domain (CD45RO) consisting of four domains with fibronectin 3 domain (FN3)-like topology. The extracellular region contains an N-terminal mucin-like domain encoded by exons 1, 2, and 3 (also called A, B, and C) that can be alternatively spliced to a core domain consisting of four domains with FN3 like topology. The mucin-like domain contains multiple O-linked glycosylation sites while the core CD45RO isoform consists solely of the four core domains and contains a number of N-linked glycosylation motifs. As a result, the size and structure of the CD45 extracellular domain is highly heterogeneous between both individual protein monomers on the surface of the cell, as well as between unique cell types expressing different CD45 splice forms.

Although the ECD of CD45 isoforms within a species may differ significantly in the sequence of their extracellular domains, their intracellular and transmembrane domains are highly conserved. The ICD of CD45 comprises two tyrosine phosphatase domains. The catalytic activity of the intracellular domain of CD45 is observed to play roles both in the activation and inactivation of immune cells. Alexander, D. R. (2000) Seminars in Immunology 12:349-359. One aspect of CD45 function is the dephosphorylation of immunoreceptor tyrosine-based activation/inactivation motifs (ITAM/ITIMs) located one the intracellular domains of a variety of protein. This dephosphorylation activity can lead to activation (e.g. Src kinases required for antigen receptor signaling) or inhibition (e.g. JAK kinases involved in cytokine signaling) of the functions of such proteins. For example, CD45 activates the Src kinase Lck by dephosphorylating an autoinhibitory tyrosine at position 505 site of LCK resulting in LCK activation, an early and essential step in the signaling cascade initiated by TCR ligation. CD45 has also been shown to dephosphorylate tyrosine-based phosphorylation sites on cytokine receptors (Irie-Sasaki et al., 2001).

The extracellular domain of CD45 also possesses functional activity and plays a role in CD45 function. The extracellular domain of the CD45 (CD45RABC) molecule is unusually large, particularly with its heavily glycosylated extracellular domain which extends linearly out from the cell surface. The unusual size of to the CD45RABC extracellular domain has led to the development of what is now largely accepted of the “kinetic segregation model” which postulates that the large size of the extracellular domain of CD45 results in its exclusion from the TCR immunological synapse thereby facilitating increased ITAM phosphorylation and concomitant activation of TCR signaling. Carbone, et al. (2017) Proc Natl Acad Sci USA. 114(44): E9338-E9345; James, J. R., and Vale, R. D. (2012) Nature 487:64-69.

The different CD45 isoforms show varying presentation based on T cell type as well as the state of differentiation. For example, antigenically na\check{i}ve T cells display a CD45RA isoform comprising the A extracellular domain and are commonly characterized as CD45RA positive (CD45RA+) T cells. In contrast activated memory T cells express the CD45RO isoform which lacks the A, B and C domains. Consequently, the cell surface display of particular CD45 isoforms may be used as markers to identify particular subsets of immune cells for identification or targeting. Antibodies against the various isoforms of CD45 are used in immunohistochemistry to differentiate among immune cell types and to differentiate lymphomas and carcinomas in tissue samples. The CD45RO isoform appears to be restricted to a subset of activated T cells, memory cells and cortical thymocytes and is not detected on B cells. Terry, e al. (1988) Immunology 64:331-336. A number of studies indicate that naive T lymphocytes prior to antigen activation express CD45RA and switch to expression of CD45RO following antigen activation. CD45RO is therefore widely regarded as a marker for antigen-experienced or memory T cells. Zola, et al. (1992) Cellular Immunology 145, 175-186 (1992)

The study of CD45 activity and the development of therapeutic and imaging agents targeting CD45 has been hampered by a small set of available antibodies. The present disclosure describes that several existing anti-CD45 antibodies of the literature were evaluated and found to be specific to the N-terminal FN3 domains of CD45RO. The present disclosure does not characterize the binding of the UCHL1 antibody, which reportedly birds to the CD45RO. Smith, et al. (1986) Immunology 58:63-70. A humanized form of UCHL1 antibody and Fab fragments thereof is described in Thie, et al., U.S. Pat. No. 9,701,756 issued Jul. 11, 2017 and published as US20160152733A1 on Jun. 2, 2016. Neither of these references describe a single domain antibody such as the CL45RO VHHs provided in the present disclosure. As previously noted, the display of CD45 isoforms is characteristic of particular cell types and states of development. Consequently, the limited pool of available antibodies limits the ability to identify and/or target the presence of certain cell types and/or the state of such cells. The present disclosure provides polypeptide binding molecules targeting each of the four FN3-like domains of CD45RO.

Although monoclonal antibodies are the most widely used reagents for the detection and quantification of proteins, monoclonal antibodies are large molecules of about 150 kDa and it sometimes limits their use in assays with several reagents competing for close epitopes recognition. A unique class of immunoglobulin containing a heavy chain domain and lacking a light chain domain (commonly referred to as heavy chain” antibodies (HCAbs) is present in camelids, including dromedary camels, Bactrian camels, wild Bactrian camels, llamas, alpacas, vicuñas, and guanacos as well as cartilaginous fishes such as sharks. The isolated variable domain region of HCAbs is known as a VHH (an abbreviation for “variable-heavy-heavy” reflecting their architecture) or Nanobody® (Ablynx). In contrast, the single domain VHH antibody possesses the advantage of small size (˜12-14 kD), approximately one-tenth the molecular weight a conventional mammalian IgG class antibody which allows one to access unique therapeutic spaces when compared to a standard monoclonal IgG format (Ingram et al., 2018). Because of these limitations, the use of smaller antibody-derived molecules has emerged. Furthermore, VHH single domain antibodies are frequently characterized by high thermal stability facilitating pharmaceutical distribution to geographic areas where maintenance of the cold chain is difficult or impossible. Combined with simple phage display discovery methods that do not require heavy/light chain pairing, straightforward humanization, and simple manufacture (e.g. in bacterial expression systems) makes VHH single domain antibodies useful in a variety of healthcare applications including the development of imaging agents and as a component of bispecific agents for imaging or therapeutic applications. Antibody discovery and engineering of VHH s has become an important part of the antibody engineering toolkit, both for discovery of research and diagnostic tools as well as next generation antibody therapeutics (Meyer et al., 2014).

### SUMMARY OF THE INVENTION

The present disclosure provides polypeptides that specifically bind to the extracellular domain of mammalian CD45.

The present disclosure provides a CD45 binding molecule that selectively bind to the extracellular domain of hCD45. In some embodiments, the CD45 binding molecule specifically binds to the D1 domain of hCD45. In some embodiments, the CD45 binding molecule specifically binds to the D2 domain of hCD45. In some embodiments, the CD45 binding molecule specifically binds to the D3 domain of hCD45. In some embodiments, the CD45 binding molecule specifically binds to the D4 domain of hCD45.

In some embodiments, the CD45 binding molecule comprises a single domain antibody (sdAb) that selectively binds to the extracellular domain of hCD45. In some embodiments, the sdAb of the CD45 binding molecule specifically binds to the D1 domain of hCD45. In some embodiments, the sdAb of the CD45 binding molecule specifically binds to the D2 domain of hCD45. In some embodiments, the sdAb of the CD45 binding molecule specifically binds to the D3 domain of hCD45. In some embodiments, the sdAb of the CD45 binding molecule specifically binds to the D4 domain of hCD45RO.

In some embodiments, the CD45 binding molecule consists of, optionally consists essentially of, or optionally comprises a single domain antibody (sdAb) having at least 80%, alternatively at least 85%, alternatively at least 90%, alternatively at least 95%, alternatively at least 98%, alternatively at least 99% identity (or being identical except for 1, 2, 3, or 4 amino acids that optionally are conserved substitutions) to a polypeptide sequence selected from the group consisting of: SEQ ID NO: 2, SEQ ID NO: 34, SEQ ID NO: 38, SEQ ID NO: 78, SEQ ID NO: 82, SEQ ID NO: 94, SEQ ID NO: 106, SEQ ID NO: 134, SEQ ID NO: 126, and SEQ ID NO: 138.

In some embodiments, the CD45 binding molecule comprises a single domain antibody (sdAb) that selectively binds to the extracellular domain of hCD45 wherein the sdAb is a VHH selected from the group consisting of: SEQ ID NO: 2, SEQ ID NO: 34, SEQ ID NO: 38, SEQ ID NO: 78, SEQ ID NO: 82, SEQ ID NO: 94, SEQ ID NO: 106, SEQ ID NO: 134, SEQ ID NO: 126, and SEQ ID NO: 138.

In some embodiments, the CD45 binding molecule comprises a sdAb that specifically binds to the D1 domain of hCD45. In some embodiments, the sdAb that specifically binds to the D1 domain of hCD45 is a polypeptide having at least 80%, alternatively at least 85%, alternatively at least 90%, alternatively at least 95% identity, alternatively at least 98%, alternatively at least 99% identity (or being identical except for 1, 2, 3, or 4 amino acids that optionally are conserved substitutions) to a polypeptide sequence of SEQ ID NO: 2. In some embodiments, the CD45 binding molecule comprises a VHH that specifically binds to the D1 domain of hCD45 wherein the VHH comprises the polypeptide sequence of SEQ ID NO:2.

In some embodiments, the CD45 binding molecule comprises a sdAb that specifically binds to the D1/D2 domain of hCD45. In some embodiments, the sdAb that specifically binds to the D1/D2 domain of hCD45 is a polypeptide having at least 80%, alternatively at least 85%, alternatively at least 90%, alternatively at least 95% identity, alternatively at least 98%, alternatively at least 99% identity (or being identical except for 1, 2, 3, or 4 amino acids that optionally are conserved substitutions) to a polypeptide sequence of SEQ ID NO: 34 or SEQ ID NO: 38. In some embodiments, the CD45 binding molecule comprises a VHH that specifically binds to the D1/D2 domain of hCD45 wherein the VHH comprises the polypeptide sequence of sequence of SEQ ID NO: 34 or SEQ ID NO: 38.

In some embodiments, the CD45 binding molecule comprises a sdAb that specifically binds to the D2 domain of hCD45. In some embodiments, the sdAb that specifically binds to the D2 domain of hCD45 is a polypeptide having at least 80%, alternatively at least 85%, alternatively at least 90%, alternatively at least 95% identity, alternatively at least 98%, alternatively at least 99% identity (or being identical except for 1, 2, 3, or 4 amino acids that optionally are conserved substitutions) to a polypeptide sequence selected from the group consisting of SEQ ID NO: 78 and SEQ ID NO: 82. In some embodiments, the CD45 binding molecule comprises a VHH that specifically binds to the D2 domain of hCD45 wherein the VHH comprises the polypeptide sequence selected from the group consisting of SEQ ID NO: 78 and SEQ ID NO: 82.

In some embodiments, the CD45 binding molecule comprises a sdAb that specifically binds to the D3 domain of hCD45. In some embodiments, the sdAb that specifically binds to the D3 domain of hCD45 is a polypeptide having at least 80%, alternatively at least 85%, alternatively at least 90%, alternatively at least 95% identity, alternatively at least 98%, alternatively at least 99% identity (or being identical except for 1, 2, 3, or 4 amino acids that optionally are conserved substitutions) to a polypeptide sequence selected from the group consisting of SEQ ID NO: 94 and SEQ ID NO: 106. In some embodiments, the CD45 binding molecule comprises a VHH that specifically binds to the D3 domain of hCD45 wherein the VHH comprises the polypeptide sequence selected from the group consisting of SEQ ID NO: 94 and SEQ ID NO: 106.

In some embodiments, the CD45 binding molecule comprises a sdAb that specifically binds to the D4 domain of hCD45. In some embodiments, the sdAb that specifically binds to the D4 domain of hCD45 is a polypeptide having at least 80%, alternatively at least 85%, alternatively at least 90%, alternatively at least 95% identity, alternatively at least 98%, alternatively at least 99% identity (or being identical except for 1, 2, 3, or 4 amino acids that optionally are conserved substitutions) to a polypeptide sequence selected from the group consisting of SEQ ID NO: 143, SEQ ID NO: 126 and SEQ ID NO: 138. In some embodiments, the CD45 binding molecule comprises a VHH that specifically binds to the D5 domain of hCD45RO wherein the VHH comprises the polypeptide sequence selected from the group consisting of SEQ ID NO: 143, SEQ ID NO: 126 and SEQ ID NO: 138.

In another aspect, the CD45 binding molecule comprises a first domain comprising a polypeptide that specifically binds to an isoform of CD45 in stable association with a second domain comprising a labeling agent, an imaging agent and/or a therapeutic agent.

In another aspect, the CD45 binding molecule is a multivalent binding molecule comprising a first domain comprising a polypeptide that specifically binds to an isoform of CD45 in stable association with a second domain that specifically binds to the extracellular domain of a cell surface molecule. In some embodiments the CD45 binding molecule is a RIPR molecule. In some embodiments the RIPR molecule comprises a CD45 sdAb of the present disclosure operably linked to a molecule that binds a receptor selected from the group consisting of checkpoint molecules, growth hormone receptors.

The present disclosure provides CD45 binding molecules comprising a domain that selectively binds to the extracellular domain of the human CD45RO isoform and methods of use thereof in the treatment or prevention of diseases, disorders or conditions associated with dysregulated T cell and/or B cell activity in a subject.

The present disclosure provides hCD45 binding molecules comprising a domain that selectively binds to the extracellular domain of the human CD45RO isoform and methods of use thereof in the treatment or prevention of neoplastic diseases, disorders or conditions in a subject.

The present disclosure provides hCD45 binding molecules comprising a domain that selectively binds to the extracellular domain of the human CD45RO isoform and methods of use thereof in the treatment or prevention of autoimmune diseases, disorders or conditions in a subject.

The present disclosure provides hCD45 binding molecules comprising a domain that selectively binds to the extracellular domain of the human CD45RO isoform and methods of use thereof in the isolation, depletion or enrichment of CD45RO+ cells a biological sample.

The present disclosure provides hCD45 binding molecules comprising a domain that selectively binds to the extracellular domain of the human CD45RO isoform and methods of use thereof in the treatment of HCMV infection.

The present disclosure provides hCD45 binding molecules comprising a domain that selectively binds to the extracellular domain of the human CD45RO isoform and methods of use thereof in the treatment of adenovirus infection.

In some embodiments, the present disclosure provides a sdAb, said sdAb comprising a triad of CDRs selected from the group consisting of:
SEQ ID NO: 3, SEQ ID NO: 4, and SEQ ID NO: 5;
SEQ ID NO: 7, SEQ ID NO: 8, and SEQ ID NO: 9;
SEQ ID NO: 11, SEQ ID NO: 12, and SEQ ID NO: 13;
SEQ ID NO: 15, SEQ ID NO: 16, and SEQ ID NO: 17;
SEQ ID NO: 19, SEQ ID NO: 20, and SEQ ID NO: 21;
SEQ ID NO: 23, SEQ ID NO: 24, and SEQ ID NO: 25;
SEQ ID NO: 27, SEQ ID NO: 28, and SEQ ID NO: 29;
SEQ ID NO: 31, SEQ ID NO: 32, and SEQ ID NO: 33;
SEQ ID NO: 35, SEQ ID NO: 36, and SEQ ID NO: 37;
SEQ ID NO: 39, SEQ ID NO: 40, and SEQ ID NO: 41;
SEQ ID NO: 43, SEQ ID NO: 44, and SEQ ID NO: 45;
SEQ ID NO: 47, SEQ ID NO: 48, and SEQ ID NO: 49;
SEQ ID NO: 51, SEQ ID NO: 52, and SEQ ID NO: 53;
SEQ ID NO: 55, SEQ ID NO: 56, and SEQ ID NO: 57;
SEQ ID NO: 59, SEQ ID NO: 60, and SEQ ID NO: 61;
SEQ ID NO: 63, SEQ ID NO: 64, and SEQ ID NO: 65;
SEQ ID NO: 67, SEQ ID NO: 68, and SEQ ID NO: 69;
SEQ ID NO: 71, SEQ ID NO: 72, and SEQ ID NO: 73;
SEQ ID NO: 75, SEQ ID NO: 76, and SEQ ID NO: 77;
SEQ ID NO: 79, SEQ ID NO: 80, and SEQ ID NO: 81;
SEQ ID NO: 83, SEQ ID NO: 84, and SEQ ID NO: 85;
SEQ ID NO: 87, SEQ ID NO: 88, and SEQ ID NO: 89;
SEQ ID NO: 91, SEQ ID NO: 92, and SEQ ID NO: 93;
SEQ ID NO: 95, SEQ ID NO: 96, and SEQ ID NO: 97;
SEQ ID NO: 99, SEQ ID NO: 100, and SEQ ID NO: 101;
SEQ ID NO: 103, SEQ ID NO: 104, and SEQ ID NO: 105;
SEQ ID NO: 107, SEQ ID NO: 108, and SEQ ID NO: 109;
SEQ ID NO: 111, SEQ ID NO: 112, and SEQ ID NO: 113;
SEQ ID NO: 115, SEQ ID NO: 116, and SEQ ID NO: 117;
SEQ ID NO: 119, SEQ ID NO: 120, and SEQ ID NO: 121;
SEQ ID NO: 123, SEQ ID NO: 124, and SEQ ID NO: 125;
SEQ ID NO: 127, SEQ ID NO: 128, and SEQ ID NO: 129;
SEQ ID NO: 131, SEQ ID NO: 132, and SEQ ID NO: 133;
SEQ ID NO: 135, SEQ ID NO: 136, and SEQ ID NO: 137;
SEQ ID NO: 139, SEQ ID NO: 140, and SEQ ID NO: 141;
SEQ ID NO: 143, SEQ ID NO: 144, and SEQ ID NO: 145;
SEQ ID NO: 147, SEQ ID NO: 148, and SEQ ID NO: 149;
SEQ ID NO: 151, SEQ ID NO: 152, and SEQ ID NO: 153;
SEQ ID NO: 155, SEQ ID NO: 156, and SEQ ID NO: 157;
SEQ ID NO: 159, SEQ ID NO: 160, and SEQ ID NO: 161;
SEQ ID NO: 163, SEQ ID NO: 164, and SEQ ID NO: 165;
SEQ ID NO: 167, SEQ ID NO: 168, and SEQ ID NO: 169;
SEQ ID NO: 171, SEQ ID NO: 172, and SEQ ID NO: 173;
SEQ ID NO: 175, SEQ ID NO: 176, and SEQ ID NO: 177;
SEQ ID NO: 179, SEQ ID NO: 180, and SEQ ID NO: 181;
SEQ ID NO: 183, SEQ ID NO: 184, and SEQ ID NO: 185;
SEQ ID NO: 187, SEQ ID NO: 188, and SEQ ID NO: 189;
SEQ ID NO: 191, SEQ ID NO: 192, and SEQ ID NO: 193;
SEQ ID NO: 195, SEQ ID NO: 196, and SEQ ID NO: 197;
SEQ ID NO:199, SEQ ID NO:200, and SEQ ID NO:201;
SEQ ID NO:203, SEQ ID NO:204, and SEQ ID NO:205;
SEQ ID NO:207, SEQ ID NO:208, and SEQ ID NO:209;
SEQ ID NO:211, SEQ ID NO:212, and SEQ ID NO:213;
SEQ ID NO:215, SEQ ID NO:216, and SEQ ID NO:217;
SEQ ID NO:219, SEQ ID NO:220, and SEQ ID NO:221;
SEQ ID NO:223, SEQ ID NO:224, and SEQ ID NO:225;
SEQ ID NO:227, SEQ ID NO:228, and SEQ ID NO:229;
SEQ ID NO:231, SEQ ID NO:232, and SEQ ID NO:233;
SEQ ID NO:235, SEQ ID NO:236, and SEQ ID NO:237;
SEQ ID NO:239, SEQ ID NO:240, and SEQ ID NO:241;
SEQ ID NO:243, SEQ ID NO:244, and SEQ ID NO:245;
SEQ ID NO:247, SEQ ID NO:248, and SEQ ID NO:249;
SEQ ID NO:251, SEQ ID NO:252, and SEQ ID NO:253;
SEQ ID NO:255, SEQ ID NO:256, and SEQ ID NO:257;
SEQ ID NO:259, SEQ ID NO:260, and SEQ ID NO:261;
SEQ ID NO:263, SEQ ID NO:264, and SEQ ID NO:265;
SEQ ID NO:267, SEQ ID NO:268, and SEQ ID NO:269;
SEQ ID NO:271, SEQ ID NO:272, and SEQ ID NO:273;
SEQ ID NO:275, SEQ ID NO:276, and SEQ ID NO:277;
SEQ ID NO:279, SEQ ID NO:280, and SEQ ID NO:281;
SEQ ID NO:283, SEQ ID NO:284, and SEQ ID NO:285;
SEQ ID NO:287, SEQ ID NO:288, and SEQ ID NO:289;
SEQ ID NO:291, SEQ ID NO:292, and SEQ ID NO:293;
SEQ ID NO:295, SEQ ID NO:296, and SEQ ID NO:297;
SEQ ID NO:299, SEQ ID NO:300, and SEQ ID NO:301;
SEQ ID NO:303, SEQ ID NO:304, and SEQ ID NO:305;
SEQ ID NO:307, SEQ ID NO:308, and SEQ ID NO:309;
SEQ ID NO:311, SEQ ID NO:312, and SEQ ID NO:313;
SEQ ID NO:315, SEQ ID NO:316, and SEQ ID NO:317;
SEQ ID NO:319, SEQ ID NO:320, and SEQ ID NO:321;
SEQ ID NO:323, SEQ ID NO:324, and SEQ ID NO:325;
SEQ ID NO:327, SEQ ID NO:328, and SEQ ID NO:329;
SEQ ID NO:331, SEQ ID NO:332, and SEQ ID NO:333;
SEQ ID NO:335, SEQ ID NO:336, and SEQ ID NO:337;
SEQ ID NO:339, SEQ ID NO:340, and SEQ ID NO:341;
SEQ ID NO:343, SEQ ID NO:344, and SEQ ID NO:345;
SEQ ID NO:347, SEQ ID NO:348, and SEQ ID NO:349;
SEQ ID NO:351, SEQ ID NO:352, and SEQ ID NO:353;
SEQ ID NO:355, SEQ ID NO:356, and SEQ ID NO:357;
SEQ ID NO:359, SEQ ID NO:360, and SEQ ID NO:361;
SEQ ID NO:363, SEQ ID NO:364, and SEQ ID NO:365;
SEQ ID NO:367, SEQ ID NO:368, and SEQ ID NO:369;
SEQ ID NO:371, SEQ ID NO:372, and SEQ ID NO:373;
SEQ ID NO:375, SEQ ID NO:376, and SEQ ID NO:377;
SEQ ID NO:379, SEQ ID NO:380, and SEQ ID NO:381;
SEQ ID NO:383, SEQ ID NO:384, and SEQ ID NO:385;
SEQ ID NO:387, SEQ ID NO:388, and SEQ ID NO:389;
SEQ ID NO:391, SEQ ID NO:392, and SEQ ID NO:393;
SEQ ID NO:395, SEQ ID NO:396, and SEQ ID NO:397;
SEQ ID NO:399, SEQ ID NO:400, and SEQ ID NO:401;
SEQ ID NO:403, SEQ ID NO:404, and SEQ ID NO:405;
SEQ ID NO:407, SEQ ID NO:408, and SEQ ID NO:409;
SEQ ID NO:411, SEQ ID NO:412, and SEQ ID NO:413;
SEQ ID NO:415, SEQ ID NO:416, and SEQ ID NO:417;
SEQ ID NO:419, SEQ ID NO:420, and SEQ ID NO:421;
SEQ ID NO:423, SEQ ID NO:424, and SEQ ID NO:425;
SEQ ID NO:427, SEQ ID NO:428, and SEQ ID NO:429;
SEQ ID NO:431, SEQ ID NO:432, and SEQ ID NO:433;
SEQ ID NO:435, SEQ ID NO:436, and SEQ ID NO:437;
SEQ ID NO:439, SEQ ID NO:440, and SEQ ID NO:441;
SEQ ID NO:443, SEQ ID NO:444, and SEQ ID NO:445;
SEQ ID NO:447, SEQ ID NO:448, and SEQ ID NO:449;
SEQ ID NO:451, SEQ ID NO:452, and SEQ ID NO:453;
SEQ ID NO:455, SEQ ID NO:456, and SEQ ID NO:457;
SEQ ID NO:459, SEQ ID NO:460, and SEQ ID NO:461;
SEQ ID NO:463, SEQ ID NO:464, and SEQ ID NO:465;
SEQ ID NO:467, SEQ ID NO:468, and SEQ ID NO:469;
SEQ ID NO:471, SEQ ID NO:472, and SEQ ID NO:473;
SEQ ID NO:475, SEQ ID NO:476, and SEQ ID NO:477;
SEQ ID NO:479, SEQ ID NO:480, and SEQ ID NO:481;
SEQ ID NO:483, SEQ ID NO:484, and SEQ ID NO:485;
SEQ ID NO:487, SEQ ID NO:488, and SEQ ID NO:489;
SEQ ID NO:491, SEQ ID NO:492, and SEQ ID NO:493;
SEQ ID NO:495, SEQ ID NO:496, and SEQ ID NO:497;
SEQ ID NO:499, SEQ ID NO:500, and SEQ ID NO:501;
SEQ ID NO:503, SEQ ID NO:504, and SEQ ID NO:505;
SEQ ID NO:507, SEQ ID NO:508, and SEQ ID NO:509;
SEQ ID NO:511, SEQ ID NO:512, and SEQ ID NO:513;
SEQ ID NO:515, SEQ ID NO:516, and SEQ ID NO:517;
SEQ ID NO:519, SEQ ID NO:520, and SEQ ID NO:521; and
SEQ ID NO:523, SEQ ID NO:524, and SEQ ID NO:525.

In some embodiments, the present disclosure provides a sdAb, said sdAb comprising a triad of CDRs selected from the group consisting of:
SEQ ID NO: 3, SEQ ID NO: 4, and SEQ ID NO: 5;
SEQ ID NO: 35, SEQ ID NO: 36, and SEQ ID NO: 37;
SEQ ID NO: 39, SEQ ID NO: 40, and SEQ ID NO: 41;
SEQ ID NO: 79, SEQ ID NO: 80, and SEQ ID NO: 81;
SEQ ID NO: 83, SEQ ID NO: 84, and SEQ ID NO: 85;
SEQ ID NO: 95, SEQ ID NO: 96, and SEQ ID NO: 97;
SEQ ID NO: 127, SEQ ID NO: 128, and SEQ ID NO: 129;
SEQ ID NO: 135, SEQ ID NO: 136, and SEQ ID NO: 137; and
SEQ ID NO: 139, SEQ ID NO: 140, and SEQ ID NO: 141.

The disclosure further provides a polypeptide having at least 80%, alternatively at least 85%, alternatively at least 90%, alternatively at least 95% identity, alternatively at least 98%, alternatively at least 99% identity (or being identical except for 1, 2, 3, or 4 amino acids that optionally are conserved substitutions) to a polypeptide sequence selected from the group consisting of: SEQ ID NO: 2, SEQ ID NO: 34, SEQ ID NO: 38, SEQ ID NO: 78, SEQ ID NO: 82, SEQ ID NO: 94, SEQ ID NO: 106, SEQ ID NO: 134, SEQ ID NO: 126, and SEQ ID NO: 138. The invention further provides a polypeptide having at least 80%, alternatively at least 85%, alternatively at least 90%, alternatively at least 95%, alternatively at least 98%, alternatively at least 99% identity (or being identical except for 1, 2, 3, or 4 amino acids that optionally are conserved substitutions) to a polypeptide sequence selected from the group consisting of: SEQ ID NO: 2, SEQ ID NO: 6, SEQ ID NO: 10, SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, SEQ ID NO: 90, SEQ ID NO: 94, SEQ ID NO: 98, SEQ ID NO: 102, SEQ ID NO: 106, SEQ ID NO: 110, SEQ ID NO: 114, SEQ ID NO: 118, SEQ ID NO: 122, SEQ ID NO: 126, SEQ ID NO: 130, SEQ ID NO: 134, SEQ ID NO: 138, SEQ ID NO: 142, SEQ ID NO: 146, SEQ ID NO: 150, SEQ ID NO: 154, SEQ ID NO: 158, SEQ ID NO: 162, SEQ ID NO: 166, SEQ ID NO: 170, SEQ ID NO: 174, SEQ ID NO: 178, SEQ ID NO: 182, SEQ ID NO: 186, SEQ ID NO: 190, SEQ ID NO: 194, SEQ ID NO: 198, SEQ ID NO:204, SEQ ID NO: 208, SEQ ID NO: 212, SEQ ID NO: 216, SEQ ID NO: 220, SEQ ID NO: 224, SEQ ID NO:228, SEQ ID NO: 232, SEQ ID NO: 236, SEQ ID NO: 240, SEQ ID NO: 244, SEQ ID NO: 248, SEQ ID NO: 252, SEQ ID NO: 256, SEQ ID NO: 260, SEQ ID NO: 264, SEQ ID NO: 268, SEQ ID NO: 272, SEQ ID NO: 276, SEQ ID NO: 280, SEQ ID NO: 284, SEQ ID NO: 288, SEQ ID NO: 292, SEQ ID NO: 296, SEQ ID NO: 300, SEQ ID NO: 304, SEQ ID NO: 308, SEQ ID NO: 312, SEQ ID NO: 316, SEQ ID NO:320, SEQ ID NO: 324, SEQ ID NO: 328, SEQ ID NO: 332, SEQ ID NO: 336, SEQ ID NO: 340, SEQ ID NO: 344, SEQ ID NO: 348, SEQ ID NO: 352, SEQ ID NO: 356, SEQ ID NO: 360, SEQ ID NO: 364, SEQ ID NO: 368, SEQ ID NO: 372, SEQ ID NO: 376, SEQ ID NO: 380, SEQ ID NO: 384, SEQ ID NO: 388, SEQ ID NO: 392, SEQ ID NO: 396, SEQ ID NO: 400, SEQ ID NO: 404, SEQ ID NO: 408, SEQ ID NO: 412, SEQ ID NO: 416, SEQ ID NO: 420, SEQ ID NO: 424, SEQ ID NO: 428, SEQ ID NO: 432, SEQ ID NO: 436, SEQ ID NO: 440, SEQ ID NO: 444, SEQ ID NO: 448, SEQ ID NO: 452, SEQ ID NO: 456, SEQ ID NO: 460, SEQ ID NO: 464, SEQ ID NO: 470, SEQ ID NO: 474, SEQ ID NO: 478, SEQ ID NO: 482, SEQ ID NO: 486, SEQ ID NO: 490, SEQ ID NO: 494, SEQ ID NO: 498, SEQ ID NO: 502, SEQ ID NO: 506, SEQ ID NO: 510, SEQ ID NO: 514, SEQ ID NO: 518, and SEQ ID NO: 522.

The disclosure further provides a polypeptide selected from the group consisting of: SEQ ID NO: 2, SEQ ID NO: 34, SEQ ID NO: 38, SEQ ID NO: 78, SEQ ID NO: 82, SEQ ID NO: 94, SEQ ID NO: 106, SEQ ID NO: 134, SEQ ID NO: 126, and SEQ ID NO: 138.

The disclosure further provides methods of chemical or recombinant processes for the preparation of the CD45 binding molecules of the present disclosure.

The disclosure further provides nucleic acids encoding the CD45 binding molecules of the present disclosure or the polypeptide domains of the CD45 binding molecules of the present disclosure.

The disclosure further provides recombinant viral and non-viral vectors comprising a nucleic acid the CD45 binding molecules of the present disclosure or the polypeptide domains of the CD45 binding molecules of the present disclosure.

The disclosure further provides host cells comprising recombinant viral and non-viral vectors comprising a nucleic acid the CD45 binding molecules of the present disclosure or the polypeptide domains of the CD45 binding molecules of the present disclosure.

The disclosure further provides host cells comprising recombinant viral and non-viral vectors comprising a nucleic acid the CD45 binding molecules of the present disclosure or the polypeptide domains of the CD45 binding molecules of the present disclosure.

The disclosure further provides pharmaceutical formulations comprising the recombinant viral and non-viral vectors comprising a nucleic acid the CD45 binding molecules of the present disclosure and methods of use thereof in the treatment or prevention of diseases, disorders or conditions in a mammalian subject.

The disclosure further kits comprising the CD45 binding molecules of the present disclosure.

### BRIEF DESCRIPTION OF THE DRAWINGS

FIG. 1 provides a schematic illustration of some of the major features of the various hCD45 isoforms with respect to the cell membrane illustrating the A, B, and C domains of the mucin like domain in the various isoforms, the 30 amino acid serine/threonine rich domain (ST) and the four fibronectin like domains (D1-D4) that comprise the hCD45RO ECD domain, the transmembrane domain (TM) and the two intracellular phosphatase domains (P1 and P2) of CD45.

FIG. 2 provides a graphical depiction of the CD45 domains used for yeast display domain mapping by fusion to Aga2 protein as more fully described in Example 3

FIG. 3 provides the data from the domain mapping and affinity measurement of the VHE, GAP8.3, 4B2 AND 9.4 anti-human CD45RO antibodies as more fully described in Example 3 herein illustrating that these antibodies bind to the D1 and D2 domains of the CD45RO ECD.

FIG. 4 provides a clonotype analysis of heavy chain antibodies obtained from an immunization of a llama with the polypeptide having the amino acid sequence of the extracellular domain of hCD45RO as more fully described in Example 1.

FIG. 5 provides a graphical depiction of the variation in length of the CDR3 domains obtained from the llama immunization of the as more fully described in example 1

FIG. 6 provides schematic illustrations various arrangements of polypeptide components that may be employed in context of a RIPR CD45 binding molecule of the present disclosure. Panel A provides a schematic representation of an illustrative embodiment of two polypeptide chain RIPR CD45 binding molecule of the present disclosure the two chains associated using Fc domains associated with the knob/hole format, a first polypeptide construct arranged, amino to carboxyl: a CD45RO single domain antibody (CD45RO sdAb), a first polypeptide linker (L1), a scFv antibody that specifically binds to the ECD of human PD1 (α-PD1 scFv), a second polypeptide linker (L2), and a first Fc monomer comprising a knob domain (Fc knob), a second polypeptide comprising an Fc monomer comprising a hole domain (Fc hole). Panel B provides a schematic representation of another illustrative embodiment of single polypeptide chain RIPR CD45 binding molecule of the present disclosure, the polypeptide construct arranged, amino to carboxyl: a CD45RO single domain antibody (CD45RO sdAb), a polypeptide linker (L), a scFv antibody that specifically binds to the ECD of human PD1 (α-PD1 scFv), and a chelating peptide (CP) near the carboxy terminus. Panel C provides a schematic representation of an alternative two polypeptide chain embodiment illustrative embodiment of two polypeptide chain RIPR CD45 binding molecule of the present disclosure the two chains associated using Fc domains associated with the knob/hole format, the first polypeptide construct arranged, amino to carboxyl: a CD45RO single domain antibody (CD45RO sdAb), a first polypeptide linker (L1), a first Fc monomer comprising a knob domain (Fc knob), a second polypeptide Fc monomer comprising a hole domain (Fc hole), a second polypeptide linker (L2), and an scFv antibody that specifically binds to the ECD of human PD1 (α-PD1 scFv), the construct further containing two interchain disulfide bonds (—S—S—).

FIG. 7 provides schematic illustrations various arrangements of polypeptide component domains that may be employed in context of a RIPR CD45 binding molecule of the present disclosure. Panel A provides a schematic representation of an illustrative embodiment of single polypeptide chain RIPR CD45 binding molecule of the present disclosure, the polypeptide construct arranged, amino to carboxyl: a CD45RO single domain antibody (CD45RO sdAb), a polypeptide linker (L), an scFv antibody that specifically binds to the ECD of tumor associated antigen comprising an intracellular domain that is regulated by phosphorylation (α-TAA scFv), and a chelating peptide (CP) near the carboxyl terminus. Panel B provides a schematic representation of an illustrative embodiment of two polypeptide chain RIPR CD45 binding molecule of the present disclosure RIPR CD45 binding molecule domains the first polypeptide construct arranged, amino to carboxyl: a first polypeptide linker (L1), a first Fc monomer comprising a knob domain (Fc knob), a second polypeptide chain arranged amino to carboxyl of an Fc monomer comprising a hole domain (Fc hole), a second polypeptide linker (L2), an scFv antibody that specifically binds to the ECD of tumor associated antigen comprising an intracellular domain that is regulated by phosphorylation (α-TAA scFv), the construct further containing two interchain disulfide bonds (—S—S—).

FIG. 8 provides a schematic illustrations of a RIPR CD45 binding molecule of the present disclosure wherein an antibody that specifically binds to the extracellular domain of human PD1 (α-PD-1 Igg) has been modified to display one or more a CD45RO single domain antibodies [CD45RO sdAb]_{a-f} at the amino or carboxy termini of the light chains and the amino termini of the heavy chains of the IgG, the wherein each of a-f is independently selected from 0 or 1, provided that at least one of a-f is equal to 1, such that the illustration provides a formula depicting an IgG antibody that possesses from 1 to 6 CD45RO sdAbs which may be present or absent from any of the particular termini of the illustration.

FIG. 9 provides schematic illustrations of multivalent CD45RO binding molecules of the present disclosure comprising multiple single domain antibodies (sdAbs). Panel A provides an illustrative embodiment of a linear single chain trivalent CD45 binding molecule construct comprising three single domain antibodies (sdAb) conjugated via two intervening linkers, L1 and L2, with the understanding that at least one of the sdAbs is a CD45RO sdAb. Panel B provides a generic formula for linear single chain multivalent polymeric construct comprising n single domain antibodies each separated by an intervening linker Lₙ with the understanding that at least one of the sdAbs is a CD45RO sdAb. In some embodiments, one of the non-CD45RO sdAbs could be an sdAb that specifically binds to the ECD of receptor that is modulated by intracellular phosphorylation such that the multivalent linear molecule is a RIPR molecule.

FIG. 10 provides schematic illustrations of circularized multivalent CD45RO binding molecules of the present disclosure comprising multiple single domain antibodies (sdAbs). Panel A provides an illustrative embodiment of a bivalent circularized CD45 binding molecule construct having a first sdAb that is a CD45RO sdAb and a second single domain antibodies (sdAb), the first and second sdAbs conjugated two intervening linkers, L1 and L2. Panel B provides an illustrative embodiment of a trivalent circularized CD45 binding molecule construct having a one sdAb that is a CD45RO sdAb and two additional single domain antibodies (sdAb), the sdAbs conjugated via three intervening linkers, L1, L2 and L3. Panel C provides a generic formula for linear single chain multivalent polymeric construct each monomer comprising n single domain antibodies (sdAbn) each separated by an intervening linker Lₙ, the polymeric construct circularized via a linker linker Lₙ₊₁. In some embodiments, one of the non-CD45RO sdAbs of the circularized multivalent constructs illustrated could be an sdAb that specifically binds to the ECD of receptor that is modulated by intracellular phosphorylation such that the multivalent linear molecule is a RIPR molecule.

### DETAILED DESCRIPTION OF THE INVENTION

### A. Introduction

In order for the present disclosure to be more readily understood, certain terms and phrases are defined below as well as throughout the specification. The definitions provided herein are non-limiting and should be read in view of the knowledge of one of skill in the art would know.

Before the present methods and compositions are described, it is to be understood that this disclosure is not limited to particular method or composition described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

It will be appreciated that throughout this disclosure reference is made to amino acids according to the single letter or three letter codes. For the reader's convenience, the single and three letter amino acid codes are provided in Table 1 below:

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

### B. Definitions

Unless otherwise indicated, the following terms are intended to have the meaning set forth below. Other terms are defined elsewhere throughout the specification.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand.

Activity: As used herein, the term “activity” is used with respect to a molecule to describe a property of the molecule with respect to a test system (e.g. an assay) or biological or chemical property (e.g. the degree of binding of the molecule to another molecule) or of a physical property of a material or cell (e.g. modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT5 phosphorylation]/[mg protein], [proliferation]/[mg protein], plaque forming units (pfu), etc. As used herein, the term proliferative activity refers to an activity that promotes cell proliferation and replication, including dysregulated cell division such as that observed in neoplastic diseases, inflammatory diseases, fibrosis, dysplasia, cell transformation, metastasis, and angiogenesis.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g. an a CD45 binding molecule or an engineered cell expressing an CD45 binding molecule, a chemotherapeutic agent, an antibody, or a pharmaceutical formulation comprising one or more of the foregoing). Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, intracranial injection, intratumoral injection, transdermal, transmucosal, iontophoretic delivery, intralymphatic injection, intragastric infusion, intraprostatic injection, intravesical infusion (e.g., bladder), inhalation (e.g. respiratory inhalers including dry-powder inhalers), intraocular injection, intraabdominal injection, intralesional injection, intraovarian injection, intracerebral infusion or injection, intracerebroventricular injection (ICVI), and the like. The term “administration” includes contact of an agent to the cell, tissue or organ as well as the contact of an agent to a fluid, where the fluid is in contact with the cell, tissue or organ.

Affinity: As used herein the term “affinity” refers to the degree of specific binding of a first molecule (e.g. a ligand) to a second molecule (e.g. a receptor) and is measured by the binding kinetics expressed as K_{d}, a ratio of the dissociation constant between the molecule and the its target (K_{off}) and the association constant between the molecule and its target (Kₒₙ).

Agonist: As used herein, the term “agonist” refers a first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways, that may result in cell proliferation or pathways that result in cell cycle arrest or cell death such as by apoptosis. In some embodiments, an agonist is an agent that binds to a receptor and alters the receptor state resulting in a biological response that mimics the effect of the endogenous ligand of the receptor. The term “agonist” includes partial agonists, full agonists and superagonists. An agonist may be described as a “full agonist” when such agonist which leads to a substantially full biological response (i.e. the response associated with the naturally occurring ligand/receptor binding interaction) induced by receptor under study, or a partial agonist. A “superagonist” is a type of agonist that is capable of producing a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay. It should be noted that the biological effects associated with the full agonist may differ in degree and/or in kind from those biological effects of partial or superagonists. In contrast to agonists, antagonists may specifically bind to a receptor but do not result the signal cascade typically initiated by the receptor and may to modify the actions of an agonist at that receptor. Inverse agonists are agents that produce a pharmacological response that is opposite in direction to that of an agonist.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway including an immune checkpoint pathway, or cell.

Antibody: As used herein, the term “antibody” refers collectively to: (a) a glycosylated or non-glycosylated immunoglobulin that specifically binds to target molecule, and (b) immunoglobulin derivatives thereof, including but not limited to antibody fragments such as single domain antibodies, wherein the immunoglobulin derivative competes with the immunoglobulin from which it was derived for binding to the target molecule. The term antibody is not restricted to immunoglobulins derived from any particular species and includes murine, human, equine, camelids, antibodies of cartilaginous fishes including, but not limited to, sharks. The term “antibody” encompasses antibodies isolatable from natural sources or from animals following immunization with an antigen and as well as engineered antibodies including monoclonal antibodies, bispecific antibodies, tri-specific, chimeric antibodies, humanized antibodies, human antibodies, CDR-grafted, veneered, or deimmunized (e.g., to remove T-cell epitopes) antibodies, camelized (in the case of VHHs), or molecules comprising binding domains of antibodies (e.g. CDRs) in non-immunoglobulin scaffolds. The term “antibody” should not be construed as limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries. In one embodiment, an “antibody” is a mammalian immunoglobulin. In some embodiments, the antibody is a “full length antibody” comprising variable and constant domains providing binding and effector functions. The term “single domain antibody” (sdAb) as used herein refers an antibody fragment consisting of a monomeric variable antibody domain that is able to bind selectively to an antigen and compete for binding with the parent antibody from which it is derived. As used herein, the term “VHH” refers to a single domain antibody derived from camelid antibody typically obtained from immunization of camelids (including camels, llamas and alpacas (see, e.g. Hamers-Casterman, et al. (1993) Nature 363:446-448). VHHs are also referred to as heavy chain antibodies or Nanobodies® as Single domain antibodies may also be derived from non-mammalian sources such as VHHs obtained from IgNAR antibodies immunization of cartilaginous fishes including, but not limited to, sharks.

Biological Sample: As used herein, the term “biological sample” or “sample” refers to a sample obtained (or derived) from a subject. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), fluids of the eye (e.g., vitreous fluid, aqueous humor), lymph fluid, lymph node tissue, spleen tissue, bone marrow, and an immunoglobulin enriched fraction derived from one or more of these tissues.

CD45RO cell: The terms “CD45RO cell”, “CD45RO-expressing cell”, “CD45RO-positive cell” and “CD45RO+” cell are used interchangeably herein to refer to a cell which expresses and displays the CD45RO antigen on the extracellular surface of the cell membrane. Similarly, the terms “CD45RO-negative cell”, “CD45RO-cells” as are used interchangeably herein to describe cells which do not express or display CD45RO antigen on the cell surface.

CDR: As used herein, the term “CDR” or “complementarity determining region” is intended to mean the non-contiguous antigen combining sites found within the variable region of both heavy and light chain immunoglobulin polypeptides. CDRs have been described by Kabat et al., J. Biol. Chem. 252:6609-6616 (1977); Kabat, et al., U.S. Dept. of Health and Human Services publication entitled “Sequences of proteins of immunological interest” (1991) (also referred to herein as “Kabat 1991” or “Kabat”); by Chothia, et al. (1987) J. Mol. Biol. 196:901-917 (also referred to herein as “Chothia”); and MacCallum, et al. (1996) J. Mol. Biol. 262:732-745, where the definitions include overlapping or subsets of amino acid residues when compared against each other. Nevertheless, application of either definition to refer to a CDR of an antibody or grafted antibodies or variants thereof is intended to be within the scope of the term as defined and used herein. The term “Chothia Numbering” as used herein is recognized in the arts and refers to a system of numbering amino acid residues based on the location of the structural loop regions (Chothia et al. 1986, Science 233:755-758; Chothia & Lesk 1987, JMB 196:901-917; Chothia et al. 1992, JMB 227:799-817). For purposes of the present disclosure, unless otherwise specifically identified, the positioning of CDRs2 and 3 in the variable region of an antibody follows Kabat numbering or simply, “Kabat.” The positioning of CDR1 in the variable region of an antibody follows a hybrid of Kabat and Chothia numbering schemes.

Clonotype: As used herein, a clonotype refers to a collection of binding molecules that originate from the same B-cell progenitor cell. The term “clonotype” is used to refer to a collection of antigen binding molecules that belong to the same germline family, have the same CDR3 lengths, and have 70% or greater homology in CDR3 sequence

Circulating Tumor Cell: As used herein the term “circulating tumor cell (CTC)” refers to tumor cells that have been shed from a tumor mass into the peripheral circulation.

Comparable: As used herein, the term “comparable” is used to describe the degree of difference in two measurements of an evaluable quantitative or qualitative parameter. For example, where a first measurement of an evaluable quantitative parameter and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 35%, alternatively by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Derived From: As used herein in the term “derived from”, in the context of an amino acid sequence is meant to indicate that the polypeptide or nucleic acid has a sequence that is based on that of a reference polypeptide or nucleic acid and is not meant to be limiting as to the source or method in which the protein or nucleic acid is made. By way of example, the term “derived from” includes homologs or variants of reference amino acid or DNA sequences.

Effective Concentration (EC): As used herein, the terms “effective concentration” or its abbreviation “EC” are used interchangeably to refer to the concentration of an agent in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological response observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results the maximal (100%) response of a measurable parameter in response to such agent. Similarly, the term EC₅₀ (which is commonly used in the field of pharmacodynamics) refers to the concentration of an agent sufficient to results in the half-maximal (50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect.

Enriched: As used herein in the term “enriched” refers to a sample that is non-naturally manipulated so that a species (e.g. a molecule or cell) of interest is present in: (a) a greater concentration (e.g., at least 3-fold greater, alternatively at least 5-fold greater, alternatively at least 10-fold greater, alternatively at least 50-fold greater, alternatively at least 100-fold greater, or alternatively at least 1000-fold greater) than the concentration of the species in the starting sample, such as a biological sample (e.g., a sample in which the molecule naturally occurs or in which it is present after administration); or (b) a concentration greater than the environment in which the molecule was made (e.g., a recombinantly modified bacterial or mammalian cell).

Extracellular Domain: As used herein the term “extracellular domain” or its abbreviation “ECD” refers to the portion of a cell surface protein (e.g., a cell surface receptor) which is external to of the plasma membrane of a cell. The cell surface protein may be transmembrane protein, a cell surface or membrane associated protein.

Identity: The term “identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux, et al., (1984) Nucleic Acids Res. 12:387), BLASTP, BLASTN, FASTA (Atschul, et al. (1990) J. Molecular Biol. 215:403-410). Algorithms that are suitable for determining percent sequence identity and sequence similarity are the BLAST and BLAST 2.0 algorithms, which are described in Altschul et al. (1990) J. Mol. Biol. 215: 403-410 and Altschul, et al. (1977) Nucleic Acids Res. 25: 3389-3402. Software for performing BLAST analyses is publicly available through the National Center for Biotechnology Information (NCBI) web site. The algorithm involves first identifying high scoring sequence pairs (HSPs) by identifying short words of length W of the query sequence, which either match or satisfy some positive-valued threshold score “T” when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (the reward score for a pair of matching residues; always >0) and “N” (the penalty score for mismatching residues; always <0). For amino acid sequences, a scoring matrix is used to calculate the cumulative score. Extension of the word hits in each direction are halted when: (a) the cumulative alignment score falls off by the quantity X from its maximum achieved value; the cumulative score goes to zero or below, due to the accumulation of one or more negative-scoring residue alignments; or (b) the end of either sequence is reached. The BLAST algorithm parameters “W”, “T”, and “X” determine the sensitivity and speed of the alignment. The BLASTN program (for nucleotide sequences) functions similarly but uses as defaults a word size (“W”) of 28, an expectation (“E”) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, (1989) PNAS(USA) 89: 10915-10919).

In An Amount Sufficient Amount to Effect a Response: As used herein the phrase “in an amount sufficient to cause a response” is used in reference to the amount of a test agent sufficient to provide a detectable change in the level of an indicator measured before (e.g., a baseline level) and after the application of a test agent to a test system. In some embodiments, the test system is a cell, tissue or organism. In some embodiments, the test system is an in vitro test system such as a fluorescent assay. In some embodiments, the test system is an in vivo system which involves the measurement of a change in the level a parameter of a cell, tissue, or organism reflective of a biological function before and after the application of the test agent to the cell, tissue, or organism. In some embodiments, the indicator is reflective of biological function or state of development of a cell evaluated in an assay in response to the administration of a quantity of the test agent. In some embodiments, the test system involves the measurement of a change in the level an indicator of a cell, tissue, or organism reflective of a biological condition before and after the application of one or more test agents to the cell, tissue, or organism. The term “in an amount sufficient to effect a response” may be sufficient to be a therapeutically effective amount but may also be more or less than a therapeutically effective amount.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

In Need of Prevention: As used herein the term “in need of prevention” refers to a judgment made by a physician or other caregiver with respect to a subject that the subject requires or will potentially benefit from preventative care. This judgment is made based upon a variety of factors that are in the realm of a physician's or caregiver's expertise.

Inhibitor: As used herein the term “inhibitor” refers to a molecule that decreases, blocks, prevents, delays activation of, inactivates, desensitizes, or down-regulates, e.g., a gene, protein, ligand, receptor, or cell. An inhibitor can also be defined as a molecule that reduces, blocks, or inactivates a constitutive activity of a cell or organism.

Intracellular Domain: As used herein the term “intracellular domain” or its abbreviation “ICD” refers to the portion of a cell surface protein (e.g. a cell surface receptor) which is inside of the plasma membrane of a cell. The ICD may include the entire cytoplasmic portion of a transmembrane protein or membrane associated protein, or intracellular protein.

Isolated: As used herein the term “isolated” is used in reference to a polypeptide of interest that, if naturally occurring, is in an environment different from that in which it can naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was synthesized, for example isolated from a recombinant cell culture comprising cells engineered to express the polypeptide or by a solution resulting from solid phase synthetic means.

Kabat Numbering: The term “Kabat numbering” as used herein is recognized in the art and refers to a system of numbering amino acid residues which are more variable than other amino acid residues (e.g., hypervariable) in the heavy and light chain regions of immunoglobulins (Kabat, et al., (1971) Ann. NY Acad. Sci. 190:382-93; Kabat, et al., (1991) Sequences of Proteins of Immunological Interest, Fifth Edition, U.S. Department of Health and Human Services, NIH Publication No. 91-3242). For purposes of the present disclosure, the positioning of CDRs in the variable region of an antibody follows Kabat numbering or simply, “Kabat.”

Ligand: As used herein, the term “ligand” refers to a molecule that specifically binds a receptor and causes a change in the receptor so as to effect a change in the activity of the receptor or a response in cell that expresses that receptor. In one embodiment, the term “ligand” refers to a molecule or complex thereof that can act as an agonist or antagonist of a receptor. As used herein, the term “ligand” encompasses natural and synthetic ligands. “Ligand” also encompasses small molecules, peptide mimetics of cytokines and antibodies. The complex of a ligand and receptor is termed a “ligand-receptor complex.” A ligand may comprise one domain of a polyprotein or fusion protein (e.g., either domain of an antibody/ligand fusion protein).

Modulate: As used herein, the terms “modulate”, “modulation” and the like refer to the ability of a test agent to cause a response, either positive or negative or directly or indirectly, in a system, including a biological system, or biochemical pathway. The term modulator includes both agonists (including partial agonists, full agonists and superagonists) and antagonists.

Neoplastic Disease: As used herein, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign (2) pre-malignant (or “pre-cancerous”); and (3) malignant (or “cancerous”). The term “neoplastic disease” includes neoplastic-related diseases, disorders and conditions referring to conditions that are associated, directly or indirectly, with neoplastic disease, and includes, e.g., angiogenesis and precancerous conditions such as dysplasia or smoldering multiple myeloma. Examples of benign disorders arising from dysregulated cell replication include hypertrophic scars such as keloid scars.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

Numbered in accordance with hCD45: The term “numbered in accordance with hCD45” as used herein refers to the identification of a location of particular amino acid with reference to the position at which that amino acid normally occurs in the sequence of the canonical hCD45RABC isoform (UniProt Sequence P08575; https://www.uniprot.org/uniprot/P08575, SEQ ID NO:1). Amino acids 1-25 correspond to the signal sequence, amino acids 26-577 correspond to the extracellular domain (ECD) of the mature hCD45 protein, amino acids 578-598 correspond to the transmembrane domain, and amino acids 599-1306 correspond to the intracellular domain (ICD). For example, when in reference is made to an amino acid residue of hCD45 such as “hCD45 Ser194” would refer the serine residue at the one hundred ninety fourth position. A hCD45 molecule containing a substitution of an amino acid at this position with a tyrosine residue would be abbreviated “hCD45 Ser195Tyr”. The amino acid sequences of CD45 molecules and isoforms different species have different numbers and sequences of amino acids and when reference is made herein to a CD45 molecule or isoform derived from a particular species, designation of the species from which that CD45 isoform is derived is designated by preceding letter such “h” for human CD45 (“hCD45”), “m” for murine CD45 (“mCD45”), and the like.

Operably Linked: The term “operably linked” is used herein to refer to the relationship between molecules, typically polypeptides or nucleic acids, which are arranged in a construct such that each of the functions of the component molecules is retained although the operable linkage may result in the modulation of the activity, either positively or negatively, of the individual components of the construct. For example, the operable linkage of a polyethylene glycol (PEG) molecule to a wild-type protein may result in a construct where the biological activity of the protein is diminished relative to the to the wild-type molecule, however the two are nevertheless considered operably linked. When the term “operably linked” is applied to the relationship of multiple nucleic acid sequences encoding differing functions, the multiple nucleic acid sequences when combined into a single nucleic acid molecule that, for example, when introduced into a cell using recombinant technology, provides a nucleic acid which is capable of effecting the transcription and/or translation of a particular nucleic acid sequence in a cell. For example, the nucleic acid sequence encoding a signal sequence may be considered operably linked to DNA encoding a polypeptide if it results in the expression of a preprotein whereby the signal sequence facilitates the secretion of the polypeptide; a promoter or enhancer is considered operably linked to a coding sequence if it affects the transcription of the sequence; or a ribosome binding site is considered operably linked to a coding sequence if it is positioned so as to facilitate translation. Generally in the context of nucleic acid molecules, the term “operably linked” means that the nucleic acid sequences being linked are contiguous, and, in the case of a secretory leader or associated subdomains of a molecule, contiguous and in reading phase. However, certain genetic elements such as enhancers may function at a distance and need not be contiguous with respect to the sequence to which they provide their effect but nevertheless may be considered operably linked.

Parent Polypeptide: As used herein, the terms “parent polypeptide” or “parent protein” are used interchangeably to designate the source of a second polypeptide (e.g. a derivative or variant) which is modified with respect to a first “parent” polypeptide. In some instances, the parent polypeptide is a wild-type or naturally occurring form of a protein. In some instance, the parent polypeptide may be a modified form a naturally occurring protein that is further modified. The term “parent polypeptide” may refer to the polypeptide itself or compositions that comprise the parent polypeptide (e.g. glycosylated or PEGylated forms and/or fusion proteins comprising the parent polypeptide).

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example when both a full agonist and partial agonist are present, the partial agonist acts as a competitive antagonist by competing with the full agonist for the receptor binding resulting in net decrease in receptor activation relative to the contact of the receptor with the full agonist in the absence of the partial agonist. Partial agonists can be used to activate receptors to give a desired submaximal response in a subject when inadequate amounts of the endogenous ligand are present, or they can reduce the overstimulation of receptors when excess amounts of the endogenous ligand are present. The maximum response (Eₘₐₓ) produced by a partial agonist is called its intrinsic activity and may be expressed on a percentage scale where a full agonist produced a 100% response. An partial agonist may have greater than 10% but less than 100%, alternatively greater than 20% but less than 100%, alternatively greater than 30% but less than 100%, alternatively greater than 40% but less than 100%, alternatively greater than 50% but less than 100%, alternatively greater than 60% but less than 100%, alternatively greater than 70% but less than 100%, alternatively greater than 80% but less than 100%, or alternatively greater than 90% but less than 100%, of the activity of the reference polypeptide when evaluated at similar concentrations in a given assay system.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The term polypeptide include fusion proteins, including, but not limited to, fusion proteins with a heterologous amino acid sequence; fusion proteins with heterologous and homologous leader sequences; fusion proteins with or without N-terminal methionine residues; fusion proteins with amino acid sequences that facilitate purification such as chelating peptides; fusion proteins with immunologically tagged proteins; fusion proteins comprising a peptide with immunologically active polypeptide fragment (e.g. antigenic diphtheria or tetanus toxin or toxoid fragments) and the like.

Prevent: As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof. A course of action to prevent a disease, disorder or condition in a subject is typically applied in the context of a subject who is predisposed to developing a disease, disorder or condition due to genetic, experiential or environmental factors of developing a particular disease, disorder or condition. In certain instances, the terms “prevent”, “preventing”, “prevention” are also used to refer to the slowing of the progression of a disease, disorder or condition from an existing state to a more deleterious state.

Receptor: As used herein, the term “receptor” refers to a polypeptide having a domain that specifically binds a ligand that binding of the ligand results in a change to at least one biological property of the polypeptide. In some embodiments, the receptor is a cell surface receptor that comprises and extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of a cognate ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface receptor that is not associated with any intracellular signaling alone but upon ligand binding facilitates the formation of a heteromultimeric (including heterodimeric, heterotrimeric, etc.) or homomultimeric (including homodimeric, homotrimeric, homotetrameric, etc.) complex that results in a measurable biological effect in the cell such as activation of an intracellular signaling cascade (e.g. the Jak/STAT pathway). In some embodiments, a receptor is a membrane spanning single chain polypeptide comprising ECD, TM and ICD domains wherein the ECD, TM and ICD domains are derived from the same or differing naturally occurring receptor variants or synthetic functional equivalents thereof.

Recombinant: As used herein, the term “recombinant” is used as an adjective to refer to the method by which a polypeptide, nucleic acid, or cell was modified using recombinant DNA technology. A “recombinant protein” is a protein produced using recombinant DNA technology and is frequently abbreviated with a lower case “r” preceding the protein name to denote the method by which the protein was produced (e.g., recombinantly produced human growth hormone is commonly abbreviated “rhGH”). Similarly a cell is referred to as a “recombinant cell” if the cell has been modified by the incorporation (e.g. transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation) where the change is correlated with the activation, stimulation, or treatment, with or contact with exogenous agents or internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects. A “response” may be evaluated in vitro such as through the use of assay systems, surface plasmon resonance, enzymatic activity, mass spectroscopy, amino acid or protein sequencing technologies. A “response” may be evaluated in vivo quantitatively by evaluation of objective physiological parameters such as body temperature, bodyweight, tumor volume, blood pressure, results of X-ray or other imaging technology or qualitatively through changes in reported subjective feelings of well-being, depression, agitation, or pain. In some embodiments, the level of proliferation of CD3 activated primary human T-cells may be evaluated in a bioluminescent assay that generates a luminescent signal that is proportional to the amount of ATP present which is directly proportional to the number of cells present in culture as described in Crouch, et al. (1993) J. Immunol. Methods 160: 81-8 or through the use of commercially available assays such as the CellTiter-Glo® 2.0 Cell Viability Assay or CellTiter-Glo® 3D Cell Viability kits commercially available from Promega Corporation, Madison WI 53711 as catalog numbers G9241 and G9681 in substantial accordance with the instructions provided by the manufacturer. In some embodiments, the level of activation of T cells in response to the administration of a test agent may be determined by flow cytometric methods as described as determined by the level of STAT (e.g., STAT1, STAT3, STAT5) phosphorylation in accordance with methods well known in the art. For example, STAT5 phosphorylation may be measured using flow cytometric techniques as described in Horta, et al. supra., Garcia, et al., supra, or commercially available kits such as the Phospho-STAT5 (Tyr694) kit (commercially available from Perkin-Elmer, Waltham MA as Part Number 64AT5PEG) in performed in substantial accordance with the instructions provided by the manufacturer.

Significantly Reduced Binding: As used herein, the term “exhibits significantly reduced binding” is used with respect the a variant of a first molecule (e.g. a ligand or antibody) which exhibits a significant reduction in the affinity for a second molecule (e.g. receptor or antigen) relative the parent form of the first molecule. With respect to antibody variants, an antibody variant “exhibits significantly reduced binding” if the affinity of the variant antibody for an antigen if the variant binds to the native form of the receptor with and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent antibody from which the variant was derived. Similarly, with respect to variant ligands, a variant ligand “exhibits significantly reduced binding” if the affinity of the variant ligand binds to a receptor with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent ligand from which the variant ligand was derived. Similarly, with respect to variant receptors, a variant ligand “exhibits significantly reduced binding” if the affinity of the variant receptors binds to a with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent receptor from which the variant receptor was derived.

Small Molecule(s): The term “small molecules” refers to chemical compounds (typically pharmaceutically active compounds) having a molecular weight that is less than about 10 kDa, less than about 2 kDa, or less than about 1 kDa. Small molecules include, but are not limited to, inorganic molecules, organic molecules, organic molecules containing an inorganic component, molecules comprising a radioactive atom, and synthetic molecules. The term “small molecule” is a term well understood to those of ordinary skill in the pharmaceutical arts and is typically used to distinguish organic chemical compounds from biologics.

Specifically Binds: As used herein the term “specifically binds” refers to the degree of affinity for which a first molecule exhibits with respect to a second molecule. In the context of binding pairs (e.g., ligand/receptor, antibody/antigen) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. In a particular embodiment, where the first molecule of the binding pair is an antibody, the antibody specifically binds to the antigen (or antigenic determinant (epitope) of a protein, antigen, ligand, or receptor) if the equilibrium dissociation constant (K_{D}) between antibody and the antigen is lesser than about 10⁻⁶ M, alternatively lesser than about 10⁻⁸ M, alternatively lesser than about 10⁻¹⁰ M, alternatively lesser than about 10⁻¹¹ M, lesser than about 10⁻¹² M as determined by, e.g., Scatchard analysis (Munsen, et al. (1980) Analyt. Biochem. 107:220-239). In one embodiment where the ligand is an CD45RO sdAb and the receptor comprises an CD45RO, the CD45RO sdAb specifically binds if the equilibrium dissociation constant (KO) of the CD45RO sdAb/CD45RO ECD is lesser than about 10⁻⁵M, alternatively lesser than about 10⁻⁶ M, alternatively lesser than about 10⁻⁷M, alternatively lesser than about 10⁻⁸M, alternatively lesser than about 10⁻⁹M, alternatively lesser than about 10⁻¹⁰M, or alternatively lesser than about 10⁻¹¹ M. Specific binding may be assessed using techniques known in the art including but not limited to competition ELISA assays, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays)) and surface plasmon resonance assays (see, e.g., Drescher et al., (2009) Methods Mol Biol 493:323-343 with commercially available instrumentation such as the Biacore 8+, Biacore S200, Biacore T200 (GE Healthcare Bio-Sciences, 100 Results Way, Marlborough MA 01752). In some embodiments, the present disclosure provides molecules (e.g. CD45RO sdAbs) that specifically bind to the hCD45RO isoform.

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially Pure: As used herein, the term “substantially pure” indicates that a component of a composition makes up greater than about 50%, alternatively greater than about 60%, alternatively greater than about 70%, alternatively greater than about 80%, alternatively greater than about 90%, alternatively greater than about 95%, of the total content of the composition. A protein that is “substantially pure” comprises greater than about 50%, alternatively greater than about 60%, alternatively greater than about 70%, alternatively greater than about 80%, alternatively greater than about 90%, alternatively greater than about 95%, of the total content of the composition.

Suffering From: As used herein, the term “suffering from” refers to a determination made by a physician with respect to a subject based on the available objective or subjective information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm.

T-cell: As used herein the term “T-cell” or “T cell” is used in its conventional sense to refer to a lymphocytes that differentiates in the thymus, possess specific cell surface antigen receptors, and include some that control the initiation or suppression of cell-mediated and humoral immunity and others that lyse antigen-bearing cells. In some embodiments the T cell includes without limitation na\check{i}ve CD8⁺ T cells, cytotoxic CD8⁺ T cells, na\check{i}ve CD4⁺ T cells, helper T cells, e.g. T_{H}1, T_{H}2, T_{H}9, T_{H}11, T_{H}22, T_{FH}; regulatory T cells, e.g. T_{R}1, Tregs, inducible Tregs; memory T cells, e.g. central memory T cells, effector memory T cells, NKT cells, tumor infiltrating lymphocytes (TILs) and engineered variants of such T-cells including but not limited to CAR-T cells, recombinantly modified TILs and TCR-engineered cells. In some embodiments the T cell is a T cell expressing the CD45RO isoform referred to interchangeably as CD45RO cell, CD45RO+ cell, CD45 T cell, or CD45RO+ T cell).

Terminus/Terminal: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” refers to the position of a first amino acid residue relative to a second amino acid residue in a contiguous polypeptide sequence, the first amino acid being closer to the N-terminus of the polypeptide. “Immediately C-terminal” refers to the position of a first amino acid residue relative to a second amino acid residue in a contiguous polypeptide sequence, the first amino acid being closer to the C-terminus of the polypeptide.

Therapeutically Effective Amount: As used herein to the phrase “therapeutically effective amount” refers to the quantity of an agent when administered to a subject, either alone or as part of a pharmaceutical composition or treatment regimen, in a single dose or as part of a series of doses, provides a positive effect on any quantitative or qualitative symptom, aspect, or characteristic of a disease, disorder or condition. A therapeutically effective amount can be ascertained by measuring relevant physiological effects, and it may be adjusted in connection with a dosing regimen and in response to diagnostic analysis of the subject's condition. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in Response Evaluation Criteria In Solid Tumors (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. As used herein the terms “Complete Response (CR),” “Partial Response (PR)” “Stable Disease (SD)” and “Progressive Disease (PD)” with respect to target lesions and the terms “Complete Response (CR),” “Incomplete Response/Stable Disease (SD)” and Progressive Disease (PD) with respect to non-target lesions are understood to be as defined in the RECIST criteria. As used herein the terms “immune-related Complete Response (irCR),” “immune-related Partial Response (irPR),” “immune-related Progressive Disease (irPD)” and “immune-related Stable Disease (irSD)” as defined in accordance with the Immune-Related Response Criteria (irRC). As used herein, the term “Immune-Related Response Criteria (irRC)” refers to a system for evaluation of response to immunotherapies as described in Wolchok, et al. (2009) Guidelines for the Evaluation of Immune Therapy Activity in Solid Tumors: Immune-Related Response Criteria, Clinical Cancer Research 15(23): 7412-7420. In one embodiment, a therapeutically effective amount is an amount of an agent when used alone or in combination with another agent provides an provides a positive effect on any quantitative or qualitative symptom, aspect, or characteristic of a disease, disorder or condition and does not result in non-reversible serious adverse events in the course of administration of the agent to the mammalian subject.

Transmembrane Domain: The term “transmembrane domain” or “TM” refers to a polypeptide domain of a membrane spanning polypeptide (e.g. a transmembrane receptor) which, when the membrane spanning polypeptide is associated with a cell membrane, is which is embedded in the cell membrane and is in peptidyl linkage with the extracellular domain (ECD) and the intracellular domain (ICD) of a membrane spanning polypeptide. A transmembrane domain may be homologous (naturally associated with) or heterologous (not naturally associated with) with either or both of the extracellular and/or intracellular domains. In some embodiments, where the receptor is chimeric receptor comprising the intracellular domain derived from a first parental receptor and a second extracellular domains are derived from a second different parental receptor, the transmembrane domain of the chimeric receptor is the transmembrane domain normally associated with either the ICD or the ECD of the parent receptor from which the chimeric receptor is derived.

Treat: The terms “treat”, “treating”, treatment” and the like refer to a course of action (such as contacting the subject with pharmaceutical composition comprising a CD45RO sdAb alone or in combination with a supplementary agent) that is initiated with respect to a subject in response to a diagnosis that the subject is suffering from a disease, disorder or condition, or a symptom thereof, the course of action being initiated so as to eliminate, reduce, suppress, mitigate, or ameliorate, either temporarily or permanently, at least one of: (a) the underlying causes of such disease, disorder, or condition afflicting a subject; and/or (b) at least one of the symptoms associated with such disease, disorder, or condition. In some embodiments, treating includes a course of action taken with respect to a subject suffering from a disease where the course of action results in the inhibition (e.g., arrests the development of the disease, disorder or condition or ameliorates one or more symptoms associated therewith) of the disease in the subject.

Treg Cell or Regulatory T Cell. The terms “regulatory T cell”, “Treg cell”, or “Treg” are interchangeably herein to refers to a type of CD4⁺ T cell that can suppress the responses of other T cells including but not limited to effector T cells (T_{eff}). Treg cells are characterized by expression of CD4 (CD4⁺), the CD25 subunit of the IL2 receptor (CD25+), and the transcription factor forkhead box P3 (FOXP3+) (Sakaguchi, Annu Rev Immunol 22, 531-62 (2004). In some instances, the term “conventional CD4⁺ T cells” is used to distinguish non-Treg CD4⁺ T cells from CD4⁺ Tregs.

Variant: The terms “variant”, “protein variant” or “variant protein” or “variant polypeptide” are used interchangeably herein to refer to a polypeptide that differs from a parent polypeptide by virtue of at least one amino acid modification, substitution, or deletion. The parent polypeptide may be a naturally occurring or wild-type (WT) polypeptide or may be a modified version of a WT polypeptide. The term variant polypeptide may refer to the polypeptide itself, a composition comprising the polypeptide, or the nucleic acid sequence that encodes it. In some embodiments, the variant polypeptide comprises from about one to about ten, alternatively about one to about eight, alternatively about one to about seven, alternatively about one to about five, alternatively about one to about four, alternatively from about one to about three alternatively from one to two amino acid modifications, substitutions, or deletions, or alternatively a single amino acid amino acid modification, substitution, or deletion compared to the parent polypeptide. A variant may be at least about 99% identical, alternatively at least about 98% identical, alternatively at least about 97% identical, alternatively at least about 95% identical, or alternatively at least about 90% identical to the parent polypeptide from which the variant is derived.

Wild Type: By “wild type” or “WT” or “native” herein is meant an amino acid sequence or a nucleotide sequence that is found in nature, including allelic variations. A wild-type protein, polypeptide, antibody, immunoglobulin, IgG, etc. has an amino acid sequence or a nucleotide sequence that has not been modified by the hand of man.

### C: CD45

Although there is allelic variation in sequences in naturally occurring human CD45 (used interchangeably herein to refer to the hCD45ABC isoform), the canonical full length hCD45RABC isoform polypeptide is a 1306 amino acid polypeptide having the amino acid sequence:

(SEQ ID NO: 1)
MTMYLWLKLLAFGFAFLDTEVFVTGQSPTPSPTGLTTAKMPSVPLSSDPL
PTHTTAFSPASTFERENDFSETTTSLSPDNTSTQVSPDSLDNASAFNTTG
VSSVQTPHLPTHADSQTPSAGTDTQTFSGSAANAKLNPTPGSNAISDVPG
ERSTASTFPTDPVSPLTTTLSLAHHSSAALPARTSNTTITANTSDAYLNA
SETTTLSPSGSAVISTTTIATTPSKPTCDEKYANITVDYLYNKETKLFTA
KLNVNENVECGNNTCTNNEVHNLTECKNASVSISHNSCTAPDKTLILDVP
PGVEKFQLHDCTQVEKADTTICLKWKNIETFTCDTQNITYRFQCGNMIFD
NKEIKLENLEPEHEYKCDSEILYNNHKFTNASKIIKTDFGSPGEPQIIFC
RSEAAHQGVITWNPPQRSFHNFTLCYIKETEKDCLNLDKNLIKYDLQNLK
PYTKYVLSLHAYIIAKVQRNGSAAMCHFTTKSAPPSQVWNMTVSMTSDNS
MHVKCRPPRDRNGPHERYHLEVEAGNTLVRNESHKNCDFRVKDLQYSTDY
TFKAYFHNGDYPGEPFILHHSTSYNSKALIAFLAFLIIVTSIALLVVLYK
IYDLHKKRSCNLDEQQELVERDDEKQLMNVEPIHADILLETYKRKIADEG
RLFLAEFQSIPRVFSKFPIKEARKPFNQNKNRYVDILPYDYNRVELSEIN
GDAGSNYINASYIDGFKEPRKYIAAQGPRDETVDDFWRMIWEQKATVIVM
VTRCEEGNRNKCAEYWPSMEEGTRAFGDVVVKINQHKRCPDYIIQKLNIV
NKKEKATGREVTHIQFTSWPDHGVPEDPHLLLKLRRRVNAFSNFFSGPIV
VHCSAGVGRTGTYIGIDAMLEGLEAENKVDVYGYVVKLRRQRCLMVQVEA
QYILIHQALVEYNQFGETEVNLSELHPYLHNMKKRDPPSEPSPLEAEFQR
LPSYRSWRTQHIGNQEENKSKNRNSNVIPYDYNRVPLKHELEMSKESEHD
SDESSDDDSDSEEPSKYINASFIMSYWKPEVMIAAQGPLKETIGDFWQMI
FQRKVKVIVMLTELKHGDQEICAQYWGEGKQTYGDIEVDLKDTDKSSTYT
LRVFELRHSKRKDSRTVYQYQYTNWSVEQLPAEPKELISMIQVVKQKLPQ
KNSSEGNKHHKSTPLLIHCRDGSQQTGIFCALLNLLESAETEEVVDIFQV
VKALRKARPGMVSTFEQYQFLYDVIASTYPAQNGQVKKNNHQEDKIEFDN
EVDKVKQDANCVNPLGAPEKLPEAKEQAEGSEPTSGTEGPEHSVNGPASP
ALNQGS

For purposes of the present disclosure, the numbering of amino acid residues of the human CD45 polypeptides as described herein is made in accordance with the numbering of this canonical sequence (UniProt Sequence P08575 at https://www.uniprot.org/uniprot/P08575, SEQ ID NO:1).

In all known isoforms, the ECD of CD45 has a domain comprising a serine/threonine rich domain (abbreviated herein as “S/T” or “ST”) and four contiguous fibronectin-like regions referred to herein as D1, D2, D3 and D4. D1 corresponds to amino acids 223-298 of SEQ ID NO.1, D2 corresponds to amino acids 299-392 of SEQ ID NO.1, D3 corresponds to amino acids 393-478 of SEQ ID NO.1 and D4 corresponding to amino acids 479-571 of SEQ ID NO:1.

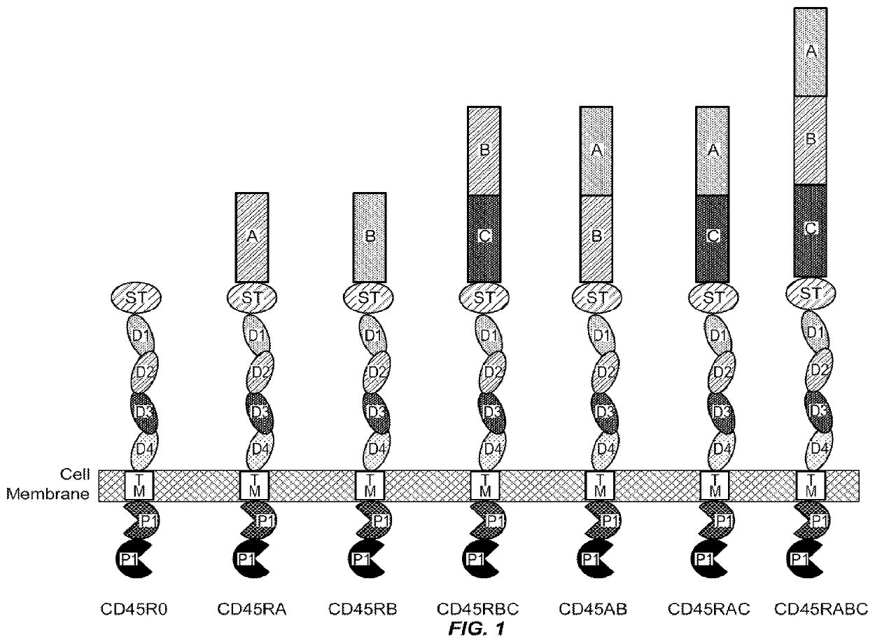

In various isoforms as illustrated in FIG. 1, there are three highly glycosylated domains referred to as Domain A (amino acids 34-99 of SEQ ID NO:1), Domain B (amino acids 100-146 of SEQ ID NO: 1) and Domain C (amino acids 147-194 of SEQ ID NO: 1) which are alternatively presented in the various isoforms illustrated.

One isoform, hCD45RO, is the shortest isoform of hCD45 having 1145 amino acids. The CD45RO isoform has a deletion of amino acids 34-194 of the canonical sequence precursor having a 25 amino acid signal peptide of amino acids 1-15 of SEQ ID NO:1 such that the ECD of the expressed mature hCD45RO molecule consists of amino acids 26-33 and 195-571 of SEQ ID NO:1. CD45RO is referred to as isoform 2 and the sequence identified at UniProt identifier P08575-4.

### D. CD45 Binding Molecules

The present disclosure relates to CD45 binding molecules comprising single domain antibodies (sdAbs) that specifically bind to the D1 to D4 FN3-like domains of the human CD45RO isoform (hCD45RO) which are found on all CD45-expressing cells. See FIG. 1.

CD45 is a commonly used cell surface marker for characterization of T cell subsets by flow cytometry and the presence of hCD45RO has been used to identify activated and memory T cell populations. In order to establish the properties of available hCD45RO antibodies and establish a basis of comparison for the heavy chain antibodies and the VHHs derived isolated from the llama immunization campaign described below, the binding properties and target regions of hCD45RO of four publicly available hybridoma cell lines that target the hCD45RO isoform, in particular the D1 to D4 FN3-like domains (FIG. 1). The four antibodies are:
(1) gap8.3 a mouse IgG2a isotype antibody derived from hybridoma ATCC No HB-12™;
(2) 4B2, a mouse IgG2a isotype antibody derived from hybridoma ATCC No. HB-196™;
(3) 9.4, a mouse IgG2a isotype antibody derived from hybridoma ATCC No. HB-10508™;
(4) VHE a humanized heavy chain variable region referred to as SEQ ID NO.9 in European Patent EP1664122B1 published Mar. 19, 2010.

For the three antibodies derived from the mouse hybridoma lines (1-3), the VH and VL domains from the hybridomas were sequenced and cloned into a mouse IgG2A backbone. With respect to VHE (4), the VH and VL sequences were cloned into a human IgG1 backbone. Single chain Fv (scFv) variants of each of these molecules were also generated by expression of the VH and VL domains joined with an 18 amino acid Gly-Ser linker. Recombinant forms of the IgG antibodies as well as the scFvs linked to a human IgG1 Fc domain were generated by transient expression in Expi293™ cells followed by purification on protein A resin. These IgG and scFv molecules were used for domain mapping experiments and biophysical analysis.

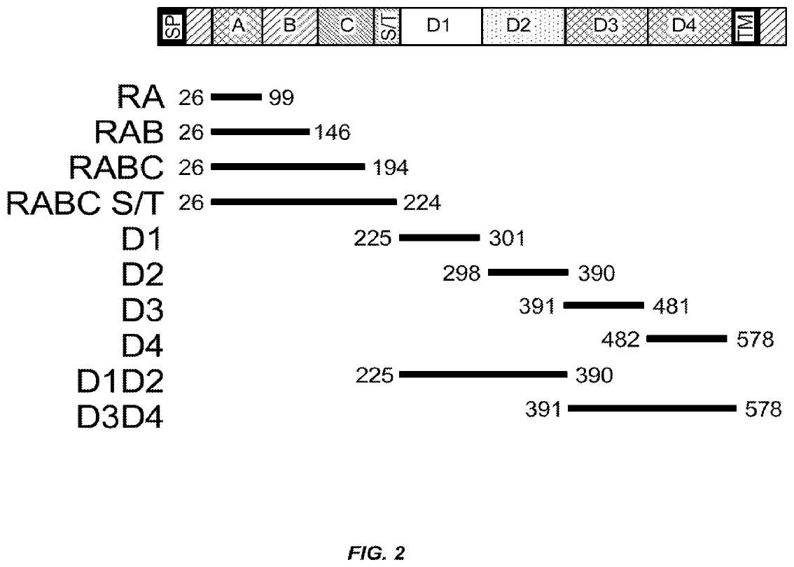

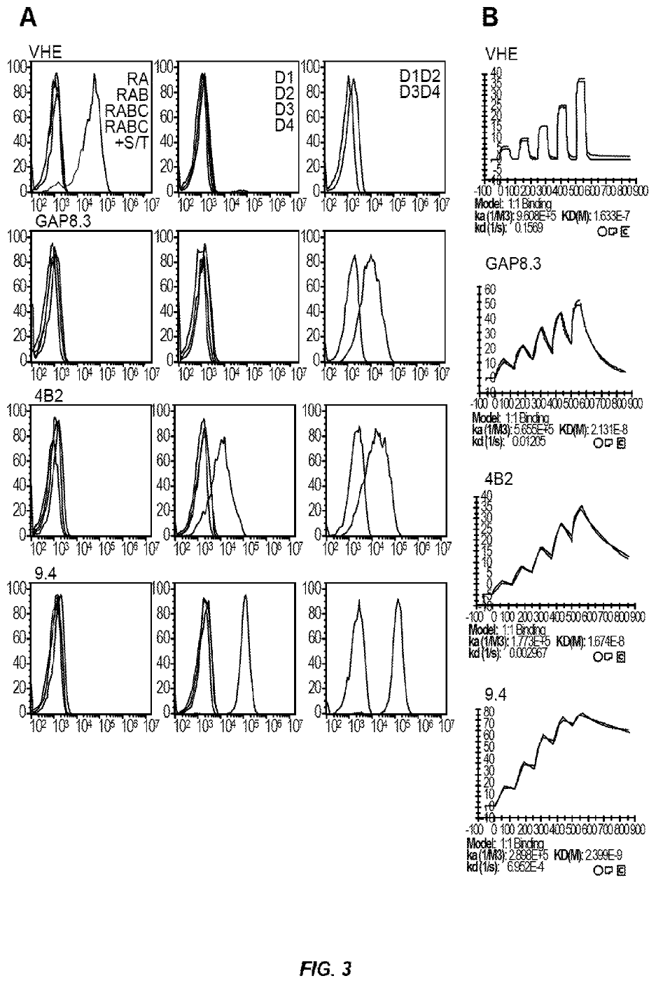

In order to better elucidate the extracellular domains of human CD45 targeted by these four antibodies, a yeast display system for domain mapping was employed in substantial accordance with the teaching of Cochran, et al. (2004) J. Immunol Methods 287:147-158. Ten yeast strains containing hCD45 domains illustrated in FIG. 2 of the attached drawings were fused to the C-terminus of the Aga2p anchor protein for yeast surface expression (Boder and Wittrup (1997) Nature 15:553-557; Boder and Wittrup (2000) Methods In Enzymology 328:430-444). These constructs included each of the mucin domain exons (RA=exon 1, RAB=exons 1 and 2, RABC=exons 1, 2, and 3), RABC plus S/T (a 30 amino acid serine/threonine-rich linker between RABC and D1), each of the individual FN3-like domains (D1, D2, D3, D4), as well as two two-domain modules (D1D2 and D3D4). The Aga2-CD45 fusions also included an internal FLAG® epitope tag (Munro and Pelham (1984) EMBO J 3(13):3087-93; and Hopp, et al. (1988) Bio/Technology 6(10):1204-1210) between Aga2 and CD45 as well as a C-terminal c-Myc (EQKLISEEDL (SEQ ID NO: 608)) epitope tag. Surface expression for each of the Aga2-CD45 fusions was confirmed by FLAG® or c-Myc staining in accordance with known procedures. The recombinant anti-CD45 IgG molecules were then used to stain each of the yeast strains, followed by flow cytometry to identify to those strain(s) to which the antibodies bound. The results of these studies are provided in FIG. 3 of the attached drawings and summarized in Table 2 below.

TABLE 2
Affinity of Anti-hCD45RO IgG Antibodies and scFv Derivatives

| | | IgG | | scFv | |
|---|---|---|---|---|---|
| Clone | domain | Affinity (K_{D}) | | Affinity (K_{D}) | |
| VHE | S/T | 163 | nM | 260 | nM |
| 4B2 | D1 | 17 | nM | 12 | nM |
| 9.4 | D1 | 2.4 | nM | 18 | nM |
| Gap8.3 | D1D2 | 21 | nM | 40 | nM |

As indicated, the VHE antibody bound to S/T, the 30 amino acid Ser/Thr-rich domain (amino acids 194-224) between the RABC and the D1 domains. The gap8.3 antibody bound only to the yeast expressing the D1D2 domain construct but neither D1 or D2 individually suggesting an epitope that is composed of surfaces from both the D1 and D2 domains. The 4B2 and 9.4 antibodies bound the D1 domain alone. Therefore, all four antibodies bound to CD45 RO with epitopes focused in the N-terminal portion of the ECD of hCD45 illustrating that the anti-CD45 RO antibodies available in the public domain target the N-terminal portions (i.e., the, S/T, D1 and/or D2 domains) of the hCD45RO ECD which are somewhat distant from the cell membrane. This binding pattern reflects their primary usage in the art as tools for immune cell phenotyping by flow cytometry, which typically selects for IgG-accessible N-terminal epitopes.

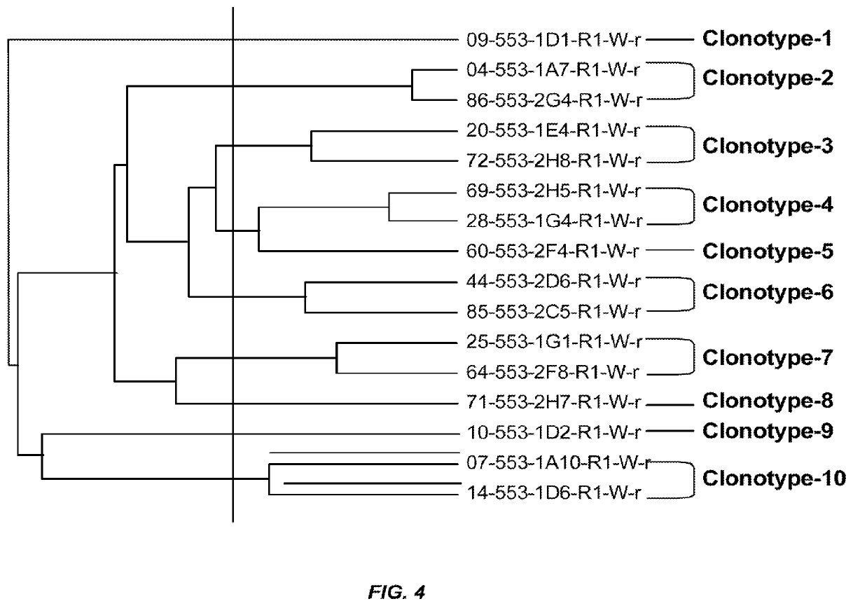

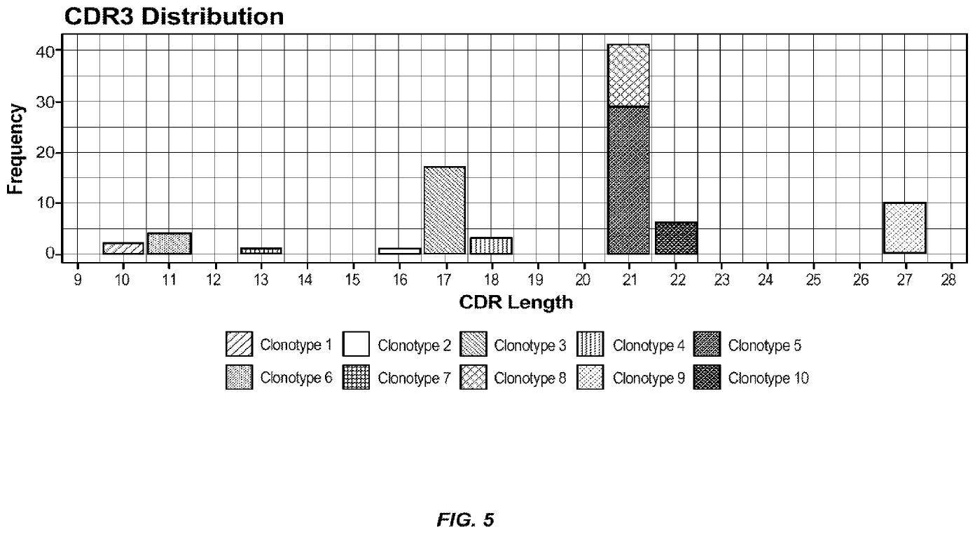

A llama CD45 immunization program was conducted to identify new antibodies (and single domain VHH antibodies derived therefrom) to hCD45RO and identify the hCD45RO domains targeted by the antibodies to select for molecules that bound hCD45RO isoform and particularly to generate antibodies which bind to the D3 and D4 regions of hCD45RO which are closer to the membrane surface. Briefly, a llama was immunized with the full-length human CD45 ECD (amino acids 26-578) as an antigen. After the immunization protocol was complete, PBMCs were isolated and a phage display library constructed from the post-immune llama antibody repertoire. After one round of selection, excellent reactivity of the library to the CD45 ECD was observed with >96% of the clones showing positive results in an ELISA against the hCD45 ECD immunogen. Of the 93 ELISA positive clones sequenced, 51 unique, high quality VHH sequences were obtained. Sequence analysis identified ten unique clonotypes (as shown FIG. 4 of the attached drawings and Table 4 below) with varied CDR3 lengths from 10 to 27 amino acids (FIG. 5 and Table 4).

A subset of molecules representing each clonotype was recombinantly expressed with a monomeric Fc tag and tested for binding to the hCD45RO (amino acids 194-578) domain by surface plasmon resonance (SPR) using a Biacore® T-200. Of the ten clonotypes identified, seven clonotypes bound to hCD45RO with an affinity range (K_{D}) of 630 pM to 522 nM as summarized in Table 3 below. The yeast display system as described above was employed to domain map the VHH binding sites. For labeling, His-tagged VHH proteins were incubated with the yeast and detected using an anti-VHH antibody. Seven VHH clonotypes bound across all four domains of CD45RO, with clonotypes 3, 4, and 7 targeting the D1 and D2 domains, clonotype 10 binding the D3 domain, clonotypes 2, 6, and 8 interacting with the D4 domain (Table 3).

TABLE 3
Characterization of hCD45RO VHHs

| | | CD45 | | | |
|---|---|---|---|---|---|
| Clone | Clonotype | domain | Affinity (K_{D}) | | SEQ ID NO |
| 1G1 | 7 | D1 | 28 | nM | 2 |
| 1E4 | 3 | D1D2 | 299 | nM | 50 |
| 2F8 | 7 | D1D2 | 69 | nM | 54 |
| 1G4 | 4 | D2 | 0.87 | nM | 94 |
| 2H5 | 4 | D2 | 0.63 | nM | 98 |
| 1A10 | 10 | D3 | 32 | nM | 110 |
| 1D6 | 10 | D3 | 522 | nM | 122 |
| 2G4 | 2 | D4 | 8.3 | nM | 150 |
| 2C5 | 6 | D4 | 1.4 | nM | 142 |
| 2H7 | 8 | D4 | 0.88 | nM | 154 |

The VHHs were sequenced and the CDRs thereof identified (all CDRs determined by method of Kabat) as summarized in the following Table 4.

TABLE 4
Amino Acid Sequence of VHHs and CDRs

| SEQ | | |
|---|---|---|
| ID | | |
| NO: | Name | Amino Acid Sequence |
| 2. | 1G1-VHH | QVQLQASGGGLVQAGGSLRLSCAASGRTVSSDA |
| | | MGWFRQAPGKEREFVAAINWNGASTYYGDSAQ |
| | | GRFTISRDNAKNTVYLQMNSLKSEDTAVYYCAAS |
| | | DWVVGGISLMNGAEYKYWGQGTLVTVSS |
| 3. | 1G1-CDR1 | SDAMG |
| 4. | 1G1-CDR2 | AINWNGASTYYGDSAQG |
| 5 | 1G1-CDR3 | SDWVVGGISLMNGAEYKY |
| 6. | 2H8-VHH | QVQLQESGGGLVQAGGSLRLSCVASGRTFSSYGM |
| | | GWFRQAPGKEREFVASISWSGGSTYYPDSVLGRF |
| | | TISRDNAKNVLYLQMNSLKPEDTAVYYCAASST |
| | | WNNNDPHEYDYWGQGTLVTVSS |
| 7. | 2H8-CDR1 | SYGMG |
| 8. | 2H8-CDR2 | SISWSGGSTYYPDSVLG |
| 9. | 2H8-CDR3 | SSTWNNNDPHEYDY |
| 10. | 1E1-VHH | QVQLQESGGGLVQPGGSLRLSCAASGRTFSSYGM |
| | | GWFRQAPGKEREFVSSISWSGRSTYYTNAVRGRF |
| | | TISRDNAKNTVYLQMNSLKPEDTAVYYCAASST |
| | | WNNNDPTEYDYWGQGTLVTVSS |
| 11. | 1E1-CDR1 | SYGMG |
| 12. | 1E1-CDR2 | SISWSGRSTYYTNAVRG |
| 13. | 1E1-CDR3 | SSTWNNNDPTEYDY |
| 14. | 1E5-VHH | QVQLQEFGGGLVQPGGSLRLSCAASGRTFSDYAM |
| | | GWFRQAPGKEREFVASISWSGGSLYYAGAVRGRF |
| | | TISRDNAKNTVYLQMNSLKPEDTAVYYCAASST |
| | | WNNNDPHEYDYWGQGTQVTVSS |
| 15. | 1E5-CDR1 | DYGMG |
| 16. | 1E5-CDR2 | SSSWSGRSTYYAAAVRG |
| 17. | 1E5-CDR3 | SSSWNNNDPSEYEY |
| 18. | 1G7-VHH | QVQLQQSGGDLVQPGGSLRLSCAASGRTFSDYG |
| | | MGWFRQAPGKEREFVASSSWSGRSTYYAAAVRG |
| | | RFTISRDNAKSTVYLQMNSLKAEDTAVYYCAASS |
| | | SWNNNDPSEYEYWGQGTQVTVSS |
| 19. | 1G7-CDR1 | DYGMG |
| 20. | 1G7-CDR2 | SSSWSGRSTYYAAAVRG |
| 21. | 1G7-CDR3 | SSSWNNNDPSEYEY |
| 22. | 1H6-VHH | QVQLQAFGGGTVQAGGSLRLSCTASGGTFSDYG |
| | | MGWFRQAPGKEREFVSSISWSGNSVYYAGAVRG |
| | | RFTISRDNAKNTVYLQMNSLQPEDTAVYYCAASS |
| | | TWNNNDPTEYEYWGQGTQVTVSS |
| 23. | 1H6-CDR1 | DYGMG |
| 24. | 1H6-CDR2 | SISWSGNSVYYAGAVRG |
| 25. | 1H6-CDR3 | SSTWNNNDPTEYEY |
| 26. | 1H7-VHH | QVQLQQFGGGLVQPGGSLRLSCAASGRTFSDYG |
| | | MGWFRQAPGKEREFVASSSWSGRSTYYAAAVRG |
| | | RFTISRDNAKSTVYLQMNSLKAEDTAVYYCAASS |
| | | SWNNNDPSEYEYWGQGTLVTVSS |
| 27. | 1H7-CDR1 | DYGMG |
| 28. | 1H7-CDR2 | SSSWSGRSTYYAAAVRG |
| 29. | 1H7-CDR3 | SSSWNNNDPSEYEY |
| 30. | 2C6-VHH | QVQLQEFGGGLVQPGGSLRLSCAASGRTFSDYGM |
| | | GWFRQAPGKEREFVASSSWSGRSTYYAAAVRGR |
| | | FTISRDNAKSTVYLQMNSLKAEDTAVYYCAASSS |
| | | WNNNDPSEYEYWGQGTQVTVSS |
| 31. | 2C6-CDR1 | DYGMG |
| 32. | 2C6-CDR2 | SSSWSGRSTYYAAAVRG |
| 33. | 2C6-CDR3 | SSSWNNNDPSEYEY |
| 34. | 2C7-VHH | QVQLQAFGGGLVQPGGSLRLSCAASGGTFSDYG |
| | | MGWFRQAPGKEREFVASSSWSGRSTYYAAAVRG |
| | | RFTISRDNAKSLVYLQMNNLKPEDTAVYYCAASS |
| | | TWNNNDPSEYEYWGQGTQVTVSS |
| 35. | 2C7-CDR1 | DYGMG |
| 36. | 2C7-CDR2 | SSSWSGRSTYYAAAVRG |
| 37. | 2C7-CDR3 | SSTWNNNDPSEYEY |
| 38. | 2D4-VHH | QVQLQESGGGLVEAGGSLRLSCAASGGTFSDYG |
| | | MGWFRQAPGKEREFVSSISWSGNSVYYAGAVRG |
| | | RFTISRDNAKNTVYLQMNSLQPEDTAVYYCAASS |
| | | TWNNNDPTEYEYWGQGTQVTVSS |
| 39. | 2D4-CDR1 | DYGMG |
| 40. | 2D4-CDR2 | SISWSGNSVYYAGAVRG |
| 41. | 2D4-CDR3 | SSTWNNNDPTEYEY |
| 42. | 2E1-VHH | QVQLQASGGGLVQPGGSLRLSCAASGRTFSSYGM |
| | | GWFRQAPGKEREFVASISWSGRSTYYTNAVRGRF |
| | | TISRDNAKNTVYLQMNSLKPEDTAVYYCAASST |
| | | WNNNDPTEYEYWGQGTQVTVSS |
| 43. | 2E1-CDR1 | SYGMG |
| 44. | 2E1-CDR2 | SISWSGRSTYYTNAVRG |
| 45. | 2E1-CDR3 | SSTWNNNDPTEYEY |
| 46. | 2E5-VHH | QVQLQESGGGLVQPGGSLRLSCAASGRTFSSYGM |
| | | GWFRQAPGKEREFVSSISWSGRSTYYTNAVRGRF |
| | | TISRDNAKNTVYLQMNSLKPEDTAVYYCAASST |
| | | WNNNDPTEYEYWGQGTQVTVSS |
| 47. | 2E5-CDR1 | SYGMG |
| 48. | 2E5-CDR2 | SISWSGRSTYYTNAVRG |
| 49. | 2E5-CDR3 | SSTWNNNDPTEYEY |
| 50. | 1E4-VHH | QVQLQESGGGLVQPGGSLRLSCAASGGTFSDYG |
| | | MGWFRQAPGKEREFVSSISWSGNSVYYAGAVRG |
| | | RFTISRDNAKNTVYLQMNSLQPEDTAVYYCAASS |
| | | TWNNNDPTEYEYWGQGTLVTVSS |
| 51. | 1E4-CDR1 | DYGMG |
| 52. | 1E4-CDR2 | SISWSGNSVYYAGAVRG |
| 53. | 1E4-CDR3 | SSTWNNNDPTEYEY |
| 54. | 2F8-VHH | QVQLQQFGGGLVQAGGSLRLSCAASGRTFSGYA |
| | | MGWFRQAPGKEREFVAAINWSGGSTYYADSVKG |
| | | RYTISRDNAKNTGYLQMNSLKSEDTAVYYCAAS |
| | | DWVVGGISLMNGAEYKYWGQGTLVTVSS |
| 55. | 2F8-CDR1 | GYAMG |
| 56. | 2F8-CDR2 | AINWSGGSTYYADSVKG |
| 57. | 2F8-CDR3 | SDWVVGGISLMNGAEYKY |
| 58. | 1D7-VHH | QVQLQESGGGLVQAGGSLRLSCAASGRTVSSDA |
| | | MGWFRQAPGKEREFVAAINWNGASTYYADSVK |
| | | GRFTISRDNAKNTVYLQMNSLKSEDTAVYYCAAS |
| | | DWVVGGISLMNGAEYAYWGQGTQVTVSS |
| 59. | 1D7-CDR1 | SDAMG |
| 60. | 1D7-CDR2 | AINWNGASTYYADSVKG |
| 61. | 1D7-CDR3 | SDWVVGGISLMNGAEYAY |
| 62. | 1E7-VHH | QVQLQQFGGGLVQAGGSLRLSCAASGRTVSSDA |
| | | MGWFRQAPGKEREFVAAINWNGASTYYADSVK |
| | | GRFTISRDNAKNTVYLQMNSLKSEDTAVYYCAAS |
| | | DWVVGGISLMNGAEYAYWGQGTQVTVSS |
| 63. | 1E7-CDR1 | SDAMG |
| 64. | 1E7-CDR2 | AINWNGASTYYADSVKG |
| 65. | 1E7-CDR3 | SDWVVGGISLMNGAEYAY |
| 66. | 1E8-VHH | QVQLQPSGGGLVQAGGSLRLSCAASGRTVSSDA |
| | | MGWFRQAPGKEREFVAAINWNGASTYYADSVK |
| | | GRFTISRDNAKNTVYLQMNSLKSEDTAVYYCAAS |
| | | DWVVGGISLMNGAEYAYWGQGTQVTVSS |
| 67. | 1E8-CDR1 | SDAMG |
| 68. | 1E8-CDR2 | AINWNGASTYYADSVKG |
| 69. | 1E8-CDR3 | SDWVVGGISLMNGAEYAY |
| 70. | 1G2-VHH | QVQLQAFGGGLVQAGGSLRLSCKASGRTVSSDA |
| | | MGWFRQAPGKEREFVAAINWNGASTYYADSVK |
| | | GRFTISRDNAKNTVYLQMNSLKSEDTAVYYCAVS |
| | | DWVVGGISLMNGAEYKYWGQGTQVTVSS |
| 71. | 1G2-CDR1 | SDAMG |
| 72. | 1G2-CDR2 | AINWNGASTYYADSVKG |
| 73. | 1G2-CDR3 | SDWVVGGISLMNGAEYKY |
| 74. | 1G8-VHH | QVQLQEFGGGLVQAGGSLRLSCKASGRTVSSDA |
| | | MGWFRQAPGKEREFVAAINWNGASTYYADSVK |
| | | GRFTISRDNAKNTVYLQMNSLKSEDTAVYYCAVS |
| | | DWVVGGISLMNGAEYKYWGQGTQVTVSS |
| 75. | 1G8-CDR1 | SDAMG |
| 76. | 1G8-CDR2 | AINWNGASTYYADSVKG |
| 77. | 1G8-CDR3 | SDWVVGGISLMNGAEYKY |
| 78. | 2C4-VHH | QVQLQAFGGGLVQAGGSLRLSCAASGRTVSSDA |
| | | MGWFRQAPGKEREFVAAINWNGASTYYADSVK |
| | | GRFTISRDNAKNTVYLQMNSLKSEDTAVYYCAAS |
| | | DWVVGGISLMNGVEYKYWGQGTQVTVSS |
| 79. | 2C4-CDR1 | SDAMG |
| 80. | 2C4-CDR2 | AINWNGASTYYADSVKG |
| 81. | 2C4-CDR3 | SDWVVGGISLMNGVEYKY |
| 82. | 2D7-VHH | QVQLAESGGGLVQAGGSLRLSCKASGRTVSSDA |
| | | MGWFRQAPGKEREFVAAINWNGASTYYADSVK |
| | | GRFTISRDNAKNTVYLQMNSLKSEDTAVYYCAVS |
| | | DWVVGGISLMNGAEYKYWGQGTQVTVSS |
| 83. | 2D7-CDR1 | SDAMG |
| 84. | 2D7-CDR2 | AINWNGASTYYADSVKG |
| 85. | 2D7-CDR3 | SDWVVGGISLMNGAEYKY |
| 86. | 2F5-VHH | QVQLQESGGDLVQRGGSLRLSCKASGRTVSSDA |
| | | MGWFRQAPGKEREFVAAINWNGASTYYADSVK |
| | | GRFTISRDNAKNTVYLQMNSLKSEDTAVYYCAVS |
| | | DWVVGGISLMNGAEYKYWGQGTQVTVSS |
| 87. | 2F5-CDR1 | SDAMG |
| 88. | 2F5-CDR2 | AINWNGASTYYADSVKG |
| 89. | 2F5-CDR3 | SDWVVGGISLMNGAEYKY |
| 90. | 2H2-VHH | QVQLQESGGGLVQAGGSLRLSCKASGRTVSSDA |
| | | MGWFRQAPGKEREFVAAINWNGASTYYADSVK |
| | | GRFTISRDNAKNTVYLQMNSLKSEDTAVYYCAVS |
| | | DWVVGGISLMNGAEYKYWGQGTQVTVSS |
| 91. | 2H2-CDR1 | SDAMG |
| 92. | 2H2-CDR2 | AINWNGASTYYADSVKG |
| 93. | 2H2-CDR3 | SDWVVGGISLMNGAEYKY |
| 94. | 1G4-VHH | QVQLQESGGGLVRPGGSLRLSCAASGGTFSSYAM |
| | | GWFRQAPGKEREFVAAIGGSGDSTYYADSVKGRF |
| | | TISRDNAKNTAYLQMNSLKPEDTAVYYCQADPT |
| | | MFHKLYYGIHPNEYEYWGQGTLVTVSS |
| 95. | 1G4-CDR1 | SYAMG |
| 96. | 1G4-CDR2 | AIGGSGDSTYYADSVKG |
| 97. | 1G4-CDR3 | DPTMFHKLYYGIHPNEYEY |
| 98. | 2H5-VHH | QVQLQESGGGLVQTGGSLTLSCVASGGTFSSYAM |
| | | GWFRQAPGKEREFVAAIGGSGDSTYYADSVKGRF |
| | | TISRDNAKNSVYLQMNSLKPEDTAVYYCQADPT |
| | | MFHKLYYGINPNEYDYWGQGTLVTVSS |
| 99. | 2H5-CDR1 | SYAMG |
| 100. | 2H5-CDR2 | AIGGSGDSTYYADSVKG |
| 101. | 2H5-CDR3 | DPTMFHKLYYGINPNEYDY |
| 102. | 2C3-VHH | QVQLQESGGGLVKGGGSLRLSCVASGGTFSSYA |
| | | MGWFRQAPGKEREFVAAIGGSGDSTYYADSVKG |
| | | RFTISRDNAKNSVYLQMNSLKPEDTAVYYCQADP |
| | | TMFHKLYYGINPNEYDYWGQGTLVTVSS |
| 103 | 2C3-CDR1 | SYAMG |
| 104. | 2C3-CDR2 | AIGGSGDSTYYADSVKG |
| 105. | 2C3-CDR3 | DPTMFHKLYYGINPNEYDY |
| 106. | 2F7-VHH | QVQLQESGGGLVKGGGSLRLSCVASGGTFSSYA |
| | | MGWFRQAPGKEREFVAAIGGSGDSTYYADSVKG |
| | | RFTISRDNAKNSVYLQMNSLKPEDTAVYYCQADP |
| | | TMFHKLYYGINPNEYDYWGQGTQVTVSS |
| 107. | 2F7-CDR1 | SYAMG |
| 108 | 2F7-CDR2 | AIGGSGDSTYYADSVKG |
| 109. | 2F7-CDR3 | DPTMFHKLYYGINPNEYDY |
| 110. | 1A10-VHH | QVQLQAFGGGLVQAGGSLGLSCSASGTIFNLDYM |
| | | GWYRQALGKQRELVATITSDGRTNYADSVKGRF |
| | | TIFRDGAKNAILMQMNSLKPEDTAVYYCKAYLSG |
| | | RSDYWGQGTQVTVSS |
| 111. | 1A10-CDR1 | LDYMG |
| 112. | 1A10-CDR2 | TITSDGRTNYADSVK |
| 113. | 1A10-CDR3 | YLSGRSDY |
| 114. | 1D1-VHH | QVQLQASGGRLVRPGESLTLSCVVSGRTSGTTAM |
| | | GWFRQARGKERQFLAQISYSDGSTYYAASVKGRF |
| | | NISRDNAGNTVYLQMDSLTSEDTATYYCAPTRGE |
| | | GSRNVNWGQGTQVTVSS |
| 115. | 1D1-CDR1 | TTAMG |
| 116. | 1D1-CDR2 | QISYSDGSTYYAASVKG |
| 117. | 1D1-CDR3 | TRGEGSRNVN |
| 118. | 1D2-VHH | QVQLQAFGGGSVQAGGSMKLSCKVDGVSISGNA |
| | | MRWYRQLPGKERTWAAIVLSNGNEHYANSVKG |
| | | RFVISRDDAKNTVDLQMNNLKPEDTGTYYCNLQS |
| | | PQGQYWGQGTQVTVSS |
| 119. | 1D2-CDR1 | GNAMR |
| 120. | 1D2-CDR2 | IVLSNGNEHYANSVKG |
| 121. | 1D2-CDR3 | QSPQGQY |
| 122. | 1D6-VHH | QVQLQESGGGSVQAGGSLGLFCSASGTIFNIDVM |
| | | GWYRQAPGKQRELVATITSDGRTNYADSVKGRF |
| | | TISRDGAKNAVHVQMNSLKPEDTAVYYCKAYLS |
| | | GRSDYWGQGTQVTVSS |
| 123. | 1D6-CDR1 | IDVMG |
| 124. | 1D6-CDR2 | TITSDGRTNYADSVKG |
| 125. | 1D6-CDR3 | YLSGRSDY |
| 126. | 1D3-VHH | QVQLQASGGGLVQAGGSLGLSCSASGTIFNLDYM |
| | | GWYRQALGKQRELVATITSDGRTNYADSVKGRF |
| | | TIFRDGAKNAILMQMNSLKPEDTAVYYCKAYLSG |
| | | RSDYWGQGTLVTVSS |
| 127 | 1D3-CDR1 | LDYMG |
| 128. | 1D3-CDR2 | TITSDGRTNYADSVKG |
| 129. | 1D3-CDR3 | YLSGRSDY |
| 130. | 2G2-VHH | QVQLQESGGGLVQAGGSLGLSCSASGTIFNLDYM |
| | | GWYRQALGKQRELVATITSDGRTNYADSVKGRF |
| | | TIFRDGAKNAILMQMNSLKPEDTAVYYCKAYLSG |
| | | RSDYWGQGTQVTVSS |
| 131. | 2G2-CDR1 | LDYMG |
| 132. | 2G2-CDR2 | TITSDGRTNYADSVKG |
| 133. | 2G2-CDR3 | YLSGRSDY |
| 134. | 2F4-VHH | QVQLQESGGGLVKGGGSLRLSCVASGGTFSSYA |
| | | MGWFRQAPGKEREFVAAIGGSGDSTYYADSVKG |
| | | RFTISRDNAKNSVYLQMNNLKPEDTAIYYCAAAR |
| | | ESSVYYTSTDPEEYGYWGQGTLVTVSS |
| 135. | 2F4-CDR1 | SYAMG |
| 136. | 2F4-CDR2 | AIGGSGDSTYYADSVKG |
| 137. | 2F4-CDR3 | ARESSVYYTSTDPEEYGY |
| 138. | 1A7-VHH | QVQLQAFGGGLVQAGGSLRLSCAASGSIFSINAM |
| | | GWYRQAPGKQRELVAVITSGGSTHYADSVKGRF |
| | | TISRDNTKNTLYLQMNSLKPEDTAVYYCNGRRKT |
| | | TGWGTLREWEGSRDPDDYDYWGQGTLVTVSS |
| 139. | 1A7-CDR1 | INAMG |
| 140. | 1A7-CDR2 | VITSGGSTHYADSVKG |
| 141. | 1A7-CDR3 | RRKTTGWGTLREWEGSRDPDDYDY |
| 142. | 2C5-VHH | QVQLQEFGGGLVQAGASLRLSCAASGGTFSYFM |
| | | GWFRQAPGKEREFVATINRNGGATGYADSVKGR |
| | | FTISRDNAKNTLYLQMDSVTPEDTAVYYCAAARE |
| | | SSVYYTSTDPAEYGYWGQGTQVTVSS |
| 143. | 2C5-CDR1 | YFMG |
| 144. | 2C5-CDR2 | TINRNGGATGYADSVKG |
| 145. | 2C5-CDR3 | ARESSVYYTSTDPAEYGY |
| 146. | 2D6-VHH | QVQLQESGGGLVQAGASLRLSCAASGRTFSSFMG |
| | | WFRQAPGKERKFVATINWNGGATGYADSVKGRF |
| | | TISRDNAKNTLYLQMNSLKPEDTAVYYCAAART |
| | | GSAYYTSSDAGEYNYWGQGTQVTVSS |
| 147. | 2D6-CDR1 | SFMG |
| 148. | 2D6-CDR2 | TINWNGGATGYADSVKG |
| 149. | 2D6-CDR3 | ARTGSAYYTSSDAGEYNY |
| 150. | 2G4-VHH | QVQLQEFGGGLVQAGGSLRLSCAASGSISRINAM |
| | | GWYRQAPGKQRELVAVITSGGSTHYADSVKGRF |
| | | TISRDNAKNTLYLQMNSLKPEDTAVYYCNGRRKT |
| | | TGWGSLREWEGSRDPDEYNYWGQGTLVTVSS |
| 151. | 2G4-CDR1 | INAMG |
| 152. | 2G4-CDR2 | VITSGGSTHYADSVKG |
| 153. | 2G4-CDR3 | RRKTTGWGSLREWEGSRDPDEYNY |
| 154. | 2H7-VHH | QVQLQESGGGLVQAGGSLRLSCAASGRTFSTHA |
| | | MGWFRQAPGKEREFVAAIAWSGGSTYYADSVKG |
| | | RFTISRDISKNTIYLQMNSLKPEDTALYYCAATNG |
| | | GAWHYHERYFGSWGQGTLVTVSS |
| 155. | 2H7-CDR1 | THAMG |
| 156. | 2H7-CDR2 | AIAWSGGSTYYADSVKG |
| 157. | 2H7-CDR3 | TNGGAWHYHERYFGS |
| 158. | 1A8-VHH | QVQLQESGGGLVQAGTSLRLSCAASGRTFSSFMG |
| | | WFRQAPGKEREFVATINRSGGATGYADSVKGRFT |
| | | ISRDNAKNTLYLQMNSLKPEDTAVYYCAAARGSS |
| | | VYYTSSDPEEYGHWGQGTLVTVSS |
| 159. | 1A8-CDR1 | SFMG |
| 160. | 1A8-CDR2 | TINRSGGATGYADSVKG |
| 161. | 1A8-CDR3 | ARGSSVYYTSSDPEEYGH |
| 162. | 1B7-VHH | QVQLQESGGGLVQAGGSLRLSCAASGSIFSINGM |
| | | G |
| | | WYRQAPGKQRELVAVITSGGSTHYADSVKGRFTI |
| | | S |
| | | RDNAKNTLYLQMNSLKPEDTAVYYCNGRRKTTG |
| | | WGTLREWEGSRDPDDYDYWGQGTQVTVSS |
| 163. | 1B7-CDR1 | INGMG |
| 164. | 1B7-CDR2 | VITSGGSTHYADSVKG |
| 165 | 1B7-CDR3 | RRKTTGWGTLREWEGSRDPDDYDY |
| 166. | 1E2-VHH | QVQLQESGGGLVQAGGSLRLSCAASELTFSTHAM |
| | | GWFRQAPGKEREFVAAIAWSGGSTYYADSVKGR |
| | | FTISRDISKNTIYLQMNSLKPEDTALYYCAATNGG |
| | | AWHYHERYFGSWGQGTQVTVSS |
| 167. | 1E2-CDR1 | THAMG |
| 168. | 1E2-CDR2 | AIAWSGGSTYYADSVKG |
| 169. | 1E2-CDR3 | TNGGAWHYHERYFGS |
| 170. | 1E6-VHH | QVQLQEFGGGLVQAGASLRLSCAASGRTLSRFMG |
| | | WFRQAPGKERRFVATINWNGGATGYADSVKGRF |
| | | TISRDNAKNTLYLQMNSLKPDDTAVYYCAAARTS |
| | | SIYY |
| | | TSSDAEEYDYWGQGTQVTVSS |
| 171. | 1E6-CDR1 | RFMG |
| 172. | 1E6-CDR2 | TINWNGGATGYADSVKG |
| 173. | 1E6-CDR3 | ARTSSIYYTSSDAEEYDY |
| 174. | 1H4-VHH | QVQLQESGGGLVQAGTSLRLSCAASGRTFSSFMG |
| | | WFRQAPGKEREFVATINRSGGATGYADSVKGRFT |
| | | ISRDNAKNTLYLQMNSLKPEDTAVYYCAAARGSS |
| | | VYYTSTDPEEYGHWGQGTQVTVSS |
| 175. | 1H4-CDR1 | SFMG |
| 176. | 1H4-CDR2 | TINRSGGATGYADSVKG |
| 177. | 1H4-CDR3 | ARGSSVYYTSTDPEEYGH |
| 178. | 1H5-VHH | QVQLQESGGGLVQAGASLRLSCQASGRTFSSFMG |
| | | WFRQAPGKERKFVATINRSGGATGYADSVKGRFT |
| | | ISRDNAKNTLYLQMNSLRPEDTAVYYCAAARTSS |
| | | SYYTSSDPEEYNYWGQGTQVTVSS |
| 179. | 1H5-CDR1 | SFMG |
| 180. | 1H5-CDR2 | TINRSGGATGYADSVKG |
| 181. | 1H5-CDR3 | ARTSSSYYTSSDPEEYNY |
| 182. | 2D8-VHH | QVQLQESGGGLVQAGASLRLSCAASGRTFSSFMG |
| | | WFRQAPGKEREFVATINRNGGATGYSDSVKGRFT |
| | | ISRDNARSTLYLQMNSLKPEDTAVYYCAAARGSS |
| | | SYYTSTDPEEYGHWGQGTLVTVSS |
| 183. | 2D8-CDR1 | SFMG |
| 184. | 2D8-CDR2 | TINRNGGATGYSDSVKG |
| 185. | 2D8-CDR3 | ARGSSSYYTSTDPEEYGH |
| 186. | 2D9-VHH | QVQLQESGGGLVQAGASLRLSCAASGRTFSSFMA |
| | | WFRQAPGKERKFVATINWNGGATGYADSVKGRF |
| | | TISRDNAKNTLYLQMNSLKPEDTAVYYCAAARPS |
| | | SSYYTSTDAEEYNYWGQGTQVTVSS |
| 187. | 2D9-CDR1 | SFMA |
| 188. | 2D9-CDR2 | TINWNGGATGYADSVKG |
| 189. | 2D9-CDR3 | ARPSSSYYTSTDAEEYNY |
| 190. | 2E8-VHH | QVQLQESGGGLVQAGASLRLSCAASGRTSSSFMG |
| | | WFRQAPGKERKFVATINWNGGATGYADSVKGRF |
| | | TISRDNAKNTLYLQMNSLKPEDTAVYYCAAARTS |
| | | SYYYTSTDAEEYNYWGQGTLVTVSS |
| 191. | 2E8-CDR1 | SFMG |
| 192. | 2E8-CDR2 | TINWNGGATGYADSVKG |
| 193. | 2E8-CDR3 | ARTSSYYYTSTDAEEYNY |
| 194. | 2F2-VHH | QVQLQESGGGLVQAGGSLRLSCAASGSIFSINGM |
| | | G |
| | | WYRQAPGKQRELVALITSGGSTHYADSVKGRFTI |
| | | S |
| | | RDNIKNTLYLQMNSLKPEDTAVYYCNGRRKTTG |
| | | WG |
| | | TLREWEGSRDPDDHDYWGQGTQVTVSS |
| 195. | 2F2-CDR1 | INGMG |
| 196. | 2F2-CDR2 | LITSGGSTHYADSVKG |
| 197. | 2F2-CDR3 | NGRRKTTGWGTLREWEGSRDPDDHDY |

The VHHs were sequenced and the CDRs thereof identified (the positioning of CDRs2 and 3 in the variable region of an antibody follows Kabat numbering and the positioning of CDR1 in the variable region of an antibody follows a hybrid of Kabat and Chothia numbering schemes) as summarized in the following Table 5. Some of the sequences listed in Table 5 occur in Table 4 as well.

TABLE 5

| Name | Sequence | CDR1 | CDR2 | CDR3 |
|---|---|---|---|---|
| 1A4 | QVQLQESGGGLVQAGGSVRLSCAASGSIFSINAMGWYRQAPG | SIFS | VITSGG | RRKTTGWG |
| | KQRELVAVITSGGSTHYADSVKGRFTISRDNAKNTLYLQMNS | INAM | STHYAD | SLREWEGS |
| | LKPEDTAVYYCNGRRKTTGWGSLREWEGSRDPDDYDYWGQGT | G (SEQ | SVKG (SEQ | RDPDDYDY |
| | QVTVSS (SEQ ID NO: 198) | ID | ID | (SEQ ID |
| | | NO: 199) | NO: 200) | NO: 201) |
| 1A5 | QVQLQEFGGTLVQPGGSLRLSCAASGRTFSDYGMGWFRQAPG | RTFS | SSSWSG | SSSWNNND |
| | KEREFVASSSWSGRSTYYAAAVRGRFTISRDNAKSTVYLQMN | DYGM | RSTYYA | PSEYEY (SEQ |
| | SLKAEDTAVYYCAASSSWNNNDPSEYEYWGQGTLVTVSS | G (SEQ | AAVRG | ID |
| | (SEQ ID NO: 202) | ID | (SEQ ID | NO: 205) |
| | | NO: 203) | NO: 204) | |
| 1A6 | QVQLQESGGGLVQAGGSLRLSCAASGSIFSINGMSWYRQAPG | SIFS | VITSGG | RRKTTGWG |
| | KQRELVAVITSGGSTHYADSVKGRFTISRDNAKNTLYLQMNS | INGM | STHYAD | TLREWEGS |
| | LKPEDTAVYYCNGRRKTTGWGTLREWEGSRDPDDYDYWGQGT | S (SEQ | SVKG (SEQ | RDPDDYDY |
| | QVTVSS (SEQ ID NO: 206) | ID | ID | (SEQ ID |
| | | NO: 207) | NO: 208) | NO: 209) |
| 1A7 | QVQLQAFGGGLVQAGGSLRLSCAASGSIFSINAMGWYRQAPG | SIFS | VITSGG | RRKTTGWG |
| | KQRELVAVITSGGSTHYADSVKGRFTISRDNTKNTLYLQMNS | INAM | STHYAD | TLREWEGS |
| | LKPEDTAVYYCNGRRKTTGWGTLREWEGSRDPDDYDYWGQGT | G (SEQ | SVKG (SEQ | RDPDDYDY |
| | LVTVSS (SEQ ID NO: 210) | ID | ID | (SEQ ID |
| | | NO: 211) | NO: 212) | NO: 213) |
| 1A8 | QVQLQESGGGLVQAGTSLRLSCAASGRTFSSFMGWFRQAPGK | RTFS | TINRSG | ARGSSVYY |
| | EREFVATINRSGGATGYADSVKGRFTISRDNAKNTLYLQMNS | SFMG | GATGYA | TSSDPEEY |
| | LKPEDTAVYYCAAARGSSVYYTSSDPEEYGHWGQGTLVTVSS | (SEQ | DSVKG | GH (SEQ |
| | (SEQ ID NO: 214) | ID | (SEQ ID | ID |
| | | NO: 215) | NO: 216) | NO: 217) |
| 1A9 | QVQLQASGGGLVQAGGSLRLSCKASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAVSDWVVGGISLMNGAEYKYWGQGTQVTVS | G (SEQ | DSVKG | KY (SEQ |
| | S (SEQ ID NO: 218) | ID | (SEQ ID | ID |
| | | NO: 219) | NO: 220) | NO: 221) |
| 1A10 | QVQLQAFGGGLVQAGGSLGLSCSASGTIFNLDYMGWYRQALG | TIFN | TITSDG | YLSGRSDY |
| | KQRELVATITSDGRTNYADSVKGRFTIFRDGAKNAILMQMNS | LDYM | RTNYAD | (SEQ ID |
| | LKPEDTAVYYCKAYLSGRSDYWGQGTQVTVSS (SEQ ID | G (SEQ | SVKG (SEQ | NO: 225) |
| | NO: 222) | ID | ID | |
| | | NO: 223) | NO: 224) | |
| 1A11 | QVQLQEFGGGLVQAGGSLRLSCVASGRTFSSYGMGWFRQAPG | RTFS | SISWSG | SSTWNNND |
| | KEREFVASISWSGGSTYYPDSVLGRFTISRDNAKNVLYLQMN | SYGM | GSTYYP | PHEYDY (SEQ |
| | SLKPEDTAVYYCAASSTWNNNDPHEYDYWGQGTLVTVSS | G (SEQ | DSVLG | ID |
| | (SEQ ID NO: 226) | ID | (SEQ ID | NO: 229) |
| | | NO: 227) | NO: 228) | |
| 1B1 | QVQLQESGGGLVEGGGSLRLSCAASGRTFSTYAMGWFRQAPG | RTFS | AINWNG | SDWVVGGI |
| | KEREFVAAINWNGASTYYADSVKGRFTISRDNAKNTVYLQMN | TYAM | ASTYYA | SLMNGAEY |
| | SLKSEDTAVYYCAASDWVVGGISLMNGAEYAYWGQGTLVTVS | G (SEQ | DSVKG | AY (SEQ |
| | S (SEQ ID NO: 230) | ID | (SEQ ID | ID |
| | | NO: 231) | NO: 232) | NO: 233) |
| 1B2 | QVQLQEFGGGLVQAGGSLRLSCAASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAASDWVVGGISLMNGAEYAYWGQGTQVTVS | G (SEQ | DSVKG | AY (SEQ |
| | S (SEQ ID NO: 234) | ID | (SEQ ID | ID |
| | | NO: 235) | NO: 236) | NO: 237) |
| 1D2 | QVQLQAFGGGSVQAGGSMKLSCKVDGVSISGNAMRWYRQLPG | VSIS | IVLSNG | QSPQGQY |
| | KERTWAAIVLSNGNEHYANSVKGRFVISRDDAKNTVDLQMNN | GNAM | NEHYAN | (SEQ ID |
| | LKPEDTGTYYCNLQSPQGQYWGQGTQVTVSS (SEQ ID | R (SEQ | SVKG (SEQ | NO: 241) |
| | NO: 238) | ID | ID | |
| | | NO: 239) | NO: 240) | |
| 1B4 | QVQLQESGGGLVQGGGSLRLSCAASGRTVSSDAMGWFRQAPG | RTVS | AINWNG | SDWVVGGI |
| | KEREFVAAINWNGGNTYYADSVKGRFTISRDNAKNTVYLQMN | SDAM | GNTYYA | SLMNGAEY |
| | SLKSEDTAVYYCAVSDWVVGGISLMNGAEYKYWGQGTLVTVS | G (SEQ | DSVKG | KY (SEQ |
| | S (SEQ ID NO: 242) | ID | (SEQ ID | ID |
| | | NO: 243) | NO: 244) | NO: 245) |
| 1B5 | QVQLQESGGGLVQAGGSLRLSCAASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKFEDTAVYYCAASDWVVGGISLMNGAEYKYWGQGTQVTVS | G (SEQ | DSVKG | KY (SEQ |
| | S (SEQ ID NO: 246) | ID | (SEQ ID | ID |
| | | NO: 247) | NO: 248) | NO: 249) |
| 1B6 | QVQLQESGGGLVKGGGSLRLSCVASGGTFSSYAMGWFRQAPG | GTFS | AIGGSG | DPTMFHKL |
| | KEREFVAAIGGSGDSTYYADSVKGRFTISRDNAKNSVYLQMN | SYAM | DSTYYA | YYGINPNE |
| | SLKPEDTAVYYCQADPTMFHKLYYGINPNEYDYWGQGTLVTV | G (SEQ | DSVKG | YDY (SEQ |
| | SS (SEQ ID NO: 250) | ID | (SEQ ID | ID |
| | | NO: 251) | NO: 252) | NO: 253) |
| 1B7 | QVQLQESGGGLVQAGGSLRLSCAASGSIFSINGMGWYRQAPG | SIFS | VITSGG | RRKTTGWG |
| | KQRELVAVITSGGSTHYADSVKGRFTISRDNAKNTLYLQMNS | INGM | STHYAD | TLREWEGS |
| | LKPEDTAVYYCNGRRKTTGWGTLREWEGSRDPDDYDYWGQGT | G (SEQ | SVKG (SEQ | RDPDDYDY |
| | QVTVSS (SEQ ID NO: 254) | ID | ID | (SEQ ID |
| | | NO: 255) | NO: 256) | NO: 257) |
| 1D1 | QVQLQASGGRLVRPGESLTLSCVVSGRTSGTTAMGWFRQARG | RTSG | QISYSD | TRGEGSRN |
| | KERQFLAQISYSDGSTYYAASVKGRFNISRDNAGNTVYLQMD | TTAM | GSTYYA | VN (SEQ |
| | SLTSEDTATYYCAPTRGEGSRNVNWGQGTQVTVSS (SEQ ID | G (SEQ | ASVKG | ID |
| | NO: 258) | ID | (SEQ ID | NO: 261) |
| | | NO: 259) | NO: 260) | |
| 1D3 | QVQLQASGGGLVQAGGSLGLSCSASGTIFNLDYMGWYRQALG | TIFN | TITSDG | YLSGRSDY |
| | KQRELVATITSDGRTNYADSVKGRFTIFRDGAKNAILMQMNS | LDYM | RTNYAD | (SEQ ID |
| | LKPEDTAVYYCKAYLSGRSDYWGQGTLVTVSS (SEQ ID | G (SEQ | SVKG (SEQ | NO: 265) |
| | NO: 262) | ID | ID | |
| | | NO: 263) | NO: 264) | |
| 1D4 | QVQLQESGGGLVQAGGSLRLSCKASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAVSDWVVGGISLMNGAEYKYWGQGTQVTVS | G (SEQ | DSVKG | KY (SEQ |
| | S (SEQ ID NO: 266) | ID | (SEQ ID | ID |
| | | NO: 267) | NO: 268) | NO: 269) |
| 1D6 | QVQLQESGGGSVQAGGSLGLFCSASGTIFNIDVMGWYRQAPG | TIFN | TITSDG | YLSGRSDY |
| | KQRELVATITSDGRTNYADSVKGRFTISRDGAKNAVHVQMNS | IDVM | RTNYAD | (SEQ ID |
| | LKPEDTAVYYCKAYLSGRSDYWGQGTQVTVSS (SEQ ID | G (SEQ | SVKG (SEQ | NO: 273) |
| | NO: 270) | ID | ID | |
| | | NO: 271) | NO: 272) | |
| 1D7 | QVQLQESGGGLVQAGGSLRLSCAASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAASDWVVGGISLMNGAEYAYWGQGTQVTVS | G (SEQ | DSVKG | AY (SEQ |
| | S (SEQ ID NO: 274) | ID | (SEQ ID | ID |
| | | NO: 275) | NO: 276) | NO: 277) |
| 1D8 | QVQLQEFGGGLVQAGGSLRLSCAASGSIFSINGMGWYRQAPG | SIFS | VITSGG | RRKTTGWG |
| | KQRELVAVITSGGSTYYADSVKGRFTISRDNAKNTLYLQMKS | INGM | STYYAD | TLREWEGS |
| | LKPEDTAVYYCNGRRKTTGWGTLREWEGSRDPDDYDYWGQGT | G (SEQ | SVKG (SEQ | RDPDDYDY |
| | LVTVSS (SEQ ID NO: 278) | ID | ID | (SEQ ID |
| | | NO: 279) | NO: 280) | NO: 281) |
| 1D12 | QVQLQESGGGLVKGGGSLRLSCVASGGTFSSYAMGWFRQAPG | GTFS | AIGGSG | DPTMFHKL |
| | KERELVAAIGGSGDSTYYADSVKGRFTISRDNAKNSVYLQMN | SYAM | DSTYYA | YYGINPNE |
| | SLKPEDTAVYYCQADPTMFHKLYYGINPNEYDYWGQGTQVTV | G (SEQ | DSVKG | YDY (SEQ |
| | SS (SEQ ID NO: 282) | ID | (SEQ ID | ID |
| | | NO: 283) | NO: 284) | NO: 285) |
| 1E1 | QVQLQESGGGLVQPGGSLRLSCAASGRTFSSYGMGWFRQAPG | RTFS | SISWSG | SSTWNNND |
| | KEREFVSSISWSGRSTYYTNAVRGRFTISRDNAKNTVYLQMN | SYGM | RSTYYT | PTEYDY (SEQ |
| | SLKPEDTAVYYCAASSTWNNNDPTEYDYWGQGTLVTVSS | G (SEQ | NAVRG | ID |
| | (SEQ ID NO: 286) | ID | (SEQ ID | NO: 289) |
| | | NO: 287) | NO: 288) | |
| 1E2 | QVQLQESGGGLVQAGGSLRLSCAASELTFSTHAMGWFRQAPG | LTFS | AIAWSG | TNGGAWHY |
| | KEREFVAAIAWSGGSTYYADSVKGRFTISRDISKNTIYLQMN | THAM | GSTYYA | HERYFGS |
| | SLKPEDTALYYCAATNGGAWHYHERYFGSWGQGTQVTVSS | G (SEQ | DSVKG | (SEQ ID |
| | (SEQ ID NO: 290) | ID | (SEQ ID | NO: 293) |
| | | NO: 291) | NO: 292) | |
| 1E3 | QVQLQESGGGLVQAGGSLRLSCTASGSISSINAMGWYRQAPG | SISS | AITSGG | RRITTGWG |
| | KQRELVAAITSGGSTHYADSVKGRFTISRDNAKNTMYLQMNS | INAM | STHYAD | TMREWEGS |
| | LKPEDTAVYYCNARRITTGWGTMREWEGSRDPYEYDYWGQGT | G (SEQ | SVKG (SEQ | RDPYEYDY |
| | LVTVSS (SEQ ID NO: 294) | ID | ID | (SEQ ID |
| | | NO: 295) | NO: 296) | NO: 297) |
| 1E4 | QVQLQESGGGLVQPGGSLRLSCAASGGTFSDYGMGWFRQAPG | GTFS | SISWSG | SSTWNNND |
| | KEREFVSSISWSGNSVYYAGAVRGRFTISRDNAKNTVYLQMN | DYGM | NSVYYA | PTEYEY (SEQ |
| | SLQPEDTAVYYCAASSTWNNNDPTEYEYWGQGTLVTVSS | G (SEQ | GAVRG | ID |
| | (SEQ ID NO: 298) | ID | (SEQ ID | NO: 301) |
| | | NO: 299) | NO: 300) | |
| 1E5 | QVQLQEFGGGLVQPGGSLRLSCAASGRTFSDYAMGWFRQAPG | RTFS | SISWSG | SSTWNNND |
| | KEREFVASISWSGGSLYYAGAVRGRFTISRDNAKNTVYLQMN | DYAM | GSLYYA | PHEYDY (SEQ |
| | SLKPEDTAVYYCAASSTWNNNDPHEYDYWGQGTQVTVSS | G (SEQ | GAVRG | ID |
| | (SEQ ID NO: 302) | ID | (SEQ ID | NO: 305) |
| | | NO: 303) | NO: 304) | |
| 1E6 | QVQLQEFGGGLVQAGASLRLSCAASGRTLSRFMGWFRQAPGK | RTLS | TINWNG | ARTSSIYY |
| | ERRFVATINWNGGATGYADSVKGRFTISRDNAKNTLYLQMNS | RFMG | GATGYA | TSSDAEEY |
| | LKPDDTAVYYCAAARTSSIYYTSSDAEEYDYWGQGTQVTVSS | (SEQ | DSVKG | DY (SEQ |
| | (SEQ ID NO: 306) | ID | (SEQ ID | ID |
| | | NO: 307) | NO: 308) | NO: 309) |
| 1E7 | QVQLQQFGGGLVQAGGSLRLSCAASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAASDWVVGGISLMNGAEYAYWGQGTQVTVS | G (SEQ | DSVKG | AY (SEQ |
| | S (SEQ ID NO: 310) | ID | (SEQ ID | ID |
| | | NO: 311) | NO: 312) | NO: 313) |
| 1E8 | QVQLQPSGGGLVQAGGSLRLSCAASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAASDWVVGGISLMNGAEYAYWGQGTQVTVS | G (SEQ | DSVKG | AY (SEQ |
| | S (SEQ ID NO: 314) | ID | (SEQ ID | ID |
| | | NO: 315) | NO: 316) | NO: 317) |
| 1G1 | QVQLQASGGGLVQAGGSLRLSCAASGRTVSSDAMGWFRQAPG | RTVS | AINWNG | SDWVVGGI |
| | KEREFVAAINWNGASTYYGDSAQGRFTISRDNAKNTVYLQMN | SDAM | ASTYYG | SLMNGAEY |
| | SLKSEDTAVYYCAASDWVVGGISLMNGAEYKYWGQGTLVTVS | G (SEQ | DSAQG | KY (SEQ |
| | S (SEQ ID NO: 318) | ID | (SEQ ID | ID |
| | | NO: 319) | NO: 320) | NO: 321) |
| 1G2 | QVQLQAFGGGLVQAGGSLRLSCKASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAVSDWVVGGISLMNGAEYKYWGQGTQVTVS | G (SEQ | DSVKG | KY (SEQ |
| | S (SEQ ID NO: 322) | ID | (SEQ ID | ID |
| | | NO: 323) | NO: 324) | NO: 325) |
| 1G3 | QVQLAESGGGLVQAGGSLRLSCKASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAVSDWVVGGISLMNGAEYEYWGQGTLVTVS | G (SEQ | DSVKG | EY (SEQ |
| | S (SEQ ID NO: 326) | ID | (SEQ ID | ID |
| | | NO: 327) | NO: 328) | NO: 329) |
| 1G4 | QVQLQESGGGLVRPGGSLRLSCAASGGTFSSYAMGWFRQAPG | GTFS | AIGGSG | DPTMFHKL |
| | KEREFVAAIGGSGDSTYYADSVKGRFTISRDNAKNTAYLQMN | SYAM | DSTYYA | YYGIHPNE |
| | SLKPEDTAVYYCQADPTMFHKLYYGIHPNEYEYWGQGTLVTV | G (SEQ | DSVKG | YEY (SEQ |
| | SS (SEQ ID NO: 330) | ID | (SEQ ID | ID |
| | | NO: 331) | NO: 332) | NO: 333) |
| 1G5 | QVQLQEFGGGLVQGGGSLRLSCAASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAASDWVVGGISLMNGAEYAFWGQGTQVTVS | G (SEQ | DSVKG | AF (SEQ |
| | S (SEQ ID NO: 334) | ID | (SEQ ID | ID |
| | | NO: 335) | NO: 336) | NO: 337) |
| 1G6 | QVQLAESGGGLVQAGGSLRLSCKASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAVSDWVVGGISLMNGAEYKYWGQGTQVTVS | G (SEQ | DSVKG | KY (SEQ |
| | S (SEQ ID NO: 338) | ID | (SEQ ID | ID |
| | | NO: 339) | NO: 340) | NO: 341) |
| 1G7 | QVQLQQSGGDLVQPGGSLRLSCAASGRTFSDYGMGWFRQAPG | RTFS | SSSWSG | SSSWNNND |
| | KEREFVASSSWSGRSTYYAAAVRGRFTISRDNAKSTVYLQMN | DYGM | RSTYYA | PSEYEY (SEQ |
| | SLKAEDTAVYYCAASSSWNNNDPSEYEYWGQGTQVTVSS | G (SEQ | AAVRG | ID |
| | (SEQ ID NO: 342) | ID | (SEQ ID | NO: 345) |
| | | NO: 343) | NO: 344) | |
| 1G8 | QVQLQEFGGGLVQAGGSLRLSCKASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAVSDWVVGGISLMNGAEYKYWGQGTQVTVS | G (SEQ | DSVKG | KY (SEQ |
| | S (SEQ ID NO: 346) | ID | (SEQ ID | ID |
| | | NO: 347) | NO: 348) | NO: 349) |
| 1H2 | QVQLQESGGGLVQAGGSLRLSCAASGSIFSINAMGWYRQAPG | SIFS | VITSGG | RRKTTGWG |
| | KQRELVAVITSGGSTHYADSVKGRFTISRDNAKNTLYLQMNS | INAM | STHYAD | TLREWEGS |
| | LKPEDTAVYYCNGRRKTTGWGTLREWEGSRDPDDYDYWGQGT | G (SEQ | SVKG (SEQ | RDPDDYDY |
| | LVTVSS (SEQ ID NO: 350) | ID | ID | (SEQ ID |
| | | NO: 351) | NO: 352) | NO: 353) |
| 1H3 | QVQLQQSGGGLVQAGGSMRLSCAASGRTVSSDAMGWFRQAPG | RTVS | AINWNG | SDWVVGGI |
| | KEREFVAAINWNGANTYYADSVKGRFTISRDNAKNTVYLQMN | SDAM | ANTYYA | SLMNGAEY |
| | SLKSEDTAVYYCAVSDWVVGGISLMNGAEYKYWGQGTQVTVS | G (SEQ | DSVKG | KY (SEQ |
| | S (SEQ ID NO: 354) | ID | (SEQ ID | ID |
| | | NO: 355) | NO: 356) | NO: 357) |
| 1H4 | QVQLQESGGGLVQAGTSLRLSCAASGRTFSSFMGWFRQAPGK | RTFS | TINRSG | ARGSSVYY |
| | EREFVATINRSGGATGYADSVKGRFTISRDNAKNTLYLQMNS | SFMG | GATGYA | TSTDPEEY |
| | LKPEDTAVYYCAAARGSSVYYTSTDPEEYGHWGQGTQVTVSS | (SEQ | DSVKG | GH (SEQ |
| | (SEQ ID NO: 358) | ID | (SEQ ID | ID |
| | | NO: 359) | NO: 360) | NO: 361) |
| 1H5 | QVQLQESGGGLVQAGASLRLSCQASGRTFSSFMGWFRQAPGK | RTFS | TINRSG | ARTSSSYY |
| | ERKFVATINRSGGATGYADSVKGRFTISRDNAKNTLYLQMNS | SFMG | GATGYA | TSSDPEEY |
| | LRPEDTAVYYCAAARTSSSYYTSSDPEEYNYWGQGTQVTVSS | (SEQ | DSVKG | NY (SEQ |
| | (SEQ ID NO: 362) | ID | (SEQ ID | ID |
| | | NO: 363) | NO: 364) | NO: 365) |
| 1H6 | QVQLQAFGGGTVQAGGSLRLSCTASGGTFSDYGMGWFRQAPG | GTFS | SISWSG | SSTWNNND |
| | KEREFVSSISWSGNSVYYAGAVRGRFTISRDNAKNTVYLQMN | DYGM | NSVYYA | PTEYEY (SEQ |
| | SLQPEDTAVYYCAASSTWNNNDPTEYEYWGQGTQVTVSS | G (SEQ | GAVRG | ID |
| | (SEQ ID NO: 366) | ID | (SEQ ID | NO: 369) |
| | | NO: 367) | NO: 368) | |
| 1H7 | QVQLQQFGGGLVQPGGSLRLSCAASGRTFSDYGMGWFRQAPG | RTFS | SSSWSG | SSSWNNND |
| | KEREFVASSSWSGRSTYYAAAVRGRFTISRDNAKSTVYLQMN | DYGM | RSTYYA | PSEYEY (SEQ |
| | SLKAEDTAVYYCAASSSWNNNDPSEYEYWGQGTLVTVSS | G (SEQ | AAVRG | ID |
| | (SEQ ID NO: 370) | ID | (SEQ ID | NO: 373) |
| | | NO: 371) | NO: 372) | |
| 1H8 | QVQLQESGGGLVQAGGSLRLSCKASGRTVSSDAMGWFRQAPG | RTVS | AINWNG | SDWVVGGF |
| | KEREFVAAINWNGASTYYADSVKGRFTISRDNAKNTVYLQMN | SDAM | ASTYYA | SLMNGAEY |
| | SLKSEDTAVYYCAVSDWVVGGFSLMNGAEYKYWGQGTQVTVS | G (SEQ | DSVKG | KY (SEQ |
| | S (SEQ ID NO: 374) | ID | (SEQ ID | ID |
| | | NO: 375) | NO: 376) | NO: 377) |
| 2A4 | QVQLQESGGGLVQAGGSLRLSCAASGRTFSTHSMGRFRQAPG | RTFS | AIAWSG | TNGGACHY |
| | KEREFVAAIAWSGGSTYYADSVKCRFTISRDISKNTIYLQMN | THSM | GSTYYA | HERYYGS |
| | SVKPEDTALYYCAATNGGACHYHERYYGSWGQGTQVTVSS | G (SEQ | DSVKC | (SEQ ID |
| | (SEQ ID NO: 378) | ID | (SEQ ID | NO: 381) |
| | | NQ379:) | NO: 380) | |
| 2B1 | QVQLQEFGGGLVQAGGSLRLSCAASGRTASSDAMGWFRQTPG | RTAS | AINWNG | SNWIVGGI |
| | KEREFVAAINWNGASTYYADSVKGRFTISRDNAKNTVYLQMN | SDAM | ASTYYA | SLMNGAEY |
| | SLKSEDTAVYYCAASNWIVGGISLMNGAEYNFWGQGTQVTVS | G (SEQ | DSVKG | NF (SEQ |
| | S(SEQ ID NO: 382) | ID | (SEQ ID | ID |
| | | NO: 383) | NO: 384) | NO: 385) |
| 2C1 | QVQLQESGGGLVQGGGSLRLSCAASGRTVSSDAMGWFRQAPG | RTVS | AINWNG | SDWVVGGI |
| | KEREFVAAINWNGANTYYADSVKGRFTISRDNAKNTVYLQMN | SDAM | ANTYYA | SLMNGAEY |
| | SLKSEDTAVYYCAVSDWVVGGISLMNGAEYKYWGQGTQVTVS | G (SEQ | DSVKG | KY (SEQ |
| | S (SEQ ID NO: 386) | ID | (SEQ ID | ID |
| | | NO: 387) | NO: 388) | NO: 389) |
| 2C4 | QVQLQAFGGGLVQAGGSLRLSCAASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGVEY |
| | SLKSEDTAVYYCAASDWVVGGISLMNGVEYKYWGQGTQVTVS | G (SEQ | DSVKG | KY (SEQ |
| | S (SEQ ID NO: 390) | ID | (SEQ ID | ID |
| | | NO: 391) | NO: 392) | NO: 393) |
| 2C5 | QVQLQEFGGGLVQAGASLRLSCAASGGTFSYFMGWFRQAPGK | GTFS | TINRNG | ARESSVYY |
| | EREFVATINRNGGATGYADSVKGRFTISRDNAKNTLYLQMDS | YFMG | GATGYA | TSTDPAEY |
| | VTPEDTAVYYCAAARESSVYYTSTDPAEYGYWGQGTQVTVSS | (SEQ | DSVKG | GY (SEQ |
| | (SEQ ID NO: 394) | ID | (SEQ ID | ID |
| | | NO: 395) | NO: 396) | NO: 397) |
| 2C6 | QVQLQEFGGGLVQPGGSLRLSCAASGRTFSDYGMGWFRQAPG | RTFS | SSSWSG | SSSWNNND |
| | KEREFVASSSWSGRSTYYAAAVRGRFTISRDNAKSTVYLQMN | DYGM | RSTYYA | PSEYEY (SEQ |
| | SLKAEDTAVYYCAASSSWNNNDPSEYEYWGQGTQVTVSS | G (SEQ | AAVRG | ID |
| | (SEQ ID NO: 398) | ID | (SEQ ID | NO: 401) |
| | | NO: 399) | NO: 400) | |
| 2C7 | QVQLQAFGGGLVQPGGSLRLSCAASGGTFSDYGMGWFRQAPG | GTFS | SSSWSG | SSTWNNND |
| | KEREFVASSSWSGRSTYYAAAVRGRFTISRDNAKSLVYLQMN | DYGM | RSTYYA | PSEYEY (SEQ |
| | NLKPEDTAVYYCAASSTWNNNDPSEYEYWGQGTQVTVSS | G (SEQ | AAVRG | ID |
| | (SEQ ID NO: 402) | ID | (SEQ ID | NO: 405) |
| | | NO: 403) | NO: 404) | |
| 2D3 | QVQLQASGGGLVQAGGSLRLSCVASGRTFSSYGMGWFRQAPG | RTFS | SISWSG | SSTWNNND |
| | KEREFVASISWSGGSTYYPDSVLGRFTISRDNAKNVLYLQMN | SYGM | GSTYYP | PHEYDY (SEQ |
| | SLKPEDTAVYYCAASSTWNNNDPHEYDYWGQGTLVTVSS | G (SEQ | DSVLG | ID |
| | (SEQ ID NO: 406) | ID | (SEQ ID | NO: 409) |
| | | NO: 407) | NO: 408) | |
| 2D4 | QVQLQESGGGLVEAGGSLRLSCAASGGTFSDYGMGWFRQAPG | GTFS | SISWSG | SSTWNNND |
| | KEREFVSSISWSGNSVYYAGAVRGRFTISRDNAKNTVYLQMN | DYGM | NSVYYA | PTEYEY (SEQ |
| | SLQPEDTAVYYCAASSTWNNNDPTEYEYWGQGTQVTVSS | G (SEQ | GAVRG | ID |
| | (SEQ ID NO: 410) | ID | (SEQ ID | NO: 413) |
| | | NO: 411) | NO: 412) | |
| 2D5 | QVQLQESGGGLVQAGASLRLSCAASGRTFSSFMAWFRQAPGK | RTFS | TINWNG | ARPSSAYY |
| | ERKFVATINWNGGATGYADPVKGRFTISRDNAKNTLYLQMNS | SFMA | GATGYA | TSTDAEEY |
| | LKPEDTAVYYCAAARPSSAYYTSTDAEEYNYWGQGTLVTVSS | (SEQ | DPVKG | NY (SEQ |
| | (SEQ ID NO: 414) | ID | (SEQ ID | ID |
| | | NO: 415) | NO: 416) | NO: 417) |
| 2D6 | QVQLQESGGGLVQAGASLRLSCAASGRTFSSFMGWFRQAPGK | RTFS | TINWNG | ARTGSAYY |
| | ERKFVATINWNGGATGYADSVKGRFTISRDNAKNTLYLQMNS | SFMG | GATGYA | TSSDAGEY |
| | LKPEDTAVYYCAAARTGSAYYTSSDAGEYNYWGQGTQVTVSS | (SEQ | DSVKG | NY (SEQ |
| | (SEQ ID NO: 418) | ID | (SEQ ID | ID |
| | | NO: 419) | NO: 420) | NO: 421) |
| 2D8 | QVQLQESGGGLVQAGASLRLSCAASGRTFSSFMGWFRQAPGK | RTFS | TINRNG | ARGSSSYY |
| | EREFVATINRNGGATGYSDSVKGRFTISRDNARSTLYLQMNS | SFMG | GATGYS | TSTDPEEY |
| | LKPEDTAVYYCAAARGSSSYYTSTDPEEYGHWGQGTLVTVSS | (SEQ | DSVKG | GH (SEQ |
| | (SEQ ID NO: 422) | ID | (SEQ ID | ID |
| | | NO: 423) | NO: 424) | NO: 425) |
| 2D9 | QVQLQESGGGLVQAGASLRLSCAASGRTFSSFMAWFRQAPGK | RTFS | TINWNG | ARPSSSYY |
| | ERKFVATINWNGGATGYADSVKGRFTISRDNAKNTLYLQMNS | SFMA | GATGYA | TSTDAEEY |
| | LKPEDTAVYYCAAARPSSSYYTSTDAEEYNYWGQGTQVTVSS | (SEQ | DSVKG | NY (SEQ |
| | (SEQ ID NO: 426) | ID | (SEQ ID | ID |
| | | NO: 427) | NO: 428) | NO: 429) |
| 2D10 | QVQLQESGGGLVQPGGSLRLSCAASGRTFSDYGMGWFRQAPG | RTFS | SSSWSG | SSSWNNND |
| | KEREFVASSSWSGRSTYYAAAVRGRFTISRDNAKSTVYLQMN | DYGM | RSTYYA | PSEYEY (SEQ |
| | SLKAEDTAVYYCAASSSWNNNDPSEYEYWGQGTLVTVSS | G (SEQ | AAVRG | ID |
| | (SEQ ID NO: 430) | ID | (SEQ ID | NO: 433) |
| | | NO: 431) | NO: 432) | |
| 2E1 | QVQLQASGGGLVQPGGSLRLSCAASGRTFSSYGMGWFRQAPG | RTFS | SISWSG | SSTWNNND |
| | KEREFVASISWSGRSTYYTNAVRGRFTISRDNAKNTVYLQMN | SYGM | RSTYYT | PTEYEY (SEQ |
| | SLKPEDTAVYYCAASSTWNNNDPTEYEYWGQGTQVTVSS | G (SEQ | NAVRG | ID |
| | (SEQ ID NO: 434) | ID | (SEQ ID | NO: 437) |
| | | NO: 435) | NO: 436) | |
| 2E2 | QVQLQASGGGLVQPGGSLRLSCAASGRTFSSYGMGWFRQAPG | RTFS | SISWSG | SSTWNNND |
| | KEREFVASISWSGRSTYYTNAVRGRFTISRDNAKNTVYLQMN | SYGM | RSTYYT | PTEYEF (SEQ |
| | SLKPEDTAVYYCAASSTWNNNDPTEYEFWGQGTQVTVSS | G (SEQ | NAVRG | ID |
| | (SEQ ID 438:) | ID | (SEQ ID | NO: 441) |
| | | NO: 439) | NO: 440) | |
| 2E3 | QVQLQESGGGLVQAGGSLRLSCAASGSIFSINAMGWYRQAPG | SIFS | AITSGG | RRITTGWG |
| | KQRELVAAITSGGNTYYVDSVKGRFSISRDNAMNTVDLQMNS | INAM | NTYYVD | TPREWEGS |
| | LKPEDTAVYYCNARRITTGWGTPREWEGSRDPNDNVFWGQGT | G (SEQ | SVKG (SEQ | RDPNDNVF |
| | QVTVSS (SEQ ID NO: 442) | ID | ID | (SEQ ID |
| | | NO: 443) | NO: 444) | NO: 445) |
| 2E4 | QVQLQASGGGLVQGGGSLRLSCKASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAVSDWVVGGISLMNGAEYKYWGQGTLVTVS | G (SEQ | DSVKG | KY (SEQ |
| | S (SEQ ID NO: 446) | ID | (SEQ ID | ID |
| | | NO: 447) | NO: 448) | NO: 449) |
| 2E5 | QVQLQESGGGLVQPGGSLRLSCAASGRTFSSYGMGWFRQAPG | RTFS | SISWSG | SSTWNNND |
| | KEREFVSSISWSGRSTYYTNAVRGRFTISRDNAKNTVYLQMN | SYGM | RSTYYT | PTEYEY (SEQ |
| | SLKPEDTAVYYCAASSTWNNNDPTEYEYWGQGTQVTVSS | G (SEQ | NAVRG | ID |
| | (SEQ ID NO: 450) | ID | (SEQ ID | NO: 453) |
| | | NO: 451) | NO: 452) | |
| 2E7 | QVQLQESGGGLVQAGASLRLSCAASGRTFSSFMAWFRQAPGK | RTFS | NINWNG | ARPSSRYY |
| | ERELVANINWNGGATGYADSVKGRFTISRDNAKNTLYIQMNS | SFMA | GATGYA | TSNDAEEY |
| | LKPEDTAVYYCAAARPSSRYYTSNDAEEYNYWGQGTQVTVSS | (SEQ | DSVKG | NY (SEQ |
| | (SEQ ID NO: 454) | ID | (SEQ ID | ID |
| | | NO: 455) | NO: 456) | NO: 457) |
| 2E8 | QVQLQESGGGLVQAGASLRLSCAASGRTSSSFMGWFRQAPGK | RTSS | TINWNG | ARTSSYYY |
| | ERKFVATINWNGGATGYADSVKGRFTISRDNAKNTLYLQMNS | SFMG | GATGYA | TSTDAEEY |
| | LKPEDTAVYYCAAARTSSYYYTSTDAEEYNYWGQGTLVTVSS | (SEQ | DSVKG | NY (SEQ |
| | (SEQ ID NO: 458) | ID | (SEQ ID | ID |
| | | NO: 459) | NO: 460) | NO: 461) |
| 2F1 | QVQLQAYGGGLVQAGGSLRLSCKASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKYEDTAVYYCAVSDWVVGGISLMNGAEYKYWGQGTLVTVS | G (SEQ | DSVKG | KY (SEQ |
| | S (SEQ ID NO: 462) | ID | (SEQ ID | ID |
| | | NO: 463) | NO: 464) | NO: 465) |
| 2F2 | QVQLQESGGGLVQAGGSLRLSCAASGSIFSINGMGWYRQAPG | SIFS | LITSGG | RRKTTGWG |
| | KQRELVALITSGGSTHYADSVKGRFTISRDNIKNTLYLQMNS | INGM | STHYAD | TLREWEGS |
| | LKPEDTAVYYCNGRRKTTGWGTLREWEGSRDPDDHDYWGQGT | G (SEQ | SVKG (SEQ | RDPDDHDY |
| | QVTVSS (SEQ ID NO: 466) | ID | ID | (SEQ ID |
| | | NO: 467) | NO: 468) | NO: 469) |
| 2F3 | QVQLQASGGGLVQGGGSMRLSCKASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAVSDWVVGGISLMNGAEYKYWGQGTLVTVS | G (SEQ | DSVKG | KY (SEQ |
| | S (SEQ ID NO: 470) | ID | (SEQ ID | ID |
| | | NO: 471) | NO: 472) | NO: 473) |
| 2F4 | QVQLQESGGGLVKGGGSLRLSCVASGGTFSSYAMGWFRQAPG | GTFS | AIGGSG | ARESSVYY |
| | KEREFVAAIGGSGDSTYYADSVKGRFTISRDNAKNSVYLQMN | SYAM | DSTYYA | TSTDPEEY |
| | NLKPEDTAIYYCAAARESSVYYTSTDPEEYGYWGQGTLVTVS | G (SEQ | DSVKG | GY (SEQ |
| | S (SEQ ID NO: 474) | ID | (SEQ ID | ID |
| | | NO: 475) | NO: 476) | NO: 477) |
| 2F5 | QVQLQESGGDLVQRGGSLRLSCKASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAVSDWVVGGISLMNGAEYKYWGQGTQVTVS | G (SEQ | DSVKG | KY (SEQ |
| | S (SEQ ID NO: 478) | ID | (SEQ ID | ID |
| | | NO: 479) | NO: 480) | NO: 481) |
| 2F6 | QVQLQESGGGLVETGGSLSLSCAASGRTFSDYGMGWFRQAPG | RTFS | SSSWSG | SSTWNNND |
| | KEREFVASSSWSGRSTYYAAAVRGRFTVSRDNAKSTVYLQMN | DYGM | RSTYYA | PSEYEY (SEQ |
| | SLKPEDTAVYYCAASSTWNNNDPSEYEYWGQGTQVTVSS | G (SEQ | AAVRG | ID |
| | (SEQ ID NO: 482) | ID | (SEQ ID | NO: 485) |
| | | NO: 483) | NO: 484) | |
| 2F7 | QVQLQESGGGLVKGGGSLRLSCVASGGTFSSYAMGWFRQAPG | GTFS | AIGGSG | DPTMFHKL |
| | KEREFVAAIGGSGDSTYYADSVKGRFTISRDNAKNSVYLQMN | SYAM | DSTYYA | YYGINPNE |
| | SLKPEDTAVYYCQADPTMFHKLYYGINPNEYDYWGQGTQVTV | G (SEQ | DSVKG | YDY (SEQ |
| | SS (SEQ ID NO: 486) | ID | (SEQ ID | ID |
| | | NO: 487) | NO: 488) | NO: 489) |
| 2F8 | QVQLQQFGGGLVQAGGSLRLSCAASGRTFSGYAMGWFRQAPG | RTFS | AINWSG | SDWVVGGI |
| | KEREFVAAINWSGGSTYYADSVKGRYTISRDNAKNTGYLQMN | GYAM | GSTYYA | SLMNGAEY |
| | SLKSEDTAVYYCAASDWVVGGISLMNGAEYKYWGQGTLVTVS | G (SEQ | DSVKG | KY (SEQ |
| | S (SEQ ID NO: 490) | ID | (SEQ ID | ID |
| | | NO: 491) | NO: 492) | NO: 493) |
| 2G2 | QVQLQESGGGLVQAGGSLGLSCSASGTIFNLDYMGWYRQALG | TIFN | TITSDG | YLSGRSDY |
| | KQRELVATITSDGRTNYADSVKGRFTIFRDGAKNAILMQMNS | LDYM | RTNYAD | (SEQ ID |
| | LKPEDTAVYYCKAYLSGRSDYWGQGTQVTVSS (SEQ ID | G (SEQ | SVKG (SEQ | NO: 497) |
| | NO: 494) | ID | ID | |
| | | NO: 495) | NO: 496) | |
| 2G4 | QVQLQEFGGGLVQAGGSLRLSCAASGSISRINAMGWYRQAPG | SISR | VITSGG | RRKTTGWG |
| | KQRELVAVITSGGSTHYADSVKGRFTISRDNAKNTLYLQMNS | INAM | STHYAD | SLREWEGS |
| | LKPEDTAVYYCNGRRKTTGWGSLREWEGSRDPDEYNYWGQGT | G (SEQ | SVKG (SEQ | RDPDEYNY |
| | LVTVSS (SEQ ID NO: 498) | ID | ID | (SEQ ID |
| | | NO: 499) | NO: 500) | NO: 501) |
| 2H1 | QVQLQASGGGLAQGGGSLRLSCKASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNGAEY |
| | SLKSEDTAVYYCAVSDWVVGGISLMNGAEYKYWGQGTLVTVS | G (SEQ | DSVKG | KY (SEQ |
| | S (SEQ ID NO: 502) | ID | (SEQ ID | ID |
| | | NO: 503) | NO: 504) | NO: 505) |
| 2H5 | QVQLQESGGGLVQTGGSLTLSCVASGGTFSSYAMGWFRQAPG | GTFS | AIGGSG | DPTMFHKL |
| | KEREFVAAIGGSGDSTYYADSVKGRFTISRDNAKNSVYLQMN | SYAM | DSTYYA | YYGINPNE |
| | SLKPEDTAVYYCQADPTMFHKLYYGINPNEYDYWGQGTLVTV | G (SEQ | DSVKG | YDY (SEQ |
| | SS (SEQ ID NO: 506) | ID | (SEQ ID | ID |
| | | NO: 507) | NO: 508) | NO: 509) |
| 2H6 | QVQLQESGGGLVQAGGSLRLSCAASGRTFSTHAMGWFRQAPG | RTFS | AIAWSG | TNGGAWHY |
| | KEREFVAAIAWSGGSTYYADSVKGRFTISRDISKNTIYLQMN | THAM | GSTYYA | HERYFGS |
| | SLKPEDTALYYCAATNGGAWHYHERYFGSWGQGTQVTVSS | G (SEQ | DSVKG | (SEQ ID |
| | (SEQ ID NO: 510) | ID | (SEQ ID | NO: 513) |
| | | NO: 511) | NO: 512) | |
| 2H7 | QVQLQESGGGLVQAGGSLRLSCAASGRTFSTHAMGWFRQAPG | RTFS | AIAWSG | TNGGAWHY |
| | KEREFVAAIAWSGGSTYYADSVKGRFTISRDISKNTIYLQMN | THAM | GSTYYA | HERYFGS |
| | SLKPEDTALYYCAATNGGAWHYHERYFGSWGQGTLVTVSS | G (SEQ | DSVKG | (SEQ ID |
| | (SEQ ID NO: 514) | ID | (SEQ ID | NO: 517) |
| | | NO: 515) | NO: 516) | |
| 2H8 | QVQLQESGGGLVQAGGSLRLSCVASGRTFSSYGMGWFRQAPG | RTFS | SISWSG | SSTWNNND |
| | KEREFVASISWSGGSTYYPDSVLGRFTISRDNAKNVLYLQMN | SYGM | GSTYYP | PHEYDY (SEQ |
| | SLKPEDTAVYYCAASSTWNNNDPHEYDYWGQGTLVTVSS | G (SEQ | DSVLG | ID |
| | (SEQ ID NO: 518) | ID | (SEQ ID | NO: 521) |
| | | NO: 519) | NO: 520) | |
| 2H11 | QVQLQASGGGLVQGGGSMRLSCAASGRTVSSDAMGWFRQAPG | RTVS | AINWNG | SDWVVGGI |
| | KEREFVAAINWNGASTYYADSVKGRFTISRDNAKNTVYLQMN | SDAM | ASTYYA | SLMNAAEY |
| | SLKSEDTAVYYCAASDWVVGGISLMNAAEYKYWGQGTQVTVS | G (SEQ | DSVKG | KY (SEQ |
| | S (SEQ ID NO: 522) | ID | (SEQ ID | ID |
| | | NO: 523) | NO: 524) | NO: 525) |

Nucleic acids encoding the VHH proteins described herein are provided. Exemplary sequences encoding the amino acid sequences in Table 5 are depicted in Table 6 below.

TABLE 6

| Name | Sequence |
|---|---|
| 1A4 | CAGGTGCAGTTGCAAGAGTCTGGTGGCGGTCTGGTGCAGGCTGGTGGGTCCGTGAGGCTGAGCTG |
| | TGCCGCATCAGGTAGCATCTTTAGCATCAATGCAATGGGCTGGTATCGCCAGGCTCCAGGGAAGC |
| | AGCGCGAACTCGTCGCCGTTATTACCTCCGGCGGTAGCACGCACTATGCTGATAGCGTGAAGGGC |
| | CGGTTTACTATTTCCCGTGACAATGCCAAAAACACGCTGTACCTGCAAATGAACAGCTTGAAGCC |
| | CGAGGACACAGCTGTCTATTACTGTAACGGTAGACGCAAGACTACCGGATGGGGCTCCCTGCGTG |
| | AGTGGGAAGGGAGTCGTGACCCTGACGATTACGACTATTGGGGCCAGGGCACTCAGGTCACCGTG |
| | AGTAGT (SEQ ID NO: 526) |
| 1A5 | CAAGTGCAGCTCCAGGAGTTTGGTGGAACCCTGGTCCAGCCAGGGGGAAGTCTGCGCCTCAGCTG |
| | CGCTGCCTCTGGCCGTACATTTTCCGACTATGGAATGGGCTGGTTCCGTCAGGCACCCGGAAAGG |
| | AACGTGAGTTCGTGGCGTCCAGCTCCTGGTCTGGACGTAGCACATATTACGCAGCTGCCGTGCGC |
| | GGGAGATTTACAATCTCCCGCGACAACGCTAAATCCACCGTGTACTTGCAGATGAACTCACTGAA |
| | AGCCGAGGATACAGCTGTGTATTACTGCGCAGCTTCCAGCTCCTGGAACAATAACGACCCATCCG |
| | AGTACGAATACTGGGGCCAGGGTACACTGGTTACCGTATCTTCC (SEQ ID NO: 527) |
| 1A6 | CAGGTCCAGTTGCAGGAATCAGGGGGCGGACTGGTCCAGGCAGGGGGCTCCCTGCGCCTTAGCTG |
| | CGCGGCTTCCGGCTCTATCTTCAGCATCAACGGTATGTCTTGGTATCGCCAGGCTCCAGGAAAGC |
| | AGCGCGAGCTGGTGGCCGTCATCACCTCAGGTGGCAGTACCCACTACGCAGACTCTGTCAAGGGT |
| | CGCTTCACCATCTCCCGCGACAACGCGAAAAACACACTGTACCTCCAGATGAACTCCCTGAAACC |
| | CGAAGATACAGCCGTGTATTACTGTAACGGTCGTCGGAAGACGACCGGATGGGGCACGTTGCGCG |
| | AGTGGGAAGGCTCTCGCGACCCGGACGATTATGACTACTGGGGCCAGGGCACCCAAGTGACGGTC |
| | TCCTCC (SEQ ID NO: 528) |
| 1A7 | CAGGTGCAGCTCCAGGCATTTGGTGGCGGGCTGGTCCAGGCTGGAGGTTCCTTGCGCCTCTCATG |
| | CGCAGCCTCTGGCAGCATCTTTAGCATCAACGCTATGGGCTGGTATCGCCAGGCACCGGGAAAGC |
| | AGAGAGAGCTGGTTGCTGTCATTACTTCCGGTGGCAGCACCCACTACGCCGATAGCGTGAAGGGC |
| | AGGTTTACCATCAGCCGCGATAACACCAAGAACACTCTGTATCTGCAAATGAACTCCCTGAAGCC |
| | TGAGGATACCGCCGTGTATTACTGCAATGGCAGGCGCAAAACCACTGGTTGGGGCACTCTGAGAG |
| | AATGGGAGGGCTCTAGGGACCCTGATGACTATGACTACTGGGGACAGGGCACCCTCGTCACAGTT |
| | TCTTCT (SEQ ID NO: 529) |
| 1A8 | CAGGTTCAACTGCAAGAATCTGGAGGCGGTCTGGTCCAGGCGGGCACATCCCTGCGCCTTTCCTG |
| | TGCTGCAAGCGGGCGGACATTCTCTAGCTTCATGGGCTGGTTCCGTCAAGCTCCTGGCAAAGAGC |
| | GCGAGTTCGTCGCGACGATTAACCGCAGCGGTGGGGCGACAGGCTACGCCGATTCCGTGAAAGGT |
| | AGATTCACTATTAGCCGCGATAACGCCAAGAACACTCTGTATCTCCAGATGAACTCCCTCAAACC |
| | TGAGGATACCGCTGTGTACTATTGCGCTGCGGCACGTGGCAGCTCCGTATATTACACCAGCTCCG |
| | ACCCCGAAGAGTATGGTCATTGGGGCCAGGGCACGCTGGTGACAGTGTCCTCT (SEQ ID |
| | NO: 530) |
| 1A9 | CAGGTTCAGTTGCAGGCCAGTGGCGGGGGCTTGGTGCAGGCTGGCGGTTCTCTGCGCCTGAGCTG |
| | CAAAGCGAGCGGTAGGACGGTCTCTTCAGACGCTATGGGATGGTTTCGTCAGGCCCCAGGTAAGG |
| | AGCGTGAGTTCGTCGCTGCCATAAATTGGAATGGAGCGTCTACCTACTATGCCGACTCCGTCAAA |
| | GGGCGGTTTACTATTTCTCGCGATAACGCCAAGAATACGGTGTACCTCCAGATGAACAGCCTGAA |
| | ATCTGAGGACACCGCCGTTTATTACTGTGCCGTTTCCGATTGGGTGGTCGGTGGCATTAGTCTGA |
| | TGAATGGTGCCGAGTACAAGTATTGGGGTCAAGGTACTCAAGTCACAGTGAGTTCC (SEQ ID |
| | NO: 531) |
| 1A10 | CAAGTGCAGCTCCAGGCATTCGGCGGAGGCCTGGTGCAAGCAGGAGGCAGCCTGGGTCTGAGCTG |
| | CTCTGCCAGCGGCACTATCTTTAACCTTGACTACATGGGGTGGTATCGCCAGGCCCTTGGCAAGC |
| | AGCGCGAACTGGTTGCCACAATTACCTCTGATGGCCGCACTAACTACGCCGACTCCGTTAAGGGC |
| | CGTTTCACCATTTTTCGTGATGGCGCTAAGAACGCCATTTTGATGCAGATGAATAGTCTGAAGCC |
| | AGAGGATACCGCTGTGTATTACTGCAAGGCGTACCTGTCCGGGCGCAGCGATTATTGGGGTCAGG |
| | GAACCCAGGTCACCGTGAGCAGC (SEQ ID NO: 532) |
| 1A11 | CAGGTCCAGTTGCAAGAGTTCGGAGGTGGCTTGGTTCAGGCCGGTGGCTCCTTGCGCCTGTCCTG |
| | CGTGGCTTCTGGCCGCACTTTCAGTTCATACGGTATGGGCTGGTTTAGACAAGCGCCGGGTAAGG |
| | AGCGCGAGTTCGTGGCATCTATCTCTTGGAGCGGAGGTTCCACATATTACCCGGATAGTGTTCTG |
| | GGGCGCTTCACTATCTCTAGGGATAACGCGAAGAATGTGCTTTATCTTCAGATGAATAGCCTGAA |
| | ACCAGAAGACACGGCGGTTTATTACTGCGCTGCAAGCAGTACCTGGAACAATAACGATCCCCACG |
| | AGTACGACTATTGGGGTCAAGGCACCCTCGTGACTGTGTCCAGC (SEQ ID NO: 533) |
| 1B1 | CAGGTGCAGCTCCAGGAGAGTGGTGGAGGCCTGGTTGAGGGAGGCGGATCTTTGCGGCTGTCATG |
| | CGCCGCATCTGGGCGCACGTTCTCCACCTATGCTATGGGATGGTTCAGACAAGCGCCTGGTAAGG |
| | AGAGGGAGTTCGTGGCCGCTATTAACTGGAACGGGGCCAGCACTTATTACGCTGACTCCGTCAAG |
| | GGACGGTTCACCATCTCCCGCGACAACGCTAAGAATACTGTGTATCTCCAGATGAACAGCCTCAA |
| | GAGCGAGGATACTGCCGTTTATTACTGTGCGGCCTCAGATTGGGTCGTGGGAGGTATCAGCTTGA |
| | TGAACGGTGCTGAATACGCCTACTGGGGTCAGGGAACCCTGGTTACTGTGTCCAGT (SEQ ID |
| | NO: 534) |
| 1B2 | CAGGTCCAGCTCCAGGAATTTGGGGGTGGCCTGGTGCAGGCCGGAGGTTCCCTCCGGCTGTCCTG |
| | CGCTGCCAGCGGACGCACCGTCAGCTCTGACGCAATGGGCTGGTTCAGACAGGCCCCCGGTAAGG |
| | AGCGCGAGTTCGTCGCCGCTATCAACTGGAATGGTGCCTCTACCTATTACGCTGACTCTGTTAAG |
| | GGCCGTTTCACCATCTCTCGTGATAACGCCAAGAATACTGTGTATCTCCAGATGAATAGTCTCAA |
| | GTCCGAAGACACCGCCGTTTATTACTGTGCGGCTTCTGACTGGGTGGTCGGAGGCATCTCCCTGA |
| | TGAATGGAGCCGAATACGCTTATTGGGGGCAGGGCACACAGGTAACTGTCTCTTCC (SEQ ID |
| | NO: 535) |
| 1B4 | CAAGTGCAGCTCCAAGAGAGTGGCGGAGGCTTGGTGCAAGGTGGGGGCTCCTTGAGATTGTCTTG |
| | TGCGGCTTCCGGTAGAACCGTCAGCTCCGATGCTATGGGCTGGTTCCGCCAGGCTCCTGGAAAGG |
| | AAAGAGAGTTCGTGGCGGCTATTAACTGGAACGGGGGAAACACCTATTACGCAGACTCCGTAAAA |
| | GGCCGCTTCACAATCAGCCGCGACAACGCTAAGAACACTGTTTATCTTCAGATGAACTCCCTCAA |
| | GTCCGAAGATACGGCAGTCTATTACTGTGCTGTCTCTGACTGGGTTGTAGGCGGGATTTCCCTGA |
| | TGAACGGTGCAGAGTACAAGTACTGGGGTCAGGGCACCCTCGTGACTGTCTCAAGC (SEQ ID |
| | NO: 536) |
| 1B5 | CAGGTGCAGCTCCAGGAAAGTGGCGGTGGGCTGGTACAGGCTGGCGGGTCTTTGCGTCTGAGCTG |
| | CGCTGCGAGTGGTCGCACCGTCTCTTCCGACGCTATGGGGTGGTTTCGCCAGGCTCCCGGTAAGG |
| | AGCGCGAATTTGTGGCTGCCATTAACTGGAATGGTGCAAGCACCTACTATGCGGACTCTGTTAAG |
| | GGACGTTTTACCATCAGTAGAGATAACGCCAAAAACACGGTTTACCTCCAGATGAACTCTCTCAA |
| | ATTCGAGGATACTGCGGTGTACTATTGTGCGGCCAGTGACTGGGTTGTGGGCGGTATTTCCTTGA |
| | TGAACGGTGCCGAGTACAAGTACTGGGGCCAGGGTACACAAGTGACAGTTTCCAGC (SEQ ID |
| | NO: 537) |
| 1B6 | CAGGTTCAGCTCCAGGAGAGCGGCGGTGGCCTGGTGAAAGGTGGAGGCTCCCTCCGGTTGAGCTG |
| | CGTGGCTTCCGGGGGCACATTCAGCTCTTACGCTATGGGCTGGTTTCGTCAGGCCCCCGGTAAGG |
| | AGCGCGAGTTCGTGGCTGCCATCGGAGGCTCAGGCGACTCTACTTATTACGCTGACTCAGTGAAG |
| | GGTAGGTTCACCATCTCCCGCGATAACGCCAAGAACTCTGTGTACCTTCAGATGAACTCTCTGAA |
| | ACCCGAGGACACGGCAGTCTATTACTGCCAGGCAGATCCCACAATGTTCCACAAGCTGTATTACG |
| | GCATCAATCCAAACGAATACGACTATTGGGGACAGGGTACGCTGGTGACGGTCTCCTCT (SEQ |
| | ID NO: 538) |
| 1B7 | CAGGTGCAGCTCCAGGAATCCGGTGGGGGCCTGGTACAGGCAGGCGGGTCACTCAGACTCTCTTG |
| | CGCGGCCAGCGGCTCCATCTTTAGCATCAACGGCATGGGCTGGTACAGACAGGCTCCCGGCAAGC |
| | AGCGCGAGTTGGTGGCTGTCATCACTTCTGGTGGCAGTACCCACTACGCGGACTCCGTGAAGGGT |
| | CGCTTCACCATCTCTCGCGACAATGCCAAGAACACTCTCTACCTGCAAATGAACTCCCTGAAGCC |
| | AGAAGACACCGCCGTTTATTACTGCAACGGGAGACGCAAGACAACGGGATGGGGCACCCTCCGTG |
| | AGTGGGAGGGCAGCCGCGACCCAGATGACTATGACTACTGGGGGCAGGGAACCCAGGTGACAGTA |
| | TCATCC (SEQ ID NO: 539) |
| 1D1 | CAGGTGCAACTTCAGGCCAGCGGCGGTCGCCTGGTGAGGCCAGGCGAGAGTCTCACCCTGAGCTG |
| | CGTCGTTTCAGGCCGCACGTCAGGTACGACCGCGATGGGATGGTTTCGCCAGGCTCGTGGCAAGG |
| | AAAGGCAGTTCCTCGCGCAAATCTCCTACTCCGACGGCTCCACTTATTACGCCGCGAGCGTTAAG |
| | GGCCGCTTCAACATTAGCCGTGACAATGCGGGCAACACTGTGTATTTGCAGATGGACTCCCTGAC |
| | CAGTGAGGACACTGCCACCTACTATTGCGCTCCCACTCGCGGAGAAGGTAGTCGCAACGTCAACT |
| | GGGGACAGGGGACACAGGTCACAGTATCCAGC (SEQ ID NO: 540) |
| 1D2 | CAGGTCCAGCTCCAGGCTTTTGGCGGGGGTTCCGTCCAGGCCGGAGGTAGCATGAAGCTCAGCTG |
| | TAAGGTTGACGGGGTGAGTATCTCAGGAAACGCCATGCGTTGGTATCGCCAGCTTCCAGGTAAGG |
| | AACGTACTTGGGCCGCTATCGTGCTCTCCAATGGTAATGAGCACTACGCCAACAGCGTGAAGGGC |
| | CGGTTTGTCATCTCCCGCGATGACGCTAAGAACACCGTGGACCTCCAGATGAACAATCTGAAGCC |
| | TGAAGATACAGGAACTTACTATTGCAACCTCCAGTCCCCTCAGGGGCAATACTGGGGCCAGGGCA |
| | CCCAGGTGACAGTTTCTTCT (SEQ ID NO: 541) |
| 1D3 | CAGGTTCAGCTCCAGGCCAGTGGGGGCGGACTGGTCCAAGCCGGTGGCTCACTGGGACTGTCCTG |
| | TTCCGCGTCAGGGACCATTTTCAATCTGGACTACATGGGCTGGTATAGGCAGGCCCTGGGAAAGC |
| | AGCGCGAGCTGGTCGCCACCATTACTTCCGATGGCCGCACTAATTACGCTGACAGCGTGAAGGGC |
| | AGGTTCACCATCTTCAGAGACGGAGCCAAAAACGCCATTCTGATGCAGATGAACAGCCTGAAGCC |
| | AGAGGATACTGCCGTCTATTACTGTAAAGCCTACCTTTCTGGCCGCTCTGACTACTGGGGGCAGG |
| | GCACCCTCGTGACCGTATCCAGT (SEQ ID NO: 542) |
| 1D4 | CAAGTGCAGCTCCAGGAGAGCGGCGGGGGACTGGTGCAGGCGGGCGGGAGCCTGCGCTTGTCCTG |
| | CAAGGCCTCTGGCCGCACCGTAAGCTCCGATGCTATGGGATGGTTTCGTCAGGCTCCGGGTAAGG |
| | AGCGGGAGTTTGTTGCTGCGATCAACTGGAATGGGGCCAGCACTTATTACGCTGATTCTGTGAAG |
| | GGAAGATTCACAATCAGCAGAGATAACGCTAAGAACACCGTGTACCTTCAGATGAACTCTCTGAA |
| | GAGCGAGGACACCGCCGTGTACTATTGTGCCGTCTCTGATTGGGTAGTTGGAGGCATCAGCCTGA |
| | TGAATGGTGCCGAGTACAAATATTGGGGGCAGGGCACCCAAGTGACCGTGTCATCC (SEQ ID |
| | NO: 543) |
| 1D6 | CAAGTGCAACTGCAAGAGAGCGGCGGTGGCTCTGTGCAGGCCGGTGGCTCCCTCGGACTCTTCTG |
| | CTCCGCCTCCGGGACTATCTTCAATATTGATGTGATGGGCTGGTATCGCCAGGCCCCTGGGAAGC |
| | AACGCGAGCTGGTTGCGACCATTACTTCCGACGGACGCACCAACTACGCCGACTCAGTTAAGGGG |
| | CGCTTTACTATCTCTCGCGACGGGGCTAAAAATGCAGTCCACGTCCAGATGAATAGTCTGAAGCC |
| | GGAGGACACGGCTGTGTATTACTGTAAGGCTTACCTGAGCGGGCGTAGCGACTATTGGGGGCAGG |
| | GAACCCAGGTTACAGTATCCAGC (SEQ ID NO: 544) |
| 1D7 | CAGGTCCAACTGCAAGAGTCTGGGGGAGGCCTCGTGCAGGCTGGGGGTTCTCTGCGGCTGTCCTG |
| | CGCGGCTAGTGGCCGCACAGTGTCTAGCGACGCGATGGGATGGTTCCGCCAGGCTCCAGGGAAGG |
| | AGCGCGAGTTTGTGGCTGCCATCAACTGGAACGGTGCCAGTACCTATTACGCAGACAGCGTGAAG |
| | GGCCGTTTTACCATTTCCCGCGATAACGCCAAGAACACCGTGTACTTGCAGATGAACTCCCTGAA |
| | GTCCGAGGACACTGCTGTCTATTACTGTGCAGCGAGTGATTGGGTGGTCGGTGGCATCTCCCTGA |
| | TGAACGGTGCTGAATACGCTTACTGGGGCCAGGGCACCCAGGTGACCGTGTCTAGC (SEQ ID |
| | NO: 545) |
| 1D8 | CAGGTCCAGTTGCAGGAGTTTGGCGGGGGCCTGGTGCAGGCGGGCGGATCTCTGCGCCTCAGCTG |
| | TGCCGCGTCCGGCTCCATCTTCTCTATCAATGGCATGGGCTGGTATCGGCAGGCCCCTGGAAAGC |
| | AGCGCGAACTCGTGGCTGTTATCACCAGTGGAGGCTCCACATATTACGCAGATTCCGTGAAAGGG |
| | CGCTTCACGATCTCCCGCGACAACGCCAAGAACACCCTGTACCTCCAGATGAAGAGCTTGAAGCC |
| | GGAGGACACCGCCGTGTACTATTGCAACGGTAGACGGAAAACCACAGGTTGGGGTACTCTCCGCG |
| | AGTGGGAGGGCAGCAGAGACCCAGATGACTACGATTATTGGGGCCAGGGGACGCTGGTCACCGTC |
| | TCTAGC (SEQ ID NO: 546) |
| 1D12 | CAGGTCCAGCTCCAGGAATCTGGGGGCGGTCTGGTCAAGGGCGGTGGGTCCCTCCGGCTCTCATG |
| | TGTGGCCAGCGGAGGCACCTTCTCCTCATACGCTATGGGCTGGTTTAGGCAGGCACCTGGCAAGG |
| | AGAGGGAGCTGGTGGCTGCCATTGGAGGCTCCGGCGACTCTACATACTATGCCGACAGCGTCAAG |
| | GGGCGCTTTACGATTAGCCGCGACAACGCCAAAAACAGCGTGTACCTGCAAATGAACAGCCTGAA |
| | GCCTGAGGATACCGCTGTGTATTACTGCCAGGCCGACCCGACCATGTTTCACAAACTGTATTACG |
| | GAATTAACCCGAACGAATATGACTACTGGGGCCAAGGGACCCAGGTAACAGTGTCCTCC (SEQ |
| | ID NO: 547) |
| 1E1 | CAGGTTCAACTCCAGGAGAGCGGCGGTGGCCTGGTCCAGCCAGGTGGCAGTCTGCGCCTCTCATG |
| | CGCTGCATCAGGCCGTACATTCTCTAGTTACGGGATGGGCTGGTTTAGGCAGGCTCCTGGCAAGG |
| | AAAGGGAATTTGTCAGTTCAATCTCTTGGAGTGGACGCAGCACCTACTATACCAACGCCGTGAGA |
| | GGCAGGTTTACTATCTCCCGCGACAATGCCAAGAACACTGTTTACCTCCAGATGAACTCTCTGAA |
| | ACCTGAGGACACCGCAGTGTATTACTGCGCCGCTTCTAGCACCTGGAACAATAACGACCCCACCG |
| | AGTACGATTATTGGGGCCAAGGTACACTTGTGACTGTGTCCAGT (SEQ ID NO: 548) |
| 1E2 | CAGGTTCAGTTGCAGGAGTCCGGTGGCGGTCTGGTGCAGGCGGGAGGGAGCCTGCGCCTGTCATG |
| | TGCTGCCAGCGAACTCACGTTCTCCACCCACGCTATGGGGTGGTTTCGCCAGGCCCCCGGCAAAG |
| | AGCGCGAGTTCGTGGCTGCAATCGCCTGGAGCGGAGGCAGCACATATTACGCCGACTCTGTGAAG |
| | GGACGTTTCACTATCAGTCGTGACATCTCCAAGAATACCATCTATCTTCAGATGAACAGCCTCAA |
| | ACCGGAGGATACCGCGCTGTATTACTGCGCTGCCACCAACGGTGGAGCCTGGCACTATCACGAAC |
| | GCTATTTTGGCTCATGGGGTCAGGGCACCCAGGTGACCGTCAGCAGT (SEQ ID NO: 549) |
| 1E3 | CAGGTGCAGCTCCAGGAGTCCGGGGGTGGACTCGTCCAGGCCGGTGGCTCCCTGCGCCTGTCTTG |
| | CACAGCCAGTGGCAGCATCTCCAGTATCAACGCAATGGGCTGGTATCGTCAGGCCCCTGGAAAGC |
| | AGCGCGAGCTGGTGGCGGCCATCACATCCGGCGGATCTACTCACTATGCGGATTCCGTTAAGGGT |
| | AGATTCACCATCAGCCGTGATAACGCGAAGAACACAATGTACTTGCAGATGAACAGTCTCAAGCC |
| | GGAAGACACCGCCGTGTATTACTGCAACGCTAGGCGCATCACAACCGGATGGGGCACAATGCGCG |
| | AGTGGGAGGGCTCCCGCGACCCCTACGAATATGATTATTGGGGACAGGGCACCCTTGTGACCGTG |
| | TCCAGC (SEQ ID NO: 550) |
| 1E4 | CAGGTGCAACTCCAAGAGTCTGGTGGCGGACTGGTGCAGCCCGGCGGAAGCCTGCGTCTGTCATG |
| | TGCAGCTTCCGGCGGTACTTTTTCCGATTACGGCATGGGATGGTTCCGCCAGGCCCCCGGTAAGG |
| | AACGCGAGTTTGTGAGCAGTATCTCCTGGAGCGGCAATTCCGTGTACTATGCCGGAGCGGTTCGG |
| | GGCCGCTTTACTATTAGCCGCGATAATGCTAAAAATACTGTCTATCTCCAGATGAACTCTCTCCA |
| | GCCAGAAGACACAGCTGTCTATTACTGCGCGGCTTCCAGCACCTGGAATAACAATGATCCTACTG |
| | AATATGAGTATTGGGGGCAAGGGACGCTGGTTACTGTCAGTTCT (SEQ ID NO: 551) |
| 1E5 | CAAGTGCAGCTTCAGGAATTTGGCGGGGGCCTCGTGCAGCCTGGTGGCAGTCTGCGCCTGAGTTG |
| | CGCTGCCTCCGGCAGGACCTTCTCCGACTATGCAATGGGTTGGTTCCGTCAGGCCCCAGGCAAAG |
| | AACGTGAGTTTGTGGCCAGCATCTCCTGGTCAGGCGGATCTCTTTATTACGCAGGCGCGGTGAGG |
| | GGCCGCTTTACAATCTCCCGTGATAATGCTAAGAACACCGTGTACCTGCAAATGAACTCCCTCAA |
| | GCCCGAAGATACCGCCGTGTATTACTGTGCCGCGTCCAGCACGTGGAACAATAACGATCCGCATG |
| | AATACGACTACTGGGGCCAGGGCACCCAGGTTACCGTCTCCTCC (SEQ ID NO: 552) |
| 1E6 | CAGGTTCAGTTGCAGGAGTTCGGTGGAGGCCTGGTGCAGGCTGGTGCGTCCCTCCGCCTGAGCTG |
| | CGCCGCATCTGGTCGCACTCTGTCCCGCTTCATGGGTTGGTTCAGGCAGGCCCCCGGCAAGGAGA |
| | GGCGCTTTGTTGCCACTATTAACTGGAATGGCGGTGCCACCGGATATGCCGATAGTGTGAAGGGT |
| | CGGTTCACAATCAGCCGCGACAACGCCAAGAACACACTGTACCTCCAGATGAACTCTCTGAAGCC |
| | CGATGACACAGCTGTGTATTACTGTGCAGCCGCTCGCACTAGCAGTATCTATTACACCAGCTCTG |
| | ACGCGGAGGAATATGACTATTGGGGCCAGGGCACCCAGGTCACCGTGAGCAGT (SEQ ID |
| | NO: 553) |
| 1E7 | CAGGTGCAGCTGCAACAGTTTGGCGGAGGCCTGGTACAGGCAGGCGGGTCCCTTCGCTTGTCTTG |
| | CGCGGCATCCGGTCGTACTGTGTCCAGCGACGCAATGGGGTGGTTTCGCCAGGCACCCGGCAAGG |
| | AAAGGGAGTTTGTTGCAGCTATAAATTGGAACGGCGCGTCCACATACTATGCCGACAGCGTGAAG |
| | GGCCGTTTCACAATCTCACGTGACAACGCTAAGAACACAGTGTATCTGCAAATGAACTCTCTGAA |
| | GTCCGAGGACACTGCCGTGTATTACTGCGCTGCGTCCGACTGGGTAGTGGGCGGAATCTCCCTGA |
| | TGAACGGGGCCGAATATGCTTACTGGGGCCAGGGAACCCAGGTAACCGTCTCAAGT (SEQ ID |
| | NO: 554) |
| 1E8 | CAAGTGCAGCTCCAGCCTAGCGGCGGGGGCCTGGTCCAGGCTGGTGGATCTCTCCGGCTGTCTTG |
| | TGCCGCGTCCGGGAGGACCGTCTCTTCCGACGCTATGGGATGGTTCCGTCAGGCTCCCGGCAAAG |
| | AGCGCGAGTTCGTGGCTGCCATTAACTGGAACGGAGCCTCCACCTATTACGCTGATTCCGTGAAA |
| | GGCCGCTTTACCATTAGCAGAGACAACGCTAAAAACACTGTGTACTTGCAGATGAACTCCTTGAA |
| | GAGCGAGGACACAGCCGTGTACTATTGCGCCGCAAGTGATTGGGTCGTGGGTGGAATCAGCCTGA |
| | TGAACGGAGCCGAATACGCTTACTGGGGCCAAGGAACACAGGTGACCGTCTCTAGC (SEQ ID |
| | NO: 555) |
| 1G1 | CAGGTGCAGCTCCAGGCCAGCGGCGGAGGTCTCGTCCAGGCTGGGGGCAGCCTGCGCCTGTCCTG |
| | TGCAGCTTCTGGCCGCACCGTGTCCAGCGACGCGATGGGGTGGTTCAGACAGGCCCCTGGAAAGG |
| | AGCGCGAGTTCGTGGCCGCAATCAACTGGAATGGCGCTTCCACCTATTACGGTGACAGTGCTCAG |
| | GGTCGTTTTACCATCAGCCGCGACAACGCGAAGAACACAGTTTACCTTCAGATGAACAGCCTGAA |
| | GAGTGAGGACACTGCCGTGTATTACTGCGCTGCATCCGACTGGGTCGTGGGCGGGATTTCCCTTA |
| | TGAACGGCGCAGAATATAAGTATTGGGGACAGGGCACACTGGTGACAGTTAGTTCC (SEQ ID |
| | NO: 556) |
| 1G2 | CAGGTGCAGTTGCAGGCTTTTGGCGGAGGCCTCGTCCAGGCTGGCGGTTCTCTCCGTCTGTCTTG |
| | CAAGGCTAGTGGGCGTACTGTGTCCTCAGATGCTATGGGCTGGTTTCGCCAAGCACCCGGAAAAG |
| | AGAGGGAGTTCGTGGCTGCCATCAACTGGAACGGGGCCTCAACTTATTACGCCGACAGCGTGAAG |
| | GGTCGGTTCACCATCTCTCGCGATAACGCTAAGAACACGGTGTATCTCCAGATGAACAGTTTGAA |
| | GAGTGAGGACACAGCTGTCTATTACTGTGCTGTGTCCGACTGGGTGGTCGGCGGTATCTCTCTGA |
| | TGAACGGGGCAGAATACAAGTACTGGGGTCAGGGAACCCAGGTCACCGTCAGCTCA (SEQ ID |
| | NO: 557) |
| 1G3 | CAGGTCCAGCTGGCCGAAAGCGGCGGTGGCCTGGTCCAGGCCGGGGGCAGCCTGCGGCTCTCTTG |
| | CAAGGCGTCTGGCCGCACCGTCAGCTCAGACGCGATGGGCTGGTTTCGCCAGGCTCCGGGTAAGG |
| | AAAGGGAGTTCGTGGCAGCGATAAATTGGAACGGAGCGTCCACATATTACGCTGATTCTGTGAAG |
| | GGCCGCTTCACTATCTCCAGAGACAACGCTAAGAACACCGTGTACCTCCAGATGAACTCCTTGAA |
| | GTCTGAGGACACCGCCGTGTATTACTGTGCGGTTTCCGATTGGGTGGTCGGCGGTATCTCCCTGA |
| | TGAACGGAGCCGAATATGAGTACTGGGGACAGGGCACCCTCGTCACTGTGTCCTCC (SEQ ID |
| | NO: 558) |
| 1G4 | CAGGTGCAGCTCCAGGAGTCCGGCGGTGGCCTCGTGCGCCCAGGCGGAAGCCTGAGACTGTCCTG |
| | CGCTGCCAGCGGTGGCACCTTTAGCTCCTACGCGATGGGCTGGTTCCGCCAGGCTCCCGGCAAGG |
| | AGAGAGAGTTTGTAGCAGCTATCGGAGGGAGCGGAGATTCCACTTATTACGCAGACTCCGTCAAG |
| | GGCCGCTTCACCATCTCTCGCGACAATGCTAAAAACACCGCTTATTTGCAGATGAACTCCTTGAA |
| | GCCTGAAGATACGGCGGTGTACTATTGCCAGGCAGATCCTACTATGTTCCATAAGTTGTATTACG |
| | GCATTCATCCTAACGAATATGAGTACTGGGGACAAGGCACCCTGGTCACTGTGTCCTCT (SEQ |
| | ID NO: 559 |
| 1G5 | CAAGTCCAGCTCCAGGAGTTTGGCGGAGGCCTGGTCCAAGGGGGTGGCAGCCTGAGACTGTCCTG |
| | CGCGGCCTCTGGGCGCACTGTGTCCTCTGACGCGATGGGTTGGTTCCGTCAGGCACCCGGCAAGG |
| | AGCGCGAGTTCGTTGCCGCTATCAACTGGAATGGTGCCTCCACCTACTATGCTGACAGCGTGAAG |
| | GGCCGGTTCACAATCAGTAGGGACAACGCCAAGAATACCGTGTACTTGCAAATGAACTCCCTGAA |
| | GTCCGAGGATACTGCGGTTTATTACTGTGCGGCCTCTGACTGGGTCGTAGGGGGTATTTCTCTGA |
| | TGAACGGGGCTGAGTACGCCTTCTGGGGACAGGGCACCCAGGTGACAGTGTCAAGT (SEQ ID |
| | NO: 560) |
| 1G6 | CAGGTACAGCTCGCGGAGTCTGGTGGAGGCTTGGTGCAAGCAGGCGGGTCTCTGCGCCTGAGTTG |
| | TAAAGCTAGTGGGAGGACCGTGTCTAGTGACGCGATGGGTTGGTTCCGGCAGGCCCCCGGCAAGG |
| | AGCGCGAGTTTGTCGCTGCAATCAACTGGAACGGCGCTTCTACCTACTATGCCGACAGCGTCAAA |
| | GGCCGCTTTACTATCAGCAGGGACAATGCTAAAAACACTGTGTACCTGCAAATGAACTCCCTGAA |
| | GAGCGAGGATACCGCCGTATATTACTGCGCCGTGTCCGACTGGGTAGTGGGCGGAATTAGCCTGA |
| | TGAACGGGGCGGAATACAAGTACTGGGGCCAGGGCACCCAGGTCACAGTTAGCTCC (SEQ ID |
| | NO: 561) |
| 1G7 | CAGGTACAGCTGCAACAGAGCGGGGGCGATCTGGTGCAGCCTGGGGGCTCCCTTAGGTTGAGCTG |
| | CGCCGCTTCTGGCCGGACCTTCTCCGACTACGGGATGGGTTGGTTCAGACAAGCACCTGGCAAGG |
| | AACGCGAGTTCGTGGCGTCCAGCTCCTGGTCCGGCAGGTCTACCTATTACGCTGCCGCTGTTCGC |
| | GGGAGGTTCACCATCAGTCGTGATAACGCGAAAAGTACAGTTTATCTCCAGATGAACTCTCTGAA |
| | GGCCGAAGATACAGCCGTGTATTACTGTGCTGCCTCTTCCTCATGGAACAATAACGATCCTTCTG |
| | AGTACGAATATTGGGGTCAGGGCACACAGGTCACGGTGTCCTCA (SEQ ID NO: 562) |
| 1G8 | CAAGTACAGCTCCAGGAGTTCGGCGGTGGCTTGGTCCAGGCCGGGGGTTCTCTGAGACTCTCCTG |
| | CAAGGCTTCCGGTAGGACTGTGTCATCTGACGCTATGGGCTGGTTCCGGCAGGCCCCAGGGAAGG |
| | AGAGAGAGTTCGTGGCTGCGATCAACTGGAATGGAGCGAGCACCTACTATGCAGACAGCGTCAAA |
| | GGACGCTTTACTATCAGTAGAGACAACGCCAAGAACACGGTCTACTTGCAGATGAACTCCTTGAA |
| | GAGCGAGGACACTGCCGTCTACTATTGTGCTGTGAGTGACTGGGTGGTAGGGGGAATTTCCCTGA |
| | TGAACGGTGCCGAGTACAAGTACTGGGGTCAGGGCACTCAGGTCACCGTGAGCAGC (SEQ ID |
| | NO: 563) |
| 1H2 | CAGGTGCAGCTCCAGGAGTCTGGCGGTGGCCTCGTTCAGGCGGGCGGTTCCCTCAGGCTGAGCTG |
| | CGCAGCCTCCGGCAGTATCTTCTCCATCAACGCAATGGGCTGGTATCGTCAAGCGCCCGGCAAGC |
| | AGCGCGAACTGGTCGCAGTCATCACCTCCGGCGGGTCCACTCATTACGCTGACAGCGTGAAGGGT |
| | CGCTTCACCATCTCCAGAGATAACGCTAAGAATACGCTGTACCTCCAGATGAACAGCCTTAAACC |
| | CGAGGATACCGCAGTTTATTACTGCAATGGCCGCAGAAAAACAACTGGCTGGGGTACGCTGCGCG |
| | AATGGGAGGGATCTCGCGATCCAGATGACTACGATTACTGGGGCCAGGGGACCCTGGTAACCGTG |
| | TCTTCA (SEQ ID NO: 564) |
| 1H3 | CAGGTGCAGCTGCAACAGTCCGGCGGAGGTCTGGTGCAAGCCGGAGGTTCCATGCGCCTGTCCTG |
| | TGCCGCTTCAGGGCGCACTGTGTCCTCTGACGCGATGGGATGGTTCCGGCAGGCCCCCGGAAAGG |
| | AACGCGAGTTCGTGGCCGCGATCAACTGGAACGGTGCAAACACTTACTATGCAGACAGCGTAAAG |
| | GGCAGATTCACTATTAGTCGCGATAACGCGAAGAACACCGTTTACCTGCAAATGAACTCTCTGAA |
| | ATCTGAGGACACAGCTGTTTATTACTGTGCTGTAAGCGATTGGGTCGTGGGCGGGATTTCATTGA |
| | TGAACGGTGCTGAGTACAAGTATTGGGGCCAGGGAACCCAAGTAACCGTCAGCTCC (SEQ ID |
| | NO: 565) |
| 1H4 | CAGGTGCAACTCCAAGAGAGCGGCGGAGGCTTGGTGCAGGCTGGTACGTCCCTGCGGCTCAGCTG |
| | CGCGGCCAGTGGCCGTACCTTCTCTTCCTTCATGGGGTGGTTCAGACAGGCACCCGGAAAGGAAA |
| | GAGAGTTCGTGGCCACCATCAACAGGTCAGGAGGTGCCACCGGGTATGCCGATAGCGTGAAGGGC |
| | AGGTTCACAATTAGCCGTGACAACGCGAAAAACACGCTCTATCTTCAGATGAATAGCCTCAAACC |
| | TGAGGATACTGCCGTCTACTATTGTGCAGCTGCAAGAGGCTCTAGCGTGTATTACACATCAACCG |
| | ACCCAGAAGAGTACGGCCACTGGGGGCAGGGGACCCAGGTTACCGTAAGCTCA (SEQ ID |
| | NO: 566) |
| 1H5 | CAAGTCCAGTTGCAGGAGTCCGGTGGCGGTTTGGTCCAGGCCGGAGCCAGCCTCAGGCTCTCCTG |
| | CCAGGCGTCCGGTCGTACCTTTAGCTCCTTCATGGGGTGGTTCCGCCAGGCTCCAGGGAAAGAGC |
| | GGAAGTTCGTTGCCACGATCAATAGAAGCGGTGGAGCGACTGGTTATGCCGATAGCGTGAAAGGA |
| | AGATTTACAATTTCCCGCGACAACGCCAAAAACACTCTCTACTTGCAAATGAACTCTTTGCGTCC |
| | CGAAGATACGGCGGTCTACTATTGCGCCGCTGCACGCACCAGTTCAAGCTATTACACATCTTCCG |
| | ACCCGGAAGAGTACAATTACTGGGGCCAGGGTACACAAGTGACCGTTTCCTCT (SEQ ID |
| | NO: 567) |
| 1H6 | CAGGTTCAGCTCCAGGCCTTCGGAGGCGGAACCGTCCAGGCTGGGGGCTCTCTGCGTCTGTCCTG |
| | TACCGCCAGCGGTGGCACCTTTTCTGACTATGGTATGGGATGGTTCCGCCAGGCCCCTGGTAAGG |
| | AGCGCGAGTTCGTCAGCTCCATTTCTTGGTCCGGTAATTCCGTTTACTATGCTGGCGCTGTGCGC |
| | GGCAGATTCACCATCTCACGTGACAACGCCAAGAACACCGTGTACTTGCAGATGAACTCTCTTCA |
| | GCCTGAGGACACCGCAGTCTATTACTGTGCCGCTTCCAGTACCTGGAACAATAACGACCCAACCG |
| | AATACGAATACTGGGGCCAAGGCACTCAGGTGACCGTCTCCAGC (SEQ ID NO: 568) |
| 1H7 | CAGGTCCAGCTCCAACAGTTTGGTGGGGGCCTGGTTCAACCTGGGGGCTCCCTGCGTCTGTCCTG |
| | CGCAGCTTCTGGCCGTACCTTTTCCGACTACGGAATGGGCTGGTTCCGGCAAGCGCCCGGCAAAG |
| | AACGCGAGTTCGTGGCCAGTTCTTCCTGGAGTGGCCGCTCCACATATTACGCTGCCGCTGTCCGT |
| | GGCCGCTTCACTATCAGCAGGGACAATGCTAAGAGCACAGTATACTTGCAGATGAACTCTCTCAA |
| | GGCCGAGGATACCGCTGTGTATTACTGTGCGGCATCCAGTTCCTGGAACAATAACGACCCATCAG |
| | AGTATGAGTATTGGGGGCAGGGCACATTGGTGACTGTATCCTCC (SEQ ID NO: 569) |
| 1H8 | CAGGTGCAGCTCCAGGAGTCAGGCGGTGGCCTGGTCCAGGCTGGTGGATCTCTGCGCCTCAGTTG |
| | TAAGGCATCCGGGCGCACGGTCTCTTCCGACGCTATGGGCTGGTTTAGACAGGCCCCTGGGAAGG |
| | AGAGAGAGTTTGTTGCTGCCATTAACTGGAACGGTGCCTCTACATATTACGCAGACTCAGTGAAG |
| | GGCAGATTTACTATTAGCCGGGACAACGCCAAGAACACCGTGTACCTTCAGATGAATAGTCTGAA |
| | GTCCGAGGATACAGCAGTGTACTATTGTGCCGTGAGCGATTGGGTCGTGGGTGGCTTCTCACTGA |
| | TGAACGGTGCTGAGTACAAGTATTGGGGCCAGGGCACTCAGGTGACGGTGTCTAGC (SEQ ID |
| | NO: 570) |
| 2A4 | CAGGTGCAGCTCCAGGAGTCAGGAGGCGGTTTGGTGCAGGCCGGTGGCAGCCTGCGCCTGAGCTG |
| | TGCCGCTAGTGGCCGCACATTCAGTACCCACAGTATGGGCAGATTCCGTCAGGCCCCAGGCAAGG |
| | AGCGCGAGTTCGTCGCTGCCATTGCGTGGTCAGGAGGCTCAACATATTACGCCGATTCAGTGAAG |
| | TGCCGCTTCACCATCTCCCGTGACATCAGCAAAAATACAATCTATCTTCAGATGAACTCCGTCAA |
| | ACCCGAGGACACCGCCCTGTACTATTGTGCCGCTACTAACGGGGGTGCTTGTCATTACCACGAGC |
| | GCTACTATGGCTCATGGGGCCAGGGGACTCAGGTGACCGTGTCCAGC (SEQ ID NO: 571) |
| 2B1 | CAGGTGCAGCTCCAGGAGTTCGGTGGAGGCCTGGTGCAAGCGGGTGGCTCTCTCCGTCTGAGCTG |
| | CGCTGCCAGCGGTAGAACAGCGAGCAGTGACGCTATGGGCTGGTTCAGGCAGACCCCCGGTAAGG |
| | AGCGGGAGTTCGTCGCCGCTATTAACTGGAACGGCGCTTCTACATACTATGCTGACTCCGTTAAA |
| | GGCCGTTTTACAATCTCCCGCGACAACGCCAAAAATACCGTGTATCTCCAGATGAACTCACTGAA |
| | GTCTGAAGATACCGCCGTGTACTATTGTGCTGCGTCAAATTGGATTGTTGGCGGGATCAGCCTGA |
| | TGAACGGGGCCGAATACAACTTCTGGGGACAGGGAACGCAGGTTACGGTCAGCAGC (SEQ ID |
| | NO: 572) |
| 2C1 | CAAGTTCAACTTCAGGAAAGTGGAGGCGGTCTGGTGCAGGGTGGAGGCTCTCTGAGGCTGTCTTG |
| | CGCTGCCAGCGGTCGCACTGTCAGCTCAGACGCGATGGGATGGTTCCGCCAAGCACCGGGAAAGG |
| | AACGCGAGTTCGTCGCTGCCATAAATTGGAACGGTGCCAACACATATTACGCAGATTCTGTGAAG |
| | GGACGCTTCACTATCAGCCGCGATAACGCGAAGAACACTGTGTACTTGCAGATGAACTCCTTGAA |
| | AAGTGAGGACACCGCCGTGTATTACTGTGCGGTGTCTGACTGGGTCGTGGGTGGCATCTCTTTGA |
| | TGAACGGAGCTGAATATAAGTACTGGGGACAGGGCACCCAGGTGACCGTATCTAGC (SEQ ID |
| | NO: 573) |
| 2C4 | CAGGTGCAGTTGCAGGCCTTTGGCGGTGGCCTTGTCCAGGCAGGAGGGAGCCTCCGCCTGAGCTG |
| | CGCAGCGTCTGGTAGAACGGTTAGCTCAGACGCTATGGGCTGGTTCCGTCAGGCACCCGGCAAGG |
| | AACGGGAGTTTGTGGCCGCAATAAATTGGAACGGTGCCAGCACCTATTACGCCGACTCCGTGAAA |
| | GGTCGTTTCACTATCTCACGCGACAACGCCAAAAACACCGTCTACTTGCAGATGAATAGCCTGAA |
| | GAGCGAGGACACGGCGGTGTATTACTGCGCCGCTTCCGACTGGGTCGTGGGAGGTATCAGCCTGA |
| | TGAACGGTGTAGAGTACAAGTATTGGGGCCAAGGCACCCAAGTGACCGTGTCAAGT (SEQ ID |
| | NO: 574 ) |
| 2C5 | CAGGTGCAGCTTCAGGAGTTTGGCGGGGGCCTGGTCCAGGCCGGTGCCTCCCTCCGCCTGAGCTG |
| | CGCCGCTTCAGGCGGAACATTCTCCTATTTTATGGGTTGGTTCCGCCAGGCCCCAGGTAAGGAGA |
| | GAGAGTTTGTGGCCACCATCAATAGGAATGGAGGCGCAACCGGGTACGCAGATTCCGTCAAGGGT |
| | AGATTTACGATCTCCCGTGACAATGCTAAGAACACCCTGTACCTCCAGATGGACTCCGTAACACC |
| | CGAGGACACTGCCGTCTATTACTGCGCTGCCGCACGCGAAAGCTCCGTTTACTATACCTCAACCG |
| | ACCCTGCTGAGTACGGCTACTGGGGGCAAGGGACCCAGGTCACGGTCAGCTCA (SEQ ID |
| | NO: 575) |
| 2C6 | CAGGTCCAGCTTCAGGAGTTCGGGGGTGGCCTTGTCCAGCCTGGGGGTAGCCTGCGGCTGTCATG |
| | CGCTGCAAGCGGACGGACTTTCTCTGACTATGGCATGGGGTGGTTCCGCCAGGCTCCGGGCAAGG |
| | AACGCGAGTTCGTGGCCAGCTCCTCTTGGTCCGGTAGAAGCACATATTACGCTGCCGCTGTGCGT |
| | GGCCGCTTCACAATTAGCCGCGACAACGCTAAATCCACTGTGTATCTCCAGATGAACTCCCTGAA |
| | GGCGGAAGACACAGCCGTGTATTACTGTGCTGCCTCCAGTAGCTGGAACAATAACGATCCCAGCG |
| | AATACGAGTATTGGGGCCAGGGCACCCAGGTTACTGTCTCTTCT (SEQ ID NO: 576) |
| 2C7 | CAGGTGCAACTCCAGGCCTTCGGCGGGGGCCTCGTACAGCCTGGTGGCAGTCTGCGGTTGTCCTG |
| | CGCTGCCTCTGGAGGCACCTTCAGCGATTACGGTATGGGATGGTTCCGCCAGGCTCCGGGGAAAG |
| | AGCGTGAGTTCGTTGCCTCTTCCTCTTGGTCTGGCCGCAGTACATATTACGCCGCAGCCGTGAGA |
| | GGCCGTTTTACAATCTCCCGCGATAACGCCAAGTCTCTGGTGTACTTGCAGATGAACAATCTCAA |
| | GCCGGAGGACACTGCGGTCTATTACTGCGCTGCCTCTTCCACCTGGAATAACAATGATCCTTCCG |
| | AGTACGAATACTGGGGTCAGGGCACCCAGGTGACCGTTAGCTCA (SEQ ID NO: 577) |
| 2D3 | CAGGTGCAACTTCAGGCCAGCGGCGGAGGCCTGGTGCAGGCCGGTGGCAGCCTCCGCCTCTCCTG |
| | CGTTGCTAGTGGCCGGACATTCAGCTCTTACGGCATGGGCTGGTTTAGGCAAGCGCCAGGGAAGG |
| | AAAGGGAGTTCGTTGCCAGTATCTCTTGGTCCGGCGGTAGCACCTATTACCCGGACAGCGTGCTG |
| | GGCAGGTTCACCATCTCCAGAGACAACGCAAAAAATGTTTTGTACCTCCAGATGAACTCCCTCAA |
| | GCCTGAAGACACAGCGGTCTACTATTGCGCAGCCAGCTCCACTTGGAACAATAACGACCCCCATG |
| | AGTATGACTACTGGGGCCAGGGCACGCTGGTGACCGTATCAAGT (SEQ ID NO: 578) |
| 2D4 | CAGGTGCAGCTTCAGGAGAGCGGCGGAGGCTTGGTCGAGGCTGGCGGTTCTCTGCGCCTGTCCTG |
| | CGCTGCCAGCGGCGGGACCTTCAGTGACTACGGTATGGGTTGGTTTCGTCAGGCACCTGGGAAGG |
| | AGCGCGAGTTCGTGTCCAGTATCAGTTGGTCTGGTAACTCAGTCTATTACGCAGGAGCCGTCCGG |
| | GGCCGCTTCACCATCTCCCGCGATAACGCCAAGAACACAGTATACCTCCAGATGAACTCCCTGCA |
| | ACCTGAGGACACAGCCGTGTATTACTGCGCTGCCAGCTCCACCTGGAACAATAACGACCCTACCG |
| | AGTATGAGTACTGGGGCCAGGGCACCCAGGTGACCGTGTCCAGC (SEQ ID NO: 579) |
| 2D5 | CAGGTGCAGCTTCAGGAGTCAGGTGGAGGCTTGGTCCAGGCAGGAGCTTCTCTCCGCCTGTCCTG |
| | TGCTGCCAGCGGAAGGACATTCAGTTCTTTCATGGCTTGGTTTCGCCAGGCACCTGGAAAGGAAC |
| | GTAAATTTGTCGCTACCATCAACTGGAACGGTGGCGCTACCGGATACGCCGACCCTGTCAAGGGT |
| | AGGTTCACTATCTCTCGCGACAACGCTAAGAACACCCTGTACCTTCAGATGAACAGCCTCAAGCC |
| | CGAAGACACCGCTGTGTATTACTGTGCCGCTGCGCGGCCCTCTTCCGCCTATTACACCAGCACTG |
| | ATGCTGAGGAATATAACTATTGGGGCCAGGGCACCCTCGTGACTGTCTCTTCT (SEQ ID |
| | NO: 580) |
| 2D6 | CAGGTCCAGCTTCAGGAGAGTGGCGGTGGCCTGGTGCAGGCGGGGGCCTCTCTCCGTCTCTCATG |
| | TGCTGCCAGTGGCCGCACCTTCTCCTCATTTATGGGCTGGTTCCGTCAGGCCCCAGGCAAGGAGA |
| | GGAAGTTTGTGGCCACCATCAACTGGAACGGCGGTGCCACTGGCTACGCAGACTCCGTCAAGGGC |
| | CGGTTTACCATCTCAAGGGACAACGCCAAAAACACCCTTTACCTCCAGATGAACTCTCTGAAACC |
| | AGAGGATACCGCTGTTTACTATTGCGCAGCCGCTCGCACTGGTAGCGCCTATTACACTTCAAGTG |
| | ATGCGGGTGAATACAATTACTGGGGACAGGGCACCCAGGTGACCGTCTCCTCT (SEQ ID |
| | NO: 581) |
| 2D8 | CAGGTGCAACTCCAGGAGAGTGGGGGTGGCCTGGTGCAGGCGGGCGCTTCCCTCCGCCTGTCTTG |
| | TGCGGCCTCTGGTCGCACCTTCTCTTCATTTATGGGCTGGTTCCGCCAAGCGCCCGGTAAGGAGC |
| | GGGAGTTCGTGGCCACTATCAACCGTAACGGGGGCGCGACCGGCTACTCCGATAGCGTGAAGGGC |
| | CGCTTCACCATCTCACGTGACAACGCTAGGTCTACACTGTATCTCCAGATGAACAGCCTGAAACC |
| | TGAGGACACCGCCGTGTATTACTGTGCTGCCGCTCGTGGTTCAAGCTCCTACTATACGAGTACAG |
| | ACCCCGAGGAATATGGCCATTGGGGACAAGGAACCCTGGTGACCGTGAGTTCC (SEQ ID |
| | NO: 582) |
| 2D9 | CAAGTGCAACTGCAAGAGTCTGGCGGTGGCCTGGTTCAGGCTGGAGCCAGCCTGCGCCTGAGCTG |
| | TGCCGCTTCTGGACGCACATTTAGTAGCTTCATGGCTTGGTTCAGACAGGCTCCTGGAAAGGAGC |
| | GCAAGTTCGTAGCTACTATAAATTGGAACGGAGGTGCCACCGGCTATGCCGATAGCGTGAAAGGT |
| | AGGTTTACAATCAGCCGGGACAATGCTAAGAACACCCTGTACCTCCAGATGAACTCCCTGAAGCC |
| | CGAGGATACAGCAGTGTATTACTGTGCGGCAGCGCGTCCTTCATCTAGCTATTACACCTCTACCG |
| | ACGCCGAAGAGTATAACTATTGGGGGCAGGGCACGCAGGTGACTGTGTCTTCA (SEQ ID |
| | NO: 583) |
| 2D10 | CAGGTTCAGCTCCAGGAATCTGGCGGGGGCCTGGTCCAGCCTGGTGGCAGTCTCCGCCTGTCCTG |
| | CGCTGCCTCTGGCCGTACCTTCTCCGACTACGGTATGGGCTGGTTCCGTCAAGCCCCCGGCAAGG |
| | AACGCGAGTTCGTCGCCAGTAGCTCCTGGAGCGGTCGTTCTACTTACTATGCCGCTGCCGTTCGC |
| | GGAAGATTCACTATTTCTAGGGATAACGCCAAGAGCACCGTTTATCTGCAAATGAACTCCCTGAA |
| | AGCCGAGGATACTGCCGTCTATTACTGTGCTGCCAGCTCCTCTTGGAACAATAACGACCCGTCCG |
| | AGTATGAGTACTGGGGCCAGGGCACGCTCGTGACAGTTAGTAGT (SEQ ID NO: 584) |
| 2E1 | CAGGTGCAGCTTCAGGCCAGTGGGGGAGGCCTGGTGCAGCCCGGTGGCTCTCTCCGCCTGTCCTG |
| | TGCTGCCAGCGGTCGCACTTTTTCCAGTTACGGGATGGGCTGGTTCCGGCAGGCTCCGGGTAAAG |
| | AGCGCGAGTTCGTCGCAAGCATCAGCTGGAGTGGCCGCAGCACCTACTATACCAACGCTGTACGC |
| | GGACGCTTCACAATTTCTCGGGACAACGCGAAGAATACCGTGTACTTGCAGATGAACTCCCTGAA |
| | GCCTGAGGACACAGCCGTGTACTATTGCGCAGCCAGCTCCACCTGGAACAATAACGACCCGACCG |
| | AGTATGAGTACTGGGGCCAGGGCACCCAGGTAACCGTTTCTTCT (SEQ ID NO: 585) |
| 2E2 | CAGGTGCAGCTGCAAGCATCCGGCGGGGGCCTGGTGCAGCCAGGAGGGTCCCTTCGTCTTAGTTG |
| | TGCTGCCTCTGGACGTACCTTCAGCTCCTACGGCATGGGCTGGTTCCGCCAAGCGCCCGGCAAGG |
| | AGCGTGAGTTTGTGGCGTCCATTAGCTGGAGTGGCCGGTCCACTTATTACACTAATGCAGTACGT |
| | GGCCGCTTCACCATCTCCAGAGACAACGCCAAAAACACCGTCTATCTCCAGATGAACAGCCTGAA |
| | GCCTGAGGATACCGCCGTGTATTACTGCGCAGCCTCCAGTACTTGGAACAATAACGATCCCACCG |
| | AGTACGAGTTCTGGGGCCAGGGAACACAGGTCACCGTGTCCAGT (SEQ ID NO: 586) |
| 2E3 | CAGGTTCAGCTCCAGGAAAGCGGCGGAGGTCTGGTCCAGGCCGGAGGCAGTCTGAGACTGAGTTG |
| | TGCTGCATCCGGCTCCATCTTCTCCATCAATGCTATGGGATGGTATAGGCAGGCTCCGGGCAAGC |
| | AACGGGAGCTGGTAGCCGCTATCACATCAGGGGGAAACACCTACTATGTGGACAGTGTGAAGGGA |
| | AGATTTTCCATCAGCCGGGACAACGCCATGAACACCGTGGATCTGCAAATGAACAGTCTGAAACC |
| | CGAAGACACCGCTGTGTACTATTGCAACGCTCGGCGCATCACAACTGGCTGGGGGACCCCACGCG |
| | AGTGGGAGGGTAGTCGTGATCCTAACGACAACGTGTTTTGGGGCCAGGGCACTCAGGTTACTGTG |
| | TCCAGC (SEQ ID NO: 587) |
| 2E4 | CAAGTGCAGTTGCAGGCATCCGGTGGCGGTCTGGTGCAGGGCGGTGGAAGCCTGAGACTGAGTTG |
| | CAAAGCCTCCGGCAGAACTGTGAGTTCCGATGCTATGGGCTGGTTCCGTCAGGCTCCAGGCAAGG |
| | AGCGTGAGTTCGTGGCGGCCATAAATTGGAACGGAGCCAGCACCTACTATGCTGATTCCGTGAAG |
| | GGCCGCTTCACCATCAGCCGGGATAACGCGAAGAACACCGTGTACCTCCAGATGAACTCTCTGAA |
| | GTCCGAGGATACAGCTGTCTATTACTGCGCCGTATCTGATTGGGTTGTGGGCGGGATTAGCCTTA |
| | TGAACGGGGCGGAATATAAGTACTGGGGGCAGGGGACCCTGGTGACCGTGAGTAGC (SEQ ID |
| | NO: 588) |
| 2E5 | CAGGTGCAGCTTCAGGAATCCGGCGGGGGATTGGTGCAGCCTGGCGGGTCCCTCCGTCTGTCTTG |
| | TGCGGCCTCAGGCCGCACCTTTTCTAGTTATGGGATGGGTTGGTTCCGCCAGGCCCCAGGCAAGG |
| | AACGTGAGTTTGTCTCAAGCATCAGTTGGTCAGGCCGTAGTACATATTACACGAACGCTGTGAGA |
| | GGTAGGTTCACTATCTCCAGAGATAACGCAAAAAACACCGTTTACCTGCAAATGAACTCTCTGAA |
| | GCCCGAGGATACTGCCGTATACTATTGTGCAGCTTCTAGCACCTGGAATAACAATGACCCAACTG |
| | AATATGAGTACTGGGGCCAGGGCACCCAAGTCACCGTTTCCAGC (SEQ ID NO: 589) |
| 2E7 | CAGGTGCAGTTGCAGGAGTCTGGGGGCGGACTTGTGCAGGCCGGGGCCTCCCTGCGCCTGAGCTG |
| | TGCTGCCTCTGGCCGGACCTTCTCCAGCTTCATGGCTTGGTTCCGCCAGGCCCCCGGCAAGGAGC |
| | GCGAGTTGGTCGCCAATATCAACTGGAATGGCGGGGCTACGGGTTACGCTGATTCCGTGAAAGGA |
| | CGGTTCACCATCTCTCGTGACAACGCAAAGAACACCCTGTATATCCAGATGAACTCTCTGAAGCC |
| | TGAGGACACTGCCGTGTACTATTGTGCCGCAGCCCGCCCCTCCAGCCGCTATTACACTTCTAACG |
| | ACGCTGAAGAGTATAACTACTGGGGGCAGGGCACGCAGGTTACCGTGTCTTCC (SEQ ID |
| | NO: 590) |
| 2E8 | CAGGTGCAACTCCAGGAGTCTGGAGGCGGATTGGTGCAGGCCGGGGCCAGCCTGAGACTCAGTTG |
| | TGCCGCGAGCGGTCGGACATCTAGTTCCTTCATGGGCTGGTTCAGGCAGGCACCAGGCAAGGAGA |
| | GAAAGTTCGTGGCCACTATCAACTGGAACGGAGGCGCTACTGGTTACGCTGACTCTGTGAAGGGC |
| | AGGTTTACCATTAGCAGGGACAACGCGAAGAATACCCTTTATCTCCAGATGAACAGTCTCAAGCC |
| | TGAGGACACCGCTGTCTATTACTGTGCAGCGGCTCGCACCTCTAGCTATTACTATACAAGCACAG |
| | ACGCCGAGGAATACAACTACTGGGGCCAGGGAACACTGGTCACCGTGTCCAGC (SEQ ID |
| | NO: 591) |
| 2F1 | CAGGTCCAGCTTCAGGCTTACGGCGGAGGTCTGGTACAGGCTGGCGGATCTCTGAGACTCTCCTG |
| | TAAGGCCTCCGGCAGGACCGTGTCAAGCGATGCGATGGGATGGTTCAGGCAGGCTCCGGGGAAAG |
| | AGCGCGAGTTCGTCGCCGCTATCAACTGGAACGGTGCCTCCACATATTACGCCGATAGCGTGAAA |
| | GGCCGCTTCACTATCTCCCGCGATAACGCTAAGAACACAGTGTACCTGCAAATGAACTCCCTCAA |
| | GTATGAAGACACCGCTGTGTATTACTGTGCTGTTTCCGATTGGGTCGTGGGCGGTATCAGCCTGA |
| | TGAACGGAGCCGAGTATAAGTACTGGGGTCAGGGCACACTCGTGACGGTGTCCTCC (SEQ ID |
| | NO: 592) |
| 2F2 | CAGGTGCAGTTGCAGGAGTCTGGAGGCGGACTCGTGCAGGCTGGCGGTTCCCTGCGCTTGAGCTG |
| | TGCGGCCTCAGGTTCTATCTTTTCCATCAATGGTATGGGCTGGTATCGCCAAGCCCCAGGTAAGC |
| | AACGTGAACTCGTGGCGCTCATCACCAGCGGTGGCAGCACTCACTACGCCGACAGCGTGAAGGGC |
| | CGTTTCACCATCTCCCGCGATAACATTAAAAACACCCTGTACTTGCAGATGAACTCTCTGAAGCC |
| | GGAAGATACAGCTGTTTATTACTGTAATGGCCGTCGCAAAACCACGGGATGGGGGACCCTGAGGG |
| | AATGGGAGGGTAGCCGCGACCCCGATGACCACGATTATTGGGGACAGGGAACCCAGGTGACCGTA |
| | TCCTCC (SEQ ID NO: 593) |
| 2F3 | CAGGTGCAGTTGCAGGCCAGCGGCGGGGGCCTGGTGCAGGGCGGAGGCTCCATGCGTCTGTCTTG |
| | CAAGGCATCTGGTAGGACGGTTTCCAGTGATGCGATGGGATGGTTCCGCCAAGCGCCTGGCAAGG |
| | AACGGGAGTTCGTGGCTGCCATTAACTGGAACGGAGCGAGCACGTATTACGCTGATAGCGTCAAG |
| | GGACGGTTTACCATCAGTCGGGACAACGCTAAAAATACCGTGTACCTTCAGATGAACAGCCTGAA |
| | AAGCGAAGATACAGCCGTGTACTATTGTGCTGTTTCTGATTGGGTCGTAGGTGGAATCTCTCTGA |
| | TGAATGGGGCCGAGTACAAGTATTGGGGCCAGGGAACTCTGGTCACTGTCTCCTCT (SEQ ID |
| | NO: 594) |
| 2F4 | CAGGTGCAGTTGCAGGAATCTGGTGGCGGGCTCGTGAAGGGTGGAGGTAGCTTGCGCCTCAGCTG |
| | TGTGGCGTCCGGGGGCACTTTTTCCTCTTATGCGATGGGTTGGTTTCGCCAGGCTCCTGGGAAGG |
| | AAAGGGAGTTTGTAGCAGCCATCGGAGGTAGCGGAGATTCCACCTATTACGCCGACAGCGTTAAG |
| | GGCCGCTTTACCATTAGCCGCGACAACGCTAAGAACAGCGTCTATTTGCAGATGAATAACCTTAA |
| | ACCAGAGGATACTGCCATCTATTACTGTGCCGCAGCCCGTGAATCTAGCGTCTACTATACCTCTA |
| | CCGACCCTGAAGAGTATGGATATTGGGGCCAGGGCACCTTGGTGACAGTGTCTAGC (SEQ ID |
| | NO: 595) |
| 2F5 | CAGGTCCAGCTTCAGGAGTCAGGAGGCGATCTGGTCCAGCGTGGGGGCTCCCTGCGCCTGAGCTG |
| | CAAGGCAAGCGGTCGCACGGTTTCCTCTGATGCTATGGGTTGGTTCAGACAGGCTCCCGGCAAGG |
| | AAAGGGAGTTTGTGGCGGCCATCAACTGGAACGGTGCCAGTACCTACTATGCGGACAGTGTCAAG |
| | GGACGCTTCACAATTTCTCGGGACAACGCCAAGAATACCGTTTACCTTCAGATGAACAGCCTGAA |
| | GTCTGAGGACACCGCCGTATATTACTGCGCCGTGTCCGATTGGGTCGTGGGAGGTATCAGTCTGA |
| | TGAATGGCGCAGAATACAAGTACTGGGGCCAGGGAACACAAGTTACCGTGAGTTCT (SEQ ID |
| | NO: 596) |
| 2F6 | CAGGTCCAACTTCAGGAATCCGGCGGAGGCCTGGTCGAGACTGGTGGCAGTCTTTCCCTGAGCTG |
| | CGCTGCCTCTGGACGCACTTTCTCCGACTACGGCATGGGCTGGTTCCGTCAGGCACCAGGGAAGG |
| | AGCGCGAGTTCGTTGCATCCAGTAGCTGGTCTGGTCGGTCCACTTATTACGCCGCAGCCGTGCGT |
| | GGACGCTTCACCGTCAGTAGGGACAACGCAAAAAGCACCGTGTACCTTCAGATGAACTCCCTGAA |
| | GCCAGAGGACACGGCTGTGTACTATTGCGCAGCCAGTTCCACTTGGAACAATAACGACCCTTCTG |
| | AATACGAATACTGGGGACAGGGGACCCAGGTGACCGTGAGTAGT (SEQ ID NO: 597) |
| 2F7 | CAAGTCCAGTTGCAGGAGAGTGGCGGTGGGCTGGTAAAGGGTGGCGGGTCCCTGCGCCTGAGCTG |
| | TGTGGCGTCAGGTGGCACGTTCTCATCTTACGCGATGGGGTGGTTTCGCCAAGCCCCAGGGAAGG |
| | AACGCGAGTTCGTGGCCGCAATCGGGGGCTCTGGTGACTCCACATATTACGCGGACTCTGTGAAA |
| | GGGCGTTTCACAATCTCACGCGACAATGCTAAGAACTCCGTGTACTTGCAGATGAACTCCCTCAA |
| | GCCCGAGGATACAGCCGTATATTACTGCCAGGCGGACCCGACAATGTTTCACAAGCTGTATTACG |
| | GCATCAATCCTAACGAATATGACTATTGGGGGCAAGGAACCCAGGTGACCGTGAGCAGC (SEQ |
| | ID NO: 598) |
| 2F8 | CAGGTGCAGCTGCAACAGTTTGGTGGCGGTTTGGTCCAAGCAGGAGGGTCTCTCAGACTTAGCTG |
| | TGCAGCCAGTGGGCGCACATTTTCTGGGTACGCGATGGGCTGGTTCCGCCAGGCACCCGGAAAAG |
| | AACGCGAGTTCGTCGCTGCCATTAACTGGTCTGGGGGCAGCACTTATTACGCCGATTCTGTGAAG |
| | GGCCGCTACACTATCTCCCGCGACAACGCCAAGAACACGGGATACCTCCAGATGAACAGCCTGAA |
| | ATCCGAAGACACTGCGGTGTATTACTGTGCCGCTTCTGACTGGGTGGTTGGCGGGATCAGCTTGA |
| | TGAATGGCGCTGAGTATAAGTATTGGGGTCAGGGTACTCTGGTCACCGTCAGTAGC (SEQ ID |
| | NO: 599) |
| 2G2 | CAGGTGCAGCTTCAGGAGTCCGGTGGCGGTCTGGTGCAGGCGGGTGGGTCCCTCGGCCTGTCCTG |
| | CTCCGCCTCAGGAACCATCTTCAACCTGGACTACATGGGTTGGTATCGCCAGGCCCTGGGCAAGC |
| | AGCGTGAACTCGTTGCCACCATTACCTCAGATGGCAGGACTAACTACGCCGACTCCGTGAAGGGG |
| | CGCTTCACCATCTTCAGAGACGGCGCAAAGAACGCCATTTTGATGCAGATGAACTCCCTGAAGCC |
| | CGAGGATACAGCGGTCTATTACTGCAAGGCGTACCTGTCTGGCCGTAGCGACTACTGGGGCCAGG |
| | GAACGCAGGTGACTGTTTCCTCT (SEQ ID NO: 600) |
| 2G4 | CAAGTGCAGCTCCAGGAGTTCGGCGGGGGACTGGTGCAGGCCGGAGGGTCCCTTCGTCTGTCCTG |
| | TGCTGCGTCTGGCTCTATTAGCCGCATTAACGCTATGGGATGGTATCGCCAGGCTCCTGGCAAGC |
| | AGCGTGAACTGGTGGCCGTCATTACAAGCGGAGGTAGCACGCATTATGCCGACTCCGTGAAGGGC |
| | CGCTTTACCATCAGCCGGGACAACGCAAAGAACACACTGTACCTGCAAATGAACTCCCTGAAACC |
| | TGAGGACACTGCGGTATATTACTGCAATGGCCGTCGCAAGACGACCGGCTGGGGTTCATTGCGTG |
| | AGTGGGAGGGTTCCCGCGATCCCGACGAGTACAACTATTGGGGCCAAGGCACTCTGGTCACCGTG |
| | TCCAGC (SEQ ID NO: 601) |
| 2H1 | CAGGTTCAGCTCCAGGCCAGCGGAGGCGGACTGGCCCAGGGCGGAGGGTCTCTGCGCCTGAGTTG |
| | CAAGGCTTCTGGCCGCACCGTGTCTTCCGACGCTATGGGATGGTTTCGCCAGGCCCCTGGGAAGG |
| | AGAGGGAGTTTGTCGCCGCTATCAACTGGAATGGAGCCAGCACCTATTACGCAGACTCTGTCAAG |
| | GGGCGCTTCACCATCAGCCGGGACAACGCCAAGAACACCGTCTACCTCCAGATGAACTCTCTGAA |
| | GTCAGAGGACACGGCGGTGTATTACTGCGCCGTCAGCGACTGGGTAGTGGGCGGTATCAGCCTGA |
| | TGAATGGAGCGGAGTATAAGTATTGGGGCCAGGGAACCCTGGTGACTGTCTCTTCA (SEQ ID |
| | NO: 602) |
| 2H5 | CAGGTGCAACTTCAGGAATCCGGGGGTGGCCTGGTTCAGACCGGCGGTTCCCTGACACTGAGTTG |
| | TGTGGCAAGCGGCGGGACATTCAGTTCATACGCTATGGGCTGGTTCCGCCAGGCTCCTGGGAAAG |
| | AGAGAGAATTTGTCGCGGCCATCGGAGGCAGTGGTGATTCCACTTACTATGCTGATTCAGTGAAG |
| | GGGAGATTCACAATTTCACGTGATAATGCTAAAAACAGCGTTTACCTCCAGATGAACTCACTCAA |
| | GCCAGAGGATACCGCCGTGTACTATTGTCAGGCTGATCCCACCATGTTCCATAAGCTGTATTACG |
| | GGATCAACCCGAACGAGTATGACTACTGGGGCCAGGGAACCCTGGTTACTGTCTCTAGT (SEQ |
| | ID NO: 603) |
| 2H6 | CAAGTCCAGTTGCAGGAAAGCGGGGGCGGGCTGGTGCAGGCAGGGGGAAGCCTCCGGCTGAGCTG |
| | CGCCGCTTCTGGCCGCACCTTTAGCACTCACGCTATGGGCTGGTTCCGGCAAGCGCCGGGAAAGG |
| | AACGCGAGTTTGTGGCCGCTATTGCGTGGTCAGGGGGAAGTACCTACTATGCCGACTCCGTGAAG |
| | GGTCGTTTTACAATCTCACGTGACATTAGTAAGAACACCATCTACTTGCAGATGAACTCCCTGAA |
| | GCCTGAAGACACGGCGTTGTATTACTGTGCTGCCACCAACGGCGGTGCATGGCACTACCACGAGC |
| | GCTACTTTGGTTCCTGGGGCCAAGGGACCCAGGTGACTGTCTCCTCT (SEQ ID NO:604) |
| 2H7 | CAAGTTCAGCTCCAGGAGTCCGGGGGAGGCCTGGTGCAGGCGGGCGGTAGCCTGCGCTTGTCTTG |
| | CGCGGCCAGTGGACGCACTTTCAGCACTCACGCTATGGGCTGGTTCCGGCAGGCCCCTGGCAAGG |
| | AGCGCGAGTTTGTGGCGGCAATCGCATGGTCCGGTGGCTCCACCTACTATGCCGACTCCGTGAAG |
| | GGCCGCTTCACCATTTCCCGTGACATCTCCAAGAACACCATCTATCTCCAGATGAATAGCCTGAA |
| | GCCGGAAGATACGGCTCTCTACTATTGTGCCGCGACCAACGGCGGAGCCTGGCACTATCACGAAC |
| | GCTACTTCGGGTCCTGGGGGCAGGGGACCCTGGTTACCGTTAGCTCT (SEQ ID NO:605) |
| 2H8 | CAAGTGCAGCTCCAGGAGAGTGGCGGTGGACTGGTACAGGCTGGTGGCTCCCTGCGTCTCAGCTG |
| | TGTGGCCTCTGGCCGCACATTTAGTTCCTACGGCATGGGATGGTTCCGTCAGGCCCCTGGCAAGG |
| | AACGTGAGTTCGTTGCCAGCATTTCCTGGAGTGGGGGTTCCACCTACTATCCTGACAGCGTCCTG |
| | GGCCGCTTCACCATTTCTCGTGACAACGCAAAAAACGTGTTGTATTTGCAGATGAACAGCCTGAA |
| | GCCTGAAGATACCGCTGTCTACTATTGTGCGGCCTCTAGCACCTGGAATAACAATGATCCCCATG |
| | AGTACGACTACTGGGGCCAGGGCACCCTGGTGACCGTGTCTAGT (SEQ ID NO: 606) |
| 2H11 | CAGGTGCAGCTGCAAGCGAGCGGAGGTGGGCTGGTCCAGGGTGGAGGTTCCATGCGCCTCAGCTG |
| | CGCTGCATCCGGTCGCACCGTGTCCAGCGATGCTATGGGCTGGTTCCGGCAGGCCCCTGGGAAGG |
| | AGCGTGAGTTCGTCGCTGCCATCAACTGGAACGGCGCAAGCACCTACTATGCAGATTCCGTGAAA |
| | GGTCGTTTCACAATTTCTCGCGACAACGCTAAGAATACCGTCTACTTGCAGATGAACTCCCTTAA |
| | ATCAGAGGACACCGCCGTGTATTACTGCGCAGCCAGCGATTGGGTTGTGGGAGGCATCAGCCTTA |
| | TGAACGCAGCTGAATACAAATATTGGGGTCAGGGCACTCAGGTGACCGTCTCCTCT (SEQ ID |
| | NO: 607) |

In summary, in order to discover new anti-CD45-binding VHH with a more diverse epitope range, a llama immunization and selection was performed from a post-immune phage library against CD45 ECD in vitro to find epitopes across the entire ECD. The resulting VHH molecules bind all four FN3-like domains within hCD45RO ECD and had a wide range of binding affinities as well as at least five different epitopes across the CD45 RO ECD. In addition to their other activities and incorporation into alternative formats as discussed below, these new VHH-based antibodies will be useful tools for the study of CD45 function in vitro and in viva.

### E. Single Domain Antibodies

In some embodiments, a CD45 binding molecule of the present disclosure is a single domain antibody (sdAb).

A single-domain antibody (sdAb) is an antibody containing a single monomeric variable antibody domain. Like a full-length antibody, sdAbs are able to bind selectively to a specific antigen. The complementary determining regions (CDRs) of sdAbs are within a single-domain polypeptide. hCD45RO VHH single-domain antibodies can be engineered from heavy chain antibodies isolated from Camelidae mammals (e.g., camels, llamas, dromedary, alpaca, and guanaco) immunized with the extracellular domain of hCD45RO or an immunologically active fragment thereof. Descriptions of sdAbs and VHHs can be found in, e.g., De Greve et al., (2019) Curr Opin Biotechnol. 61:96-101; Ciccarese, et al., (2019) Front Genet. 10:997: Chanier and Chames (2019) Antibodies (Basel) 8(1); and De Vlieger, et al. (2018) Antibodies (Basel) 8 (1). Alternatively, hCD45RO single domain antibodies may be engineered from heavy chain antibodies isolated from the IgNAR heavy chain antibodies isolated from cartilaginous fishes immunized with the extracellular domain of hCD45RO or an immunologically active fragment thereof. hCD45RO sdAbs may also be obtained by splitting the dimeric variable domains from immunoglobulin G (IgG) isotypes from other mammalian species including humans, rats, rabbits immunized with the extracellular domain of hCD45RO or an immunologically active fragment thereof. Although most research into sdAbs is currently based on heavy chain variable domains, sdAbs derived from light chains have also been shown to bind specifically to the target proteins comprising the antigenic immunization sequence. Moller et al., J Biol Chem. 285(49):38348-38361, 2010. In some embodiments, a hCD45RO sdAb is composed of a single monomeric light chain variable IgG antibody domain from a mammalian (other than a Camelidae mammal) immunized with the extracellular domain of hCD45RO or an immunologically active fragment thereof.

In some embodiments, the sdAb is a VHH. A VHH is a type of sdAb that has a single monomeric heavy chain variable antibody domain. Similar to a traditional antibody, a VHH is able to bind selectively to a specific antigen. An exemplary VHH has a molecular weight of approximately 12-15 kDa which is much smaller than traditional mammalian antibodies (150-160 kDa) composed of two heavy chains and two light chains. VHHs can be found in or produced from Camelidae mammals (e.g., camels, llamas, dromedary, alpaca, and guanaco) which are naturally devoid of light chains.

The present disclosure provides CD45 binding molecules comprising a polypeptide having at least 75%, alternatively 80%, alternatively 90%, alternatively 95%, alternatively 98%, or alternatively 99% identity to a polypeptide selected from the group consisting of: SEQ ID NO: 2, SEQ ID NO: 6, SEQ ID NO: 10, SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, SEQ ID NO: 90, SEQ ID NO: 94, SEQ ID NO: 98, SEQ ID NO: 102, SEQ ID NO: 106, SEQ ID NO: 110, SEQ ID NO: 114, SEQ ID NO: 118, SEQ ID NO: 122, SEQ ID NO: 126, SEQ ID NO: 130, SEQ ID NO: 134, SEQ ID NO: 138, SEQ ID NO: 142, SEQ ID NO: 146, SEQ ID NO: 150, SEQ ID NO: 154, SEQ ID NO: 158, SEQ ID NO: 162, SEQ ID NO: 166, SEQ ID NO: 170, SEQ ID NO: 174, SEQ ID NO: 178, SEQ ID NO: 182, SEQ ID NO: 186, SEQ ID NO: 190, SEQ ID NO: 194, SEQ ID NO: 198, SEQ ID NO:204, SEQ ID NO: 208, SEQ ID NO: 212, SEQ ID NO: 216, SEQ ID NO: 220, SEQ ID NO: 224, SEQ ID NO:228, SEQ ID NO: 232, SEQ ID NO: 236, SEQ ID NO: 240, SEQ ID NO: 244, SEQ ID NO: 248, SEQ ID NO: 252, SEQ ID NO: 256, SEQ ID NO: 260, SEQ ID NO: 264, SEQ ID NO: 268, SEQ ID NO: 272, SEQ ID NO: 276, SEQ ID NO: 280, SEQ ID NO: 284, SEQ ID NO: 288, SEQ ID NO: 292, SEQ ID NO: 296, SEQ ID NO: 300, SEQ ID NO: 304, SEQ ID NO: 308, SEQ ID NO: 312, SEQ ID NO: 316, SEQ ID NO:320, SEQ ID NO: 324, SEQ ID NO: 328, SEQ ID NO: 332, SEQ ID NO: 336, SEQ ID NO: 340, SEQ ID NO: 344, SEQ ID NO: 348, SEQ ID NO: 352, SEQ ID NO: 356, SEQ ID NO: 360, SEQ ID NO: 364, SEQ ID NO: 368, SEQ ID NO: 372, SEQ ID NO: 376, SEQ ID NO: 380, SEQ ID NO: 384, SEQ ID NO: 388, SEQ ID NO: 392, SEQ ID NO: 396, SEQ ID NO: 400, SEQ ID NO: 404, SEQ ID NO: 408, SEQ ID NO: 412, SEQ ID NO: 416, SEQ ID NO: 420, SEQ ID NO: 424, SEQ ID NO: 428, SEQ ID NO: 432, SEQ ID NO: 436, SEQ ID NO: 440, SEQ ID NO: 444, SEQ ID NO: 448, SEQ ID NO: 452, SEQ ID NO: 456, SEQ ID NO: 460, SEQ ID NO: 464, SEQ ID NO: 470, SEQ ID NO: 474, SEQ ID NO: 478, SEQ ID NO: 482, SEQ ID NO: 486, SEQ ID NO: 490, SEQ ID NO: 494, SEQ ID NO: 498, SEQ ID NO: 502, SEQ ID NO: 506, SEQ ID NO: 510, SEQ ID NO: 514, SEQ ID NO: 518, and SEQ ID NO: 522.

### CDRs

The present disclosure further provides CDR polypeptide sequences having at least 75%, alternatively 80%, alternatively 90%, alternatively 95%, alternatively 98%, alternatively 99% identity (e.g., having all of or 1, 2, 3, or 4 amino acid changes, optionally considered changes relative) to a polypeptide selected from the group consisting of: SEQ ID NO: 3, SEQ ID NO: 4, SEQ ID NO: 5, SEQ ID NO: 7, SEQ ID NO: 8, SEQ ID NO: 9, SEQ ID NO: 11, SEQ ID NO: 12, SEQ ID NO: 13, SEQ ID NO: 15, SEQ ID NO: 16, SEQ ID NO: 17, SEQ ID NO: 19, SEQ ID NO: 20, SEQ ID NO: 21, SEQ ID NO: 23, SEQ ID NO: 24, SEQ ID NO: 25, SEQ ID NO: 27, SEQ ID NO: 28, SEQ ID NO: 29, SEQ ID NO: 31, SEQ ID NO: 32, SEQ ID NO: 33, SEQ ID NO: 35, SEQ ID NO: 36, SEQ ID NO: 37, SEQ ID NO: 39, SEQ ID NO: 40, SEQ ID NO: 41, SEQ ID NO: 43, SEQ ID NO: 44, SEQ ID NO: 45, SEQ ID NO: 47, SEQ ID NO: 48, SEQ ID NO: 49, SEQ ID NO: 51, SEQ ID NO: 52, SEQ ID NO: 53, SEQ ID NO: 55, SEQ ID NO: 56, SEQ ID NO: 57, SEQ ID NO: 59, SEQ ID NO: 60, SEQ ID NO: 61, SEQ ID NO: 63, SEQ ID NO: 64, SEQ ID NO: 65, SEQ ID NO: 67, SEQ ID NO: 68, SEQ ID NO: 69, SEQ ID NO: 71, SEQ ID NO: 72, SEQ ID NO: 73, SEQ ID NO: 75, SEQ ID NO: 76, SEQ ID NO: 77, SEQ ID NO: 79, SEQ ID NO: 80, SEQ ID NO: 81, SEQ ID NO: 83, SEQ ID NO: 84, SEQ ID NO: 85, SEQ ID NO: 87, SEQ ID NO: 88, SEQ ID NO: 89, SEQ ID NO: 91, SEQ ID NO: 92, SEQ ID NO: 93, SEQ ID NO: 95, SEQ ID NO: 96, SEQ ID NO: 97, SEQ ID NO: 99, SEQ ID NO: 100, SEQ ID NO: 101, SEQ ID NO: 103, SEQ ID NO: 104, SEQ ID NO: 105, SEQ ID NO: 107, SEQ ID NO: 108 SEQ ID NO: 109, SEQ ID NO: 111, SEQ ID NO: 112, SEQ ID NO: 113, SEQ ID NO: 115, SEQ ID NO: 116, SEQ ID NO: 117, SEQ ID NO: 119, SEQ ID NO: 120, SEQ ID NO: 121, SEQ ID NO: 123, SEQ ID NO: 124, SEQ ID NO: 125, SEQ ID NO: 127, SEQ ID NO: 128, SEQ ID NO: 129, SEQ ID NO: 131, SEQ ID NO: 132, SEQ ID NO: 133, SEQ ID NO: 135, SEQ ID NO: 136, SEQ ID NO: 137, SEQ ID NO: 139, SEQ ID NO: 140, SEQ ID NO: 141, SEQ ID NO: 143, SEQ ID NO: 144, SEQ ID NO: 145, SEQ ID NO: 147, SEQ ID NO: 148, SEQ ID NO: 149, SEQ ID NO: 151, SEQ ID NO: 152, SEQ ID NO: 153, SEQ ID NO: 155, SEQ ID NO: 156, SEQ ID NO: 157, SEQ ID NO: 159, SEQ ID NO: 160, SEQ ID NO: 161, SEQ ID NO: 163, SEQ ID NO: 164, SEQ ID NO: 165, SEQ ID NO: 167, SEQ ID NO: 168, SEQ ID NO: 169, SEQ ID NO: 171, SEQ ID NO: 172, SEQ ID NO: 173, SEQ ID NO: 175, SEQ ID NO: 176, SEQ ID NO: 177, SEQ ID NO: 179, SEQ ID NO: 180, SEQ ID NO: 181, SEQ ID NO: 183, SEQ ID NO: 184, SEQ ID NO: 185, SEQ ID NO: 187; SEQ ID NO: 188; SEQ ID NO: 189; SEQ ID NO: 191, SEQ ID NO: 192, SEQ ID NO: 193, SEQ ID NO: 195, SEQ ID NO: 196, and SEQ ID NO: 197 or any set of CDRs displayed in a row of Table 5.

### F. Modified Forms of Single Domain Antibodies

1. Humanized sdAbs:

Humanized CD45RO sdAbs derived from hCD45RO VHHs of Table 4 are considered within the scope of the present disclosure. The techniques for humanization of camelid single domain antibodies are well known in the art. See, e.g. Vincke, et al. (2009) General Strategy to Humanize a Camelid Single-domain Antibody and Identification of a Universal Humanized Nanobody Scaffold J. Biol. Chem. 284(5)3273-3284. Consequently, the present disclosure provides CD45RO binding molecules comprising humanized single domain antibody derived a hCD45RO sdAb having at least 75%, alternatively 80%, alternatively 90%, alternatively 95%, alternatively 98%, or alternatively 99% identity to a polypeptide selected from the group consisting of: selected from the group consisting of: SEQ ID NO: 2, SEQ ID NO: 6, SEQ ID NO: 10, SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, SEQ ID NO: 90, SEQ ID NO: 94, SEQ ID NO: 98, SEQ ID NO: 102, SEQ ID NO: 106, SEQ ID NO: 110, SEQ ID NO: 114, SEQ ID NO: 118, SEQ ID NO: 122, SEQ ID NO: 126, SEQ ID NO: 130, SEQ ID NO: 134, SEQ ID NO: 138, SEQ ID NO: 142, SEQ ID NO: 146, SEQ ID NO: 150, SEQ ID NO: 154, SEQ ID NO: 158, SEQ ID NO: 162, SEQ ID NO: 166, SEQ ID NO: 170, SEQ ID NO: 174, SEQ ID NO: 178, SEQ ID NO: 182, SEQ ID NO: 186, SEQ ID NO: 190, SEQ ID NO: 194, SEQ ID NO: 198, SEQ ID NO:204, SEQ ID NO: 208, SEQ ID NO: 212, SEQ ID NO: 216, SEQ ID NO: 220, SEQ ID NO: 224, SEQ ID NO:228, SEQ ID NO: 232, SEQ ID NO: 236, SEQ ID NO: 240, SEQ ID NO: 244, SEQ ID NO: 248, SEQ ID NO: 252, SEQ ID NO: 256, SEQ ID NO: 260, SEQ ID NO: 264, SEQ ID NO: 268, SEQ ID NO: 272, SEQ ID NO: 276, SEQ ID NO: 280, SEQ ID NO: 284, SEQ ID NO: 288, SEQ ID NO: 292, SEQ ID NO: 296, SEQ ID NO: 300, SEQ ID NO: 304, SEQ ID NO: 308, SEQ ID NO: 312, SEQ ID NO: 316, SEQ ID NO:320, SEQ ID NO: 324, SEQ ID NO: 328, SEQ ID NO: 332, SEQ ID NO: 336, SEQ ID NO: 340, SEQ ID NO: 344, SEQ ID NO: 348, SEQ ID NO: 352, SEQ ID NO: 356, SEQ ID NO: 360, SEQ ID NO: 364, SEQ ID NO: 368, SEQ ID NO: 372, SEQ ID NO: 376, SEQ ID NO: 380, SEQ ID NO: 384, SEQ ID NO: 388, SEQ ID NO: 392, SEQ ID NO: 396, SEQ ID NO: 400, SEQ ID NO: 404, SEQ ID NO: 408, SEQ ID NO: 412, SEQ ID NO: 416, SEQ ID NO: 420, SEQ ID NO: 424, SEQ ID NO: 428, SEQ ID NO: 432, SEQ ID NO: 436, SEQ ID NO: 440, SEQ ID NO: 444, SEQ ID NO: 448, SEQ ID NO: 452, SEQ ID NO: 456, SEQ ID NO: 460, SEQ ID NO: 464, SEQ ID NO: 470, SEQ ID NO: 474, SEQ ID NO: 478, SEQ ID NO: 482, SEQ ID NO: 486, SEQ ID NO: 490, SEQ ID NO: 494, SEQ ID NO: 498, SEQ ID NO: 502, SEQ ID NO: 506, SEQ ID NO: 510, SEQ ID NO: 514, SEQ ID NO: 518, and SEQ ID NO: 522.

2. CDR Grafted sdAbs:

In some embodiments, the hCD45RO sdAb of the present disclosure is a CDR grafted hCD45RO sdAb. CDRs obtained from antibodies, heavy chain antibodies and sdAbs derived therefrom may be grafted onto alternative frameworks as described in Saerens, et al. (2005) J. Mol Biol 352:597-607 to generate CDR-grafted sdAbs. In some embodiments, the present disclosure provides a CD45 binding molecule comprising a CDR grafted CD45RO sdAb, said CDR-grafted CD45RO sdAb comprising at least one CDR, optionally two CDRs, or optionally all three CDRs (CDR1, CDR2, and CDR3) selected from the following groups of CDRs:
SEQ ID NO: 3, SEQ ID NO: 4, and SEQ ID NO: 5;
SEQ ID NO: 7, SEQ ID NO: 8, and SEQ ID NO: 9;
SEQ ID NO: 11, SEQ ID NO: 12, and SEQ ID NO: 13;
SEQ ID NO: 15, SEQ ID NO: 16, and SEQ ID NO: 17;
SEQ ID NO: 19, SEQ ID NO: 20, and SEQ ID NO: 21;
SEQ ID NO: 23, SEQ ID NO: 24, and SEQ ID NO: 25;
SEQ ID NO: 27, SEQ ID NO: 28, and SEQ ID NO: 29;
SEQ ID NO: 31, SEQ ID NO: 32, and SEQ ID NO: 33;
SEQ ID NO: 35, SEQ ID NO: 36, and SEQ ID NO: 37;
SEQ ID NO: 39, SEQ ID NO: 40, and SEQ ID NO: 41;
SEQ ID NO: 43, SEQ ID NO: 44, and SEQ ID NO: 45;
SEQ ID NO: 47, SEQ ID NO: 48, and SEQ ID NO: 49;
SEQ ID NO: 51, SEQ ID NO: 52, and SEQ ID NO: 53;
SEQ ID NO: 55, SEQ ID NO: 56, and SEQ ID NO: 57;
SEQ ID NO: 59, SEQ ID NO: 60, and SEQ ID NO: 61;
SEQ ID NO: 63, SEQ ID NO: 64, and SEQ ID NO: 65;
SEQ ID NO: 67, SEQ ID NO: 68, and SEQ ID NO: 69;
SEQ ID NO: 71, SEQ ID NO: 72, and SEQ ID NO: 73;
SEQ ID NO: 75, SEQ ID NO: 76, and SEQ ID NO: 77;
SEQ ID NO: 79, SEQ ID NO: 80, and SEQ ID NO: 81;
SEQ ID NO: 83, SEQ ID NO: 84, and SEQ ID NO: 85;
SEQ ID NO: 87, SEQ ID NO: 88, and SEQ ID NO: 89;
SEQ ID NO: 91, SEQ ID NO: 92, and SEQ ID NO: 93;
SEQ ID NO: 95, SEQ ID NO: 96, and SEQ ID NO: 97;
SEQ ID NO: 99, SEQ ID NO: 100, and SEQ ID NO: 101;
SEQ ID NO: 103, SEQ ID NO: 104, and SEQ ID NO: 105;
SEQ ID NO: 107, SEQ ID NO: 108, and SEQ ID NO: 109;
SEQ ID NO: 111, SEQ ID NO: 112, and SEQ ID NO: 113;
SEQ ID NO: 115, SEQ ID NO: 116, and SEQ ID NO: 117;
SEQ ID NO: 119, SEQ ID NO: 120, and SEQ ID NO: 121;
SEQ ID NO: 123, SEQ ID NO: 124, and SEQ ID NO: 125;
SEQ ID NO: 127, SEQ ID NO: 128, and SEQ ID NO: 129;
SEQ ID NO: 131, SEQ ID NO: 132, and SEQ ID NO: 133;
SEQ ID NO: 135, SEQ ID NO: 136, and SEQ ID NO: 137;
SEQ ID NO: 139, SEQ ID NO: 140, and SEQ ID NO: 141;
SEQ ID NO: 143, SEQ ID NO: 144, and SEQ ID NO: 145;
SEQ ID NO: 147, SEQ ID NO: 148, and SEQ ID NO: 149;
SEQ ID NO: 151, SEQ ID NO: 152, and SEQ ID NO: 153;
SEQ ID NO: 155, SEQ ID NO: 156, and SEQ ID NO: 157;
SEQ ID NO: 159, SEQ ID NO: 160, and SEQ ID NO: 161;
SEQ ID NO: 163, SEQ ID NO: 164, and SEQ ID NO: 165;
SEQ ID NO: 167, SEQ ID NO: 168, and SEQ ID NO: 169;
SEQ ID NO: 171, SEQ ID NO: 172, and SEQ ID NO: 173;
SEQ ID NO: 175, SEQ ID NO: 176, and SEQ ID NO: 177;
SEQ ID NO: 179, SEQ ID NO: 180, and SEQ ID NO: 181;
SEQ ID NO: 183, SEQ ID NO: 184, and SEQ ID NO: 185;
SEQ ID NO: 187, SEQ ID NO: 188, and SEQ ID NO: 189;
SEQ ID NO: 191, SEQ ID NO: 192, and SEQ ID NO: 193;
SEQ ID NO: 195, SEQ ID NO: 196, and SEQ ID NO: 197;
SEQ ID NO:199, SEQ ID NO:200, and SEQ ID NO:201;
SEQ ID NO:203, SEQ ID NO:204, and SEQ ID NO:205;
SEQ ID NO:207, SEQ ID NO:208, and SEQ ID NO:209;
SEQ ID NO:211, SEQ ID NO:212, and SEQ ID NO:213;
SEQ ID NO:215, SEQ ID NO:216, and SEQ ID NO:217;
SEQ ID NO:219, SEQ ID NO:220, and SEQ ID NO:221;
SEQ ID NO:223, SEQ ID NO:224, and SEQ ID NO:225;
SEQ ID NO:227, SEQ ID NO:228, and SEQ ID NO:229;
SEQ ID NO:231, SEQ ID NO:232, and SEQ ID NO:233;
SEQ ID NO:235, SEQ ID NO:236, and SEQ ID NO:237;
SEQ ID NO:239, SEQ ID NO:240, and SEQ ID NO:241;
SEQ ID NO:243, SEQ ID NO:244, and SEQ ID NO:245;
SEQ ID NO:247, SEQ ID NO:248, and SEQ ID NO:249;
SEQ ID NO:251, SEQ ID NO:252, and SEQ ID NO:253;
SEQ ID NO:255, SEQ ID NO:256, and SEQ ID NO:257;
SEQ ID NO:259, SEQ ID NO:260, and SEQ ID NO:261;
SEQ ID NO:263, SEQ ID NO:264, and SEQ ID NO:265;
SEQ ID NO:267, SEQ ID NO:268, and SEQ ID NO:269;
SEQ ID NO:271, SEQ ID NO:272, and SEQ ID NO:273;
SEQ ID NO:275, SEQ ID NO:276, and SEQ ID NO:277;
SEQ ID NO:279, SEQ ID NO:280, and SEQ ID NO:281;
SEQ ID NO:283, SEQ ID NO:284, and SEQ ID NO:285;
SEQ ID NO:287, SEQ ID NO:288, and SEQ ID NO:289;
SEQ ID NO:291, SEQ ID NO:292, and SEQ ID NO:293;
SEQ ID NO:295, SEQ ID NO:296, and SEQ ID NO:297;
SEQ ID NO:299, SEQ ID NO:300, and SEQ ID NO:301;
SEQ ID NO:303, SEQ ID NO:304, and SEQ ID NO:305;
SEQ ID NO:307, SEQ ID NO:308, and SEQ ID NO:309;
SEQ ID NO:311, SEQ ID NO:312, and SEQ ID NO:313;
SEQ ID NO:315, SEQ ID NO:316, and SEQ ID NO:317;
SEQ ID NO:319, SEQ ID NO:320, and SEQ ID NO:321;
SEQ ID NO:323, SEQ ID NO:324, and SEQ ID NO:325;
SEQ ID NO:327, SEQ ID NO:328, and SEQ ID NO:329;
SEQ ID NO:331, SEQ ID NO:332, and SEQ ID NO:333;
SEQ ID NO:335, SEQ ID NO:336, and SEQ ID NO:337;
SEQ ID NO:339, SEQ ID NO:340, and SEQ ID NO:341;
SEQ ID NO:343, SEQ ID NO:344, and SEQ ID NO:345;
SEQ ID NO:347, SEQ ID NO:348, and SEQ ID NO:349;
SEQ ID NO:351, SEQ ID NO:352, and SEQ ID NO:353;
SEQ ID NO:355, SEQ ID NO:356, and SEQ ID NO:357;
SEQ ID NO:359, SEQ ID NO:360, and SEQ ID NO:361;
SEQ ID NO:363, SEQ ID NO:364, and SEQ ID NO:365;
SEQ ID NO:367, SEQ ID NO:368, and SEQ ID NO:369;
SEQ ID NO:371, SEQ ID NO:372, and SEQ ID NO:373;
SEQ ID NO:375, SEQ ID NO:376, and SEQ ID NO:377;
SEQ ID NO:379, SEQ ID NO:380, and SEQ ID NO:381;
SEQ ID NO:383, SEQ ID NO:384, and SEQ ID NO:385;
SEQ ID NO:387, SEQ ID NO:388, and SEQ ID NO:389;
SEQ ID NO:391, SEQ ID NO:392, and SEQ ID NO:393;
SEQ ID NO:395, SEQ ID NO:396, and SEQ ID NO:397;
SEQ ID NO:399, SEQ ID NO:400, and SEQ ID NO:401;
SEQ ID NO:403, SEQ ID NO:404, and SEQ ID NO:405;
SEQ ID NO:407, SEQ ID NO:408, and SEQ ID NO:409;
SEQ ID NO:411, SEQ ID NO:412, and SEQ ID NO:413;
SEQ ID NO:415, SEQ ID NO:416, and SEQ ID NO:417;
SEQ ID NO:419, SEQ ID NO:420, and SEQ ID NO:421;
SEQ ID NO:423, SEQ ID NO:424, and SEQ ID NO:425;
SEQ ID NO:427, SEQ ID NO:428, and SEQ ID NO:429;
SEQ ID NO:431, SEQ ID NO:432, and SEQ ID NO:433;
SEQ ID NO:435, SEQ ID NO:436, and SEQ ID NO:437;
SEQ ID NO:439, SEQ ID NO:440, and SEQ ID NO:441;
SEQ ID NO:443, SEQ ID NO:444, and SEQ ID NO:445;
SEQ ID NO:447, SEQ ID NO:448, and SEQ ID NO:449;
SEQ ID NO:451, SEQ ID NO:452, and SEQ ID NO:453;
SEQ ID NO:455, SEQ ID NO:456, and SEQ ID NO:457;
SEQ ID NO:459, SEQ ID NO:460, and SEQ ID NO:461;
SEQ ID NO:463, SEQ ID NO:464, and SEQ ID NO:465;
SEQ ID NO:467, SEQ ID NO:468, and SEQ ID NO:469;
SEQ ID NO:471, SEQ ID NO:472, and SEQ ID NO:473;
SEQ ID NO:475, SEQ ID NO:476, and SEQ ID NO:477;
SEQ ID NO:479, SEQ ID NO:480, and SEQ ID NO:481;
SEQ ID NO:483, SEQ ID NO:484, and SEQ ID NO:485;
SEQ ID NO:487, SEQ ID NO:488, and SEQ ID NO:489;
SEQ ID NO:491, SEQ ID NO:492, and SEQ ID NO:493;
SEQ ID NO:495, SEQ ID NO:496, and SEQ ID NO:497;
SEQ ID NO:499, SEQ ID NO:500, and SEQ ID NO:501;
SEQ ID NO:503, SEQ ID NO:504, and SEQ ID NO:505;
SEQ ID NO:507, SEQ ID NO:508, and SEQ ID NO:509;
SEQ ID NO:511, SEQ ID NO:512, and SEQ ID NO:513;
SEQ ID NO:515, SEQ ID NO:516, and SEQ ID NO:517;
SEQ ID NO:519, SEQ ID NO:520, and SEQ ID NO:521; and
SEQ ID NO:523, SEQ ID NO:524, and SEQ ID NO:525.

### E. CD45RO Binding Molecules Comprising Additional Agents

In some embodiments, a CD45 binding molecule of the present disclosure comprises a CD45 single domain antibody (sdAb) conjugated to one or more additional biologically, therapeutic agents, chemically, optically or radioactively active agents, including combinations thereof. The conjugation of at least one such biologically, chemically, optically or radioactively active agent confer additional biological or chemical properties to CD45RO sdAb, the combination providing a CD45 binding molecule possessing additional or alternative utilities.

For example, the additional agent may be a molecule selected from one or more of: immunomodulatory agents (e.g. immunogens); molecules that improve aqueous solubility (e.g. water soluble polymers and hydrophilic molecules such as sugars); carrier molecules that extend in vivo half-life (e.g. PEGylation, Fc fusions or acylation); generation of antibodies for use in detection assays (e.g., epitope tags), enhance ease of purification (e.g., chelating peptides such as poly-His tags); targeting domains that provide selective targeting CD45 binding molecule to a particular cell or tissue type; therapeutic agents (e.g. therapeutic agents including small molecule or polypeptide agents); agents that visibility to optical or electromagnetic sensors (e.g. radionucleotides or fluorescent agents). In some embodiments, the linker is a cleavable linker or a non-cleavable linker. The use of a cleavable linker in a CD45 binding molecule as contemplated herein facilitates the release of a therapeutic agent into the intracellular cytoplasm upon internalization of the CD45 binding molecule. A non-cleavable linker would allow release upon digestion of the CD45 binding molecule of or it could be used with an agent that does not require release from the antibody (e.g. an imaging agent).

In some embodiments, where the CD45 binding molecule comprises a CD45RO sdAb in stable association with an additional agent joined via a linker. A linker is a covalent linkage between two elements of a CD45 binding molecule (e.g. a hCD45RO VHH and PEG polymer). A linker can be a covalent bond, chemical linker or a peptide linker. Suitable linkers include “flexible linkers” which are generally of sufficient length to permit some movement between the CD45RO sdAb and the linked agent(s). Examples of chemical linkers include aryl acetylene, ethylene glycol oligomers containing 2-10 monomer units, diamines, diacids, amino acids, or combinations thereof. In some embodiments, the linker is a peptide linker. Suitable peptide linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Suitable peptide linkers are known in the art, and include, for example, peptide linkers containing flexible amino acid residues such as glycine and serine. Examples of flexible linkers include glycine polymers (G)ₙ, glycine-serine polymers, glycine-alanine polymers, alanine-serine polymers, and other flexible linkers. Glycine and glycine-serine polymers are relatively unstructured, and therefore can serve as a neutral tether between components. Further examples of flexible linkers include glycine polymers (G)ₙ, glycine-alanine polymers, alanine-serine polymers, glycine-serine polymers. Glycine and glycine-serine polymers are relatively unstructured, and therefore may serve as a neutral tether between components. A multimer (e.g., 1, 2, 3, 4, 5, 6, 7, 8, 9, 10, 10-20, 20-30, or 30-50) of such linker sequences may be linked together to provide flexible linkers that may be used to conjugate a heterologous amino acid sequence to CD45RO sdAbs disclosed herein.

1. Immunomodulatory Agents

In some embodiments, a CD45 binding molecule of the present disclosure comprises an immunomodulatory agent. Immunomodulatory agents that may conjugated to the hCD45RO sdAb of the present disclosure include, but are not limited to, inactivated virus particles, inactivated bacterial toxins such as toxoid from diphtheria, tetanus, cholera, or leukotoxin molecules, inactivated bacteria and dendritic cells.

2. Flag Tags

In one embodiment, the present disclosure provides a CD45 binding molecule comprising an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the CD45RO sdAb polypeptide further comprises a C-terminal c-myc epitope tag.

3. Chelating Peptides

In one embodiment, the present disclosure provides a CD45 binding molecule comprising one or more transition metal chelating polypeptide sequences. The incorporation of such a transition metal chelating domain facilitates purification immobilized metal affinity chromatography (IMAC) as described in Smith, et al. U.S. Pat. No. 4,569,794 issued Feb. 11, 1986. Examples of transition metal chelating polypeptides useful in the practice of the present CD45 binding molecule are described in Smith, et al. supra and Dobeli, et al. U.S. Pat. No. 5,320,663 issued May 10, 1995, the entire teachings of which are hereby incorporated by reference. Particular transition metal chelating polypeptides useful in the practice of the present CD45 binding molecule are polypeptides comprising 3-6 contiguous histidine residues (SEQ ID NO: 611) such as a six-histidine (His)₆ peptide (SEQ ID NO: 609) and are frequently referred to in the art as “His-tags.” In addition to providing a purification “handle” for the recombinant proteins, such chelating peptides may also facilitate the delivery of radioisotopes in substantial accordance with the teaching of Anderson, et al., U.S. Pat. No. 5,439,829 issued Aug. 8, 1995.

4. Protein Transduction Domain Fusion Proteins

In some embodiments, a CD45 binding molecule of the present disclosure comprises a “Protein Transduction Domain” or “PTD.” A PTD is a polypeptide, polynucleotide, carbohydrate, or organic or inorganic molecule that facilitates traversing a lipid bilayer, micelle, cell membrane, organelle membrane, or vesicle membrane. The conjugation of a PTD to a CD45 binding molecule facilitates the molecule traversing a membrane. In some embodiments, a PTD is covalently linked to the amino or carboxy terminus of an hCD45RO sdAb, optionally via a linker. In some embodiments, the PTD is incorporated as part of an hCD45RO sdAb mutein fusion protein, either at the N or C terminus of the molecule.

Exemplary protein transduction domains include, but are not limited to, a minimal decapeptide protein transduction domain (corresponding to residues 47-57 of HIV-1 TAT); a polyarginine sequence comprising a number of arginine residues sufficient to direct entry into a cell (e.g., 3, 4, 5, 6, 7, 8, 9, 10, or 10-50 arginines); a VP22 domain (Zender, et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin, et al. (2004) Pharm. Research 21:1248-1256); polylysine (Wender, et al. (2000) Proc. Natl. Acad. Sci. USA 97:13003-13008), Transportan (as described in Wierzbicki, et al., (2014) Folio Histomchemica et Cytobiologica 52(4): 270-280 and Pooga, et a (1998) FASEB J 12 (1) 67-77 and commercially available from AnaSpec as Catalog No. AS-61256); KALA (as described in Wyman et al., (1997) Biochemistry 36(10) 3008-3017 and commercially available from AnaSpec as Catalog No. AS-65459); Antennapedia Peptide (as described in Pietersz, et al., (2001) Vaccine 19:1397 and commercially available from AnaSpec as Catalog No. AS-61032); and TAT 47-57 (commercially available from AnaSpec as Catalog No. AS-60023).

5. Carrier Molecules:

In some embodiments the CD45RO sdAbs of the present disclosure may be conjugated to one or more carrier molecules. Carrier molecules are typically large, slowly metabolized macromolecules which provide for stabilization and/or extended duration of action in vivo to distinguish such molecules from conventional carrier molecules used in the preparation of pharmaceutical formulations as described below. Examples of in vivo carriers that may be incorporated into CD45RO binding molecules, but are not limited to: proteins (including but not limited to human serum albumin); fatty acids (acylation); polysaccharides (including but not limited to (N- and O-linked) sugars, sepharose, agarose, cellulose, or cellulose); polypeptdies amino acid copolymers, acylation, or polysialylation, an polyethylene glycol (PEG) polymers.

a. Water Soluble Polymers:

In some embodiments, the CD45RO sdAb is conjugated to one or more water-soluble polymers. Examples of water soluble polymers useful in the practice of the present CD45 binding molecule include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol, polysaccharides, poly-alpha-hydroxy acid, polyvinyl alcohol (PVA), polyphosphazene, polyoxazolines (POZ), poly(N-acryloylmorpholine), or a combination thereof.

i. Polyethylene Glycol:

In one embodiment, the carrier molecule a polyethylene glycol (“PEG”) polymer. Conjugation of PEG polymers to proteins (PEGylation) is a well-established method for the extension of serum half-life of biological agents. The PEGylated polypeptide may be further referred to as monopegylated, dipegylated, tripegylated (and so forth) to denote a polypeptide comprising one, two, three (or more) PEG moieties attached to the polypeptide, respectively. In some embodiments, the PEG may be covalently attached directly to the sdAb (e.g., through a lysine side chain, sulfhydryl group of a cysteine or N-terminal amine) or optionally employ a linker between the PEG and the sdAb. In some embodiments, a CD45 binding moleculecomprises more than one PEG molecule each of which is attached to a different amino acid residue. In some embodiments, the sdAb may be modified by the incorporation of non-natural amino acids with non-naturally occurring amino acid side chains to facilitate site specific PEGylation In other embodiments, cysteine residues may be substituted at various positions within the sdAb to facilitate site-specific PEGylation via the cysteine sulfhydryl side chain.

PEGs suitable for conjugation to a polypeptide sequence are generally soluble in water at room temperature, and have the general formula

R(O—CH₂—CH₂)ₙO—R,

where R is hydrogen or a protective group such as an alkyl or an alkanol group, and where n is an integer from 1 to 1000. When R is a protective group, it generally has from 1 to 8 carbons. The PEG can be linear or branched. Branched PEG derivatives, “star-PEGs” and multi-armed PEGs are contemplated by the present disclosure.

A molecular weight of the PEG used in a CD45 binding molecule is not restricted to any particular range. The PEG component of a CD45 binding molecule can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, alternatively about 30,000 to about 40,000 daltons. In one embodiment of the CD45 binding molecule, the PEG is a 40 kD branched PEG comprising two 20 kD arms.

The present disclosure also contemplates a CD45 binding molecule comprising more than one PEG moiety wherein the PEGs have different sizes values, and thus the various different PEGs are present in specific ratios. For example, in the preparation of a PEGylated CD45RO binding molecule, some compositions comprise a mixture of mono-, di-, tri-, and quadra-PEGylated sdAb conjugates. In some compositions, the percentage of mono-PEGylated species is 18-25%, the percentage of di-PEGylated species is 50-66%, the percentage of tri-pegylated species is 12-16%, and the percentage of quadra-pegylated species up to 5%. Such complex compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry.

Two widely used first generation activated monomethoxy PEGs (mPEGs) are succinimidyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

The PEG can be bound to a CD45 binding molecule of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the sdAb is facilitated by the incorporation of non-natural amino acids bearing unique side chains to facilitate site specific PEGylation. The incorporation of non-natural amino acids into polypeptides to provide functional moieties to achieve site specific PEGylation of such polypeptides is known in the art. See e.g. Ptacin, et al., PCT International Application No. PCT/US2018/045257 filed Aug. 3, 2018 and published Feb. 7, 2019 as International Publication Number WO 2019/028419A1.

The PEG moiety of the of a PEGylated CD45 binding molecule may be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g. Sunbright® ME-200AL, NOF, a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10kDA linear PEG molecules (e.g., Sunbright® GL2-200AL3, NOF), a 20 kDa 2-arm branched PEG-NHS ester the 20 kDA PEG-NHS ester comprising two 10kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20kDA linear PEG molecules (e.g., Sunbright® GL2-400AL3), a 40 kDa 2-arm branched PEG-NHS ester the 40 kDA PEG-NHS ester comprising two 20kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

ii. Fc Fusions

In some embodiments, the carrier molecule is a Fc molecule or a monomeric subunit thereof. The dimeric Fc molecule may be engineered to possess a “knob-into-hole modification.” The knob-into-hole modification is more fully described in Ridgway, et al. (1996) Protein Engineering 9(7):617-621 and U.S. Pat. No. 5,731,168, issued Mar. 24, 1998. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g. tyrosine or tryptophan) creating a projection from the surface (“knob”) and ii) in the CH3 domain of a second heavy chain, an amino acid residue is replaced with an amino acid residue having a smaller side chain (e.g. alanine or threonine), thereby generating a cavity (“hole”) within at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fe region (Carter, et al. (2001) Immunol Methods 248, 7-15). The knob-into-hole format is used to facilitate the expression of a first polypeptide (e.g. an CD45RO sdAb) on a first Fc monomer with a “knob” modification and a second polypeptide on the second Fc monomer possessing a “hole” modification to facilitate the expression of heterodimeric polypeptide conjugates.

6. Targeting Domains

In some embodiments, the CD45 binding molecule is provided as a fusion protein with a polypeptide sequence (“targeting domain”) to facilitate selective binding to particular cell type or tissue expressing a cell surface molecule that specifically binds to such targeting domain, optionally incorporating a linker between the CD45RO sdAb sequence and the sequence of the targeting domain of the fusion protein.

In some embodiments of the CD45 binding molecule, the CD45 binding molecule may be targeted to a particular cell type cell by incorporation of a targeting domain into the structure of the CD45 binding molecules. As used herein, the term targeting domain refers to a moiety that specifically binds to a molecule expressed on the surface of a target cell. The targeting domain may be any moiety that specifically binds to one or more cell surface molecules (e.g. T cell receptor) expressed on the surface of a target cell. In some embodiments, the target cell is a T cell. In some embodiments, the target cell is a CD45RO+ T cell.

In some embodiments, the targeting domain is a ligand for a receptor. In some embodiments, the targeting domain Is a ligand for a receptor expressed on the surface of a T cell. In some embodiments, the ligand is a cytokine. In some embodiments, the cytokine includes but is not limited to the group consisting interleukins, interferons, and functional derivatives thereof. In some embodiments, the cytokine includes but is not limited to the group consisting IL2, IL3, IL4, IL7, IL9, IL12, IL15, IL19, IL21, IL22, IL23, IL27, IL28, IL34, and modified versions or fragments thereof that bind to their cognate ligand expressed on the surface of a T-cell. In some embodiments, the cytokine includes but is not limited to the group consisting of interferon alpha, interferon a2b, interferon gamma, or interferon lambda and modified versions or fragments thereof that bind to their cognate ligand expressed on the surface of a T-cell.

In some embodiments, the targeting domain is a polypeptide that specifically binds to a cell surface molecule associated with a tumor cell (e.g., a cognate ligand for a tumor cell receptor) selected from the group consisting of GD2, BCMA, CD19, CD33, CD38, CD70, GD2, IL3Ra2, CD19, mesothelin, Her2, EpCam, Muc1, ROR1, CD133, CEA, EGRFRVIII, PSCA, GPC3, Pan-ErbB and FAP.

In some embodiments, the targeting domain of the CD45 binding molecule is an antibody (as defined hereinabove to include molecules such as VHHs, scFvs, etc.) Examples of antibodies that may incorporated as a targeting domain of a CD45 binding molecule include but are not limited to the group consisting of: anti-GD2 antibodies, anti-BCMA antibodies, anti-CD19 antibodies, anti-CD33 antibodies, anti-CD38 antibodies, anti-CD70 antibodies, anti-GD2 antibodies and IL3Ra2 antibodies, anti-CD19 antibodies, anti-mesothelin antibodies, anti-Her2 antibodies, anti-EpCam antibodies, anti-Muc antibodies, anti-ROR1 antibodies, anti-CD133 antibodies, anti-CEA antibodies, anti-PSMA antibodies, anti-EGRFRVIII antibodies, anti-PSCA antibodies, anti-GPC3 antibodies, anti-Pan-ErbB antibodies, and anti-FAP antibodies.

The antibody or antigen-binding fragment thereof can also be linked to another antibody to form, e.g., a bispecific or a multispecific antibody

7. Labels:

In some embodiments, CD45 binding molecules of the present disclosure comprise a label. In some embodiments, the label is incorporated to facilitate use as imaging agent, diagnostic agent, or for use in cell sorting procedures. The term labels includes but is not limited to fluorescent labels, a biologically active enzyme labels, a radioisotopes (e.g., a radioactive ions), a nuclear magnetic resonance active labels, a luminescent labels, or a magnetic compound. In one embodiment a CD45RO sdAb (e.g. a CD45RO VHH) molecule in stable association (e.g. covalent, coordinate covalent) with an imaging labels. The term imaging labels is used to describe any of a variety of compounds a signature that facilitates identification, tracing and/or localization of the CD45 sdAb (or its metabolites) using diagnostic procedures. Examples of imaging labels include, but are not limited to, fluorescent compounds, radioactive compounds, and compounds opaque to imaging methods (e.g. X-ray, ultrasound). Examples of radioactive compounds useful as imaging label include but are not limited to Technetium-99m (^{99m}Tc), Indium-111 (¹¹¹In), Iodine-131 (¹³¹I), Iodine-123 (¹²³I), Iodine-125 (¹²⁵I), Gallium-67 (⁶⁷Ga), and Lutetium-177 (¹⁷Lu), phosphorus (³²P), carbon (¹⁴C), tritium (³H), yttrium (⁹⁰Y), actinium (²²⁵Ac), astatine (²¹¹At), rhenium (¹⁸⁶ Re), bismuth (²¹²Bi or ²¹³Bi), and rhodium (¹⁸⁸Rh).

8. Therapeutic Agents

In some embodiments, CD45 binding molecules of the present disclosure comprise a therapeutic agent. Examples of therapeutic agents include therapeutic small molecule (e.g. chemotherapeutic agents) or biologic therapeutic agents including antibodies, cytoxic or cytostatic compounds, a radioisotope, molecules of plant, fungal, or bacterial origin, or biological proteins (e.g., protein toxins) or particles (e.g., nano-particles or recombinant viral particles, e.g., via a viral coat protein), therapeutic antibodies, chemotherapeutic agents, as described more fully herein.

In some embodiments, the therapeutic agent which may be incorporated into the CD45 binding molecules of the present disclosure is short-range radiation emitters, including, for example, short-range, high-energy a-emitters. Examples of such radioisotope include an alpha-emitter, a beta-emitter, a gamma-emitter or a beta/gamma emitter. Radioisotopes useful as therapeutic agents include yttrium 90 (⁹⁰Y), lutetium-177 (¹⁷⁷Lu), actinium-225 (²²⁵Ac), astatine-211 (²¹¹At), rhenium-186 (¹⁸⁶Re), bismuth-212 (²¹²Bi), bismuth-213 (²¹³Bi), and rhodium-188 (¹⁸⁸Rh).

In some embodiments, the CD45 binding molecules comprises a cytotoxic agent (or derivative thereof), such maytansinol or the DM1 maytansinoid), a taxane, or a calicheamicin, pseudomonas exotoxin A, deBouganin, ricin toxin, diphtheria toxin, an amatoxin, such as a-amanitin, saporin, maytansine, a maytansinoid, an auristatin, an anthracycline, a calicheamicin, irinotecan, SN-38, a duocarmycin, a pyrrolobenzodiazepine, a pyrrolobenzodiazepine dimer, an indolinobenzodiazepine, and an indolinobenzodiazepine dimer, or a variant thereof).

### G. RIPR Molecules

In some embodiments of the present disclosure, the CD45RO binding molecules of the present disclosure are “CD45RO RIPR molecules”. RIPR molecules are described in Garcia, et al. PCT/US2019/032737 published Nov. 21, 2019 as WO/2019/222547 entitled “Receptor Inhibition by Phosphatase Recruitment” the entire teaching of which is hereby incorporated for all purposes. Garcia, et al. describe a multivalent polypeptide (a “RIPR” molecule) which comprises: (i) a first amino acid sequence including a first polypeptide module capable of binding to one or more receptor protein-tyrosine phosphatases (RPTPs), and (ii) a second amino acid sequence including a second polypeptide module capable of binding to one or more cell surface receptors that signal through a phosphorylation mechanism, wherein the first polypeptide module is operably linked to the second polypeptide module. Garcia, et al. teach that such multivalent RIPR molecules are capable of bringing the ECD of the RPTP (e.g. a CD45 isoform) into proximity of the ECD of cell surface receptor that signals through a phosphorylation mechanism into proximity such that the intracellular domains of the two receptors are sufficiently close to enable the intracellular phosphorylase domain of the RPTP to dephosporylate the intracellular domain of the cell surface receptor that signals through a phosphorylation mechanism to modulate (e.g. downregulate) the intracellular signaling of the cell surface receptor.

In one aspect, Garcia, et al. describes multivalent polypeptide comprising a first polypeptide module capable of binding to one or more receptor protein-tyrosine phosphatases (RPTPs) including but not limited to CD45 phosphatase or a functional variant thereof. The CD45RO sdAb of the present disclosure are particularly useful in the preparation of RIPR molecules as the present CD45RO sdAbs bind to domains of the CD45 RPTP close to the cell surface facilitating the close association of the two receptors.

This ability of the CD45RO sdAbs to bind near domains near the cell surface of CD45 is particularly useful in the construction of RIPR molecules wherein the ECD of the non-RPTP receptor possesses comparatively small extracellular domains. Noteworthy examples of such cell surface receptors that signal through a phosphorylation mechanism and possess a comparatively small extracellular domain include the programmed cell death protein-1 (hPD-1, Uniprot Q15116) cell surface molecule found on activated T cells, which when activated by its ligands (CD274 and CD273 expressed on tumor cells) initiates a cascade leading to the dephosphorylation of TCR signaling molecules leading to an exhausted T cell phenotype. The extracellular domain of the mature form of human PD1 (hPD1) contains only 147 amino acids. The enormous commercial and clinical success of anti-PD1 antibody products such as nivolumab and pembrolizumab underscore the importance of molecules that are capable of downregulating the intracellular activity of PD1. Amino acids 25-34 of hPD1 define the nivolumab binding site, amino acids 70-77 are associated with CD274 binding and amino acids 74-99 define the pembrolizumab binding site. Similarly, the mature form of the checkpoint molecule hCTLA4 comprises only 126 amino acids.

Additionally, it is observed that a certain fraction of cancer patients do not respond to anti-PD1 therapy and that anti-PD1 antibodies do not eliminate the residual “tonic” signaling associated with the intracellular domain of PD1 which may be sufficient to inhibit the anti-tumor immune response. In contrast, the RIPR molecules of the present disclosure regulate PD1 expression independently of the binding of PD-1 activating ligand PDL-1 which enables the RIPR molecules of the present disclosure to diminish PD-1 tonic signaling. Additionally, because the RIPR molecule does not rely on interference with the ligand binding domain of PD-1 (amino acids 70-77), it is not necessary that the CD45 ECD-binding domain of the CD45RO RIPR interfere with this small PDL-1 binding epitope of the PD-1 ECD in such a way that to deny the access of PDL-1 to its binding site thereby expanding the potential scope of PD-1 binding molecules which may be incorporated into the CD45RO/PDI RIPR format as effective in inhibiting PD-1 intracellular signaling.

In certain embodiments of the present disclosure the CD45RO RIPR molecules binding molecules are RIPR molecules wherein the first polypeptide module of the RIPR molecule is a CD45RO sdAb of the present disclosure and the second module (and or additional modules of the multivalent construct) comprises a polypeptide (e.g. an antibody, sdAb or scFv) that specifically binds to the ECD more cell surface receptors that signal through a phosphorylation mechanism (e.g. PD1, CTLA4), wherein the CD45RO sdAb is operably linked to the second (or additional) polypeptide binding module(s) of the multivalent RIPR construct. In some embodiments, the one or more cell surface receptors that mediate signaling through a specific tyrosine-based motif selected from receptors comprising an intracellular an ITAM motif, an ITSM motif, an ITIM motif, or a related intracellular motif that serves as a substrate for phosphorylation.

In some embodiments, the second module of the multivalent RIPR polypeptide specifically binds to a cell surface receptor that signals through a phosphorylation mechanism such receptors including but not limited to immune-checkpoint receptors, cytokine receptors and growth factor receptors. In some embodiments, the immune-checkpoint receptor is one or more receptors selected from the group consisting of PD-1, CTLA-4, A2AR, B7-H3, B7-H4, BTLA, CD5, CD132, IDO, KIR, LAG3, TIM-3, TIGIT, VISTA, CD27, CD28, CD40, 0X40, GITR, ICOS, CD137, and functional variants of any thereof.

In some embodiments, the immune-checkpoint receptor is human PD-1. Examples of polypeptides which specifically bind to the ECD of the PDI T-cell inhibitory receptor including but not limited to anti-PD1 IgG class antibodies, functional fragments and derivatives thereof (e.g. anti-PD-1 scFvs). Examples of anti-PD1 antibodies include but are not limited to pembrolizumab (Keytruda®), nivolumab (Opdivo®), cemiplimab (Libtayo®), durvalumab (Imfinzi®), and avelumab (MSB0010718C, Bavencio®) Anti-PD1 antibodies are extensively described in the art such as described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex, Inc.) issued Aug. 30, 2011, U.S. Pat. No. 7,943,743 (Medarex, Inc.) issued May 17, 2011. In some embodiments of the CD45RO RIPR molecules of the present disclosure, the polypeptide that specifically binds to the ECD of the non-RPTP receptor is an aptamer. Examples of aptamer PD1 inhibitors are provided in Wang, et al. (2018) 145:125-130.

In some embodiments, the polypeptide that specifically binds to the ECD of the receptor is the cognate ligand for the receptor or a functional variant thereof. In some embodiments, where the receptor is a checkpoint receptor, the polypeptide that specifically binds to the checkpoint receptor. When the checkpoint receptor is PD-1, the binding module may be a polypeptide derived from its cognate ligands PDL1 or PDL2 that specifically binds to PD-1. Peptidyl PD-1 pathway inhibitors such as those described in Sasikumar, et al., U.S. Pat. No. 9,422,339 issued Aug. 23, 2016, and Sasilkumar, et al., U.S. Pat. No. 8,907,053 issued Dec. 9, 2014.

In some embodiments, the one or more cell non-RPTP surface receptors are selected from the group consisting of DAP 10, DAP 12, SIRPα, CD3, CD28, CD4, CD8, CD200, CD200R, ICOS, KIR, FcR, BCR, CD5, CD2, G6B, LIRs, CD7, BTNs, and functional variants of any thereof.

In some embodiments, the one or more cell surface receptors is a cytokine receptor selected from the group consisting of interleukin receptors, interferon receptors, chemokine receptors, growth hormone receptors, erythropoietin receptors (EpoRs), thymic stromal lymphopoietin receptors (TSLPRs), thrombopoetin receptors (TpoRs), granulocyte macrophage colony-stimulating factor (GM-CSF) receptors, and granulocyte colony-stimulating factor (G-CSF) receptors. In some embodiments, the growth factor receptor is a tyrosine receptor kinase (TRK) belonging to a TRK family selected from the group consisting of EGF receptor family (ErbB family), insulin receptor family, PDGF receptor family, VEGF receptors family, FGF receptor family, CCK receptor family, NGF receptor family, HGF receptor family, Eph receptor family, AXL receptor family, TIE receptor family, RYK receptor family, DDR receptor family, RET receptor family, ROS receptor family, LTK receptor family, ROR receptor family, and MuSK receptor family. In some embodiments, the growth factor receptor is a stem cell growth factor receptor (SCFR) or an epidermal growth factor receptor (EGFR) selected from the group consisting of ErbB-1, ErbB-2 (HER2), ErbB-3, ErbB-4, and c-Kit (CD117).

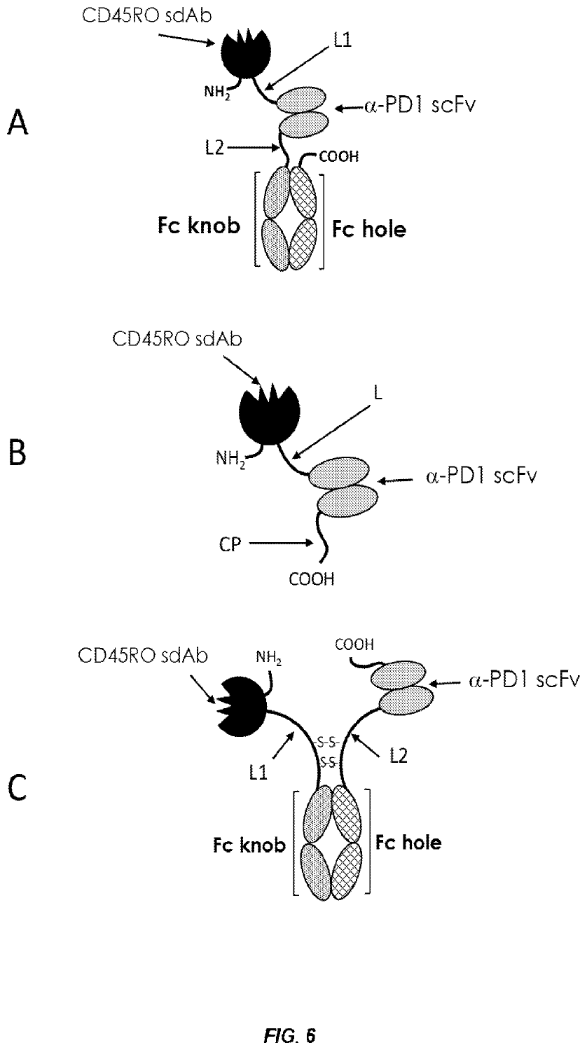

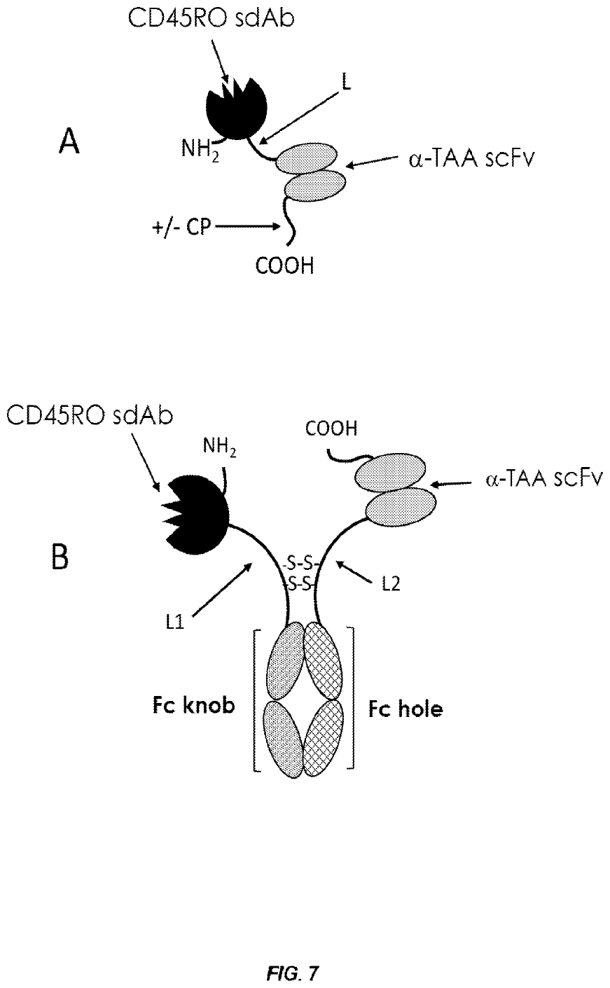

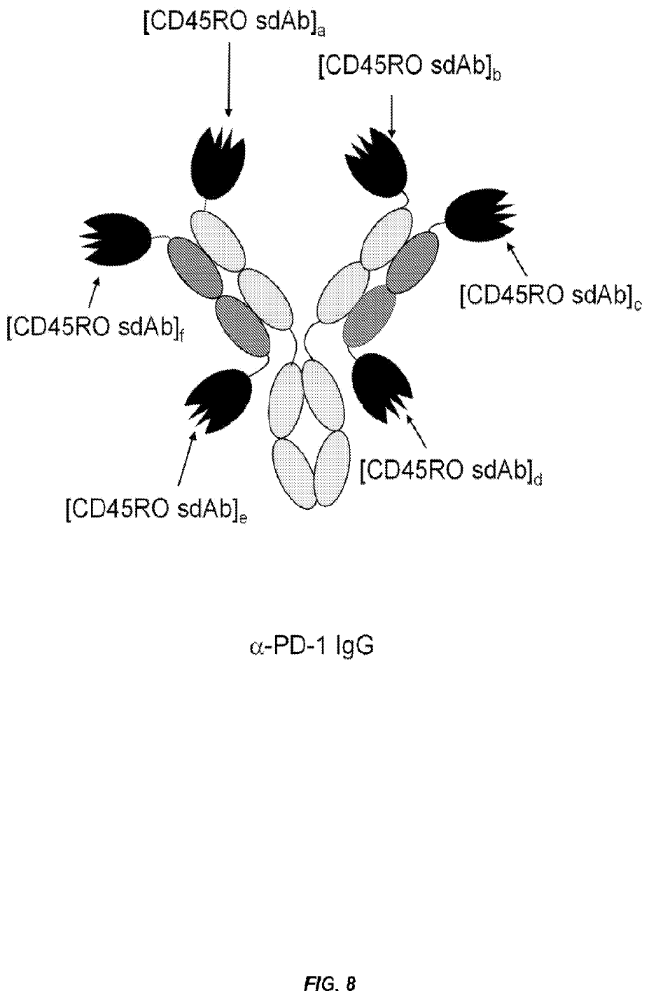

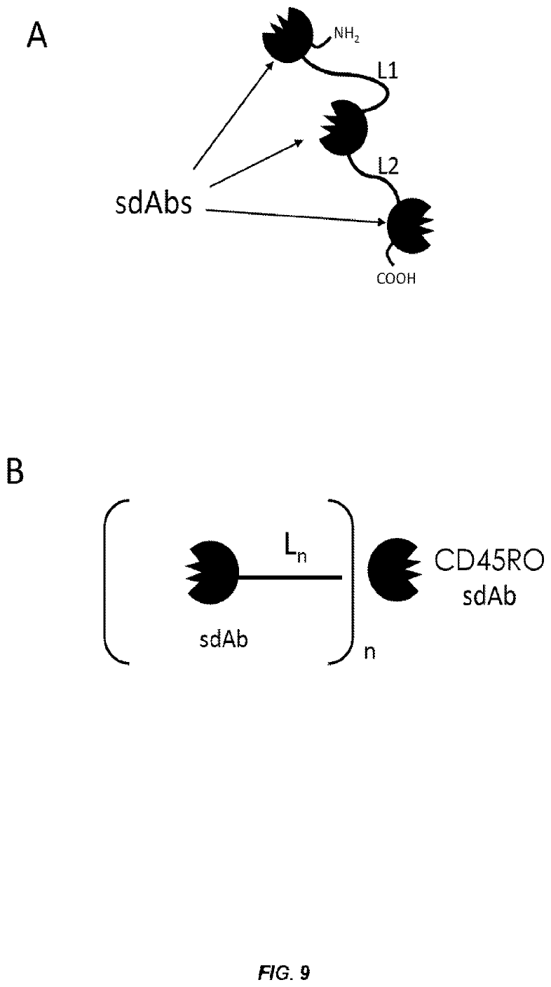

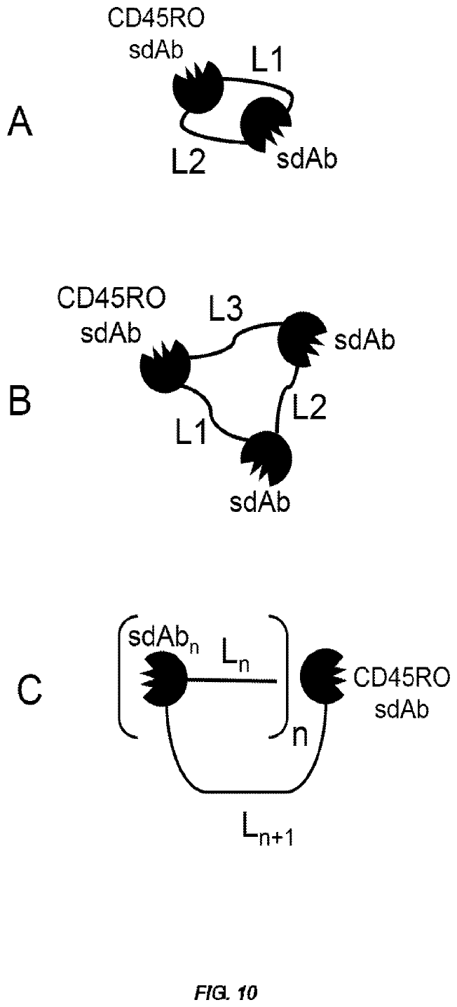

The CD45RO sdAb molecules of the present disclosure may be incorporated into any of the variety of RIPR architectures discussed by Garcia, et al., in particular those encompassed by Formula I and Formula II (paragraphs [0132] through [0136]) and Tables 1, 2 and 3 of WO201222547). The CD45RO sdAb molecules of the present disclosure may also be incorporated into any of the variety of RIPR architectures provided in FIGS. 6, 7, 8, and in some embodiments, FIGS. 9 and 10 attached drawings.

### H. Multi-Specific Binding Format

Na\check{i}ve T-cells express isoforms containing the CD45RA domain. However, following activation the T-cells express the low MW 180 kDa CD45RO isoform (‘CD45RO’ cells). The sdAbs can be combined to provide multispecific targeting and selection including in combination with bi-specific and tri-specific constructs which bind to one or more tumor associated antigens including but not limited to bispecific T cell engagers (BITEs), dual affinity retargeting (DART) constructs, and trispecific killer engager (TriKE) constructs). Examples of such multivalent binding formats incorporating a single domain CD45RO antibodies of the present CD45 disclosure are provided in FIGS. 9 and 10 of the attached drawings.

### I. Synthesis of CD45RO Binding Molecules

In some embodiments, the CD45RO binding molecules of the present disclosure are polypeptides. However, in some embodiments, only a portion of the CD45 binding molecule is a polypeptide, for example where the CD45 binding molecule comprises a non-peptidyl domain (e.g. a PEG CD45RO sdAb conjugate, a radionucleotide CD45RO sdAb conjugate, or a small molecule CD45RO sdAb conjugate). The following provides guidance to enable the solid phase and recombinant synthesis of the polypeptide portions (domains) of CD45RO binding molecules of the present disclosure. In those embodiments where only a portion of the CD45 binding molecule is a polypeptide, it will be understood that the peptidyl domain(s) of the CD45 binding molecule are an intermediate in the process which may undergo further processing to complete the synthesis of the desired CD45 binding molecules. The polypeptide domains of CD45RO binding molecules may be produced by conventional methodology for the construction of polypeptides including recombinant or solid phase syntheses as described in more detail below.

1. Chemical Synthesis:

In addition to generating mutant polypeptides via expression of nucleic acid molecules that have been altered by recombinant molecular biological techniques, polypeptide domains of CD45RO binding molecules can be chemically synthesized. Chemically synthesized polypeptides are routinely generated by those of skill in the art. Chemical synthesis includes direct synthesis of a peptide by chemical means of the polypeptide domains of CD45RO binding molecules exhibiting the properties described. This method can incorporate both natural and unnatural amino acids at desired positions that facilitate linkage of particular molecules (e.g. PEG)

In some embodiments, the polypeptide domains of CD45RO binding molecules of the present disclosure may be prepared by chemical synthesis. The chemical synthesis of the polypeptide domains of CD45RO binding molecules may proceed via liquid-phase or solid-phase. Solid-phase peptide synthesis (SPPS) allows the incorporation of unnatural amino acids and/or peptide/protein backbone modification. Various forms of SPPS are available for synthesizing the polypeptide domains of CD45RO binding molecules of the present disclosure are known in the art (e.g., Ganesan A. (2006) Mini Rev. Med. Chem. 6:3-10; and Camarero J. A. et al., (2005) Protein Pept Lett. 12:723-8). In the course of chemical synthesis, the alpha functions and any reactive side chains may protected with acid-labile or base-labile groups that are stable under the conditions for linking amide bonds but can readily be cleaved without impairing the peptide chain that has formed.

In the solid phase synthesis, either the N-terminal or C-terminal amino acid may be coupled to a suitable support material. Suitable support materials are those which are inert towards the reagents and reaction conditions for the stepwise condensation and cleavage reactions of the synthesis process and which do not dissolve in the reaction media being used. Examples of commercially available support materials include styrene/divinylbenzene copolymers which have been modified with reactive groups and/or polyethylene glycol; chloromethylated styrene/divinylbenzene copolymers; hydroxymethylated or aminomethylated styrene/divinylbenzene copolymers; and the like. The successive coupling of the protected amino acids can be carried out according to conventional methods in peptide synthesis, typically in an automated peptide synthesizer.

At the end of the solid phase synthesis, the peptide is cleaved from the support material while simultaneously cleaving the side chain protecting groups. The peptide obtained can be purified by various chromatographic methods including but not limited to hydrophobic adsorption chromatography, ion exchange chromatography, distribution chromatography, high pressure liquid chromatography (HPLC) and reversed-phase HPLC.

2. Recombinant Production:

Alternatively, polypeptide domains of CD45RO binding molecules of the present disclosure may be produced by recombinant DNA technology. In the typical practice of recombinant production of polypeptides, a nucleic acid sequence encoding the desired polypeptide is incorporated into an expression vector suitable for the host cell in which expression will be accomplish, the nucleic acid sequence being operably linked to one or more expression control sequences encoding by the vector and functional in the target host cell. The recombinant protein may be recovered through disruption of the host cell or from the cell medium if a secretion leader sequence (signal peptide) is incorporated into the polypeptide. The recombinant protein may be purified and concentrated for further use including incorporation.

3. Synthesis of Nucleic Acid Sequences Encoding the CD45RO Binding Molecule

In some embodiments, the polypeptide domains of CD45 binding molecule is produced by recombinant methods using a nucleic acid sequence encoding the polypeptide domains of CD45 binding molecule (or fusion protein comprising the polypeptide domains of CD45RO binding molecule). The nucleic acid sequence encoding the desired polypeptide domains of CD45 binding molecule can be synthesized by chemical means using an oligonucleotide synthesizer.

The nucleic acid molecules are not limited to sequences that encode polypeptides; some or all of the non-coding sequences that lie upstream or downstream from a coding sequence (e.g., the coding sequence of the polypeptide domains of CD45RO binding molecule) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the polypeptide domains of CD45 binding molecule (and fusions thereof) may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the polypeptide domains of the CD45 binding molecule may be obtained from various commercial sources that provide custom synthesis of nucleic acid sequences. Amino acid sequence variants of the hCD45 binding molecules of the present disclosure are prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion can be made to arrive at the final construct, provided that the final construct possesses the desired biological activity as defined herein.

Methods for constructing a DNA sequence encoding the polypeptide domains of CD45 binding molecule and expressing those sequences in a suitably transformed host include, but are not limited to, using a PCR-assisted mutagenesis technique. Mutations that consist of deletions or additions of amino acid residues to polypeptide domains of CD45 binding molecule can also be made with standard recombinant techniques. In the event of a deletion or addition, the nucleic acid molecule encoding polypeptide domains of CD45 binding molecule is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

A polypeptide domain of CD45 binding molecules of the present disclosure may be produced recombinantly not only directly, but also as a fusion polypeptide with a heterologous polypeptide, e.g. a signal sequence or other polypeptide having a specific cleavage site at the N-terminus or C-terminus of the mature CD45RO binding molecule. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In some embodiments, the signal sequence is the signal sequence that is natively associated with the CD45 binding molecule (i.e. the human CD45 signal sequence). The inclusion of a signal sequence depends on whether it is desired to secrete the CD45 binding molecule from the recombinant cells in which it is made. If the chosen cells are prokaryotic, it generally is preferred that the DNA sequence not encode a signal sequence. If the chosen cells are eukaryotic, it generally is preferred that a signal sequence be encoded and most preferably that the wild type IL-2 signal sequence be used. Alternatively, heterologous mammalian signal sequences may be suitable, such as signal sequences from secreted polypeptides of the same or related species, as well as viral secretory leaders, for example, the herpes simplex gD signal. When the recombinant host cell is a yeast cell such as Saccharomyces cerevisiae, the alpha mating factor secretion signal sequence may be employed to achieve extracellular secretion of the CD45 binding molecule into the culture medium as described in Singh, U.S. Pat. No. 7,198,919 B1 issued Apr. 3, 2007.

In the event the polypeptide domain of CD45 binding molecules to be expressed is to be expressed as a chimera (e.g., a fusion protein comprising an CD45 binding molecule and a heterologous polypeptide sequence), the chimeric protein can be encoded by a hybrid nucleic acid molecule comprising a first sequence that encodes all or part of the polypeptide domains of CD45 binding molecule and a second sequence that encodes all or part of the heterologous polypeptide. For example, polypeptide domains of CD45RO binding molecules described herein may be fused to a hexa-histidine tag (SEQ ID NO: 609) to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the polypeptide domains of CD45RO binding molecule. For example, the N-terminus may be linked to a targeting domain and the C-terminus linked to a hexa-histidine tag (SEQ ID NO: 609) purification handle.

The complete amino acid sequence of the polypeptide domain of CD45 binding molecules (or fusion/chimera) to be expressed can be used to construct a back-translated gene. A DNA oligomer containing a nucleotide sequence coding for the polypeptide domain of CD45 binding molecules can be synthesized. For example, several small oligonucleotides coding for portions of the desired polypeptide can be synthesized and then ligated. The individual oligonucleotides typically contain 5′ or 3′ overhangs for complementary assembly.

In some embodiments, the nucleic acid sequence encoding the polypeptide domain of CD45 binding molecules may be “codon optimized” to facilitate expression in a particular host cell type. Techniques for codon optimization in a wide variety of expression systems, including mammalian, yeast and bacterial host cells, are well known in the and there are online tools to provide for a codon optimized sequences for expression in a variety of host cell types. See e.g. Hawash, et al., (2017) 9:46-53 and Mauro and Chappell in Recombinant Protein Expression in Mammalian Cells: Methods and Protocols, edited by David Hacker (Human Press New York). Additionally, there are a variety of web based on-line software packages that are freely available to assist in the preparation of codon optimized nucleic acid sequences.

4. Expression Vectors:

Once assembled (by synthesis, site-directed mutagenesis or another method), the nucleic acid sequence encoding polypeptide domains of CD45 binding molecule will be inserted into an expression vector. A variety of expression vectors for uses in various host cells are available and are typically selected based on the host cell for expression. An expression vector typically includes, but is not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors. To facilitate efficient expression of the recombinant polypeptide, the nucleic acid sequence encoding the polypeptide sequence to be expressed is operably linked to transcriptional and translational regulatory control sequences that are functional in the chosen expression host.

Expression vectors typically contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors for polypeptide domain of CD45 binding molecules of the present disclosure contain a regulatory sequence that is recognized by the host organism and is operably linked to nucleic acid sequence encoding the polypeptide domains of CD45RO binding molecule. The terms “regulatory control sequence,” “regulatory sequence” or “expression control sequence” are used interchangeably herein to refer to promoters, enhancers, and other expression control elements (e.g., polyadenylation signals). See, for example, Goeddel (1990) in Gene Expression Technology: Methods in Enzymology 185 (Academic Press, San Diego CA USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. In selecting an expression control sequence, a variety of factors understood by one of skill in the art are to be considered. These include, for example, the relative strength of the sequence, its controllability, and its compatibility with the actual DNA sequence encoding the subject CD45 binding molecule, particularly as regards potential secondary structures.

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

Transcription from vectors in mammalian host cells may be controlled, for example, by promoters obtained from the genomes of viruses such as polyoma virus, fowlpox virus, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context. Proper assembly of the expression vector can be confirmed by nucleotide sequencing, restriction mapping, and expression of a biologically active polypeptide in a suitable host.

5. Host Cells:

The present disclosure further provides prokaryotic or eukaryotic cells that contain and express a nucleic acid molecule that encodes a polypeptide domains of CD45RO binding molecule. A cell of the present disclosure is a transfected cell, i.e., a cell into which a nucleic acid molecule, for example a nucleic acid molecule encoding a polypeptide domains of CD45 binding molecule, has been introduced by means of recombinant DNA techniques. The progeny of such a cell are also considered within the scope of the present disclosure.

Host cells are typically selected in accordance with their compatibility with the chosen expression vector, the toxicity of the product coded for by the DNA sequences of this CD45 binding molecule, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments the recombinant polypeptide domains of CD45 binding molecule or biologically active variants thereof can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerevisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)).

Examples of useful mammalian host cell lines are mouse L cells (L-M[TK-], ATCC #CRL-2648), monkey kidney CV1 line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The polypeptide domains of CD45 binding molecule can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

In some embodiments, the recombinant polypeptide domains of CD45 binding molecule may be glycosylated or unglycosylated depending on the host organism used to produce the CD45RO binding molecule. If bacteria are chosen as the host then the polypeptide domains of CD45 binding molecule produced will be unglycosylated. Eukaryotic cells, on the other hand, will glycosylate the recombinant polypeptide domains of CD45RO binding molecule.

For other additional expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif.).

6. Transfection:

The expression constructs of the can be introduced into host cells to thereby produce the recombinant polypeptide domains of CD45 binding molecule disclosed herein or to produce biologically active muteins thereof. Vector DNA can be introduced into prokaryotic or eukaryotic cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals.

In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

7. Cell Culture:

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

8. Recovery of Recombinant Proteins:

Recombinantly-produced CD45 binding polypeptides can be recovered from the culture medium as a secreted polypeptide if a secretion leader sequence is employed. Alternatively, the CD45 binding polypeptides can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

9. Purification:

Various purification steps are known in the art and find use, e.g. affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural specific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

The recombinant polypeptide domains of CD45 binding molecule produced by the transformed host can be purified according to any suitable method. CD45RO binding molecules can be isolated from inclusion bodies generated in E. coli, or from conditioned medium from either mammalian or yeast cultures producing a given CD45RO binding molecule sing cation exchange, gel filtration, and or reverse phase liquid chromatography.

The substantially purified forms of the recombinant polypeptides can be used, e.g., as therapeutic agents, as described herein.

The biological activity of the recombinant polypeptide domains of CD45 binding molecule produced in accordance with the foregoing can be confirmed by a CD45RO binding using procedures well known in the art including but not limited to competition ELISA, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET) and surface plasmon resonance assays (see, e.g., Drescher et al., Methods Mol Biol 493:323-343 (2009) with instrumentation commercially available from GE Healthcare Bio-Sciences such as the Biacore 8+, Biacore S200, Biacore T200 (GE Healthcare Bio-Sciences, 100 Results Way, Marlborough MA 01752)); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays).

### J. Methods of Use

1. Inhibition of CD45RO Dimerization:

In one embodiment, the present disclosure provides a method of modulating the activity of CD45RO+ immune cells by the administration of a CD45 binding molecule to a subject in an amount sufficient to interfere with the dimerization of CD45RO isoforms on the surface of immune cells. The present disclosure further provides a method of modulating the activity of CD45RO+ memory cells in a population of immune cells comprising contacting said population of immune cells, in vivo and/or ex vivo, with a CD45RO antibody or complex of the present disclosure to in an amount sufficient to interfere with the dimerization of CD45RO isoforms on the surface of CD45RO+ memory cells. The present disclosure provides a method of restoring the activity of quiescent CD45RO+ immune cells by the administration of a CD45 binding molecule to a subject in an amount sufficient to interfere with the dimerization of CD45RO isoforms on the surface of immune cells.

The level of CD45 phosphatase activity in CD45RO+ cells is regulated, in part, by the formation of CD45RO homodimers ([CD45RO]₂) which possess diminished phosphatase activity compared to CD45 isoform monomers. CD45 activity has also been described to be dependent on dimerization state; a model has been proposed in which dimerization results in the formation of an inhibitory structural “wedge” disrupting substrate access to the phosphatase domain Studies have suggested that the majority of CD45RA+ isoforms present on na\check{i}ve T cells exist in a monomeric state (Mittler, et al. (1991) J. Immunol. 147:3434-40) which is consistent with the presence of negative charged sugar moieties and/or the steric hinderance to dimerization caused by the presence of large O-linked sugar moieties of the ECD of CD45RA+ isoforms. Consequently, cells expressing CD45RA+ isoforms are primed for activation in response to TCR signaling. The presence of CD45RA+ isoforms remains high following initial activation phosphorylating Lck which in turn phosphorylates the cytoplasmic CD3 and ζ-chains of the TCR complex which initiates a cascade of phosphorylation eventually leading to activation of transcription factors.

However, over the course of time (several days), the CD45RO isoform replaces the CD45RA isoform on the surface of activated T cells. Since the CD45RO isoform does not possess the large O-linked sugars of the ECD of CD45RA+ isoforms, the CD45RO isoforms are able to form CD45RO homodimers. CD45RO homodimers have been detected by fluorescence resonance energy transfer (FRET) analysis in a CD45-deficient T cell line reconstituted with various CD45 isoforms. Dornan, et al. (2002) J. Biol. Chem. 277:1912-18. A model has been proposed in which CD45 phosphatase activity is dependent on dimerization state. Dimerization of CD45 results in the formation of an inhibitory structural “wedge” disrupting substrate access to the phosphatase domain. Gabaev, et al. (2011) PLoS Pathogens 7(12):e1002432; Bilwes, et al. (1996) Nature 382:555-559; Majeti, et al. (2000) Cell 103:1059-1070. Consequently, hCD45RO homodimers possess significantly diminished phosphatase activity relative to their monomeric CD45RO isoforms. In T or B cells, the dimerization of CD45RO isoforms contributes to down-regulation of the primary immune response and guards against an excessive immune response and/or inflammation which could cause tissue injury.

Without being bound to any theory of the mechanism of action, the CD45RO antibodies and complexes of the present disclosure may be administered in an amount sufficient interfere with the formation of CD45RO homodimers on CD45RO+ quiescent B or T cells and upregulating or reestablishing the immunological activity of such quiescent B or T cells.

2. Use of Multivalent CD45 Binding Molecules To Downregulate Immune Cell Activity and Use in the Treatment Of Conditions Associated with Hyper responsive T cell Function:

The present disclosure provides a compositions and methods of down-regulating immune response in hCD45RO+ positive cells by the administration of a multivalent CD45 binding molecule of the present disclosure, multivalent CD45 binding molecule comprising two or more hCD45RO sdAbs. Multivalent CD45 binding molecules of the present are capable of aggregating CD45 isoforms on the surface of CD45+ cells leading to downregulation of CD45 signaling and the activity of CD45 cells including CD45RO+ T cells. The aggregation of CD45 isoforms on the cell surface and the concomitant downregulation of CD45 signaling is useful in diseases, disorders and conditions associated with hyper-responsive T cell activity including but not limited to autoimmune diseases, disorders and conditions.

3. Treatment of HIV Infection:

The CD45RO binding molecules of the present disclosure are useful in the modulation of HIV infection and are useful in the treatment of mammalian subjects suffering from HIV infection, especially chronic HIV infection. CD45 isoforms are identified as playing a role in the modulation of HIV infection. Although early events in the HIV-1 life cycle are independent of CD45 isoform expression, it has long been appreciated that HIV preferentially replicates in CD4+CD45RO+ memory cells rather than in CD4+CD45RB+ na\check{i}ve cells. CD4+CD45RO+ T cells are identified as a major latent virus reservoir in the HIV-infected subjects. An anti-CD45RO antibody conjugated to an immunotoxin (a ricin toxin fragment) was observed to reduce the frequency of both productively and lately infected cells while sparing CD4+CD45RA+ na\check{i}ve T cells and, to some extent, CD4+CD45ROlo memory T cells. Saavedra-Lozano, et al. (2002) The Journal of Infectious Diseases, Volume 185(3): 306-314; Ghetie, et al. (1991) Journal of Immunological Methods 142(2):222-223. The CD45 binding molecules of the present disclosure are useful in the selective elimination of productively and/or lately HIV infected cells. In one embodiment, the CD45 binding molecules of the present disclosure comprise CD45RO sdAbs conjugated to cytotoxic or cytostatic agents to provide targeted delivery of the cytotoxic or cytostatic agents to productively and/or lately HIV infected CD45RO T cells resulting in selective elimination of such infected cells and ameliorating the effects of HIV infection in the subject.

4. Autoimmune and Inflammatory Diseases:

In one embodiment the present disclosure provides a method of treating a T cell mediated autoimmune disease, the method comprising the administration of a CD45 binding molecule to a subject in an amount effective to inhibit a T-cell mediated immune response. CD45 binding molecules of the present disclosure specifically bind to the ECD of the CD45RO, either alone or associated with other molecules, and are useful in modulating the function of the cells expressing the CD45RO isoform and are useful in the treatment or prevention of diseases, disorders or conditions associated with inflammation or autoimmunity where immunological memory is involved in the cause, maintenance or exacerbation of the disease, disorder or condition.

The CD45 binding molecules of the present disclosure are useful in the treatment of a T cell-mediated or B cell-mediated autoimmune disease. Lazarovits, et al., U.S. Pat. No. 6,379,668B1 issued Apr. 30, 2002, teaches that anti-CD45RO antibodies and antibody fragments are useful in the treatment of a T cell mediated autoimmune disease. Additionally, patients with Crohn's disease are observed to exhibit elevated levels of CD45RO on CD19+ lamina propria B lymphocytes consistent with a population of late stage antigen activated B lymphocytes. Yacyshyn, et al., (1993) Gut 34 1698-1704. In one embodiment, the CD45 binding molecules of the present disclosure comprise CD45RO sdAbs conjugated to cytotoxic or cytostatic agents to provide targeted delivery of the cytotoxic or cytostatic agents to CD45RO B cells resulting in selective elimination of such cells and ameliorating the effects of B cell mediated autoimmune disease in a subject.

Autoimmune and inflammatory diseases, disorders or conditions amenable to treatment or prophylaxis with the compositions of the present disclosure include autoimmune diseases, such as, rheumatoid arthritis, psoriasis, psoriatic arthritis, autoimmune thyroiditis, Graves disease, type I and type II diabetes, multiple sclerosis, Crohn's disease (CD), inflammatory bowel disease (IBD), ulcerative colitis (UC), systemic lupus erythematosus (SLE), graft versus host disease (GVHD), psoriasis, and dermatitis. The tolerogenic properties CD45RO antibodies and complexes thereof of the present disclosure are useful in the induction of tolerance to alloantigens, autoantigens, allergens, bacterial antigens and conditions arising therefore including allergic contact dermatitis, and/or allergies, including allergic asthma.

In the treatment of autoimmune diseases, the CD45 binding molecule of the present disclosure may be administered alone or combination with one or more immunomodulatory or anti-inflammatory agents. Examples of immunomodulatory or anti-inflammatory agents which may be combined with administration CD45 binding molecule or incorporation by conjugated to a CD45 binding molecule include but are not limited to cyclosporins such as cyclosporine A, cyclosporine G, FK-506, ABT-281, ASM 981; an mTOR inhibitor, e.g. rapamycin, 40-O-(2-hydroxy)ethyl-rapamycin, CCI779, ABT578, AP23573, AP23464, AP23675, AP23841, TAFA-93, biolimus-7 or bioimus-9; a corticosteroid; cyclophosphamide; azathioprine; methotrexate; a S1P receptor agonist, e.g. FTY 720 or an analogue thereof; leflunomide or analogs thereof; mizoribine; mycophenolic acid; mycophenolate mofetil; 15-deoxyspergualine or analogs thereof; immunosuppressive monoclonal antibodies, e.g., monoclonal antibodies to leukocyte receptors, e.g., MHC, CD2, CDS, CD4, CD11a/CD18. CD7, CD25, CD27, B7, CD40, CD45, CD58, CD137, ICOS, CD150 (SLAM), OX40, 4-1BB or their ligands, e.g. CD154; or other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4lg (e.g. designated ATCC 68629) or a mutant thereof, e.g. LEA29Y, or other adhesion molecule inhibitors, e.g. mAbs or low molecular weight inhibitors including LFA-1 antagonists, selectin antagonists and VLA-4 antagonists.

5. Prevention of Organ Transplant Rejection

In some embodiments the CD45 binding molecules of the present disclosure are useful in the treatment or prevention of cell, tissue or organ transplant rejection in a mammalian recipient, comprising administering CD45 binding molecule in an amount effective to inhibit a T-cell mediated immune response in the subject. In the prevention of organ transplant rejection, the CD45 binding molecule of the present disclosure may be administered alone or combination with one or more immunomodulatory or anti-inflammatory agents as described above.

6. Purging of CD34+ Hematopoietic Stem Cells

In some embodiments, a CD45 binding molecule of the present disclosure are useful in the preparation of hematopoietic stem cells for transplant therapy. In one embodiment, a population of hematopoietic stem cells is contacted ex vivo with a CD45 binding molecule comprising a cytotoxic agent (e.g., a saporin, ricin toxin, chemotherapeutic agent) resulting in reduction or elimination of CD34+ hematopoietic stem cells from a population of stem cells.

7. Isolation, Enrichment or Depletion of CD45RO+ Cells From a Biological Sample:

In one embodiment, the present disclosure provides a method of use of the CD45 binding molecules of the present disclosure useful in a process for in the isolation, enrichment or depletion of CD45RO+ cells from a biological sample comprising CD45RO+ cells. The biological sample may comprise cells of blood origin such as PBMC, T cells, B cells of cell culture origin or of tissue origin such as brain or bone marrow. In some embodiments, the CD45RO+ cells include but are not limited to TILs, CAR-T cells, TCR engineered cells, recombinantly modified TILs, and recombinantly modified Tregs. Processes suitable for the isolation, enrichment or depletion of CD45RO+ cells comprise centrifugation, filtration, magnetic cell sorting and fluorescent cell sorting by techniques well known in the art. The present disclosure further provides a method for the treatment of a subject suffering from a disease, disorder or condition by the administration of a therapeutically effective amount of a cell product enriched or depleted of CD45RO+ cells through the use of a CD45 binding molecule as described herein.

In one embodiment, the sorting procedure employs a CD45 binding molecule comprising a fluorescent label for use in FACS isolation or depletion of CD45RO+ cells from a sample. The fluorescent label may be attached to the sdAb of the CD45 binding molecule directly (e.g. by chemical conjugation optionally employing a linker) or indirectly (e.g. by biotinylation of the sdAb and binding of the biotinylated antibody to a streptavidin fluorochrome conjugate). Such fluorescently labelled CD45RO+ cells may be separated from a mixed cell population using conventional FACS technology.

In an alternative embodiment, the selection procedure employs CD45 binding molecules of the present disclosure (e.g. a CD45RC VHH) conjugated to magnetic particles which provide magnetic labeling of the CD45RO+ cells for use in magnetic cell separation procedures. In one embodiment the method comprises: (a) conjugation of one or more CD45 binding molecule of the present disclosure (e.g. a CD45RO VHH) to a magnetic particle; (b) creating a mixture by contacting the biological sample with a quantity of the magnetic particles conjugated to CD45 binding molecule; (c) subjecting to a magnetic field such that the magnetically labelled CD45RO+ cells are retained; (d) removing the non-magnetically labelled cells from the mixture; and (e) removal of the magnetic field enabling isolation of the CD45RO+ cells.

The cell selection procedure (e.g., FACS or magnetic separation) results in two products: (a) a population of cells depleted of CD45RO+ cells and (b) a population of cells enriched for CD45RO+ cells. Each of these populations may be further processed by convention procedures to identify particular CD45RO+ or CD45RO-cell subsets which may be useful in research, diagnostic or clinical applications. For example, isolation of specific CD45RO+ T cell subsets that also express one or more of CD4, CD8, CD19, CD25, and CD62L, further iterations of the using one or more antibodies that specifically bind to CD4, CD8, CD19, CD25, and CD62L antigens respectively by FACS Cr magnetic field separation by techniques well known in the art.

In one embodiment of the CD45 binding molecule a humanized antibody or fragment thereof as disclosed herein may be used for depletion of CD45RO-expressing cells from a biological sample comprising CD45RO-expressing cells such peripheral blood or lymphoid tissue which may optionally be further processed for further isolation of CD45RO+ na\check{i}ve T cell subsets, isolation human CD45RO+ memory T cells from a population of CD4+ or CD8+ cells, or isolation of human CD45RA+ na\check{i}ve T cells from presorted CD4+ or CD8+ cells by depletion of CD45RO+ cells. In one embodiment, the CD45 binding molecule provides a method of generating a population of cells enriched for na\check{i}ve Tregs from a biological sample, the method comprising depleting CD45RO+ cells using a CD45 binding molecule of the present disclosure as described above, optionally further comprising the steps of depleting CD8+ and/or CD19+ cells. The CD45RO+ depleted cell population may optionally be further expanded in vitro for particular cell types to in the preparation of a cell product comprising a therapeutically effective amount of the CD45RO+ depleted cell product which may be administered to a subject suffering from a disease, disorder or condition.

In one embodiment, the CD45 binding molecule provides a method of generating a population of cell is depleted of CD45RO+ cells and enriched for CD62+ na\check{i}ve T cells useful in the preparation of CAR-T cells. The CD45 binding molecules of the present disclosure are useful in a method for the preparation of a biological sample enriched for CD62+ na\check{i}ve T cells comprising: (a) obtaining biological sample comprising a mixed population of T cells from a mammalian subject; (b) depleting the CD45RO+ cells as from the biological sample using a CD45 binding molecule as described herein; and (c) enriching the product of step (b) enriching the for CD62+ cells. The population of cells depleted of CD45RO+ cells and enriched for CD62+ na\check{i}ve T cells useful in the preparation of CAR-T cells such as by transduction with a recombinant vector (e.g. a retroviral vector or lentiviral vector) comprising a nucleic acid sequence encoding a chimeric antigen receptor in accordance with techniques well known in the art. The population of cells depleted of CD45RO+ cells and enriched for CD62+ na\check{i}ve T cells may be used in the preparation of CAR-T cells, including but not limited to CD19 CAR-T cells, BCMA CAR-T cells, PSMA CAR-T cells, CD19/CD20 CAR-T cells, CD20 CAR-T cells.

The CD45RO+ enriched cell population may optionally be further expanded in vitro to in the preparation of a cell product comprising a therapeutically effective amount of the CD45RC+ cells which may be administered to a subject suffering from a disease, disorder or condition.

8. Downregulation of CD45RO+ Memory Cells:

The molecular mechanism of TCR triggering in the immunological synapse (IS) has been explained by size-dependent kinetic segregation model. Shaw, A. S., and Dustin, M. L. (1997) Immunity 6, 361-369. The binding of TCRs its cognate antigens presented by MHC molecules on an antigen presenting cell, CD45 molecules are excluded from immunological synapse shifting the equilibrium to the phosphorylated state of the immunoreceptor tyrosine-based activation motifs (ITAMs) in the TCR-CD3 complex and allowing productive T cell activation CD45 exclusion is crucial for initial TCR triggering events and correlates with effective TCR signaling. Chang, et al. (2016) Nature Immunology 17, 574-582; Razvag, et al (2018) Nature Communications 9, 732; Varma, et al. (2006) Immunity 25, 117-127. Artificially induced CD45 exclusion can initiate TCR triggering in a non-immune cell reconstitution system. James, J. R., & Vale, R. D. (2012) Nature, 487(7405). The binding of CD45RO antibodies of the present CD45 binding molecule when conjugated to large carrier molecules (e.g. PEG, HSA) mimics the large mucin domain of CD45ABC isoform and may be used to exclude D45RO from the immune synapse resulting in downregulation of CD45RO+ memory cells.

9. Treatment of Neoplastic Disease

The present disclosure provides methods of use of CD45 binding molecules in the treatment of subjects suffering from a neoplastic disease disorder or condition by the administration of a therapeutically effective amount of an CD45 binding molecule (or nucleic acid encoding an CD45 binding molecule including recombinant vectors encoding CD45 binding molecules) as described herein. The compositions and methods of the present disclosure are useful in the treatment of subject suffering from a neoplastic disease characterized by the presence neoplasms, including benign and malignant neoplasms, and neoplastic disease.

In humans, CD45RO is characteristic of memory T cells. Merkenschlager, et al (1988) European Journal of Immunol 18: 1653-1661. CD45RO memory T cells are generated following an initial CD45RA cell-mediated immune response. CD45RO memory T cells may survive for many months and years after the antigen is eliminated and are believed to be responsible for a more rapid and intense response to subsequent exposures to the same antigens. The presence of high densities of CD45RO+ tumor infiltrating lymphocytes (CD45RO TILs) correlates with increased survival in cancers and the presence of CD45RO+ cells in a tumor (such as may be identified by administration of CD45 binding molecule comprising an imaging agent) is useful as prognostic factor of improved survival in cancer patients.

The presence of CD45RO isoform memory T cells are associated with improved anti-tumor response. For example, Yajima, et al. CD45RO expression in TILs as a predictor of prognosis in 98 patients with breast cancer who underwent radical surgery without neoadjuvant chemotherapy. of CD45RO+ memory cells are associated with a favorable prognosis breast cancer. Yajima, et al. (2016) Breast Cancer 23:668-674. These studies suggest that CD45RO+ effector cells may both help eradicate local tumors and prevent metastases to the lymphatic systems in breast cancer patients. The presence of a high ratio of CD45RO+ TILs was associated with recurrence-free survival improvement and a trend toward cancer-specific survival improvement in breast cancer patients.

Examples of benign neoplasms amenable to treatment using the compositions and methods of the present disclosure include but are not limited to adenomas, fibromas, hemangiomas, and lipomas. Examples of pre-malignant neoplasms amenable to treatment using the compositions and methods of the present disclosure include but are not limited to hyperplasia, atypia, metaplasia, and dysplasia. Examples of malignant neoplasms amenable to treatment using the compositions and methods of the present disclosure include but are not limited to carcinomas (cancers arising from epithelial tissues such as the skin or tissues that line internal organs), leukemias, lymphomas, and sarcomas typically derived from bone fat, muscle, blood vessels or connective tissues). Also included in the term neoplasms are viral induced neoplasms such as warts and EBV induced disease (i.e., infectious mononucleosis), scar formation, hyperproliferative vascular disease including intimal smooth muscle cell hyperplasia, restenosis, and vascular occlusion and the like.

The term “neoplastic disease” includes cancers characterized by solid tumors and non-solid tumors including but not limited to breast cancers; sarcomas (including but not limited to osteosarcomas and angiosarcomas and fibrosarcomas), leukemias, lymphomas, genitourinary cancers (including but not limited to ovarian, urethral, bladder, and prostate cancers); gastrointestinal cancers (including but not limited to colon esophageal and stomach cancers); lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas, astrocytomas, myelodysplastic disorders; cervical carcinoma-in-situ; intestinal polyposes; oral leukoplakias; histiocytoses, hyperprofroliferative scars including keloid scars, hemangiomas; hyperproliferative arterial stenosis, psoriasis, inflammatory arthritis; hyperkeratoses and papulosquamous eruptions including arthritis.

The term neoplastic disease includes carcinomas. The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term neoplastic disease includes adenocarcinomas. An “adenocarcinoma” refers to a carcinoma derived from glandular tissue or in which the tumor cells form recognizable glandular structures.

As used herein, the term “hematopoietic neoplastic disorders” refers to neoplastic diseases involving hyperplastic/neoplastic cells of hematopoietic origin, e.g., arising from myeloid, lymphoid or erythroid lineages, or precursor cells thereof.

Myeloid neoplasms include, but are not limited to, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage. Exemplary myeloid disorders amenable to treatment in accordance with the present disclosure include, but are not limited to, acute promyeloid leukemia (APML), acute myelogenous leukemia (AML) and chronic myelogenous leukemia (CML).

Lymphoid neoplasms include, but are not limited to, precursor lymphoid neoplasms, mature B-cell neoplasms, mature T-cell neoplasms, Hodgkin's Lymphoma, and immunodeficiency-associated lymphoproliferative disorders. Exemplary lymphic disorders amenable to treatment in accordance with the present disclosure include, but are not limited to, acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM).

In some instances, the hematopoietic neoplastic disorder arises from poorly differentiated acute leukemias (e.g., erythroblastic leukemia and acute megakaryoblastic leukemia). As used herein, the term “hematopoietic neoplastic disorders” refers malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease and Reed-Stemberg disease.

In one embodiment, the CD45 binding molecule of the present disclosure are useful in the treatment of CD45RO+ B-cell lymphomas. Although the majority B-cell lymphomas typically express only CD45RA, a small fraction of expressed CD45RO but not CD45RA. Caldwell, et al (1991) Clin Immunol Immunopathol 58(3):377-84. The CD45RO binding molecules of the present CD45 binding molecule are useful in targeting cytotoxic or cytostatic therapeutic molecules to these CD45RO+ B cell lymphomas.

The determination of whether a subject is “suffering from a neoplastic disease” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment.

10. Treatment of HCMV Infection:

The CD45 binding molecules of the present disclosure are useful in the treatment or prevention of HCMV infection in mammalian subjects by the administration of a therapeutically effective amount of an CD45 binding molecule (or nucleic acid encoding an CD45 binding molecule including recombinant vectors encoding CD45 binding molecules) as described herein. HCMV infection can be life-threatening in immunocompromised subjects such as newborn infants, organ transplant recipients, and HIV-infected subjects. Congenital cytomegalovirus infection (i.e. in the prenatal period) can lead to significant morbidity and even death. After infection, HCMV remains latent within the body throughout life and can be reactivated at any time. In some instances, HCMV infection has been associated with carcinomas including prostate cancer and breast cancer. The HCMV U11 protein has recently been shown to interact with CD45 resulting in functional paralysis of T cells. Gabaev, et al (2011) PLoS Pathogens. 7 (12): e1002432. Binding of the CMV pUL11 protein to CD45 on T cells prevents signal transduction via the TCR and restricts T cell proliferation. The interaction of CMV pUL11 with primary T cells is not dependent upon T cell activation state, as interactions between CMV pUL11 are observed with both long (e.g. CD45RA, CD45RABC) and short isoforms of CD45 (e.g. CD45RO) expressed on both na\check{i}ve and mature T cells suggesting that the immunomodulatory effects of pUL11 in vivo may be wide ranging, potentially affecting both priming and effector functions of T cells. Gabaev, et al. (2011) PLoS Pathogens. 7 (12): e1002432. The CD45 binding molecules of the present disclosure are useful in the treatment or prevention of HCMV infection in a subject by administration of a therapeutically effective amount of CD45 binding molecule to a subject sufficient to interfere with or prevent the binding of the HCMV pUL11 protein to the CD45 isoforms expressed on T cells, in particular the binding of HCMV pUL11 protein to the hCD45RO isotype expressed on CD45RO+ mature T cells.

11. Treatment of Adenovirus Infection:

The CD45 binding molecules of the present disclosure are useful in the treatment or prevention or ameliorating the effects of adenovirus infection in mammalian subjects by the administration of a therapeutically effective amount of a CD45 binding molecule (or nucleic acid encoding an CD45 binding molecule including recombinant vectors encoding CD45 binding molecules) as described herein. Windheim, et al. (2013) PNAS(USA) 110(50):E4884-93 demonstrate that CD45 is the target of the adenovirus 19a E3/49K (sec49K) protein which inhibits the activation of NK and T cells. The sec49K was observed to inhibit the up-regulation of the activation marker CD69 after CD3 stimulation of Jurkat T cells and the addition of purified sec49K markedly suppressed or delayed phosphorylation of the TCR target protein ZAP-70 as well as phosphorylation of the downstream target ERK-1/2. In the absence of CD3 stimulation, sec49K prevented ERK phosphorylation, whereas triggering with anti-CD3 Abs (OKT3) in the presence of sec49K drastically reduced phosphorylation of ERK demonstrating that sec49K interferes with activation signaling pathways in NK and T cells and thereby inhibits the function of these cells. The sec49K protein was further observed to lead to led to a significant decrease in the percentage CD4 T cells producing IFN-γ. Taken together, these findings indicate that binding of sec49K directly inhibits T-cell functionality, which is most pronounced for cells producing IFN-γ. Windheim, et al. conclude that the adenovirus adenovirus 19a E3/49K protein binds to all CD45 isoforms by attaching to the membrane-proximal fibronectin-like (D1, D2, D3 and D4) and/or cysteine-rich (R/T) domains of CD45. Consequently, the CD45 binding molecules of the present disclosure which specifically bind to the D1, D2, D3 and D4 domains of CD45 protein may be used to interfere or prevent with the binding of adenovirus 19a E3/49K to CD45 proteins and thereby prevent the concomitant inhibition of CD45+NK and T cells by adenovirus infection.

### K. Combination with Supplementary Therapeutic Agents

The present disclosure provides for the use of the CD45 binding molecules of the present disclosure in combination with one or more additional active agents (“supplementary agents”). Such further combinations are referred to interchangeably as “supplementary combinations” or “supplementary combination therapy” and those therapeutic agents that are used in combination with CD45 binding molecules of the present disclosure are referred to as “supplementary agents.” As used herein, the term “supplementary agents” includes agents that can be administered or introduced separately, for example, formulated separately for separate administration (e.g., as may be provided in a kit) and/or therapies that can be administered or introduced in combination with the CD45 binding molecules.

As used herein, the term “in combination with” when used in reference to the administration of multiple agents to a subject refers to the administration of a first agent at least one additional (i.e. second, third, fourth, fifth, etc.) agent to a subject. For purposes of the present invention, one agent (e.g. CD45 binding molecule) is considered to be administered in combination with a second agent (e.g. a modulator of an immune checkpoint pathway) if the biological effect resulting from the administration of the first agent persists in the subject at the time of administration of the second agent such that the therapeutic effects of the first agent and second agent overlap. For example, the PD1 immune checkpoint inhibitors (e.g. nivolumab or pembrolizumab) are typically administered by IV infusion every two weeks or every three weeks while the CD45 binding molecules of the present disclosure are typically administered more frequently, e.g. daily, BID, or weekly. However, the administration of the first agent (e.g. pembrolizumab) provides a therapeutic effect over an extended time and the administration of the second agent (e.g. an CD45 binding molecule) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the CD45 binding molecule and the supplementary agent(s) are administered or applied sequentially, e.g., where one agent is administered prior to one or more other agents. In other embodiments, the CD45 binding molecule and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

1. Chemotherapeutic Agents:

In some embodiments, the supplementary agent is a chemotherapeutic agent. In some embodiments the supplementary agent is a “cocktail” of multiple chemotherapeutic agents. In some embodiments the chemotherapeutic agent or cocktail is administered in combination with one or more physical methods (e.g. radiation therapy). The term “chemotherapeutic agents” includes but is not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins such as bleomycin A₂, cactinomycin, calicheamicin, carabicin, caminomycin, carzinophilin, chromomycins, dactinomycin, daunorubicin and derivatives such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C; mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, dideazatetrahydrofolic acid, and folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; taxanes such as paclitaxel, docetaxel, cabazitaxel; carminomycin, adriamycins such as 4′-epiadriamycin, 4-adriamycin-14-benzoate, adriamycin-14-octanoate, adriamycin-14-naphthaleneacetate; cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplementary agent is one or more chemical or biological agents identified in the art as useful in the treatment of neoplastic disease, including, but not limited to, a cytokines or cytokine antagonists such as IL-12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the CD45 binding molecule is administered in combination with BRAF/MEK inhibitors, kinase inhibitors such as sunitinib, PARP inhibitors such as olaparib, EGFR inhibitors such as osimertinib (Ahn, et al. (2016) J Thorac Oncol 11:S115), IDO inhibitors such as epacadostat, and oncolytic viruses such as talimogene laherparepvec (T-VEC).

2. Therapeutic Antibodies

In some embodiments, a “supplementary agent” is a therapeutic antibody (including bi-specific and tri-specific antibodies which bind to one or more tumor associated antigens including but not limited to bispecific T cell engagers (BITEs), dual affinity retargeting (DART) constructs, and trispecific killer engager (TriKE) constructs).

In some embodiments, the therapeutic antibody is an antibody that binds to at least one tumor antigen selected from the group consisting of HER2 (e.g. trastuzumab, pertuzumab, ado-trastuzumab emtansine), nectin-4 (e.g. enfortumab), CD79 (e.g. polatuzumab vedotin), CTLA4 (e.g. ipilumumab), CD22 (e.g. moxetumomab pasudotox), CCR4 (e.g. magamuizumab), IL23p19 (e.g. tildrakizumab), PDL1 (e.g. durvalumab, avelumab, atezolizumab), IL17a (e.g. ixekizumab), CD38 (e.g. daratumumab), SLAMF7 (e.g. elotuzumab), CD20 (e.g. rituximab, tositumomab, ibritumomab and ofatumumab), CD30 (e.g. brentuximab vedotin), CD33 (e.g. gemtuzumab ozogamicin), CD52 (e.g. alemtuzumab), EpCam, CEA, fpA33, TAG-72, CAIX, PSMA, PSA, folate binding protein, GD2 (e.g. dinuntuximab), GD3, IL6 (e.g. silutxumab) GM2, Ley, VEGF (e.g. bevacizumab), VEGFR, VEGFR2 (e.g. ramucirumab), PDGFR (e.g. olartumumab), EGFR (e.g. cetuximab, panitumumab and necitumumab), ERBB2 (e.g. trastuzumab), ERBB3, MET, IGF1R, EPHA3, TRAIL R1, TRAIL R2, RANKL RAP, tenascin, integrin αVβ3, and integrin α4β1.

3. Cell Therapy Agents and Methods as Supplementary Agents:

In some embodiments, the methods of the disclosure may include the administration of a CD45 binding molecule of the present disclosure in combination with supplementary agents in the form of cell therapies for the treatment of neoplastic, autoimmune or inflammatory diseases. Examples of cell therapies that are amenable to use in combination with the methods of the present disclosure include but are not limited to engineered T cell products comprising one or more first, second, third or fourth generation. CAR-T cells, engineered TCR cells, tumor infiltrating lymphocytes (TILs), and engineered Treg cells. In some embodiments, the extracellular domain of the chimeric antigen receptor of the CAR T cell is a polypeptide that specifically binds to one or more cell surface molecules preferentially or uniquely expressed on the extracellular surface of neoplastic cell (e.g. a tumor antigen) selected from the group consisting of GD2, BCMA, CD19, PSMA, CD33, CD38, CD70, GD2, IL3R□2, CD2, mesothelin, Her2, EpCam, Muc1, ROR1, CD133, CEA, EGRFRVIII, PSCA, GPC3, Pan-ErbB and FAP.

4. Activation-Induced Cell Death Inhibitors

In some embodiments the CD45 binding molecule is administered in combination with one or more supplementary agents that inhibit Activation-Induced Cell Death (AICD). AICD is a form of programmed cell death resulting from the interaction of Fas receptors (e.g., Fas, CD95) with Fas ligands (e.g., FasL, CD95 ligand), helps to maintain peripheral immune tolerance. The AICD effector cell expresses FasL, and apoptosis is induced in the cell expressing the Fas receptor. Activation-induced cell death is a negative regulator of activated T lymphocytes resulting from repeated stimulation of their T-cell receptors. Examples of agents that inhibit AICD that may be used in combination with the CD45 binding molecules described herein include but are not limited to cyclosporin A (Shih, et al., (1989) Nature 339:625-626, IL-16 and analogs (including rhIL-16, Idziorek, et al., (1998) Clinical and Experimental Immunology 112:84-91), TGFb1 (Genesteir, et al., (1999) J Exp Med189(2): 231-239), and vitamin E (Li-Weber, et al., (2002) J Clin Investigation 110(5):681-690).

5. Physical Methods

In some embodiments, the supplementary agent is a anti-neoplastic physical methods including but not limited to radiotherapy, cryotherapy, hyperthermic therapy, surgery, laser ablation, and proton therapy.

### L. Formulations

The present disclosure further provides pharmaceutically acceptable formulations of the CD45 binding molecules of the present disclosure. The preferred formulation depends on the intended mode of administration and therapeutic application. Pharmaceutical dosage forms of the CD45RO binding molecules described herein comprise physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of polypeptides include polysaccharides such as sodium carboxymethylcellulose or methylcellulose, polyvinylpyrrolidone, polyacrylates, polyoxyethylene-polyoxypropylene-block polymers, PEG, polymeric amino acids, amino acid copolymers, and lipid aggregates (such as oil droplets or liposomes).

The pharmaceutical compositions may also comprise pharmaceutically-acceptable, non-toxic carriers, excipients, stabilizers, or diluents, which are defined as vehicles commonly used to formulate pharmaceutical compositions for animal or human administration. The diluent is selected so as not to affect the biological activity of the combination. Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Formulations to be used for in vivo administration are typically sterile. Sterilization of the compositions of the present disclosure may readily accomplished by filtration through sterile filtration membranes.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (Langer, Science 249: 1527, 1990 and Hanes, Advanced Drug Delivery Reviews 28: 97-119, 1997). The agents of this disclosure can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

In some embodiments, the present disclosure provides methods of use of CD45RO binding molecules in the treatment of a subject suffering from an disease, disorder, or condition by the administration of a pharmaceutically acceptable formulation comprising therapeutically effective amount of nucleic acid encoding a polypeptide CD45 binding molecule including recombinant vectors encoding polypeptide CD45RO binding molecules to provide the in situ expression of the polypeptide CD45RO binding molecules in the subject. Expression vectors may be viral vectors or non-viral vectors. The term “non-viral vector” refers to an autonomously replicating, extrachromosomal circular DNA molecule, distinct from the normal genome and nonessential for cell survival under nonselective conditions capable of effecting the expression of a coding sequence in the target cell. Plasmids are examples of non-viral vectors. In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, or magnetic fields (electroporation).

In one embodiment, a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee, et al. (1997) Crit Rev Ther Drug Carrier Syst. 14:173-206); polymer coated liposomes (Marin et al., U.S. Pat. No. 5,213,804, issued May 25, 1993; Woodle, et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994).

In another embodiment, the expression vector may be a viral vector. As used herein, the term viral vector is used in its conventional sense to refer to any of the obligate intracellular parasites having no protein-synthesizing or energy-generating mechanism and generally refers to any of the enveloped or non-enveloped animal viruses commonly employed to deliver exogenous transgenes to mammalian cells. A viral vector may be replication competent (e.g., substantially wild-type), conditionally replicating (recombinantly engineered to replicate under certain conditions) or replication deficient (substantially incapable of replication in the absence of a cell line capable of complementing the deleted functions of the virus). The viral vector can possess certain modifications to make it “selectively replicating,” i.e. that it replicates preferentially in certain cell types or phenotypic cell states, e.g., cancerous. Viral vector systems useful in the practice of the instant CD45 binding molecule include, for example, naturally occurring or recombinant viral vector systems. Examples of viruses useful in the practice of the present CD45 binding molecule include recombinantly modified enveloped or non-enveloped DNA and RNA viruses. For example, viral vectors can be derived from the genome of human or bovine adenoviruses, vaccinia virus, lentivirus, herpes virus, adeno-associated virus, human immunodeficiency virus, sindbis virus, and retroviruses (including but not limited to Rous sarcoma virus), and hepatitis B virus. Typically, an expression cassette comprising a nucleic acid encoding the polypeptide CD45 binding molecule operably linked to an expression control sequence (e.g. a promoter) is inserted into such vectors to allow packaging of the gene construct, typically with accompanying viral genomic sequences, followed by infection of a sensitive host cell resulting in expression of the protein of interest (e.g. a polypeptide CD45RO binding molecule).

The expression vector may optionally encode one or more polypeptides in addition to the polypeptide CD45RO binding molecule. When expressing multiple polypeptides as in the practice of the present CD45 binding molecule, each polypeptide may be operably linked to an expression control sequence (monocistronic) or multiple polypeptides may be encoded by a polycistronic construct where multiple polypeptides are expressed under the control of a single expression control sequence. In one embodiment, the expression vector comprising the nucleic acid encoding the polypeptide CD45 binding molecule may optionally further encode one or more immunological modulator polypeptides. Examples of immunological modulators useful include but are not limited to cytokines. Examples of such cytokines are interleukins including but not limited to one more or of IL-1, IL-2, IL-3, IL-4, IL-7, IL-9, IL-10, IL-12, TNF-alpha, interferon alpha, interferon alpha-2b, interferon-beta, interferon-gamma, GM-CSF, and other suitable cytokines capable of modulating immune response and functional variants or derivatives thereof. The expressed cytokines can be directed for intracellular expression or expressed with a leader sequence for surface presentation or a signal peptide to provide extracellular secretion of the cytokine.

In some embodiments, the expression vector may optionally provide an additional expression cassette comprising a nucleic acid sequence encoding a “rescue” gene. A “rescue gene” is a nucleic acid sequence, the expression of which renders the cell susceptible to killing by external factors or causes a toxic condition in the cell such that the cell is killed. Providing a rescue gene enables selective cell killing of transduced cells. Thus the rescue gene provides an additional safety precaution when said constructs are incorporated into the cells of a mammalian subject to prevent undesirable spreading of transduced cells or the effects of replication competent vector systems. In one embodiment, the rescue gene is the thymidine kinase (TK) gene (see e.g. Woo, et al. U.S. Pat. No. 5,631,236 issued May 20, 1997 and Freeman, et al. U.S. Pat. No. 5,601,818 issued Feb. 11, 1997) in which the cells expressing the TK gene product are susceptible to selective killing by the administration of gancyclovir.

Alternatively, a cell, including as a cell isolated from the subject, may also be recombinantly modified to express a polypeptide CD45 binding molecule of the present disclosure by introducing into said isolated cell a nucleic acid encoding the polypeptide CD45RO binding molecules and the recombinantly modified cell re-adminstered to the subject. Expression of the polypeptide CD45RO binding molecule, may be effected by employment of the viral or non-viral vectors described above.

### M. Dosage

The present disclosure further provides the administration of therapeutically or prophylactically effective dose of CD45 binding molecule or a recombinant vector or cell comprising a nucleic acid sequence encoding a polypeptide CD45 binding molecule to a subject suffering from or at risk of developing, respectively, a disease, disorder or condition. The dosage of the pharmaceutical composition comprising the CD45RO binding molecules, vector or cell depends on factors including the route of administration, the disease to be treated, and physical characteristics, e.g., age, weight, general health, of the subject. Typically, the amount of a CD45 binding molecule contained within a single dose may be an amount that effectively prevents, delays, or treats the disease without inducing significant toxicity. A pharmaceutical composition of the disclosure may include a dosage of a CD45 binding molecule described herein ranging from 0.01 to 500 mg/kg (e.g., from 0.01 to 450 mg, from 0.01 to 400 mg, from 0.01 to 350 mg, from 0.01 to 300 mg, from 0.01 to 250 mg, from 0.01 to 200 mg, from 0.01 to 150 mg, from 0.01 to 100 mg, from 0.01 to 50 mg, from 0.01 to 10 mg, from 0.01 to 1 mg, from 0.1 to 500 mg/kg, from 1 to 500 mg/kg, from 5 to 500 mg/kg, from 10 to 500 mg/kg, from 50 to 500 mg/kg, from 100 to 500 mg/kg, from 150 to 500 mg/kg, from 200 to 500 mg/kg, from 250 to 500 mg/kg, from 300 to 500 mg/kg, from 350 to 500 mg/kg, from 400 to 500 mg/kg, or from 450 to 500 mg/kg) and, in a more specific embodiment, about 1 to about 100 mg/kg (e.g., about 1 to about 90 mg/kg, about 1 to about 80 mg/kg, about 1 to about 70 mg/kg, about 1 to about 60 mg/kg, about 1 to about 50 mg/kg, about 1 to about 40 mg/kg, about 1 to about 30 mg/kg, about 1 to about 20 mg/kg, about 1 to about 10 mg/kg, about 10 to about 100 mg/kg, about 20 to about 100 mg/kg, about 30 to about 100 mg/kg, about 40 to about 100 mg/kg, about 50 to about 100 mg/kg, about 60 to about 100 mg/kg, about 70 to about 100 mg/kg, about 80 to about 100 mg/kg, or about 90 to about 100 mg/kg). In some embodiments, a pharmaceutical composition of the disclosure may include a dosage of a binding protein described herein ranging from 0.01 to 20 mg/kg (e.g., from 0.01 to 15 mg/kg, from 0.01 to 10 mg/kg, from 0.01 to 8 mg/kg, from 0.01 to 6 mg/kg, from 0.01 to 4 mg/kg, from 0.01 to 2 mg/kg, from 0.01 to 1 mg/kg, from 0.01 to 0.1 mg/kg, from 0.01 to 0.05 mg/kg, from 0.05 to 20 mg/kg, from 0.1 to 20 mg/kg, from 1 to 20 mg/kg, from 2 to 20 mg/kg, from 4 to 20 mg/kg, from 6 to 20 mg/kg, from 8 to 20 mg/kg, from 10 to 20 mg/kg, from 15 to 20 mg/kg). The dosage may be adapted by the physician in accordance with conventional factors such as the extent of the disease and different parameters of the subject.

A pharmaceutical composition containing a CD45 binding molecule described herein can be administered to a subject in need thereof, for example, one or more times (e.g., 1-10 times or more) daily, weekly, monthly, biannually, annually, or as medically necessary. Dosages may be provided in either a single or multiple dosage regimens. The timing between administrations may decrease as the medical condition improves or increase as the health of the patient declines. A course of therapy may be a single dose or in multiple doses over a period of time. In some embodiments, a single dose is used. In some embodiments, two or more split doses administered over a period of 1, 2, 3, 4, 5, 6, 7, 8, 9, 10, 11, 12, 13, 14, 21, 28, 30, 60, 90, 120 or 180 days are used. Each dose administered in such split dosing protocols may be the same in each administration or may be different. Multi-day dosing protocols over time periods may be provided by the skilled artisan (e.g., physician) monitoring the administration, taking into account the response of the subject to the treatment including adverse effects of the treatment and their modulation as discussed above.

For prophylactic applications, pharmaceutical compositions or medicaments are administered to a patient susceptible to, or otherwise at risk of disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

In some embodiments the condition to be treated is a chronic condition (e.g., a chronic infection, i.e., an infection that is not cleared by the host immune system within a period of up to 1 week, 2 weeks, etc.). In some cases, chronic condition involve integration of pathogen genetic elements into the host genome, e.g., retroviruses, lentiviruses, Hepatitis B virus, etc. In other cases, chronic infections, for example certain intracellular bacteria or protozoan pathogens, result from a pathogen cell residing within a host cell. Additionally, in some embodiments, the infection is in a latent stage, as with herpes viruses or human papilloma viruses. In such instances, the course of therapy may involve the administration of the CD45 binding molecule over an extended period of time including continued administration in the substantial absence of the symptoms of the chronic condition to prevent recurrence of the chronic conditions or symptoms thereof.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

### N. Routes of Administration

Administration of a CD45RO binding molecules described herein may be achieved through any of a variety of art recognized methods including but not limited to the topical, intravascular injection (including intravenous or intraarterial infusion), intradermal injection, subcutaneous injection, intramuscular injection, intraperitoneal injection, intracranial injection, intratumoral injection, intranodal injection, transdermal, transmucosal, iontophoretic delivery, intralymphatic injection (Senti and Kundig (2009) Current Opinions in Allergy and Clinical Immunology 9(6):537-543), intragastric infusion, intraprostatic injection, intravesical infusion (e.g., bladder), respiratory inhalers including nebulizers, intraocular injection, intraabdominal injection, intralesional injection, intraovarian injection, intracerebral infusion or injection, intracerebroventricular injection (ICVI), and the like. Administration to the subject may be achieved by intravenous, as a bolus or by continuous infusion over a period of time. Examples of parenteral routes of administration include, for example, intravenous, intradermal, subcutaneous, transdermal (topical), transmucosal, and rectal administration. The CD45 binding molecule can be administered once, continuously, such as by continuous pump, or at periodic (e.g. daily, bi-weekly, monthly) intervals over a period of time can occur over the period of one week, two weeks, one month, two months, three months or more. Desired time intervals of multiple doses of the CD45 binding molecule may be determined by one of skill in the art.

As described hereinabove, the compositions of the present disclosure may be used in combination with one or more additional therapeutically effective agents. As used herein, the term “in combination with” when used in reference to the administration of multiple agents to a subject refers to the administration of a first agent at least one additional (i.e. second, third, fourth, fifth, etc.) supplementary agent to a subject. For purposes of the present disclosure, one agent (e.g. a CD45RO binding molecule) is considered to be administered in combination with a supplementary agent if the biological effect resulting from the administration of the first agent persists in the subject at the time of administration of the supplementary agent such that the therapeutic effects of the first agent and second agent overlap. The administration of the first agent may provide a therapeutic effect over an extended time and the administration of the supplementary agent provides its therapeutic effect while the therapeutic effect of the first agent remains ongoing such that the supplementary agent is considered to be administered in combination with the first agent, even though the first agent may have been administered at a point in time significantly distant (e.g. days or weeks) from the time of administration of the supplementary agent. In one embodiment, one agent is considered to be administered in combination with a supplementary agent if the first and second agents are administered simultaneously (within 30 minutes of each other), contemporaneously or sequentially. In some embodiments, a first agent is deemed to be administered “contemporaneously” with a supplementary agent if first and supplementary agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a supplementary agent are co-formulated in single pharmaceutically acceptable formulation and the co-formulation is administered to a subject. In certain embodiments, first agent and the supplementary agent(s) are administered or applied sequentially, e.g., where one agent is administered prior to one or more other agents. In other embodiments, the first agent and the supplementary agent(s) are administered simultaneously, for example where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

1. Vector Delivery of Polypeptide CD45 Binding Proteins

In those embodiments where the CD45 binding protein is a polypeptide (which may include but is not limited to a RIPR molecule), such CD45RO binding proteins may also be delivered to a subject through the administration of a recombinant vectors comprising a nucleic acid sequence encoding the peptidyl CD45 binding protein operably linked to an expression control sequence in the cells of the tissues of the subject.

Expression vectors may be viral vectors or non-viral vectors. The term “nonviral vector” refers to an autonomously replicating, extrachromosomal circular DNA molecule, distinct from the normal genome and nonessential for cell survival under nonselective conditions capable of effecting the expression of an coding sequence in the target cell. Plasmids are examples of non-viral vectors. In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, magnetic fields (electroporation)

In one embodiment, a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Crit Rev Ther Drug Carrier Syst. 14:173-206); polymer coated liposomes (Marin et al., U.S. Pat. No. 5,213,804, issued May 25, 1993; Woodle, et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994).

In another embodiment, the expression vector may be a viral vector. As used herein, the term viral vector is used in its conventional sense to refer to any of the obligate intracellular parasites having no protein-synthesizing or energy-generating mechanism and generally refers to any of the enveloped or non-enveloped animal viruses commonly employed to deliver exogenous transgenes to mammalian cells. A viral vector may be replication competent (e.g., substantially wild-type), conditionally replicating (recombinantly engineered to replicate under certain conditions) or replication deficient (substantially incapable of replication in the absence of a cell line capable of complementing the deleted functions of the virus). The viral vector can possess certain modifications to make it “selectively replicating,” i.e. that it replicates preferentially in certain cell types or phenotypic cell states, e.g., cancerous. Viral vector systems useful in the practice of the instant CD45 binding molecule include, for example, naturally occurring or recombinant viral vector systems. Examples of viruses useful in the practice of the present CD45 binding molecule include recombinantly modified enveloped or non-enveloped DNA and RNA viruses. For example, viral vectors can be derived from the genome of human or bovine adenoviruses, vaccinia virus, lentivirus, herpes virus, adeno-associated virus, human immunodeficiency virus, sindbis virus, and retroviruses (including but not limited to Rous sarcoma virus), and hepatitis B virus. Typically, genes of interest are inserted into such vectors to allow packaging of the gene construct, typically with accompanying viral genomic sequences, followed by infection of a sensitive host cell resulting in expression of the gene of interest (e.g. a targeting antigen).

The expression vector may encode one or more polypeptides in addition to the targeting antigen. When expressing multiple polypeptides as in the practice of the present CD45 binding molecule, each polypeptide may be operably linked to an expression control sequence (monocistronic) or multiple polypeptides may be encoded by a polycistronic construct where multiple polypeptides are expressed under the control of a single expression control sequence. In one embodiment, the expression vector encoding the targeting antigen may optionally further encode one or more immunological modulators. Examples of immunological modulators useful in the practice of the present CD45 binding molecule include but are not limited to cytokines. Examples of such cytokines are interleukins including but not limited to one more or of IL-1, IL-2, IL-3, IL-4, IL-12, TNF-alpha, interferon alpha, interferon alpha-2b, interferon-beta, interferon-gamma, GM-CSF, MIP1-alpha, MIP1-beta, MIP3-alpha, TGF-beta and other suitable cytokines capable of modulating immune response. The expressed cytokines can be directed for intracellular expression or expressed with a signal sequence for extracellular presentation or secretion. In one embodiment, in addition to an expression cassette for a targeting antigen, the expression vector further comprises expression cassettes comprising nucleic acid sequences encoding the A(p35) and B (p40) subunits of the heterodimeric IL-12 (p70) protein. Alternative to the use of multiple expression cassettes, the nucleic acid sequences encoding the A(p35) and B (p40) subunits of IL-12 may be encoded by a polycistronic construct, the expression cassette comprising the nucleic acid sequences encoding A(p35) and B (p40) subunits employing an internal ribosome entry site (IRES) element or the foot and mouth disease virus protein 2A (FMVD2A) to facilitate co-expression in the target cell.

The expression vector may optionally provide an additional expression cassette comprising a nucleic acid sequence encoding a “rescue” gene. A “rescue gene” is a nucleic acid sequence, the expression of which renders the cell susceptible to killing by external factors or causes a toxic condition in the cell such that the cell is killed. Providing a rescue gene enables selective cell killing of transduced cells. Thus, the rescue gene provides an additional safety precaution when said constructs are incorporated into the cells of a mammalian subject to prevent undesirable spreading of transduced cells or the effects of replication competent vector systems. In one embodiment, the rescue gene is the thymidine kinase (TK) gene (see e.g. Woo, et al. U.S. Pat. No. 5,631,236 issued May 20, 1997 and Freeman, et al. U.S. Pat. No. 5,601,818 issued Feb. 11, 1997) in which the cells expressing the TK gene product are susceptible to selective killing by the administration of gancyclovir.

### O. Kits

The present disclosure also contemplates kits comprising pharmaceutical compositions of CD45 binding molecules. In some embodiments, the kit further comprises supplementary pharmaceutical compositions comprising supplementary agents as discussed above for use in combination therapy with CD45 binding molecules. The kits are generally in the form of a physical structure housing various components, as described below, and can be utilized, for example, in practicing the methods described above. A kit may comprise a CD45 binding molecule in the form of a pharmaceutical composition suitable for administration to a subject that is ready for use or in a form or requiring preparation for example, thawing, reconstitution or dilution prior to administration. When the CD45 binding molecule is in a form that requires reconstitution by a user, the kit may also comprise a sterile container providing a reconstitution medium comprising buffers, pharmaceutically acceptable excipients, and the like. A kit of the present disclosure can be designed for conditions necessary to properly maintain the components housed therein (e.g., refrigeration or freezing). A kit may further contain a label or packaging insert including identifying information for the components therein and instructions for their use. Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial). Labels or inserts may be provided in a physical form or a computer readable medium. In some embodiments, the actual instructions are not present in the kit, but rather the kit provides a means for obtaining the instructions from a remote source, e.g., via an internet site, including by secure access by providing a password (or scannable code such as a barcode or QR code on the container of the CD45 binding molecule or kit comprising) in compliance with governmental regulations (e.g., HIPAA) are provided.

### Examples

The following examples are put forth so as to provide those of ordinary skill in the art with a complete disclosure and description of how to make and use the present CD45 binding molecule, and are not intended to limit the scope of what the inventors regard as their CD45 binding molecule nor are they intended to represent that the experiments below were performed and are all of the experiments that can be performed. It is to be understood that exemplary descriptions written in the present tense were not necessarily performed, but rather that the descriptions can be performed to generate the data and the like described therein. Efforts have been made to ensure accuracy with respect to numbers used (e.g., amounts, temperature, etc.), but some experimental errors and deviations should be accounted for. Variations of the particularly described procedures employed may become apparent to individuals or skill in the art and it is expected that those skilled artisans may employ such variations as appropriate. Accordingly, it is intended that the CD45 binding molecule be practiced otherwise than as specifically described herein, and that the invention includes all modifications and equivalents of the subject matter recited in the claims appended hereto as permitted by applicable law.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; QM=monthly; HPLC=high performance liquid chromatography; BW=body weight; U=unit; ns=not statistically significant; PBS=phosphate-buffered saline; PCR=polymerase chain reaction; NHS=N-hydroxysuccinimide; HSA=human serum albumin; MSA=mouse serum albumin; DMEM=Dulbeco's Modification of Eagle's Medium; GC=genome copy; EDTA=ethylenediaminetetraacetic acid; PBMCs=primary peripheral blood mononuclear cells; FBS=fetal bovine serum; FCS=fetal calf serum; HEPES=4-(2-hydroxyethyl)-1piperazineethanesulfonic acid; LPS=lipopolysaccharide; ATCC=American Type Culture Collection

### Example 1. Llama Immunization

For llama immunization, the full length ECD of human CD45 (amino acids 26-578) with a C-terminal Avi-His8 tag was expressed and purified from HiFive insect cell supernatant via Ni-agarose and size exclusion chromatography in Hepes-buffered saline. A single llama was immunized according standard methods and a phage display library constructed from post-immune PBMCs. A single round of phage panning was performed against BirA-biotinylated form of the immunogen. 93 ELISA positive clones were sequenced, which provided 51 unique, high quality VHH sequences. These clones were binned into 10 clonotypes, with ultimately 7 clonotypes showing binding to the D1-D4 region of human CD45RO.

### Example 2. Hybridoma Sequencing

The anti-CD45RO hybridomas (gap8.3, 4B2, and 9.4) were expanded using standard hybridoma culture protocols and RNA was isolated from 1×10⁶ cells. cDNA was synthesized using oligo dT primers, and 5′ RACE PCR performed using 3′ primers specific for mouse CH1. Amplicons were sequenced by Sanger sequencing.

### Example 3. Yeast Display Epitope Mapping

CD45 domains described in FIG. 2 were fused to the C-term of Aga2 with an N-term FLAG and C-term c-Myc tag. Yeast constructs were expressed on the surface of yeast (S. cervisiae ATCC strain YVH10). Yeast were grown overnight at 30C in synthetic selective media SD-SCAA, then induced again overnight at 20° C. in SG-SCAA. Yeast were incubated with individual a-CD45 antibodies or a-CD45 VHH for 60 minutes on ice, washed, and incubated with anti-mouse/anti-human Fc or anti-VHH Alexa647 secondary antibody as well as an anti-FLAG or anti-c-Myc Alexa488 secondary antibody. Labeled yeast were analyzed by flow cytometry on a Beckman Coulter Cytoflex® and analyzed using the FlowJo® software package.

### Example 4. Affinity Measurements

Affinities were determined using a Biacore T200 instrument by measuring Kon/Koff in single cycle kinetics mode. Hybridoma clones were either reformatted and expressed in the form of the parent IgG (gap8.3, 4B2, 9.4 as murine IgG2A, VHE as human IgG1), and scFvs were expressed as human IgG1 Fc fusions. For the anti-CD45 VHH, proteins were expressed as monomeric Fc fusions. The antibody ligands were captured on a Protein A chip (GE Life Sciences) and human CD45 D1-D4 (amino acids 194-578) was flowed over the chip as an analyte diluted in HBS-EP+ buffer. Human CD45 D1-D4 was generated in house with a C-terminal 8×His tag (SEQ ID NO: 610) and expressed and purified from expi293 cells by standard Ni- and SEC chromatography methods. Kinetic affinity constants were calculated using the Biacore® T200 software package. The results of these studies are summarized in Table 3 above.

The results of the epitope mapping of the VHHs described above conducted in substantial accordance with the teaching of Example 3 and the affinity measurements conducted in substantial accordance with the teaching of Example 4 above are summarized in the following Table 7 and the sequences of the VHHs and corresponding CDRs are provide in Table 4 above:

TABLE 7
Results of Epitope Mapping and Affinity Studies

| | | | | CD45 | |
|---|---|---|---|---|---|
| | | | Clonotype | Binding | |
| Clone | SEQ ID NO: | Clonotype | frequency | Domain | Notes |
| 1D1 | 114 | 1 | 1 | D3 | Weak |
| 1A7 | 138 | 2 | 11 | D4 | Weak |
| 2G4 | 150 | 2 | | D4 | Strong |
| 1E4 | 50 | 3 | 18 | D1D2 | Strong |
| 2H8 | 6 | 3 | | D1 | Strong |
| 1G4 | 94 | 4 | 6 | D2 | Strong |
| 2H5 | 98 | 4 | | D2 | Strong |
| 2F4 | 134 | 5 | 1 | D3D4 | Weak |
| 2C5 | 143 | 6 | 11 | D4 | Strong |
| 2D6 | 2 | 6 | | D4 | Strong |
| 1G1 | 146 | 7 | 31 | D1 | Strong |
| 2F8 | 54 | 7 | | D1D2 | Strong |
| 2H7 | 154 | 8 | 4 | D4 | Strong |
| 1D2 | 118 | 9 | 2 | D3 | Weak |
| 1A10 | 110 | 10 | 4 | D3 | Weak |
| 1D6 | 122 | 10 | | D3 | Weak |

1. A CD45 binding molecule that selectively binds to one or more of the D1, D2, D3 and/or D4 extracellular domains of hCD45.

2-6. (canceled)

7. The CD45 binding molecule of claim 1 wherein the CD45 binding molecules comprises a single domain antibody (sdAb).

8. The CD45 binding molecule of claim 7 wherein the single domain antibody (sdAb) having at least 80%, alternatively at least 85%, alternatively at least 90%, alternatively at least 95%, alternatively at least 98%, alternatively at least 99% identity to a polypeptide sequence selected from the group consisting of: SEQ ID NO: 2, SEQ ID NO: 34, SEQ ID NO: 38, SEQ ID NO: 78, SEQ ID NO: 82, SEQ ID NO: 94, SEQ ID NO: 106, SEQ ID NO: 134, SEQ ID NO: 126, and SEQ ID NO: 138.

9. The CD45 binding molecule of claim 8 wherein the single domain antibody (sdAb) is a VHH selected from the group consisting of: SEQ ID NO: 2, SEQ ID NO: 34, SEQ ID NO: 38, SEQ ID NO: 78, SEQ ID NO: 82, SEQ ID NO: 94, SEQ ID NO: 106, SEQ ID NO: 134, SEQ ID NO: 126, and SEQ ID NO: 138.

10. The CD45 binding molecule of claim 7, said sdAb comprising a triad of CDRs selected from the group consisting of:
SEQ ID NO: 3, SEQ ID NO: 4, and SEQ ID NO: 5;
SEQ ID NO: 7, SEQ ID NO: 8, and SEQ ID NO: 9;
SEQ ID NO: 11, SEQ ID NO: 12, and SEQ ID NO: 13;
SEQ ID NO: 15, SEQ ID NO: 16, and SEQ ID NO: 17;
SEQ ID NO: 19, SEQ ID NO: 20, and SEQ ID NO: 21;
SEQ ID NO: 23, SEQ ID NO: 24, and SEQ ID NO: 25;
SEQ ID NO: 27, SEQ ID NO: 28, and SEQ ID NO: 29;
SEQ ID NO: 31, SEQ ID NO: 32, and SEQ ID NO: 33;
SEQ ID NO: 35, SEQ ID NO: 36, and SEQ ID NO: 37;
SEQ ID NO: 39, SEQ ID NO: 40, and SEQ ID NO: 41;
SEQ ID NO: 43, SEQ ID NO: 44, and SEQ ID NO: 45;
SEQ ID NO: 47, SEQ ID NO: 48, and SEQ ID NO: 49;
SEQ ID NO: 51, SEQ ID NO: 52, and SEQ ID NO: 53;
SEQ ID NO: 55, SEQ ID NO: 56, and SEQ ID NO: 57;
SEQ ID NO: 59, SEQ ID NO: 60, and SEQ ID NO: 61;
SEQ ID NO: 63, SEQ ID NO: 64, and SEQ ID NO: 65;
SEQ ID NO: 67, SEQ ID NO: 68, and SEQ ID NO: 69;
SEQ ID NO: 71, SEQ ID NO: 72, and SEQ ID NO: 73;
SEQ ID NO: 75, SEQ ID NO: 76, and SEQ ID NO: 77;
SEQ ID NO: 79, SEQ ID NO: 80, and SEQ ID NO: 81;
SEQ ID NO: 83, SEQ ID NO: 84, and SEQ ID NO: 85;
SEQ ID NO: 87, SEQ ID NO: 88, and SEQ ID NO: 89;
SEQ ID NO: 91, SEQ ID NO: 92, and SEQ ID NO: 93;
SEQ ID NO: 95, SEQ ID NO: 96, and SEQ ID NO: 97;
SEQ ID NO: 99, SEQ ID NO: 100, and SEQ ID NO: 101;
SEQ ID NO: 103, SEQ ID NO: 104, and SEQ ID NO: 105;
SEQ ID NO: 107, SEQ ID NO: 108, and SEQ ID NO: 109;
SEQ ID NO: 111, SEQ ID NO: 112, and SEQ ID NO: 113;
SEQ ID NO: 115, SEQ ID NO: 116, and SEQ ID NO: 117;
SEQ ID NO: 119, SEQ ID NO: 120, and SEQ ID NO: 121;
SEQ ID NO: 123, SEQ ID NO: 124, and SEQ ID NO: 125;
SEQ ID NO: 127, SEQ ID NO: 128, and SEQ ID NO: 129;
SEQ ID NO: 131, SEQ ID NO: 132, and SEQ ID NO: 133;
SEQ ID NO: 135, SEQ ID NO: 136, and SEQ ID NO: 137;
SEQ ID NO: 139, SEQ ID NO: 140, and SEQ ID NO: 141;
SEQ ID NO: 143, SEQ ID NO: 144, and SEQ ID NO: 145;
SEQ ID NO: 147, SEQ ID NO: 148, and SEQ ID NO: 149;
SEQ ID NO: 151, SEQ ID NO: 152, and SEQ ID NO: 153;
SEQ ID NO: 155, SEQ ID NO: 156, and SEQ ID NO: 157;
SEQ ID NO: 159, SEQ ID NO: 160, and SEQ ID NO: 161;
SEQ ID NO: 163, SEQ ID NO: 164, and SEQ ID NO: 165;
SEQ ID NO: 167, SEQ ID NO: 168, and SEQ ID NO: 169;
SEQ ID NO: 171, SEQ ID NO: 172, and SEQ ID NO: 173;
SEQ ID NO: 175, SEQ ID NO: 176, and SEQ ID NO: 177;
SEQ ID NO: 179, SEQ ID NO: 180, and SEQ ID NO: 181;
SEQ ID NO: 183, SEQ ID NO: 184, and SEQ ID NO: 185;
SEQ ID NO: 187, SEQ ID NO: 188, and SEQ ID NO: 189;
SEQ ID NO: 191, SEQ ID NO: 192, and SEQ ID NO: 193;
SEQ ID NO: 195, SEQ ID NO: 196, and SEQ ID NO: 197;
SEQ ID NO:199, SEQ ID NO:200, and SEQ ID NO:201;
SEQ ID NO:203, SEQ ID NO:204, and SEQ ID NO:205;
SEQ ID NO:207, SEQ ID NO:208, and SEQ ID NO:209;
SEQ ID NO:211, SEQ ID NO:212, and SEQ ID NO:213;
SEQ ID NO:215, SEQ ID NO:216, and SEQ ID NO:217;
SEQ ID NO:219, SEQ ID NO:220, and SEQ ID NO:221;
SEQ ID NO:223, SEQ ID NO:224, and SEQ ID NO:225;
SEQ ID NO:227, SEQ ID NO:228, and SEQ ID NO:229;
SEQ ID NO:231, SEQ ID NO:232, and SEQ ID NO:233;
SEQ ID NO:235, SEQ ID NO:236, and SEQ ID NO:237;
SEQ ID NO:239, SEQ ID NO:240, and SEQ ID NO:241;
SEQ ID NO:243, SEQ ID NO:244, and SEQ ID NO:245;
SEQ ID NO:247, SEQ ID NO:248, and SEQ ID NO:249;
SEQ ID NO:251, SEQ ID NO:252, and SEQ ID NO:253;
SEQ ID NO:255, SEQ ID NO:256, and SEQ ID NO:257;
SEQ ID NO:259, SEQ ID NO:260, and SEQ ID NO:261;
SEQ ID NO:263, SEQ ID NO:264, and SEQ ID NO:265;
SEQ ID NO:267, SEQ ID NO:268, and SEQ ID NO:269;
SEQ ID NO:271, SEQ ID NO:272, and SEQ ID NO:273;
SEQ ID NO:275, SEQ ID NO:276, and SEQ ID NO:277;
SEQ ID NO:279, SEQ ID NO:280, and SEQ ID NO:281;
SEQ ID NO:283, SEQ ID NO:284, and SEQ ID NO:285;
SEQ ID NO:287, SEQ ID NO:288, and SEQ ID NO:289;
SEQ ID NO:291, SEQ ID NO:292, and SEQ ID NO:293;
SEQ ID NO:295, SEQ ID NO:296, and SEQ ID NO:297;
SEQ ID NO:299, SEQ ID NO:300, and SEQ ID NO:301;
SEQ ID NO:303, SEQ ID NO:304, and SEQ ID NO:305;
SEQ ID NO:307, SEQ ID NO:308, and SEQ ID NO:309;
SEQ ID NO:311, SEQ ID NO:312, and SEQ ID NO:313;
SEQ ID NO:315, SEQ ID NO:316, and SEQ ID NO:317;
SEQ ID NO:319, SEQ ID NO:320, and SEQ ID NO:321;
SEQ ID NO:323, SEQ ID NO:324, and SEQ ID NO:325;
SEQ ID NO:327, SEQ ID NO:328, and SEQ ID NO:329;
SEQ ID NO:331, SEQ ID NO:332, and SEQ ID NO:333;
SEQ ID NO:335, SEQ ID NO:336, and SEQ ID NO:337;
SEQ ID NO:339, SEQ ID NO:340, and SEQ ID NO:341;
SEQ ID NO:343, SEQ ID NO:344, and SEQ ID NO:345;
SEQ ID NO:347, SEQ ID NO:348, and SEQ ID NO:349;
SEQ ID NO:351, SEQ ID NO:352, and SEQ ID NO:353;
SEQ ID NO:355, SEQ ID NO:356, and SEQ ID NO:357;
SEQ ID NO:359, SEQ ID NO:360, and SEQ ID NO:361;
SEQ ID NO:363, SEQ ID NO:364, and SEQ ID NO:365;
SEQ ID NO:367, SEQ ID NO:368, and SEQ ID NO:369;
SEQ ID NO:371, SEQ ID NO:372, and SEQ ID NO:373;
SEQ ID NO:375, SEQ ID NO:376, and SEQ ID NO:377;
SEQ ID NO:379, SEQ ID NO:380, and SEQ ID NO:381;
SEQ ID NO:383, SEQ ID NO:384, and SEQ ID NO:385;
SEQ ID NO:387, SEQ ID NO:388, and SEQ ID NO:389;
SEQ ID NO:391, SEQ ID NO:392, and SEQ ID NO:393;
SEQ ID NO:395, SEQ ID NO:396, and SEQ ID NO:397;
SEQ ID NO:399, SEQ ID NO:400, and SEQ ID NO:401;
SEQ ID NO:403, SEQ ID NO:404, and SEQ ID NO:405;
SEQ ID NO:407, SEQ ID NO:408, and SEQ ID NO:409;
SEQ ID NO:411, SEQ ID NO:412, and SEQ ID NO:413;
SEQ ID NO:415, SEQ ID NO:416, and SEQ ID NO:417;
SEQ ID NO:419, SEQ ID NO:420, and SEQ ID NO:421;
SEQ ID NO:423, SEQ ID NO:424, and SEQ ID NO:425;
SEQ ID NO:427, SEQ ID NO:428, and SEQ ID NO:429;
SEQ ID NO:431, SEQ ID NO:432, and SEQ ID NO:433;
SEQ ID NO:435, SEQ ID NO:436, and SEQ ID NO:437;
SEQ ID NO:439, SEQ ID NO:440, and SEQ ID NO:441;
SEQ ID NO:443, SEQ ID NO:444, and SEQ ID NO:445;
SEQ ID NO:447, SEQ ID NO:448, and SEQ ID NO:449;
SEQ ID NO:451, SEQ ID NO:452, and SEQ ID NO:453;
SEQ ID NO:455, SEQ ID NO:456, and SEQ ID NO:457;
SEQ ID NO:459, SEQ ID NO:460, and SEQ ID NO:461;
SEQ ID NO:463, SEQ ID NO:464, and SEQ ID NO:465;
SEQ ID NO:467, SEQ ID NO:468, and SEQ ID NO:469;
SEQ ID NO:471, SEQ ID NO:472, and SEQ ID NO:473;
SEQ ID NO:475, SEQ ID NO:476, and SEQ ID NO:477;
SEQ ID NO:479, SEQ ID NO:480, and SEQ ID NO:481;
SEQ ID NO:483, SEQ ID NO:484, and SEQ ID NO:485;
SEQ ID NO:487, SEQ ID NO:488, and SEQ ID NO:489;
SEQ ID NO:491, SEQ ID NO:492, and SEQ ID NO:493;
SEQ ID NO:495, SEQ ID NO:496, and SEQ ID NO:497;
SEQ ID NO:499, SEQ ID NO:500, and SEQ ID NO:501;
SEQ ID NO:503, SEQ ID NO:504, and SEQ ID NO:505;
SEQ ID NO:507, SEQ ID NO:508, and SEQ ID NO:509;
SEQ ID NO:511, SEQ ID NO:512, and SEQ ID NO:513;
SEQ ID NO:515, SEQ ID NO:516, and SEQ ID NO:517;
SEQ ID NO:519, SEQ ID NO:520, and SEQ ID NO:521; and
SEQ ID NO:523, SEQ ID NO:524, and SEQ ID NO:525.

11. The CD45 binding molecule of claim 7, said sdAb comprising a triad of CDRs selected from the group consisting of:
SEQ ID NO: 3, SEQ ID NO: 4, and SEQ ID NO: 5;
SEQ ID NO: 35, SEQ ID NO: 36, and SEQ ID NO: 37;
SEQ ID NO: 39, SEQ ID NO: 40, and SEQ ID NO: 41;
SEQ ID NO: 79, SEQ ID NO: 80, and SEQ ID NO: 81;
SEQ ID NO: 83, SEQ ID NO: 84, and SEQ ID NO: 85;
SEQ ID NO: 95, SEQ ID NO: 96, and SEQ ID NO: 97;
SEQ ID NO: 127, SEQ ID NO: 128, and SEQ ID NO: 129;
SEQ ID NO: 135, SEQ ID NO: 136, and SEQ ID NO: 137; and
SEQ ID NO: 139, SEQ ID NO: 140, and SEQ ID NO: 141.

12. The CD45 binding molecule of claim 7, said sdAb comprising a polypeptide at least 80%, alternatively at least 85%, alternatively at least 90%, alternatively at least 95%, alternatively at least 98%, alternatively at least 99% identity to a polypeptide sequence selected from the group consisting of: SEQ ID NO: 2, SEQ ID NO: 34, SEQ ID NO: 38, SEQ ID NO: 78, SEQ ID NO: 82, SEQ ID NO: 94, SEQ ID NO: 106, SEQ ID NO: 134, SEQ ID NO: 126, and SEQ ID NO: 138.

13. The CD45 binding molecule of claim 7, said sdAb comprising a polypeptide at least 80%, alternatively at least 85%, alternatively at least 90%, alternatively at least 95%, alternatively at least 98%, alternatively at least 99% identity to a polypeptide sequence selected from the group consisting of: SEQ ID NO: 2, SEQ ID NO: 6, SEQ ID NO: 10, SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, SEQ ID NO: 90, SEQ ID NO: 94, SEQ ID NO: 98, SEQ ID NO: 102, SEQ ID NO: 106, SEQ ID NO: 110, SEQ ID NO: 114, SEQ ID NO: 118, SEQ ID NO: 122, SEQ ID NO: 126, SEQ ID NO: 130, SEQ ID NO: 134, SEQ ID NO: 138, SEQ ID NO: 142, SEQ ID NO: 146, SEQ ID NO: 150, SEQ ID NO: 154, SEQ ID NO: 158, SEQ ID NO: 162, SEQ ID NO: 166, SEQ ID NO: 170, SEQ ID NO: 174, SEQ ID NO: 178, SEQ ID NO: 182, SEQ ID NO: 186, SEQ ID NO: 190, SEQ ID NO: 194, SEQ ID NO: 198, SEQ ID NO:204, SEQ ID NO: 208, SEQ ID NO: 212, SEQ ID NO: 216, SEQ ID NO: 220, SEQ ID NO: 224, SEQ ID NO:228, SEQ ID NO: 232, SEQ ID NO: 236, SEQ ID NO: 240, SEQ ID NO: 244, SEQ ID NO: 248, SEQ ID NO: 252, SEQ ID NO: 256, SEQ ID NO: 260, SEQ ID NO: 264, SEQ ID NO: 268, SEQ ID NO: 272, SEQ ID NO: 276, SEQ ID NO: 280, SEQ ID NO: 284, SEQ ID NO: 288, SEQ ID NO: 292, SEQ ID NO: 296, SEQ ID NO: 300, SEQ ID NO: 304, SEQ ID NO: 308, SEQ ID NO: 312, SEQ ID NO: 316, SEQ ID NO:320, SEQ ID NO: 324, SEQ ID NO: 328, SEQ ID NO: 332, SEQ ID NO: 336, SEQ ID NO: 340, SEQ ID NO: 344, SEQ ID NO: 348, SEQ ID NO: 352, SEQ ID NO: 356, SEQ ID NO: 360, SEQ ID NO: 364, SEQ ID NO: 368, SEQ ID NO: 372, SEQ ID NO: 376, SEQ ID NO: 380, SEQ ID NO: 384, SEQ ID NO: 388, SEQ ID NO: 392, SEQ ID NO: 396, SEQ ID NO: 400, SEQ ID NO: 404, SEQ ID NO: 408, SEQ ID NO: 412, SEQ ID NO: 416, SEQ ID NO: 420, SEQ ID NO: 424, SEQ ID NO: 428, SEQ ID NO: 432, SEQ ID NO: 436, SEQ ID NO: 440, SEQ ID NO: 444, SEQ ID NO: 448, SEQ ID NO: 452, SEQ ID NO: 456, SEQ ID NO: 460, SEQ ID NO: 464, SEQ ID NO: 470, SEQ ID NO: 474, SEQ ID NO: 478, SEQ ID NO: 482, SEQ ID NO: 486, SEQ ID NO: 490, SEQ ID NO: 494, SEQ ID NO: 498, SEQ ID NO: 502, SEQ ID NO: 506, SEQ ID NO: 510, SEQ ID NO: 514, SEQ ID NO: 518, and SEQ ID NO: 522.

14. The CD45 binding molecule of claim 7, said sdAb comprising a polypeptide selected from the group consisting of: SEQ ID NO: 2, SEQ ID NO: 34, SEQ ID NO: 38, SEQ ID NO: 78, SEQ ID NO: 82, SEQ ID NO: 94, SEQ ID NO: 106, SEQ ID NO: 134, SEQ ID NO: 126, and SEQ ID NO: 138.

15. (canceled)

16. The CD45 binding molecule of claim 1, am wherein the CD45 binding molecule is a multivalent binding molecule comprising a first domain comprising a polypeptide that specifically binds to an isoform of CD45 in stable association with a second domain that specifically binds to the extracellular domain of a cell surface molecule.

17. The CD45 binding molecule of claim 11 the second domain binds to the extracellular domain of one or more cell surface receptors that signal through a phosphorylation mechanism selected from the group consisting of immune checkpoint receptors, cytokine receptors and growth hormone receptors.

18. The CD45 binding molecule of claim 1, wherein the sdAb is humanized.

19. A method of use of the CD45 binding molecule of claim 1 for use in treatment or prevention of a disease, disorder or condition in a mammalian subject by administering to said subject a therapeutically effective amount of a CD45 binding molecule of claim 1 or a pharmaceutically acceptable formulation thereof.

20. The method of claim 19 wherein the disease, disorder or condition is a neoplastic disease, disorder or condition.

21. (canceled)

22. (canceled)

23. (canceled)

24. A method of use of the CD45 binding molecule of claim 1 for use isolation, depletion or enrichment of CD45RO+ cells a biological sample.

25. A nucleic acid sequence encoding the CD45 binding molecule or the polypeptide domains of the CD45 binding molecule of claim 1.

26. A recombinant viral or non-viral vector comprising a nucleic acid of claim 24.

27. A host cell comprising a nucleic acid of claim 24.

28. A pharmaceutical formulations comprising the viral or non-viral vector of claim 25.

29. A kit comprising the CD45 binding molecules of claim 1.


SEQUENCE LISTING (611 sequences)

SEQ ID NO: 1 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 2 - protein, 127 aa, Artificial Sequence
  Description of Artificial Sequence Synthetic polypeptide
QVQLQASGGGLVQAGGSLRLSCAASGRTVSSDAMGWFRQAPGKEREFVAAINWNGASTYYGDSAQGRFTISRDNAKNTVYLQMNSLKSEDTAVYYCAASDWVVGGISLMNGAEYKYWGQGTLVTVSS

SEQ ID NO: 3 - protein, 5 aa, Artificial Sequence
  Description of Artificial Sequence Synthetic peptide
SDAMG

SEQ ID NO: 4 - protein, 17 aa, Artificial Sequence
  Description of Artificial Sequence Synthetic peptide
AINWNGASTYYGDSAQG

SEQ ID NO: 5 - protein, 18 aa, Artificial Sequence
  Description of Artificial Sequence Synthetic peptide
SDWVVGGISLMNGAEYKY

SEQ ID NO: 6 - protein, 123 aa, Artificial Sequence
  Description of Artificial Sequence Synthetic polypeptide
QVQLQESGGGLVQAGGSLRLSCVASGRTFSSYGMGWFRQAPGKEREFVASISWSGGSTYYPDSVLGRFTISRDNAKNVLYLQMNSLKPEDTAVYYCAASSTWNNNDPHEYDYWGQGTLVTVSS

SEQ ID NO: 7 - protein, 5 aa, Artificial Sequence
  Description of Artificial Sequence Synthetic peptide
SYGMG

SEQ ID NO: 8 - protein, 17 aa, Artificial Sequence
  Description of Artificial Sequence Synthetic peptide
SISWSGGSTYYPDSVLG

SEQ ID NO: 9 - protein, 14 aa, Artificial Sequence
  Description of Artificial Sequence Synthetic peptide
SSTWNNNDPHEYDY

SEQ ID NO: 10 - protein, 123 aa, Artificial Sequence
  Description of Artificial Sequence Synthetic polypeptide
QVQLQESGGGLVQPGGSLRLSCAASGRTFSSYGMGWFRQAPGKEREFVSSISWSGRSTYYTNAVRGRFTISRDNAKNTVYLQMNSLKPEDTAVYYCAASSTWNNNDPTEYDYWGQGTLVTVSS

SEQ ID NO: 11 - protein, 5 aa, Artificial Sequence
  Description of Artificial Sequence Synthetic peptide
SYGMG

SEQ ID NO: 12 - protein, 17 aa, Artificial Sequence
  Description of Artificial Sequence Synthetic peptide
SISWSGRSTYYTNAVRG

SEQ ID NO: 13 - protein, 14 aa, Artificial Sequence
  Description of Artificial Sequence Synthetic peptide
SSTWNNNDPTEYDY

SEQ ID NO: 14 - protein, 123 aa, Artificial Sequence
  Description of Artificial Sequence Synthetic polypeptide
QVQLQEFGGGLVQPGGSLRLSCAASGRTFSDYAMGWFRQAPGKEREFVASISWSGGSLYYAGAVRGRFTISRDNAKNTVYLQMNSLKPEDTAVYYCAASSTWNNNDPHEYDYWGQGTQVTVSS

SEQ ID NO: 15 - protein, 5 aa, Artificial Sequence
  Description of Artificial Sequence Synthetic peptide
DYGMG

SEQ ID NO: 16 - protein, 17 aa, Artificial Sequence
  Description of Artificial Sequence Synthetic peptide
SSSWSGRSTYYAAAVRG

SEQ ID NO: 17 - protein, 14 aa, Artificial Sequence
  Description of Artificial Sequence Synthetic peptide
SSSWNNNDPSEYEY

SEQ ID NO: 18 - protein, 123 aa, Artificial Sequence
  Description of Artificial Sequence Synthetic polypeptide
QVQLQQSGGDLVQPGGSLRLSCAASGRTFSDYGMGWFRQAPGKEREFVASSSWSGRSTYYAAAVRGRFTISRDNAKSTVYLQMNSLKAEDTAVYYCAASSSWNNNDPSEYEYWGQGTQVTVSS

SEQ ID NO: 19 - protein, 5 aa, Artificial Sequence
  Description of Artificial Sequence Synthetic peptide
DYGMG

SEQ ID NO: 20 - protein, 17 aa, Artificial Sequence
  Description of Artificial Sequence Synthetic peptide
SSSWSGRSTYYAAAVRG

SEQ ID NO: 21 - protein, 14 aa, Artificial Sequence
  Description of Artificial Sequence Synthetic peptide
SSSWNNNDPSEYEY

SEQ ID NO: 22 - protein, 123 aa, Artificial Sequence
  Description of Artificial Sequence Synthetic polypeptide
QVQLQAFGGGTVQAGGSLRLSCTASGGTFSDYGMGWFRQAPGKEREFVSSISWSGNSVYYAGAVRGRFTISRDNAKNTVYLQMNSLQPEDTAVYYCAASSTWNNNDPTEYEYWGQGTQVTVSS

SEQ ID NO: 23 - protein, 5 aa, Artificial Sequence
  Description of Artificial Sequence Synthetic peptide
DYGMG

SEQ ID NO: 24 - protein, 17 aa, Artificial Sequence
  Description of Artificial Sequence Synthetic peptide
SISWSGNSVYYAGAVRG

SEQ ID NO: 25 - protein, 14 aa, Artificial Sequence
  Description of Artificial Sequence Synthetic peptide
SSTWNNNDPTEYEY

SEQ ID NO: 26 - protein, 123 aa, Artificial Sequence
  Description of Artificial Sequence Synthetic polypeptide
QVQLQQFGGGLVQPGGSLRLSCAASGRTFSDYGMGWFRQAPGKEREFVASSSWSGRSTYYAAAVRGRFTISRDNAKSTVYLQMNSLKAEDTAVYYCAASSSWNNNDPSEYEYWGQGTLVTVSS

SEQ ID NO: 27 - protein, 5 aa, Artificial Sequence
  Description of Artificial Sequence Synthetic peptide
DYGMG

SEQ ID NO: 28 - protein, 17 aa, Artificial Sequence
  Description of Artificial Sequence Synthetic peptide
SSSWSGRSTYYAAAVRG

SEQ ID NO: 29 - protein, 14 aa, Artificial Sequence
  Description of Artificial Sequence Synthetic peptide
SSSWNNNDPSEYEY

SEQ ID NO: 30 - protein, 123 aa, Artificial Sequence
  Description of Artificial Sequence Synthetic polypeptide
QVQLQEFGGGLVQPGGSLRLSCAASGRTFSDYGMGWFRQAPGKEREFVASSSWSGRSTYYAAAVRGRFTISRDNAKSTVYLQMNSLKAEDTAVYYCAASSSWNNNDPSEYEYWGQGTQVTVSS

SEQ ID NO: 31 - protein, 5 aa, Artificial Sequence
  Description of Artificial Sequence Synthetic peptide
DYGMG

SEQ ID NO: 32 - protein, 17 aa, Artificial Sequence
  Description of Artificial Sequence Synthetic peptide
SSSWSGRSTYYAAAVRG

SEQ ID NO: 33 - protein, 14 aa, Artificial Sequence
  Description of Artificial Sequence Synthetic peptide
SSSWNNNDPSEYEY

SEQ ID NO: 34 - protein, 123 aa, Artificial Sequence
  Description of Artificial Sequence Synthetic polypeptide
QVQLQAFGGGLVQPGGSLRLSCAASGGTFSDYGMGWFRQAPGKEREFVASSSWSGRSTYYAAAVRGRFTISRDNAKSLVYLQMNNLKPEDTAVYYCAASSTWNNNDPSEYEYWGQGTQVTVSS

SEQ ID NO: 35 - protein, 5 aa, Artificial Sequence
  Description of Artificial Sequence Synthetic peptide
DYGMG

SEQ ID NO: 36 - protein, 17 aa, Artificial Sequence
  Description of Artificial Sequence Synthetic peptide
SSSWSGRSTYYAAAVRG

SEQ ID NO: 37 - protein, 14 aa, Artificial Sequence
  Description of Artificial Sequence Synthetic peptide
SSTWNNNDPSEYEY

SEQ ID NO: 38 - protein, 123 aa, Artificial Sequence
  Description of Artificial Sequence Synthetic polypeptide
QVQLQESGGGLVEAGGSLRLSCAASGGTFSDYGMGWFRQAPGKEREFVSSISWSGNSVYYAGAVRGRFTISRDNAKNTVYLQMNSLQPEDTAVYYCAASSTWNNNDPTEYEYWGQGTQVTVSS

SEQ ID NO: 39 - protein, 5 aa, Artificial Sequence
  Description of Artificial Sequence Synthetic peptide
DYGMG

SEQ ID NO: 40 - protein, 17 aa, Artificial Sequence
  Description of Artificial Sequence Synthetic peptide
SISWSGNSVYYAGAVRG

SEQ ID NO: 41 - protein, 14 aa, Artificial Sequence
  Description of Artificial Sequence Synthetic peptide
SSTWNNNDPTEYEY

SEQ ID NO: 42 - protein, 123 aa, Artificial Sequence
  Description of Artificial Sequence Synthetic polypeptide
QVQLQASGGGLVQPGGSLRLSCAASGRTFSSYGMGWFRQAPGKEREFVASISWSGRSTYYTNAVRGRFTISRDNAKNTVYLQMNSLKPEDTAVYYCAASSTWNNNDPTEYEYWGQGTQVTVSS

SEQ ID NO: 43 - protein, 5 aa, Artificial Sequence
  Description of Artificial Sequence Synthetic peptide
SYGMG

SEQ ID NO: 44 - protein, 17 aa, Artificial Sequence
  Description of Artificial Sequence Synthetic peptide
SISWSGRSTYYTNAVRG

SEQ ID NO: 45 - protein, 14 aa, Artificial Sequence
  Description of Artificial Sequence Synthetic peptide
SSTWNNNDPTEYEY

SEQ ID NO: 46 - protein, 123 aa, Artificial Sequence
  Description of Artificial Sequence Synthetic polypeptide
QVQLQESGGGLVQPGGSLRLSCAASGRTFSSYGMGWFRQAPGKEREFVSSISWSGRSTYYTNAVRGRFTISRDNAKNTVYLQMNSLKPEDTAVYYCAASSTWNNNDPTEYEYWGQGTQVTVSS

SEQ ID NO: 47 - protein, 5 aa, Artificial Sequence
  Description of Artificial Sequence Synthetic peptide
SYGMG

SEQ ID NO: 48 - protein, 17 aa, Artificial Sequence
  Description of Artificial Sequence Synthetic peptide
SISWSGRSTYYTNAVRG

SEQ ID NO: 49 - protein, 14 aa, Artificial Sequence
  Description of Artificial Sequence Synthetic peptide
SSTWNNNDPTEYEY

SEQ ID NO: 50 - protein, 123 aa, Artificial Sequence
  Description of Artificial Sequence Synthetic polypeptide
QVQLQESGGGLVQPGGSLRLSCAASGGTFSDYGMGWFRQAPGKEREFVSSISWSGNSVYYAGAVRGRFTISRDNAKNTVYLQMNSLQPEDTAVYYCAASSTWNNNDPTEYEYWGQGTLVTVSS

SEQ ID NO: 51 - protein, 5 aa, Artificial Sequence
  Description of Artificial Sequence Synthetic peptide
DYGMG

SEQ ID NO: 52 - protein, 17 aa, Artificial Sequence
  Description of Artificial Sequence Synthetic peptide
SISWSGNSVYYAGAVRG

SEQ ID NO: 53 - protein, 14 aa, Artificial Sequence
  Description of Artificial Sequence Synthetic peptide
SSTWNNNDPTEYEY

SEQ ID NO: 54 - protein, 127 aa, Artificial Sequence
  Description of Artificial Sequence Synthetic polypeptide
QVQLQQFGGGLVQAGGSLRLSCAASGRTFSGYAMGWFRQAPGKEREFVAAINWSGGSTYYADSVKGRYTISRDNAKNTGYLQMNSLKSEDTAVYYCAASDWVVGGISLMNGAEYKYWGQGTLVTVSS

SEQ ID NO: 55 - protein, 5 aa, Artificial Sequence
  Description of Artificial Sequence Synthetic peptide
GYAMG

SEQ ID NO: 56 - protein, 17 aa, Artificial Sequence
  Description of Artificial Sequence Synthetic peptide
AINWSGGSTYYADSVKG

SEQ ID NO: 57 - protein, 18 aa, Artificial Sequence
  Description of Artificial Sequence Synthetic peptide
SDWVVGGISLMNGAEYKY

SEQ ID NO: 58 - protein, 127 aa, Artificial Sequence
  Description of Artificial Sequence Synthetic polypeptide
QVQLQESGGGLVQAGGSLRLSCAASGRTVSSDAMGWFRQAPGKEREFVAAINWNGASTYYADSVKGRFTISRDNAKNTVYLQMNSLKSEDTAVYYCAASDWVVGGISLMNGAEYAYWGQGTQVTVSS

SEQ ID NO: 59 - protein, 5 aa, Artificial Sequence
  Description of Artificial Sequence Synthetic peptide
SDAMG

SEQ ID NO: 60 - protein, 17 aa, Artificial Sequence
  Description of Artificial Sequence Synthetic peptide
AINWNGASTYYADSVKG

SEQ ID NO: 61 - protein, 18 aa, Artificial Sequence
  Description of Artificial Sequence Synthetic peptide
SDWVVGGISLMNGAEYAY

SEQ ID NO: 62 - protein, 127 aa, Artificial Sequence
  Description of Artificial Sequence Synthetic polypeptide
QVQLQQFGGGLVQAGGSLRLSCAASGRTVSSDAMGWFRQAPGKEREFVAAINWNGASTYYADSVKGRFTISRDNAKNTVYLQMNSLKSEDTAVYYCAASDWVVGGISLMNGAEYAYWGQGTQVTVSS

SEQ ID NO: 63 - protein, 5 aa, Artificial Sequence
  Description of Artificial Sequence Synthetic peptide
SDAMG

SEQ ID NO: 64 - protein, 17 aa, Artificial Sequence
  Description of Artificial Sequence Synthetic peptide
AINWNGASTYYADSVKG

SEQ ID NO: 65 - protein, 18 aa, Artificial Sequence
  Description of Artificial Sequence Synthetic peptide
SDWVVGGISLMNGAEYAY

SEQ ID NO: 66 - protein, 127 aa, Artificial Sequence
  Description of Artificial Sequence Synthetic polypeptide
QVQLQPSGGGLVQAGGSLRLSCAASGRTVSSDAMGWFRQAPGKEREFVAAINWNGASTYYADSVKGRFTISRDNAKNTVYLQMNSLKSEDTAVYYCAASDWVVGGISLMNGAEYAYWGQGTQVTVSS

SEQ ID NO: 67 - protein, 5 aa, Artificial Sequence
  Description of Artificial Sequence Synthetic peptide
SDAMG

SEQ ID NO: 68 - protein, 17 aa, Artificial Sequence
  Description of Artificial Sequence Synthetic peptide
AINWNGASTYYADSVKG

SEQ ID NO: 69 - protein, 18 aa, Artificial Sequence
  Description of Artificial Sequence Synthetic peptide
SDWVVGGISLMNGAEYAY

SEQ ID NO: 70 - protein, 127 aa, Artificial Sequence
  Description of Artificial Sequence Synthetic polypeptide
QVQLQAFGGGLVQAGGSLRLSCKASGRTVSSDAMGWFRQAPGKEREFVAAINWNGASTYYADSVKGRFTISRDNAKNTVYLQMNSLKSEDTAVYYCAVSDWVVGGISLMNGAEYKYWGQGTQVTVSS

SEQ ID NO: 71 - protein, 5 aa, Artificial Sequence
  Description of Artificial Sequence Synthetic peptide
SDAMG

SEQ ID NO: 72 - protein, 17 aa, Artificial Sequence
  Description of Artificial Sequence Synthetic peptide
AINWNGASTYYADSVKG

SEQ ID NO: 73 - protein, 18 aa, Artificial Sequence
  Description of Artificial Sequence Synthetic peptide
SDWVVGGISLMNGAEYKY

SEQ ID NO: 74 - protein, 127 aa, Artificial Sequence
  Description of Artificial Sequence Synthetic polypeptide
QVQLQEFGGGLVQAGGSLRLSCKASGRTVSSDAMGWFRQAPGKEREFVAAINWNGASTYYADSVKGRFTISRDNAKNTVYLQMNSLKSEDTAVYYCAVSDWVVGGISLMNGAEYKYWGQGTQVTVSS

SEQ ID NO: 75 - protein, 5 aa, Artificial Sequence
  Description of Artificial Sequence Synthetic peptide
SDAMG

SEQ ID NO: 76 - protein, 17 aa, Artificial Sequence
  Description of Artificial Sequence Synthetic peptide
AINWNGASTYYADSVKG

SEQ ID NO: 77 - protein, 18 aa, Artificial Sequence
  Description of Artificial Sequence Synthetic peptide
SDWVVGGISLMNGAEYKY

SEQ ID NO: 78 - protein, 127 aa, Artificial Sequence
  Description of Artificial Sequence Synthetic polypeptide
QVQLQAFGGGLVQAGGSLRLSCAASGRTVSSDAMGWFRQAPGKEREFVAAINWNGASTYYADSVKGRFTISRDNAKNTVYLQMNSLKSEDTAVYYCAASDWVVGGISLMNGVEYKYWGQGTQVTVSS

SEQ ID NO: 79 - protein, 5 aa, Artificial Sequence
  Description of Artificial Sequence Synthetic peptide
SDAMG

SEQ ID NO: 80 - protein, 17 aa, Artificial Sequence
  Description of Artificial Sequence Synthetic peptide
AINWNGASTYYADSVKG

SEQ ID NO: 81 - protein, 18 aa, Artificial Sequence
  Description of Artificial Sequence Synthetic peptide
SDWVVGGISLMNGVEYKY

SEQ ID NO: 82 - protein, 127 aa, Artificial Sequence
  Description of Artificial Sequence Synthetic polypeptide
QVQLAESGGGLVQAGGSLRLSCKASGRTVSSDAMGWFRQAPGKEREFVAAINWNGASTYYADSVKGRFTISRDNAKNTVYLQMNSLKSEDTAVYYCAVSDWVVGGISLMNGAEYKYWGQGTQVTVSS

SEQ ID NO: 83 - protein, 5 aa, Artificial Sequence
  Description of Artificial Sequence Synthetic peptide
SDAMG

SEQ ID NO: 84 - protein, 17 aa, Artificial Sequence
  Description of Artificial Sequence Synthetic peptide
AINWNGASTYYADSVKG

SEQ ID NO: 85 - protein, 18 aa, Artificial Sequence
  Description of Artificial Sequence Synthetic peptide
SDWVVGGISLMNGAEYKY

SEQ ID NO: 86 - protein, 127 aa, Artificial Sequence
  Description of Artificial Sequence Synthetic polypeptide
QVQLQESGGDLVQRGGSLRLSCKASGRTVSSDAMGWFRQAPGKEREFVAAINWNGASTYYADSVKGRFTISRDNAKNTVYLQMNSLKSEDTAVYYCAVSDWVVGGISLMNGAEYKYWGQGTQVTVSS

SEQ ID NO: 87 - protein, 5 aa, Artificial Sequence
  Description of Artificial Sequence Synthetic peptide
SDAMG

SEQ ID NO: 88 - protein, 17 aa, Artificial Sequence
  Description of Artificial Sequence Synthetic peptide
AINWNGASTYYADSVKG

SEQ ID NO: 89 - protein, 18 aa, Artificial Sequence
  Description of Artificial Sequence Synthetic peptide
SDWVVGGISLMNGAEYKY

SEQ ID NO: 90 - protein, 127 aa, Artificial Sequence
  Description of Artificial Sequence Synthetic polypeptide
QVQLQESGGGLVQAGGSLRLSCKASGRTVSSDAMGWFRQAPGKEREFVAAINWNGASTYYADSVKGRFTISRDNAKNTVYLQMNSLKSEDTAVYYCAVSDWVVGGISLMNGAEYKYWGQGTQVTVSS

SEQ ID NO: 91 - protein, 5 aa, Artificial Sequence
  Description of Artificial Sequence Synthetic peptide
SDAMG

SEQ ID NO: 92 - protein, 17 aa, Artificial Sequence
  Description of Artificial Sequence Synthetic peptide
AINWNGASTYYADSVKG

SEQ ID NO: 93 - protein, 18 aa, Artificial Sequence
  Description of Artificial Sequence Synthetic peptide
SDWVVGGISLMNGAEYKY

SEQ ID NO: 94 - protein, 128 aa, Artificial Sequence
  Description of Artificial Sequence Synthetic polypeptide
QVQLQESGGGLVRPGGSLRLSCAASGGTFSSYAMGWFRQAPGKEREFVAAIGGSGDSTYYADSVKGRFTISRDNAKNTAYLQMNSLKPEDTAVYYCQADPTMFHKLYYGIHPNEYEYWGQGTLVTVSS

SEQ ID NO: 95 - protein, 5 aa, Artificial Sequence
  Description of Artificial Sequence Synthetic peptide
SYAMG

SEQ ID NO: 96 - protein, 17 aa, Artificial Sequence
  Description of Artificial Sequence Synthetic peptide
AIGGSGDSTYYADSVKG

SEQ ID NO: 97 - protein, 19 aa, Artificial Sequence
  Description of Artificial Sequence Synthetic peptide
DPTMFHKLYYGIHPNEYEY

SEQ ID NO: 98 - protein, 128 aa, Artificial Sequence
  Description of Artificial Sequence Synthetic polypeptide
QVQLQESGGGLVQTGGSLTLSCVASGGTFSSYAMGWFRQAPGKEREFVAAIGGSGDSTYYADSVKGRFTISRDNAKNSVYLQMNSLKPEDTAVYYCQADPTMFHKLYYGINPNEYDYWGQGTLVTVSS

SEQ ID NO: 99 - protein, 5 aa, Artificial Sequence
  Description of Artificial Sequence Synthetic peptide
SYAMG

SEQ ID NO: 100 - protein, 17 aa, Artificial Sequence
  Description of Artificial Sequence Synthetic peptide
AIGGSGDSTYYADSVKG

SEQ ID NO: 101 - protein, 19 aa, Artificial Sequence
  Description of Artificial Sequence Synthetic peptide
DPTMFHKLYYGINPNEYDY

SEQ ID NO: 102 - protein, 128 aa, Artificial Sequence
  Description of Artificial Sequence Synthetic polypeptide
QVQLQESGGGLVKGGGSLRLSCVASGGTFSSYAMGWFRQAPGKEREFVAAIGGSGDSTYYADSVKGRFTISRDNAKNSVYLQMNSLKPEDTAVYYCQADPTMFHKLYYGINPNEYDYWGQGTLVTVSS

SEQ ID NO: 103 - protein, 5 aa, Artificial Sequence
  Description of Artificial Sequence Synthetic peptide
SYAMG

SEQ ID NO: 104 - protein, 17 aa, Artificial Sequence
  Description of Artificial Sequence Synthetic peptide
AIGGSGDSTYYADSVKG

SEQ ID NO: 105 - protein, 19 aa, Artificial Sequence
  Description of Artificial Sequence Synthetic peptide
DPTMFHKLYYGINPNEYDY

SEQ ID NO: 106 - protein, 128 aa, Artificial Sequence
  Description of Artificial Sequence Synthetic polypeptide
QVQLQESGGGLVKGGGSLRLSCVASGGTFSSYAMGWFRQAPGKEREFVAAIGGSGDSTYYADSVKGRFTISRDNAKNSVYLQMNSLKPEDTAVYYCQADPTMFHKLYYGINPNEYDYWGQGTQVTVSS

SEQ ID NO: 107 - protein, 5 aa, Artificial Sequence
  Description of Artificial Sequence Synthetic peptide
SYAMG

SEQ ID NO: 108 - protein, 17 aa, Artificial Sequence
  Description of Artificial Sequence Synthetic peptide
AIGGSGDSTYYADSVKG

SEQ ID NO: 109 - protein, 19 aa, Artificial Sequence
  Description of Artificial Sequence Synthetic peptide
DPTMFHKLYYGINPNEYDY

SEQ ID NO: 110 - protein, 116 aa, Artificial Sequence
  Description of Artificial Sequence Synthetic polypeptide
QVQLQAFGGGLVQAGGSLGLSCSASGTIFNLDYMGWYRQALGKQRELVATITSDGRTNYADSVKGRFTIFRDGAKNAILMQMNSLKPEDTAVYYCKAYLSGRSDYWGQGTQVTVSS

SEQ ID NO: 111 - protein, 5 aa, Artificial Sequence
  Description of Artificial Sequence Synthetic peptide
LDYMG

SEQ ID NO: 112 - protein, 15 aa, Artificial Sequence
  Description of Artificial Sequence Synthetic peptide
TITSDGRTNYADSVK

SEQ ID NO: 113 - protein, 8 aa, Artificial Sequence
  Description of Artificial Sequence Synthetic peptide
YLSGRSDY

SEQ ID NO: 114 - protein, 119 aa, Artificial Sequence
  Description of Artificial Sequence Synthetic polypeptide
QVQLQASGGRLVRPGESLTLSCVVSGRTSGTTAMGWFRQARGKERQFLAQISYSDGSTYYAASVKGRFNISRDNAGNTVYLQMDSLTSEDTATYYCAPTRGEGSRNVNWGQGTQVTVSS

SEQ ID NO: 115 - protein, 5 aa, Artificial Sequence
  Description of Artificial Sequence Synthetic peptide
TTAMG

SEQ ID NO: 116 - protein, 17 aa, Artificial Sequence
  Description of Artificial Sequence Synthetic peptide
QISYSDGSTYYAASVKG

SEQ ID NO: 117 - protein, 10 aa, Artificial Sequence
  Description of Artificial Sequence Synthetic peptide
TRGEGSRNVN

SEQ ID NO: 118 - protein, 115 aa, Artificial Sequence
  Description of Artificial Sequence Synthetic polypeptide
QVQLQAFGGGSVQAGGSMKLSCKVDGVSISGNAMRWYRQLPGKERTWAAIVLSNGNEHYANSVKGRFVISRDDAKNTVDLQMNNLKPEDTGTYYCNLQSPQGQYWGQGTQVTVSS

SEQ ID NO: 119 - protein, 5 aa, Artificial Sequence
  Description of Artificial Sequence Synthetic peptide
GNAMR

SEQ ID NO: 120 - protein, 16 aa, Artificial Sequence
  Description of Artificial Sequence Synthetic peptide
IVLSNGNEHYANSVKG

SEQ ID NO: 121 - protein, 7 aa, Artificial Sequence
  Description of Artificial Sequence Synthetic peptide
QSPQGQY

SEQ ID NO: 122 - protein, 116 aa, Artificial Sequence
  Description of Artificial Sequence Synthetic polypeptide
QVQLQESGGGSVQAGGSLGLFCSASGTIFNIDVMGWYRQAPGKQRELVATITSDGRTNYADSVKGRFTISRDGAKNAVHVQMNSLKPEDTAVYYCKAYLSGRSDYWGQGTQVTVSS

SEQ ID NO: 123 - protein, 5 aa, Artificial Sequence
  Description of Artificial Sequence Synthetic peptide
IDVMG

SEQ ID NO: 124 - protein, 16 aa, Artificial Sequence
  Description of Artificial Sequence Synthetic peptide
TITSDGRTNYADSVKG

SEQ ID NO: 125 - protein, 8 aa, Artificial Sequence
  Description of Artificial Sequence Synthetic peptide
YLSGRSDY

SEQ ID NO: 126 - protein, 116 aa, Artificial Sequence
  Description of Artificial Sequence Synthetic polypeptide
QVQLQASGGGLVQAGGSLGLSCSASGTIFNLDYMGWYRQALGKQRELVATITSDGRTNYADSVKGRFTIFRDGAKNAILMQMNSLKPEDTAVYYCKAYLSGRSDYWGQGTLVTVSS

SEQ ID NO: 127 - protein, 5 aa, Artificial Sequence
  Description of Artificial Sequence Synthetic peptide
LDYMG

SEQ ID NO: 128 - protein, 16 aa, Artificial Sequence
  Description of Artificial Sequence Synthetic peptide
TITSDGRTNYADSVKG

SEQ ID NO: 129 - protein, 8 aa, Artificial Sequence
  Description of Artificial Sequence Synthetic peptide
YLSGRSDY

SEQ ID NO: 130 - protein, 116 aa, Artificial Sequence
  Description of Artificial Sequence Synthetic polypeptide
QVQLQESGGGLVQAGGSLGLSCSASGTIFNLDYMGWYRQALGKQRELVATITSDGRTNYADSVKGRFTIFRDGAKNAILMQMNSLKPEDTAVYYCKAYLSGRSDYWGQGTQVTVSS

SEQ ID NO: 131 - protein, 5 aa, Artificial Sequence
  Description of Artificial Sequence Synthetic peptide
LDYMG

SEQ ID NO: 132 - protein, 16 aa, Artificial Sequence
  Description of Artificial Sequence Synthetic peptide
TITSDGRTNYADSVKG

SEQ ID NO: 133 - protein, 8 aa, Artificial Sequence
  Description of Artificial Sequence Synthetic peptide
YLSGRSDY

SEQ ID NO: 134 - protein, 127 aa, Artificial Sequence
  Description of Artificial Sequence Synthetic polypeptide
QVQLQESGGGLVKGGGSLRLSCVASGGTFSSYAMGWFRQAPGKEREFVAAIGGSGDSTYYADSVKGRFTISRDNAKNSVYLQMNNLKPEDTAIYYCAAARESSVYYTSTDPEEYGYWGQGTLVTVSS

SEQ ID NO: 135 - protein, 5 aa, Artificial Sequence
  Description of Artificial Sequence Synthetic peptide
SYAMG

SEQ ID NO: 136 - protein, 17 aa, Artificial Sequence
  Description of Artificial Sequence Synthetic peptide
AIGGSGDSTYYADSVKG

SEQ ID NO: 137 - protein, 18 aa, Artificial Sequence
  Description of Artificial Sequence Synthetic peptide
ARESSVYYTSTDPEEYGY

SEQ ID NO: 138 - protein, 132 aa, Artificial Sequence
  Description of Artificial Sequence Synthetic polypeptide
QVQLQAFGGGLVQAGGSLRLSCAASGSIFSINAMGWYRQAPGKQRELVAVITSGGSTHYADSVKGRFTISRDNTKNTLYLQMNSLKPEDTAVYYCNGRRKTTGWGTLREWEGSRDPDDYDYWGQGTLVTVSS

SEQ ID NO: 139 - protein, 5 aa, Artificial Sequence
  Description of Artificial Sequence Synthetic peptide
INAMG

SEQ ID NO: 140 - protein, 16 aa, Artificial Sequence
  Description of Artificial Sequence Synthetic peptide
VITSGGSTHYADSVKG

SEQ ID NO: 141 - protein, 24 aa, Artificial Sequence
  Description of Artificial Sequence Synthetic peptide
RRKTTGWGTLREWEGSRDPDDYDY

SEQ ID NO: 142 - protein, 126 aa, Artificial Sequence
  Description of Artificial Sequence Synthetic polypeptide
QVQLQEFGGGLVQAGASLRLSCAASGGTFSYFMGWFRQAPGKEREFVATINRNGGATGYADSVKGRFTISRDNAKNTLYLQMDSVTPEDTAVYYCAAARESSVYYTSTDPAEYGYWGQGTQVTVSS

SEQ ID NO: 143 - protein, 4 aa, Artificial Sequence
  Description of Artificial Sequence Synthetic peptide
YFMG

SEQ ID NO: 144 - protein, 17 aa, Artificial Sequence
  Description of Artificial Sequence Synthetic peptide
TINRNGGATGYADSVKG

SEQ ID NO: 145 - protein, 18 aa, Artificial Sequence
  Description of Artificial Sequence Synthetic peptide
ARESSVYYTSTDPAEYGY

SEQ ID NO: 146 - protein, 126 aa, Artificial Sequence
  Description of Artificial Sequence Synthetic polypeptide
QVQLQESGGGLVQAGASLRLSCAASGRTFSSFMGWFRQAPGKERKFVATINWNGGATGYADSVKGRFTISRDNAKNTLYLQMNSLKPEDTAVYYCAAARTGSAYYTSSDAGEYNYWGQGTQVTVSS

SEQ ID NO: 147 - protein, 4 aa, Artificial Sequence
  Description of Artificial Sequence Synthetic peptide
SFMG

SEQ ID NO: 148 - protein, 17 aa, Artificial Sequence
  Description of Artificial Sequence Synthetic peptide
TINWNGGATGYADSVKG

SEQ ID NO: 149 - protein, 18 aa, Artificial Sequence
  Description of Artificial Sequence Synthetic peptide
ARTGSAYYTSSDAGEYNY

SEQ ID NO: 150 - protein, 132 aa, Artificial Sequence
  Description of Artificial Sequence Synthetic polypeptide
QVQLQEFGGGLVQAGGSLRLSCAASGSISRINAMGWYRQAPGKQRELVAVITSGGSTHYADSVKGRFTISRDNAKNTLYLQMNSLKPEDTAVYYCNGRRKTTGWGSLREWEGSRDPDEYNYWGQGTLVTVSS

SEQ ID NO: 151 - protein, 5 aa, Artificial Sequence
  Description of Artificial Sequence Synthetic peptide
INAMG

SEQ ID NO: 152 - protein, 16 aa, Artificial Sequence
  Description of Artificial Sequence Synthetic peptide
VITSGGSTHYADSVKG

SEQ ID NO: 153 - protein, 24 aa, Artificial Sequence
  Description of Artificial Sequence Synthetic peptide
RRKTTGWGSLREWEGSRDPDEYNY

SEQ ID NO: 154 - protein, 124 aa, Artificial Sequence
  Description of Artificial Sequence Synthetic polypeptide
QVQLQESGGGLVQAGGSLRLSCAASGRTFSTHAMGWFRQAPGKEREFVAAIAWSGGSTYYADSVKGRFTISRDISKNTIYLQMNSLKPEDTALYYCAATNGGAWHYHERYFGSWGQGTLVTVSS

SEQ ID NO: 155 - protein, 5 aa, Artificial Sequence
  Description of Artificial Sequence Synthetic peptide
THAMG

SEQ ID NO: 156 - protein, 17 aa, Artificial Sequence
  Description of Artificial Sequence Synthetic peptide
AIAWSGGSTYYADSVKG

SEQ ID NO: 157 - protein, 15 aa, Artificial Sequence
  Description of Artificial Sequence Synthetic peptide
TNGGAWHYHERYFGS

SEQ ID NO: 158 - protein, 126 aa, Artificial Sequence
  Description of Artificial Sequence Synthetic polypeptide
QVQLQESGGGLVQAGTSLRLSCAASGRTFSSFMGWFRQAPGKEREFVATINRSGGATGYADSVKGRFTISRDNAKNTLYLQMNSLKPEDTAVYYCAAARGSSVYYTSSDPEEYGHWGQGTLVTVSS

SEQ ID NO: 159 - protein, 4 aa, Artificial Sequence
  Description of Artificial Sequence Synthetic peptide
SFMG

SEQ ID NO: 160 - protein, 17 aa, Artificial Sequence
  Description of Artificial Sequence Synthetic peptide
TINRSGGATGYADSVKG

SEQ ID NO: 161 - protein, 18 aa, Artificial Sequence
  Description of Artificial Sequence Synthetic peptide
ARGSSVYYTSSDPEEYGH

SEQ ID NO: 162 - protein, 132 aa, Artificial Sequence
  Description of Artificial Sequence Synthetic polypeptide
QVQLQESGGGLVQAGGSLRLSCAASGSIFSINGMGWYRQAPGKQRELVAVITSGGSTHYADSVKGRFTISRDNAKNTLYLQMNSLKPEDTAVYYCNGRRKTTGWGTLREWEGSRDPDDYDYWGQGTQVTVSS

SEQ ID NO: 163 - protein, 5 aa, Artificial Sequence
  Description of Artificial Sequence Synthetic peptide
INGMG

SEQ ID NO: 164 - protein, 16 aa, Artificial Sequence
  Description of Artificial Sequence Synthetic peptide
VITSGGSTHYADSVKG

SEQ ID NO: 165 - protein, 24 aa, Artificial Sequence
  Description of Artificial Sequence Synthetic peptide
RRKTTGWGTLREWEGSRDPDDYDY

SEQ ID NO: 166 - protein, 124 aa, Artificial Sequence
  Description of Artificial Sequence Synthetic polypeptide
QVQLQESGGGLVQAGGSLRLSCAASELTFSTHAMGWFRQAPGKEREFVAAIAWSGGSTYYADSVKGRFTISRDISKNTIYLQMNSLKPEDTALYYCAATNGGAWHYHERYFGSWGQGTQVTVSS

SEQ ID NO: 167 - protein, 5 aa, Artificial Sequence
  Description of Artificial Sequence Synthetic peptide
THAMG

SEQ ID NO: 168 - protein, 17 aa, Artificial Sequence
  Description of Artificial Sequence Synthetic peptide
AIAWSGGSTYYADSVKG

SEQ ID NO: 169 - protein, 15 aa, Artificial Sequence
  Description of Artificial Sequence Synthetic peptide
TNGGAWHYHERYFGS

SEQ ID NO: 170 - protein, 126 aa, Artificial Sequence
  Description of Artificial Sequence Synthetic polypeptide
QVQLQEFGGGLVQAGASLRLSCAASGRTLSRFMGWFRQAPGKERRFVATINWNGGATGYADSVKGRFTISRDNAKNTLYLQMNSLKPDDTAVYYCAAARTSSIYYTSSDAEEYDYWGQGTQVTVSS

SEQ ID NO: 171 - protein, 4 aa, Artificial Sequence
  Description of Artificial Sequence Synthetic peptide
RFMG

SEQ ID NO: 172 - protein, 17 aa, Artificial Sequence
  Description of Artificial Sequence Synthetic peptide
TINWNGGATGYADSVKG

SEQ ID NO: 173 - protein, 18 aa, Artificial Sequence
  Description of Artificial Sequence Synthetic peptide
ARTSSIYYTSSDAEEYDY

SEQ ID NO: 174 - protein, 126 aa, Artificial Sequence
  Description of Artificial Sequence Synthetic polypeptide
QVQLQESGGGLVQAGTSLRLSCAASGRTFSSFMGWFRQAPGKEREFVATINRSGGATGYADSVKGRFTISRDNAKNTLYLQMNSLKPEDTAVYYCAAARGSSVYYTSTDPEEYGHWGQGTQVTVSS

SEQ ID NO: 175 - protein, 4 aa, Artificial Sequence
  Description of Artificial Sequence Synthetic peptide
SFMG

SEQ ID NO: 176 - protein, 17 aa, Artificial Sequence
  Description of Artificial Sequence Synthetic peptide
TINRSGGATGYADSVKG

SEQ ID NO: 177 - protein, 18 aa, Artificial Sequence
  Description of Artificial Sequence Synthetic peptide
ARGSSVYYTSTDPEEYGH

SEQ ID NO: 178 - protein, 126 aa, Artificial Sequence
  Description of Artificial Sequence Synthetic polypeptide
QVQLQESGGGLVQAGASLRLSCQASGRTFSSFMGWFRQAPGKERKFVATINRSGGATGYADSVKGRFTISRDNAKNTLYLQMNSLRPEDTAVYYCAAARTSSSYYTSSDPEEYNYWGQGTQVTVSS

SEQ ID NO: 179 - protein, 4 aa, Artificial Sequence
  Description of Artificial Sequence Synthetic peptide
SFMG

SEQ ID NO: 180 - protein, 17 aa, Artificial Sequence
  Description of Artificial Sequence Synthetic peptide
TINRSGGATGYADSVKG

SEQ ID NO: 181 - protein, 18 aa, Artificial Sequence
  Description of Artificial Sequence Synthetic peptide
ARTSSSYYTSSDPEEYNY

SEQ ID NO: 182 - protein, 126 aa, Artificial Sequence
  Description of Artificial Sequence Synthetic polypeptide
QVQLQESGGGLVQAGASLRLSCAASGRTFSSFMGWFRQAPGKEREFVATINRNGGATGYSDSVKGRFTISRDNARSTLYLQMNSLKPEDTAVYYCAAARGSSSYYTSTDPEEYGHWGQGTLVTVSS

SEQ ID NO: 183 - protein, 4 aa, Artificial Sequence
  Description of Artificial Sequence Synthetic peptide
SFMG

SEQ ID NO: 184 - protein, 17 aa, Artificial Sequence
  Description of Artificial Sequence Synthetic peptide
TINRNGGATGYSDSVKG

SEQ ID NO: 185 - protein, 18 aa, Artificial Sequence
  Description of Artificial Sequence Synthetic peptide
ARGSSSYYTSTDPEEYGH

SEQ ID NO: 186 - protein, 126 aa, Artificial Sequence
  Description of Artificial Sequence Synthetic polypeptide
QVQLQESGGGLVQAGASLRLSCAASGRTFSSFMAWFRQAPGKERKFVATINWNGGATGYADSVKGRFTISRDNAKNTLYLQMNSLKPEDTAVYYCAAARPSSSYYTSTDAEEYNYWGQGTQVTVSS

SEQ ID NO: 187 - protein, 4 aa, Artificial Sequence
  Description of Artificial Sequence Synthetic peptide
SFMA

SEQ ID NO: 188 - protein, 17 aa, Artificial Sequence
  Description of Artificial Sequence Synthetic peptide
TINWNGGATGYADSVKG

SEQ ID NO: 189 - protein, 18 aa, Artificial Sequence
  Description of Artificial Sequence Synthetic peptide
ARPSSSYYTSTDAEEYNY

SEQ ID NO: 190 - protein, 126 aa, Artificial Sequence
  Description of Artificial Sequence Synthetic polypeptide
QVQLQESGGGLVQAGASLRLSCAASGRTSSSFMGWFRQAPGKERKFVATINWNGGATGYADSVKGRFTISRDNAKNTLYLQMNSLKPEDTAVYYCAAARTSSYYYTSTDAEEYNYWGQGTLVTVSS

SEQ ID NO: 191 - protein, 4 aa, Artificial Sequence
  Description of Artificial Sequence Synthetic peptide
SFMG

SEQ ID NO: 192 - protein, 17 aa, Artificial Sequence
  Description of Artificial Sequence Synthetic peptide
TINWNGGATGYADSVKG

SEQ ID NO: 193 - protein, 18 aa, Artificial Sequence
  Description of Artificial Sequence Synthetic peptide
ARTSSYYYTSTDAEEYNY

SEQ ID NO: 194 - protein, 132 aa, Artificial Sequence
  Description of Artificial Sequence Synthetic polypeptide
QVQLQESGGGLVQAGGSLRLSCAASGSIFSINGMGWYRQAPGKQRELVALITSGGSTHYADSVKGRFTISRDNIKNTLYLQMNSLKPEDTAVYYCNGRRKTTGWGTLREWEGSRDPDDHDYWGQGTQVTVSS

SEQ ID NO: 195 - protein, 5 aa, Artificial Sequence
  Description of Artificial Sequence Synthetic peptide
INGMG

SEQ ID NO: 196 - protein, 16 aa, Artificial Sequence
  Description of Artificial Sequence Synthetic peptide
LITSGGSTHYADSVKG

SEQ ID NO: 197 - protein, 26 aa, Artificial Sequence
  Description of Artificial Sequence Synthetic peptide
NGRRKTTGWGTLREWEGSRDPDDHDY

SEQ ID NO: 198 - protein, 132 aa, Artificial Sequence
  Description of Artificial Sequence Synthetic polypeptide
QVQLQESGGGLVQAGGSVRLSCAASGSIFSINAMGWYRQAPGKQRELVAVITSGGSTHYADSVKGRFTISRDNAKNTLYLQMNSLKPEDTAVYYCNGRRKTTGWGSLREWEGSRDPDDYDYWGQGTQVTVSS

SEQ ID NO: 199 - protein, 9 aa, Artificial Sequence
  Description of Artificial Sequence Synthetic peptide
SIFSINAMG

SEQ ID NO: 200 - protein, 16 aa, Artificial Sequence
  Description of Artificial Sequence Synthetic peptide
VITSGGSTHYADSVKG

SEQ ID NO: 201 - protein, 24 aa, Artificial Sequence
  Description of Artificial Sequence Synthetic peptide
RRKTTGWGSLREWEGSRDPDDYDY

SEQ ID NO: 202 - protein, 123 aa, Artificial Sequence
  Description of Artificial Sequence Synthetic polypeptide
QVQLQEFGGTLVQPGGSLRLSCAASGRTFSDYGMGWFRQAPGKEREFVASSSWSGRSTYYAAAVRGRFTISRDNAKSTVYLQMNSLKAEDTAVYYCAASSSWNNNDPSEYEYWGQGTLVTVSS

SEQ ID NO: 203 - protein, 9 aa, Artificial Sequence
  Description of Artificial Sequence Synthetic peptide
RTFSDYGMG

SEQ ID NO: 204 - protein, 17 aa, Artificial Sequence
  Description of Artificial Sequence Synthetic peptide
SSSWSGRSTYYAAAVRG

SEQ ID NO: 205 - protein, 14 aa, Artificial Sequence
  Description of Artificial Sequence Synthetic peptide
SSSWNNNDPSEYEY

SEQ ID NO: 206 - protein, 132 aa, Artificial Sequence
  Description of Artificial Sequence Synthetic polypeptide
QVQLQESGGGLVQAGGSLRLSCAASGSIFSINGMSWYRQAPGKQRELVAVITSGGSTHYADSVKGRFTISRDNAKNTLYLQMNSLKPEDTAVYYCNGRRKTTGWGTLREWEGSRDPDDYDYWGQGTQVTVSS

SEQ ID NO: 207 - protein, 9 aa, Artificial Sequence
  Description of Artificial Sequence Synthetic peptide
SIFSINGMS

SEQ ID NO: 208 - protein, 16 aa, Artificial Sequence
  Description of Artificial Sequence Synthetic peptide
VITSGGSTHYADSVKG

SEQ ID NO: 209 - protein, 24 aa, Artificial Sequence
  Description of Artificial Sequence Synthetic peptide
RRKTTGWGTLREWEGSRDPDDYDY

SEQ ID NO: 210 - protein, 132 aa, Artificial Sequence; identical to SEQ ID NO: 138
  Description of Artificial Sequence Synthetic polypeptide

SEQ ID NO: 211 - protein, 9 aa, Artificial Sequence
  Description of Artificial Sequence Synthetic peptide
SIFSINAMG

SEQ ID NO: 212 - protein, 16 aa, Artificial Sequence
  Description of Artificial Sequence Synthetic peptide
VITSGGSTHYADSVKG

SEQ ID NO: 213 - protein, 24 aa, Artificial Sequence
  Description of Artificial Sequence Synthetic peptide
RRKTTGWGTLREWEGSRDPDDYDY

SEQ ID NO: 214 - protein, 126 aa, Artificial Sequence; identical to SEQ ID NO: 158
  Description of Artificial Sequence Synthetic polypeptide

SEQ ID NO: 215 - protein, 8 aa, Artificial Sequence
  Description of Artificial Sequence Synthetic peptide
RTFSSFMG

SEQ ID NO: 216 - protein, 17 aa, Artificial Sequence
  Description of Artificial Sequence Synthetic peptide
TINRSGGATGYADSVKG

SEQ ID NO: 217 - protein, 18 aa, Artificial Sequence
  Description of Artificial Sequence Synthetic peptide
ARGSSVYYTSSDPEEYGH

SEQ ID NO: 218 - protein, 127 aa, Artificial Sequence
  Description of Artificial Sequence Synthetic polypeptide
QVQLQASGGGLVQAGGSLRLSCKASGRTVSSDAMGWFRQAPGKEREFVAAINWNGASTYYADSVKGRFTISRDNAKNTVYLQMNSLKSEDTAVYYCAVSDWVVGGISLMNGAEYKYWGQGTQVTVSS

SEQ ID NO: 219 - protein, 9 aa, Artificial Sequence
  Description of Artificial Sequence Synthetic peptide
RTVSSDAMG

SEQ ID NO: 220 - protein, 17 aa, Artificial Sequence
  Description of Artificial Sequence Synthetic peptide
AINWNGASTYYADSVKG

SEQ ID NO: 221 - protein, 18 aa, Artificial Sequence
  Description of Artificial Sequence Synthetic peptide
SDWVVGGISLMNGAEYKY

SEQ ID NO: 222 - protein, 116 aa, Artificial Sequence; identical to SEQ ID NO: 110
  Description of Artificial Sequence Synthetic polypeptide

SEQ ID NO: 223 - protein, 9 aa, Artificial Sequence
  Description of Artificial Sequence Synthetic peptide
TIFNLDYMG

SEQ ID NO: 224 - protein, 16 aa, Artificial Sequence
  Description of Artificial Sequence Synthetic peptide
TITSDGRTNYADSVKG

SEQ ID NO: 225 - protein, 8 aa, Artificial Sequence
  Description of Artificial Sequence Synthetic peptide
YLSGRSDY

SEQ ID NO: 226 - protein, 123 aa, Artificial Sequence
  Description of Artificial Sequence Synthetic polypeptide
QVQLQEFGGGLVQAGGSLRLSCVASGRTFSSYGMGWFRQAPGKEREFVASISWSGGSTYYPDSVLGRFTISRDNAKNVLYLQMNSLKPEDTAVYYCAASSTWNNNDPHEYDYWGQGTLVTVSS

SEQ ID NO: 227 - protein, 9 aa, Artificial Sequence
  Description of Artificial Sequence Synthetic peptide
RTFSSYGMG

SEQ ID NO: 228 - protein, 17 aa, Artificial Sequence
  Description of Artificial Sequence Synthetic peptide
SISWSGGSTYYPDSVLG

SEQ ID NO: 229 - protein, 14 aa, Artificial Sequence
  Description of Artificial Sequence Synthetic peptide
SSTWNNNDPHEYDY

SEQ ID NO: 230 - protein, 127 aa, Artificial Sequence
  Description of Artificial Sequence Synthetic polypeptide
QVQLQESGGGLVEGGGSLRLSCAASGRTFSTYAMGWFRQAPGKEREFVAAINWNGASTYYADSVKGRFTISRDNAKNTVYLQMNSLKSEDTAVYYCAASDWVVGGISLMNGAEYAYWGQGTLVTVSS

SEQ ID NO: 231 - protein, 9 aa, Artificial Sequence
  Description of Artificial Sequence Synthetic peptide
RTFSTYAMG

SEQ ID NO: 232 - protein, 17 aa, Artificial Sequence
  Description of Artificial Sequence Synthetic peptide
AINWNGASTYYADSVKG

SEQ ID NO: 233 - protein, 18 aa, Artificial Sequence
  Description of Artificial Sequence Synthetic peptide
SDWVVGGISLMNGAEYAY

SEQ ID NO: 234 - protein, 127 aa, Artificial Sequence
  Description of Artificial Sequence Synthetic polypeptide
QVQLQEFGGGLVQAGGSLRLSCAASGRTVSSDAMGWFRQAPGKEREFVAAINWNGASTYYADSVKGRFTISRDNAKNTVYLQMNSLKSEDTAVYYCAASDWVVGGISLMNGAEYAYWGQGTQVTVSS

SEQ ID NO: 235 - protein, 9 aa, Artificial Sequence
  Description of Artificial Sequence Synthetic peptide
RTVSSDAMG

SEQ ID NO: 236 - protein, 17 aa, Artificial Sequence
  Description of Artificial Sequence Synthetic peptide
AINWNGASTYYADSVKG

SEQ ID NO: 237 - protein, 18 aa, Artificial Sequence
  Description of Artificial Sequence Synthetic peptide
SDWVVGGISLMNGAEYAY

SEQ ID NO: 238 - protein, 115 aa, Artificial Sequence; identical to SEQ ID NO: 118
  Description of Artificial Sequence Synthetic polypeptide

SEQ ID NO: 239 - protein, 9 aa, Artificial Sequence
  Description of Artificial Sequence Synthetic peptide
VSISGNAMR

SEQ ID NO: 240 - protein, 16 aa, Artificial Sequence
  Description of Artificial Sequence Synthetic peptide
IVLSNGNEHYANSVKG

SEQ ID NO: 241 - protein, 7 aa, Artificial Sequence
  Description of Artificial Sequence Synthetic peptide
QSPQGQY

SEQ ID NO: 242 - protein, 127 aa, Artificial Sequence
  Description of Artificial Sequence Synthetic polypeptide
QVQLQESGGGLVQGGGSLRLSCAASGRTVSSDAMGWFRQAPGKEREFVAAINWNGGNTYYADSVKGRFTISRDNAKNTVYLQMNSLKSEDTAVYYCAVSDWVVGGISLMNGAEYKYWGQGTLVTVSS

SEQ ID NO: 243 - protein, 9 aa, Artificial Sequence
  Description of Artificial Sequence Synthetic peptide
RTVSSDAMG

SEQ ID NO: 244 - protein, 17 aa, Artificial Sequence
  Description of Artificial Sequence Synthetic peptide
AINWNGGNTYYADSVKG

SEQ ID NO: 245 - protein, 18 aa, Artificial Sequence
  Description of Artificial Sequence Synthetic peptide
SDWVVGGISLMNGAEYKY

SEQ ID NO: 246 - protein, 127 aa, Artificial Sequence
  Description of Artificial Sequence Synthetic polypeptide
QVQLQESGGGLVQAGGSLRLSCAASGRTVSSDAMGWFRQAPGKEREFVAAINWNGASTYYADSVKGRFTISRDNAKNTVYLQMNSLKFEDTAVYYCAASDWVVGGISLMNGAEYKYWGQGTQVTVSS

SEQ ID NO: 247 - protein, 9 aa, Artificial Sequence
  Description of Artificial Sequence Synthetic peptide
RTVSSDAMG

SEQ ID NO: 248 - protein, 17 aa, Artificial Sequence
  Description of Artificial Sequence Synthetic peptide
AINWNGASTYYADSVKG

SEQ ID NO: 249 - protein, 18 aa, Artificial Sequence
  Description of Artificial Sequence Synthetic peptide
SDWVVGGISLMNGAEYKY

SEQ ID NO: 250 - protein, 128 aa, Artificial Sequence; identical to SEQ ID NO: 102
  Description of Artificial Sequence Synthetic polypeptide

SEQ ID NO: 251 - protein, 9 aa, Artificial Sequence
  Description of Artificial Sequence Synthetic peptide
GTFSSYAMG

SEQ ID NO: 252 - protein, 17 aa, Artificial Sequence
  Description of Artificial Sequence Synthetic peptide
AIGGSGDSTYYADSVKG

SEQ ID NO: 253 - protein, 19 aa, Artificial Sequence
  Description of Artificial Sequence Synthetic peptide
DPTMFHKLYYGINPNEYDY

SEQ ID NO: 254 - protein, 132 aa, Artificial Sequence; identical to SEQ ID NO: 162
  Description of Artificial Sequence Synthetic polypeptide

SEQ ID NO: 255 - protein, 9 aa, Artificial Sequence
  Description of Artificial Sequence Synthetic peptide
SIFSINGMG

SEQ ID NO: 256 - protein, 16 aa, Artificial Sequence
  Description of Artificial Sequence Synthetic peptide
VITSGGSTHYADSVKG

SEQ ID NO: 257 - protein, 24 aa, Artificial Sequence
  Description of Artificial Sequence Synthetic peptide
RRKTTGWGTLREWEGSRDPDDYDY

SEQ ID NO: 258 - protein, 119 aa, Artificial Sequence; identical to SEQ ID NO: 114
  Description of Artificial Sequence Synthetic polypeptide

SEQ ID NO: 259 - protein, 9 aa, Artificial Sequence
  Description of Artificial Sequence Synthetic peptide
RTSGTTAMG

SEQ ID NO: 260 - protein, 17 aa, Artificial Sequence
  Description of Artificial Sequence Synthetic peptide
QISYSDGSTYYAASVKG

SEQ ID NO: 261 - protein, 10 aa, Artificial Sequence
  Description of Artificial Sequence Synthetic peptide
TRGEGSRNVN

SEQ ID NO: 262 - protein, 116 aa, Artificial Sequence; identical to SEQ ID NO: 126
  Description of Artificial Sequence Synthetic polypeptide

SEQ ID NO: 263 - protein, 9 aa, Artificial Sequence
  Description of Artificial Sequence Synthetic peptide
TIFNLDYMG

SEQ ID NO: 264 - protein, 16 aa, Artificial Sequence
  Description of Artificial Sequence Synthetic peptide
TITSDGRTNYADSVKG

SEQ ID NO: 265 - protein, 8 aa, Artificial Sequence
  Description of Artificial Sequence Synthetic peptide
YLSGRSDY

SEQ ID NO: 266 - protein, 127 aa, Artificial Sequence; identical to SEQ ID NO: 90
  Description of Artificial Sequence Synthetic polypeptide

SEQ ID NO: 267 - protein, 9 aa, Artificial Sequence
  Description of Artificial Sequence Synthetic peptide
RTVSSDAMG

SEQ ID NO: 268 - protein, 17 aa, Artificial Sequence
  Description of Artificial Sequence Synthetic peptide
AINWNGASTYYADSVKG

SEQ ID NO: 269 - protein, 18 aa, Artificial Sequence
  Description of Artificial Sequence Synthetic peptide
SDWVVGGISLMNGAEYKY

SEQ ID NO: 270 - protein, 116 aa, Artificial Sequence; identical to SEQ ID NO: 122
  Description of Artificial Sequence Synthetic polypeptide

SEQ ID NO: 271 - protein, 9 aa, Artificial Sequence
  Description of Artificial Sequence Synthetic peptide
TIFNIDVMG

SEQ ID NO: 272 - protein, 16 aa, Artificial Sequence
  Description of Artificial Sequence Synthetic peptide
TITSDGRTNYADSVKG

SEQ ID NO: 273 - protein, 8 aa, Artificial Sequence
  Description of Artificial Sequence Synthetic peptide
YLSGRSDY

SEQ ID NO: 274 - protein, 127 aa, Artificial Sequence; identical to SEQ ID NO: 58
  Description of Artificial Sequence Synthetic polypeptide

SEQ ID NO: 275 - protein, 9 aa, Artificial Sequence
  Description of Artificial Sequence Synthetic peptide
RTVSSDAMG

SEQ ID NO: 276 - protein, 17 aa, Artificial Sequence
  Description of Artificial Sequence Synthetic peptide
AINWNGASTYYADSVKG

SEQ ID NO: 277 - protein, 18 aa, Artificial Sequence
  Description of Artificial Sequence Synthetic peptide
SDWVVGGISLMNGAEYAY

SEQ ID NO: 278 - protein, 132 aa, Artificial Sequence
  Description of Artificial Sequence Synthetic polypeptide
QVQLQEFGGGLVQAGGSLRLSCAASGSIFSINGMGWYRQAPGKQRELVAVITSGGSTYYADSVKGRFTISRDNAKNTLYLQMKSLKPEDTAVYYCNGRRKTTGWGTLREWEGSRDPDDYDYWGQGTLVTVSS

SEQ ID NO: 279 - protein, 9 aa, Artificial Sequence
  Description of Artificial Sequence Synthetic peptide
SIFSINGMG

SEQ ID NO: 280 - protein, 16 aa, Artificial Sequence
  Description of Artificial Sequence Synthetic peptide
VITSGGSTYYADSVKG

SEQ ID NO: 281 - protein, 24 aa, Artificial Sequence
  Description of Artificial Sequence Synthetic peptide
RRKTTGWGTLREWEGSRDPDDYDY

SEQ ID NO: 282 - protein, 128 aa, Artificial Sequence
  Description of Artificial Sequence Synthetic polypeptide
QVQLQESGGGLVKGGGSLRLSCVASGGTFSSYAMGWFRQAPGKERELVAAIGGSGDSTYYADSVKGRFTISRDNAKNSVYLQMNSLKPEDTAVYYCQADPTMFHKLYYGINPNEYDYWGQGTQVTVSS

SEQ ID NO: 283 - protein, 9 aa, Artificial Sequence
  Description of Artificial Sequence Synthetic peptide
GTFSSYAMG

SEQ ID NO: 284 - protein, 17 aa, Artificial Sequence
  Description of Artificial Sequence Synthetic peptide
AIGGSGDSTYYADSVKG

SEQ ID NO: 285 - protein, 19 aa, Artificial Sequence
  Description of Artificial Sequence Synthetic peptide
DPTMFHKLYYGINPNEYDY

SEQ ID NO: 286 - protein, 123 aa, Artificial Sequence; identical to SEQ ID NO: 10
  Description of Artificial Sequence Synthetic polypeptide

SEQ ID NO: 287 - protein, 9 aa, Artificial Sequence
  Description of Artificial Sequence Synthetic peptide
RTFSSYGMG

SEQ ID NO: 288 - protein, 17 aa, Artificial Sequence
  Description of Artificial Sequence Synthetic peptide
SISWSGRSTYYTNAVRG

SEQ ID NO: 289 - protein, 14 aa, Artificial Sequence
  Description of Artificial Sequence Synthetic peptide
SSTWNNNDPTEYDY

SEQ ID NO: 290 - protein, 124 aa, Artificial Sequence; identical to SEQ ID NO: 166
  Description of Artificial Sequence Synthetic polypeptide

SEQ ID NO: 291 - protein, 9 aa, Artificial Sequence
  Description of Artificial Sequence Synthetic peptide
LTFSTHAMG

SEQ ID NO: 292 - protein, 17 aa, Artificial Sequence
  Description of Artificial Sequence Synthetic peptide
AIAWSGGSTYYADSVKG

SEQ ID NO: 293 - protein, 15 aa, Artificial Sequence
  Description of Artificial Sequence Synthetic peptide
TNGGAWHYHERYFGS

SEQ ID NO: 294 - protein, 132 aa, Artificial Sequence
  Description of Artificial Sequence Synthetic polypeptide
QVQLQESGGGLVQAGGSLRLSCTASGSISSINAMGWYRQAPGKQRELVAAITSGGSTHYADSVKGRFTISRDNAKNTMYLQMNSLKPEDTAVYYCNARRITTGWGTMREWEGSRDPYEYDYWGQGTLVTVSS

SEQ ID NO: 295 - protein, 9 aa, Artificial Sequence
  Description of Artificial Sequence Synthetic peptide
SISSINAMG

SEQ ID NO: 296 - protein, 16 aa, Artificial Sequence
  Description of Artificial Sequence Synthetic peptide
AITSGGSTHYADSVKG

SEQ ID NO: 297 - protein, 24 aa, Artificial Sequence
  Description of Artificial Sequence Synthetic peptide
RRITTGWGTMREWEGSRDPYEYDY

SEQ ID NO: 298 - protein, 123 aa, Artificial Sequence; identical to SEQ ID NO: 50
  Description of Artificial Sequence Synthetic polypeptide

SEQ ID NO: 299 - protein, 9 aa, Artificial Sequence
  Description of Artificial Sequence Synthetic peptide
GTFSDYGMG

SEQ ID NO: 300 - protein, 17 aa, Artificial Sequence
  Description of Artificial Sequence Synthetic peptide
SISWSGNSVYYAGAVRG

SEQ ID NO: 301 - protein, 14 aa, Artificial Sequence
  Description of Artificial Sequence Synthetic peptide
SSTWNNNDPTEYEY

SEQ ID NO: 302 - protein, 123 aa, Artificial Sequence; identical to SEQ ID NO: 14
  Description of Artificial Sequence Synthetic polypeptide

SEQ ID NO: 303 - protein, 9 aa, Artificial Sequence
  Description of Artificial Sequence Synthetic peptide
RTFSDYAMG

SEQ ID NO: 304 - protein, 17 aa, Artificial Sequence
  Description of Artificial Sequence Synthetic peptide
SISWSGGSLYYAGAVRG

SEQ ID NO: 305 - protein, 14 aa, Artificial Sequence
  Description of Artificial Sequence Synthetic peptide
SSTWNNNDPHEYDY

SEQ ID NO: 306 - protein, 126 aa, Artificial Sequence; identical to SEQ ID NO: 170
  Description of Artificial Sequence Synthetic polypeptide

SEQ ID NO: 307 - protein, 8 aa, Artificial Sequence
  Description of Artificial Sequence Synthetic peptide
RTLSRFMG

SEQ ID NO: 308 - protein, 17 aa, Artificial Sequence
  Description of Artificial Sequence Synthetic peptide
TINWNGGATGYADSVKG

SEQ ID NO: 309 - protein, 18 aa, Artificial Sequence
  Description of Artificial Sequence Synthetic peptide
ARTSSIYYTSSDAEEYDY

SEQ ID NO: 310 - protein, 127 aa, Artificial Sequence; identical to SEQ ID NO: 62
  Description of Artificial Sequence Synthetic polypeptide

SEQ ID NO: 311 - protein, 9 aa, Artificial Sequence
  Description of Artificial Sequence Synthetic peptide
RTVSSDAMG

SEQ ID NO: 312 - protein, 17 aa, Artificial Sequence
  Description of Artificial Sequence Synthetic peptide
AINWNGASTYYADSVKG

SEQ ID NO: 313 - protein, 18 aa, Artificial Sequence
  Description of Artificial Sequence Synthetic peptide
SDWVVGGISLMNGAEYAY

SEQ ID NO: 314 - protein, 127 aa, Artificial Sequence; identical to SEQ ID NO: 66
  Description of Artificial Sequence Synthetic polypeptide

SEQ ID NO: 315 - protein, 9 aa, Artificial Sequence
  Description of Artificial Sequence Synthetic peptide
RTVSSDAMG

SEQ ID NO: 316 - protein, 17 aa, Artificial Sequence
  Description of Artificial Sequence Synthetic peptide
AINWNGASTYYADSVKG

SEQ ID NO: 317 - protein, 18 aa, Artificial Sequence
  Description of Artificial Sequence Synthetic peptide
SDWVVGGISLMNGAEYAY

SEQ ID NO: 318 - protein, 127 aa, Artificial Sequence; identical to SEQ ID NO: 2
  Description of Artificial Sequence Synthetic polypeptide

SEQ ID NO: 319 - protein, 9 aa, Artificial Sequence
  Description of Artificial Sequence Synthetic peptide
RTVSSDAMG

SEQ ID NO: 320 - protein, 17 aa, Artificial Sequence
  Description of Artificial Sequence Synthetic peptide
AINWNGASTYYGDSAQG

SEQ ID NO: 321 - protein, 18 aa, Artificial Sequence
  Description of Artificial Sequence Synthetic peptide
SDWVVGGISLMNGAEYKY

SEQ ID NO: 322 - protein, 127 aa, Artificial Sequence; identical to SEQ ID NO: 70
  Description of Artificial Sequence Synthetic polypeptide

SEQ ID NO: 323 - protein, 9 aa, Artificial Sequence
  Description of Artificial Sequence Synthetic peptide
RTVSSDAMG

SEQ ID NO: 324 - protein, 17 aa, Artificial Sequence
  Description of Artificial Sequence Synthetic peptide
AINWNGASTYYADSVKG

SEQ ID NO: 325 - protein, 18 aa, Artificial Sequence
  Description of Artificial Sequence Synthetic peptide
SDWVVGGISLMNGAEYKY

SEQ ID NO: 326 - protein, 127 aa, Artificial Sequence
  Description of Artificial Sequence Synthetic polypeptide
QVQLAESGGGLVQAGGSLRLSCKASGRTVSSDAMGWFRQAPGKEREFVAAINWNGASTYYADSVKGRFTISRDNAKNTVYLQMNSLKSEDTAVYYCAVSDWVVGGISLMNGAEYEYWGQGTLVTVSS

SEQ ID NO: 327 - protein, 9 aa, Artificial Sequence
  Description of Artificial Sequence Synthetic peptide
RTVSSDAMG

SEQ ID NO: 328 - protein, 17 aa, Artificial Sequence
  Description of Artificial Sequence Synthetic peptide
AINWNGASTYYADSVKG

SEQ ID NO: 329 - protein, 18 aa, Artificial Sequence
  Description of Artificial Sequence Synthetic peptide
SDWVVGGISLMNGAEYEY

SEQ ID NO: 330 - protein, 128 aa, Artificial Sequence; identical to SEQ ID NO: 94
  Description of Artificial Sequence Synthetic polypeptide

SEQ ID NO: 331 - protein, 9 aa, Artificial Sequence
  Description of Artificial Sequence Synthetic peptide
GTFSSYAMG

SEQ ID NO: 332 - protein, 17 aa, Artificial Sequence
  Description of Artificial Sequence Synthetic peptide
AIGGSGDSTYYADSVKG

SEQ ID NO: 333 - protein, 19 aa, Artificial Sequence
  Description of Artificial Sequence Synthetic peptide
DPTMFHKLYYGIHPNEYEY

SEQ ID NO: 334 - protein, 127 aa, Artificial Sequence
  Description of Artificial Sequence Synthetic polypeptide
QVQLQEFGGGLVQGGGSLRLSCAASGRTVSSDAMGWFRQAPGKEREFVAAINWNGASTYYADSVKGRFTISRDNAKNTVYLQMNSLKSEDTAVYYCAASDWVVGGISLMNGAEYAFWGQGTQVTVSS

SEQ ID NO: 335 - protein, 9 aa, Artificial Sequence
  Description of Artificial Sequence Synthetic peptide
RTVSSDAMG

SEQ ID NO: 336 - protein, 17 aa, Artificial Sequence
  Description of Artificial Sequence Synthetic peptide
AINWNGASTYYADSVKG

SEQ ID NO: 337 - protein, 18 aa, Artificial Sequence
  Description of Artificial Sequence Synthetic peptide
SDWVVGGISLMNGAEYAF

SEQ ID NO: 338 - protein, 127 aa, Artificial Sequence; identical to SEQ ID NO: 82
  Description of Artificial Sequence Synthetic polypeptide

SEQ ID NO: 339 - protein, 9 aa, Artificial Sequence
  Description of Artificial Sequence Synthetic peptide
RTVSSDAMG

SEQ ID NO: 340 - protein, 17 aa, Artificial Sequence
  Description of Artificial Sequence Synthetic peptide
AINWNGASTYYADSVKG

SEQ ID NO: 341 - protein, 18 aa, Artificial Sequence
  Description of Artificial Sequence Synthetic peptide
SDWVVGGISLMNGAEYKY

SEQ ID NO: 342 - protein, 123 aa, Artificial Sequence; identical to SEQ ID NO: 18
  Description of Artificial Sequence Synthetic polypeptide

SEQ ID NO: 343 - protein, 9 aa, Artificial Sequence
  Description of Artificial Sequence Synthetic peptide
RTFSDYGMG

SEQ ID NO: 344 - protein, 17 aa, Artificial Sequence
  Description of Artificial Sequence Synthetic peptide
SSSWSGRSTYYAAAVRG

SEQ ID NO: 345 - protein, 14 aa, Artificial Sequence
  Description of Artificial Sequence Synthetic peptide
SSSWNNNDPSEYEY

SEQ ID NO: 346 - protein, 127 aa, Artificial Sequence; identical to SEQ ID NO: 74
  Description of Artificial Sequence Synthetic polypeptide

SEQ ID NO: 347 - protein, 9 aa, Artificial Sequence
  Description of Artificial Sequence Synthetic peptide
RTVSSDAMG

SEQ ID NO: 348 - protein, 17 aa, Artificial Sequence
  Description of Artificial Sequence Synthetic peptide
AINWNGASTYYADSVKG

SEQ ID NO: 349 - protein, 18 aa, Artificial Sequence
  Description of Artificial Sequence Synthetic peptide
SDWVVGGISLMNGAEYKY

SEQ ID NO: 350 - protein, 132 aa, Artificial Sequence
  Description of Artificial Sequence Synthetic polypeptide
QVQLQESGGGLVQAGGSLRLSCAASGSIFSINAMGWYRQAPGKQRELVAVITSGGSTHYADSVKGRFTISRDNAKNTLYLQMNSLKPEDTAVYYCNGRRKTTGWGTLREWEGSRDPDDYDYWGQGTLVTVSS

SEQ ID NO: 351 - protein, 9 aa, Artificial Sequence
  Description of Artificial Sequence Synthetic peptide
SIFSINAMG

SEQ ID NO: 352 - protein, 16 aa, Artificial Sequence
  Description of Artificial Sequence Synthetic peptide
VITSGGSTHYADSVKG

SEQ ID NO: 353 - protein, 24 aa, Artificial Sequence
  Description of Artificial Sequence Synthetic peptide
RRKTTGWGTLREWEGSRDPDDYDY

SEQ ID NO: 354 - protein, 127 aa, Artificial Sequence
  Description of Artificial Sequence Synthetic polypeptide
QVQLQQSGGGLVQAGGSMRLSCAASGRTVSSDAMGWFRQAPGKEREFVAAINWNGANTYYADSVKGRFTISRDNAKNTVYLQMNSLKSEDTAVYYCAVSDWVVGGISLMNGAEYKYWGQGTQVTVSS

SEQ ID NO: 355 - protein, 9 aa, Artificial Sequence
  Description of Artificial Sequence Synthetic peptide
RTVSSDAMG

SEQ ID NO: 356 - protein, 17 aa, Artificial Sequence
  Description of Artificial Sequence Synthetic peptide
AINWNGANTYYADSVKG

SEQ ID NO: 357 - protein, 18 aa, Artificial Sequence
  Description of Artificial Sequence Synthetic peptide
SDWVVGGISLMNGAEYKY

SEQ ID NO: 358 - protein, 126 aa, Artificial Sequence; identical to SEQ ID NO: 174
  Description of Artificial Sequence Synthetic polypeptide

SEQ ID NO: 359 - protein, 8 aa, Artificial Sequence
  Description of Artificial Sequence Synthetic peptide
RTFSSFMG

SEQ ID NO: 360 - protein, 17 aa, Artificial Sequence
  Description of Artificial Sequence Synthetic peptide
TINRSGGATGYADSVKG

SEQ ID NO: 361 - protein, 18 aa, Artificial Sequence
  Description of Artificial Sequence Synthetic peptide
ARGSSVYYTSTDPEEYGH

SEQ ID NO: 362 - protein, 126 aa, Artificial Sequence; identical to SEQ ID NO: 178
  Description of Artificial Sequence Synthetic polypeptide

SEQ ID NO: 363 - protein, 8 aa, Artificial Sequence
  Description of Artificial Sequence Synthetic peptide
RTFSSFMG

SEQ ID NO: 364 - protein, 17 aa, Artificial Sequence
  Description of Artificial Sequence Synthetic peptide
TINRSGGATGYADSVKG

SEQ ID NO: 365 - protein, 18 aa, Artificial Sequence
  Description of Artificial Sequence Synthetic peptide
ARTSSSYYTSSDPEEYNY

SEQ ID NO: 366 - protein, 123 aa, Artificial Sequence; identical to SEQ ID NO: 22
  Description of Artificial Sequence Synthetic polypeptide

SEQ ID NO: 367 - protein, 9 aa, Artificial Sequence
  Description of Artificial Sequence Synthetic peptide
GTFSDYGMG

SEQ ID NO: 368 - protein, 17 aa, Artificial Sequence
  Description of Artificial Sequence Synthetic peptide
SISWSGNSVYYAGAVRG

SEQ ID NO: 369 - protein, 14 aa, Artificial Sequence
  Description of Artificial Sequence Synthetic peptide
SSTWNNNDPTEYEY

SEQ ID NO: 370 - protein, 123 aa, Artificial Sequence; identical to SEQ ID NO: 26
  Description of Artificial Sequence Synthetic polypeptide

SEQ ID NO: 371 - protein, 9 aa, Artificial Sequence
  Description of Artificial Sequence Synthetic peptide
RTFSDYGMG

SEQ ID NO: 372 - protein, 17 aa, Artificial Sequence
  Description of Artificial Sequence Synthetic peptide
SSSWSGRSTYYAAAVRG

SEQ ID NO: 373 - protein, 14 aa, Artificial Sequence
  Description of Artificial Sequence Synthetic peptide
SSSWNNNDPSEYEY

SEQ ID NO: 374 - protein, 127 aa, Artificial Sequence
  Description of Artificial Sequence Synthetic polypeptide
QVQLQESGGGLVQAGGSLRLSCKASGRTVSSDAMGWFRQAPGKEREFVAAINWNGASTYYADSVKGRFTISRDNAKNTVYLQMNSLKSEDTAVYYCAVSDWVVGGFSLMNGAEYKYWGQGTQVTVSS

SEQ ID NO: 375 - protein, 9 aa, Artificial Sequence
  Description of Artificial Sequence Synthetic peptide
RTVSSDAMG

SEQ ID NO: 376 - protein, 17 aa, Artificial Sequence
  Description of Artificial Sequence Synthetic peptide
AINWNGASTYYADSVKG

SEQ ID NO: 377 - protein, 18 aa, Artificial Sequence
  Description of Artificial Sequence Synthetic peptide
SDWVVGGFSLMNGAEYKY

SEQ ID NO: 378 - protein, 124 aa, Artificial Sequence
  Description of Artificial Sequence Synthetic polypeptide
QVQLQESGGGLVQAGGSLRLSCAASGRTFSTHSMGRFRQAPGKEREFVAAIAWSGGSTYYADSVKCRFTISRDISKNTIYLQMNSVKPEDTALYYCAATNGGACHYHERYYGSWGQGTQVTVSS

SEQ ID NO: 379 - protein, 9 aa, Artificial Sequence
  Description of Artificial Sequence Synthetic peptide
RTFSTHSMG

SEQ ID NO: 380 - protein, 17 aa, Artificial Sequence
  Description of Artificial Sequence Synthetic peptide
AIAWSGGSTYYADSVKC

SEQ ID NO: 381 - protein, 15 aa, Artificial Sequence
  Description of Artificial Sequence Synthetic peptide
TNGGACHYHERYYGS

SEQ ID NO: 382 - protein, 127 aa, Artificial Sequence
  Description of Artificial Sequence Synthetic polypeptide
QVQLQEFGGGLVQAGGSLRLSCAASGRTASSDAMGWFRQTPGKEREFVAAINWNGASTYYADSVKGRFTISRDNAKNTVYLQMNSLKSEDTAVYYCAASNWIVGGISLMNGAEYNFWGQGTQVTVSS

SEQ ID NO: 383 - protein, 9 aa, Artificial Sequence
  Description of Artificial Sequence Synthetic peptide
RTASSDAMG

SEQ ID NO: 384 - protein, 17 aa, Artificial Sequence
  Description of Artificial Sequence Synthetic peptide
AINWNGASTYYADSVKG

SEQ ID NO: 385 - protein, 18 aa, Artificial Sequence
  Description of Artificial Sequence Synthetic peptide
SNWIVGGISLMNGAEYNF

SEQ ID NO: 386 - protein, 127 aa, Artificial Sequence
  Description of Artificial Sequence Synthetic polypeptide
QVQLQESGGGLVQGGGSLRLSCAASGRTVSSDAMGWFRQAPGKEREFVAAINWNGANTYYADSVKGRFTISRDNAKNTVYLQMNSLKSEDTAVYYCAVSDWVVGGISLMNGAEYKYWGQGTQVTVSS

SEQ ID NO: 387 - protein, 9 aa, Artificial Sequence
  Description of Artificial Sequence Synthetic peptide
RTVSSDAMG

SEQ ID NO: 388 - protein, 17 aa, Artificial Sequence
  Description of Artificial Sequence Synthetic peptide
AINWNGANTYYADSVKG

SEQ ID NO: 389 - protein, 18 aa, Artificial Sequence
  Description of Artificial Sequence Synthetic peptide
SDWVVGGISLMNGAEYKY

SEQ ID NO: 390 - protein, 127 aa, Artificial Sequence; identical to SEQ ID NO: 78
  Description of Artificial Sequence Synthetic polypeptide

SEQ ID NO: 391 - protein, 9 aa, Artificial Sequence
  Description of Artificial Sequence Synthetic peptide
RTVSSDAMG

SEQ ID NO: 392 - protein, 17 aa, Artificial Sequence
  Description of Artificial Sequence Synthetic peptide
AINWNGASTYYADSVKG

SEQ ID NO: 393 - protein, 18 aa, Artificial Sequence
  Description of Artificial Sequence Synthetic peptide
SDWVVGGISLMNGVEYKY

SEQ ID NO: 394 - protein, 126 aa, Artificial Sequence; identical to SEQ ID NO: 142
  Description of Artificial Sequence Synthetic polypeptide

SEQ ID NO: 395 - protein, 8 aa, Artificial Sequence
  Description of Artificial Sequence Synthetic peptide
GTFSYFMG

SEQ ID NO: 396 - protein, 17 aa, Artificial Sequence
  Description of Artificial Sequence Synthetic peptide
TINRNGGATGYADSVKG

SEQ ID NO: 397 - protein, 18 aa, Artificial Sequence
  Description of Artificial Sequence Synthetic peptide
ARESSVYYTSTDPAEYGY

SEQ ID NO: 398 - protein, 123 aa, Artificial Sequence; identical to SEQ ID NO: 30
  Description of Artificial Sequence Synthetic polypeptide

SEQ ID NO: 399 - protein, 9 aa, Artificial Sequence
  Description of Artificial Sequence Synthetic peptide
RTFSDYGMG

SEQ ID NO: 400 - protein, 17 aa, Artificial Sequence
  Description of Artificial Sequence Synthetic peptide
SSSWSGRSTYYAAAVRG

SEQ ID NO: 401 - protein, 14 aa, Artificial Sequence
  Description of Artificial Sequence Synthetic peptide
SSSWNNNDPSEYEY

SEQ ID NO: 402 - protein, 123 aa, Artificial Sequence; identical to SEQ ID NO: 34
  Description of Artificial Sequence Synthetic polypeptide

SEQ ID NO: 403 - protein, 9 aa, Artificial Sequence
  Description of Artificial Sequence Synthetic peptide
GTFSDYGMG

SEQ ID NO: 404 - protein, 17 aa, Artificial Sequence
  Description of Artificial Sequence Synthetic peptide
SSSWSGRSTYYAAAVRG

SEQ ID NO: 405 - protein, 14 aa, Artificial Sequence
  Description of Artificial Sequence Synthetic peptide
SSTWNNNDPSEYEY

SEQ ID NO: 406 - protein, 123 aa, Artificial Sequence
  Description of Artificial Sequence Synthetic polypeptide
QVQLQASGGGLVQAGGSLRLSCVASGRTFSSYGMGWFRQAPGKEREFVASISWSGGSTYYPDSVLGRFTISRDNAKNVLYLQMNSLKPEDTAVYYCAASSTWNNNDPHEYDYWGQGTLVTVSS

SEQ ID NO: 407 - protein, 9 aa, Artificial Sequence
  Description of Artificial Sequence Synthetic peptide
RTFSSYGMG

SEQ ID NO: 408 - protein, 17 aa, Artificial Sequence
  Description of Artificial Sequence Synthetic peptide
SISWSGGSTYYPDSVLG

SEQ ID NO: 409 - protein, 14 aa, Artificial Sequence
  Description of Artificial Sequence Synthetic peptide
SSTWNNNDPHEYDY

SEQ ID NO: 410 - protein, 123 aa, Artificial Sequence; identical to SEQ ID NO: 38
  Description of Artificial Sequence Synthetic polypeptide

SEQ ID NO: 411 - protein, 9 aa, Artificial Sequence
  Description of Artificial Sequence Synthetic peptide
GTFSDYGMG

SEQ ID NO: 412 - protein, 17 aa, Artificial Sequence
  Description of Artificial Sequence Synthetic peptide
SISWSGNSVYYAGAVRG

SEQ ID NO: 413 - protein, 14 aa, Artificial Sequence
  Description of Artificial Sequence Synthetic peptide
SSTWNNNDPTEYEY

SEQ ID NO: 414 - protein, 126 aa, Artificial Sequence
  Description of Artificial Sequence Synthetic polypeptide
QVQLQESGGGLVQAGASLRLSCAASGRTFSSFMAWFRQAPGKERKFVATINWNGGATGYADPVKGRFTISRDNAKNTLYLQMNSLKPEDTAVYYCAAARPSSAYYTSTDAEEYNYWGQGTLVTVSS

SEQ ID NO: 415 - protein, 8 aa, Artificial Sequence
  Description of Artificial Sequence Synthetic peptide
RTFSSFMA

SEQ ID NO: 416 - protein, 17 aa, Artificial Sequence
  Description of Artificial Sequence Synthetic peptide
TINWNGGATGYADPVKG

SEQ ID NO: 417 - protein, 18 aa, Artificial Sequence
  Description of Artificial Sequence Synthetic peptide
ARPSSAYYTSTDAEEYNY

SEQ ID NO: 418 - protein, 126 aa, Artificial Sequence; identical to SEQ ID NO: 146
  Description of Artificial Sequence Synthetic polypeptide

SEQ ID NO: 419 - protein, 8 aa, Artificial Sequence
  Description of Artificial Sequence Synthetic peptide
RTFSSFMG

SEQ ID NO: 420 - protein, 17 aa, Artificial Sequence
  Description of Artificial Sequence Synthetic peptide
TINWNGGATGYADSVKG

SEQ ID NO: 421 - protein, 18 aa, Artificial Sequence
  Description of Artificial Sequence Synthetic peptide
ARTGSAYYTSSDAGEYNY

SEQ ID NO: 422 - protein, 126 aa, Artificial Sequence; identical to SEQ ID NO: 182
  Description of Artificial Sequence Synthetic polypeptide

SEQ ID NO: 423 - protein, 8 aa, Artificial Sequence
  Description of Artificial Sequence Synthetic peptide
RTFSSFMG

SEQ ID NO: 424 - protein, 17 aa, Artificial Sequence
  Description of Artificial Sequence Synthetic peptide
TINRNGGATGYSDSVKG

SEQ ID NO: 425 - protein, 18 aa, Artificial Sequence
  Description of Artificial Sequence Synthetic peptide
ARGSSSYYTSTDPEEYGH

SEQ ID NO: 426 - protein, 126 aa, Artificial Sequence; identical to SEQ ID NO: 186
  Description of Artificial Sequence Synthetic polypeptide

SEQ ID NO: 427 - protein, 8 aa, Artificial Sequence
  Description of Artificial Sequence Synthetic peptide
RTFSSFMA

SEQ ID NO: 428 - protein, 17 aa, Artificial Sequence
  Description of Artificial Sequence Synthetic peptide
TINWNGGATGYADSVKG

SEQ ID NO: 429 - protein, 18 aa, Artificial Sequence
  Description of Artificial Sequence Synthetic peptide
ARPSSSYYTSTDAEEYNY

SEQ ID NO: 430 - protein, 123 aa, Artificial Sequence
  Description of Artificial Sequence Synthetic polypeptide
QVQLQESGGGLVQPGGSLRLSCAASGRTFSDYGMGWFRQAPGKEREFVASSSWSGRSTYYAAAVRGRFTISRDNAKSTVYLQMNSLKAEDTAVYYCAASSSWNNNDPSEYEYWGQGTLVTVSS

SEQ ID NO: 431 - protein, 9 aa, Artificial Sequence
  Description of Artificial Sequence Synthetic peptide
RTFSDYGMG

SEQ ID NO: 432 - protein, 17 aa, Artificial Sequence
  Description of Artificial Sequence Synthetic peptide
SSSWSGRSTYYAAAVRG

SEQ ID NO: 433 - protein, 14 aa, Artificial Sequence
  Description of Artificial Sequence Synthetic peptide
SSSWNNNDPSEYEY

SEQ ID NO: 434 - protein, 123 aa, Artificial Sequence; identical to SEQ ID NO: 42
  Description of Artificial Sequence Synthetic polypeptide

SEQ ID NO: 435 - protein, 9 aa, Artificial Sequence
  Description of Artificial Sequence Synthetic peptide
RTFSSYGMG

SEQ ID NO: 436 - protein, 17 aa, Artificial Sequence
  Description of Artificial Sequence Synthetic peptide
SISWSGRSTYYTNAVRG

SEQ ID NO: 437 - protein, 14 aa, Artificial Sequence
  Description of Artificial Sequence Synthetic peptide
SSTWNNNDPTEYEY

SEQ ID NO: 438 - protein, 123 aa, Artificial Sequence
  Description of Artificial Sequence Synthetic polypeptide
QVQLQASGGGLVQPGGSLRLSCAASGRTFSSYGMGWFRQAPGKEREFVASISWSGRSTYYTNAVRGRFTISRDNAKNTVYLQMNSLKPEDTAVYYCAASSTWNNNDPTEYEFWGQGTQVTVSS

SEQ ID NO: 439 - protein, 9 aa, Artificial Sequence
  Description of Artificial Sequence Synthetic peptide
RTFSSYGMG

SEQ ID NO: 440 - protein, 17 aa, Artificial Sequence
  Description of Artificial Sequence Synthetic peptide
SISWSGRSTYYTNAVRG

SEQ ID NO: 441 - protein, 14 aa, Artificial Sequence
  Description of Artificial Sequence Synthetic peptide
SSTWNNNDPTEYEF

SEQ ID NO: 442 - protein, 132 aa, Artificial Sequence
  Description of Artificial Sequence Synthetic polypeptide
QVQLQESGGGLVQAGGSLRLSCAASGSIFSINAMGWYRQAPGKQRELVAAITSGGNTYYVDSVKGRFSISRDNAMNTVDLQMNSLKPEDTAVYYCNARRITTGWGTPREWEGSRDPNDNVFWGQGTQVTVSS

SEQ ID NO: 443 - protein, 9 aa, Artificial Sequence
  Description of Artificial Sequence Synthetic peptide
SIFSINAMG

SEQ ID NO: 444 - protein, 16 aa, Artificial Sequence
  Description of Artificial Sequence Synthetic peptide
AITSGGNTYYVDSVKG

SEQ ID NO: 445 - protein, 24 aa, Artificial Sequence
  Description of Artificial Sequence Synthetic peptide
RRITTGWGTPREWEGSRDPNDNVF

SEQ ID NO: 446 - protein, 127 aa, Artificial Sequence
  Description of Artificial Sequence Synthetic polypeptide
QVQLQASGGGLVQGGGSLRLSCKASGRTVSSDAMGWFRQAPGKEREFVAAINWNGASTYYADSVKGRFTISRDNAKNTVYLQMNSLKSEDTAVYYCAVSDWVVGGISLMNGAEYKYWGQGTLVTVSS

SEQ ID NO: 447 - protein, 9 aa, Artificial Sequence
  Description of Artificial Sequence Synthetic peptide
RTVSSDAMG

SEQ ID NO: 448 - protein, 17 aa, Artificial Sequence
  Description of Artificial Sequence Synthetic peptide
AINWNGASTYYADSVKG

SEQ ID NO: 449 - protein, 18 aa, Artificial Sequence
  Description of Artificial Sequence Synthetic peptide
SDWVVGGISLMNGAEYKY

SEQ ID NO: 450 - protein, 123 aa, Artificial Sequence; identical to SEQ ID NO: 46
  Description of Artificial Sequence Synthetic polypeptide

SEQ ID NO: 451 - protein, 9 aa, Artificial Sequence
  Description of Artificial Sequence Synthetic peptide
RTFSSYGMG

SEQ ID NO: 452 - protein, 17 aa, Artificial Sequence
  Description of Artificial Sequence Synthetic peptide
SISWSGRSTYYTNAVRG

SEQ ID NO: 453 - protein, 14 aa, Artificial Sequence
  Description of Artificial Sequence Synthetic peptide
SSTWNNNDPTEYEY

SEQ ID NO: 454 - protein, 126 aa, Artificial Sequence
  Description of Artificial Sequence Synthetic polypeptide
QVQLQESGGGLVQAGASLRLSCAASGRTFSSFMAWFRQAPGKERELVANINWNGGATGYADSVKGRFTISRDNAKNTLYIQMNSLKPEDTAVYYCAAARPSSRYYTSNDAEEYNYWGQGTQVTVSS

SEQ ID NO: 455 - protein, 8 aa, Artificial Sequence
  Description of Artificial Sequence Synthetic peptide
RTFSSFMA

SEQ ID NO: 456 - protein, 17 aa, Artificial Sequence
  Description of Artificial Sequence Synthetic peptide
NINWNGGATGYADSVKG

SEQ ID NO: 457 - protein, 18 aa, Artificial Sequence
  Description of Artificial Sequence Synthetic peptide
ARPSSRYYTSNDAEEYNY

SEQ ID NO: 458 - protein, 126 aa, Artificial Sequence; identical to SEQ ID NO: 190
  Description of Artificial Sequence Synthetic polypeptide

SEQ ID NO: 459 - protein, 8 aa, Artificial Sequence
  Description of Artificial Sequence Synthetic peptide
RTSSSFMG

SEQ ID NO: 460 - protein, 17 aa, Artificial Sequence
  Description of Artificial Sequence Synthetic peptide
TINWNGGATGYADSVKG

SEQ ID NO: 461 - protein, 18 aa, Artificial Sequence
  Description of Artificial Sequence Synthetic peptide
ARTSSYYYTSTDAEEYNY

SEQ ID NO: 462 - protein, 127 aa, Artificial Sequence
  Description of Artificial Sequence Synthetic polypeptide
QVQLQAYGGGLVQAGGSLRLSCKASGRTVSSDAMGWFRQAPGKEREFVAAINWNGASTYYADSVKGRFTISRDNAKNTVYLQMNSLKYEDTAVYYCAVSDWVVGGISLMNGAEYKYWGQGTLVTVSS

SEQ ID NO: 463 - protein, 9 aa, Artificial Sequence
  Description of Artificial Sequence Synthetic peptide
RTVSSDAMG

SEQ ID NO: 464 - protein, 17 aa, Artificial Sequence
  Description of Artificial Sequence Synthetic peptide
AINWNGASTYYADSVKG

SEQ ID NO: 465 - protein, 18 aa, Artificial Sequence
  Description of Artificial Sequence Synthetic peptide
SDWVVGGISLMNGAEYKY

SEQ ID NO: 466 - protein, 132 aa, Artificial Sequence; identical to SEQ ID NO: 194
  Description of Artificial Sequence Synthetic polypeptide

SEQ ID NO: 467 - protein, 9 aa, Artificial Sequence
  Description of Artificial Sequence Synthetic peptide
SIFSINGMG

SEQ ID NO: 468 - protein, 16 aa, Artificial Sequence
  Description of Artificial Sequence Synthetic peptide
LITSGGSTHYADSVKG

SEQ ID NO: 469 - protein, 24 aa, Artificial Sequence
  Description of Artificial Sequence Synthetic peptide
RRKTTGWGTLREWEGSRDPDDHDY

SEQ ID NO: 470 - protein, 127 aa, Artificial Sequence
  Description of Artificial Sequence Synthetic polypeptide
QVQLQASGGGLVQGGGSMRLSCKASGRTVSSDAMGWFRQAPGKEREFVAAINWNGASTYYADSVKGRFTISRDNAKNTVYLQMNSLKSEDTAVYYCAVSDWVVGGISLMNGAEYKYWGQGTLVTVSS

SEQ ID NO: 471 - protein, 9 aa, Artificial Sequence
  Description of Artificial Sequence Synthetic peptide
RTVSSDAMG

SEQ ID NO: 472 - protein, 17 aa, Artificial Sequence
  Description of Artificial Sequence Synthetic peptide
AINWNGASTYYADSVKG

SEQ ID NO: 473 - protein, 18 aa, Artificial Sequence
  Description of Artificial Sequence Synthetic peptide
SDWVVGGISLMNGAEYKY

SEQ ID NO: 474 - protein, 127 aa, Artificial Sequence; identical to SEQ ID NO: 134
  Description of Artificial Sequence Synthetic polypeptide

SEQ ID NO: 475 - protein, 9 aa, Artificial Sequence
  Description of Artificial Sequence Synthetic peptide
GTFSSYAMG

SEQ ID NO: 476 - protein, 17 aa, Artificial Sequence
  Description of Artificial Sequence Synthetic peptide
AIGGSGDSTYYADSVKG

SEQ ID NO: 477 - protein, 18 aa, Artificial Sequence
  Description of Artificial Sequence Synthetic peptide
ARESSVYYTSTDPEEYGY

SEQ ID NO: 478 - protein, 127 aa, Artificial Sequence; identical to SEQ ID NO: 86
  Description of Artificial Sequence Synthetic polypeptide

SEQ ID NO: 479 - protein, 9 aa, Artificial Sequence
  Description of Artificial Sequence Synthetic peptide
RTVSSDAMG

SEQ ID NO: 480 - protein, 17 aa, Artificial Sequence
  Description of Artificial Sequence Synthetic peptide
AINWNGASTYYADSVKG

SEQ ID NO: 481 - protein, 18 aa, Artificial Sequence
  Description of Artificial Sequence Synthetic peptide
SDWVVGGISLMNGAEYKY

SEQ ID NO: 482 - protein, 123 aa, Artificial Sequence
  Description of Artificial Sequence Synthetic polypeptide
QVQLQESGGGLVETGGSLSLSCAASGRTFSDYGMGWFRQAPGKEREFVASSSWSGRSTYYAAAVRGRFTVSRDNAKSTVYLQMNSLKPEDTAVYYCAASSTWNNNDPSEYEYWGQGTQVTVSS

SEQ ID NO: 483 - protein, 9 aa, Artificial Sequence
  Description of Artificial Sequence Synthetic peptide
RTFSDYGMG

SEQ ID NO: 484 - protein, 17 aa, Artificial Sequence
  Description of Artificial Sequence Synthetic peptide
SSSWSGRSTYYAAAVRG

SEQ ID NO: 485 - protein, 14 aa, Artificial Sequence
  Description of Artificial Sequence Synthetic peptide
SSTWNNNDPSEYEY

SEQ ID NO: 486 - protein, 128 aa, Artificial Sequence; identical to SEQ ID NO: 106
  Description of Artificial Sequence Synthetic polypeptide

SEQ ID NO: 487 - protein, 9 aa, Artificial Sequence
  Description of Artificial Sequence Synthetic peptide
GTFSSYAMG

SEQ ID NO: 488 - protein, 17 aa, Artificial Sequence
  Description of Artificial Sequence Synthetic peptide
AIGGSGDSTYYADSVKG

SEQ ID NO: 489 - protein, 19 aa, Artificial Sequence
  Description of Artificial Sequence Synthetic peptide
DPTMFHKLYYGINPNEYDY

SEQ ID NO: 490 - protein, 127 aa, Artificial Sequence; identical to SEQ ID NO: 54
  Description of Artificial Sequence Synthetic polypeptide

SEQ ID NO: 491 - protein, 9 aa, Artificial Sequence
  Description of Artificial Sequence Synthetic peptide
RTFSGYAMG

SEQ ID NO: 492 - protein, 17 aa, Artificial Sequence
  Description of Artificial Sequence Synthetic peptide
AINWSGGSTYYADSVKG

SEQ ID NO: 493 - protein, 18 aa, Artificial Sequence
  Description of Artificial Sequence Synthetic peptide
SDWVVGGISLMNGAEYKY

SEQ ID NO: 494 - protein, 116 aa, Artificial Sequence; identical to SEQ ID NO: 130
  Description of Artificial Sequence Synthetic polypeptide

SEQ ID NO: 495 - protein, 9 aa, Artificial Sequence
  Description of Artificial Sequence Synthetic peptide
TIFNLDYMG

SEQ ID NO: 496 - protein, 16 aa, Artificial Sequence
  Description of Artificial Sequence Synthetic peptide
TITSDGRTNYADSVKG

SEQ ID NO: 497 - protein, 8 aa, Artificial Sequence
  Description of Artificial Sequence Synthetic peptide
YLSGRSDY

SEQ ID NO: 498 - protein, 132 aa, Artificial Sequence; identical to SEQ ID NO: 150
  Description of Artificial Sequence Synthetic polypeptide

SEQ ID NO: 499 - protein, 9 aa, Artificial Sequence
  Description of Artificial Sequence Synthetic peptide
SISRINAMG

SEQ ID NO: 500 - protein, 16 aa, Artificial Sequence
  Description of Artificial Sequence Synthetic peptide
VITSGGSTHYADSVKG

SEQ ID NO: 501 - protein, 24 aa, Artificial Sequence
  Description of Artificial Sequence Synthetic peptide
RRKTTGWGSLREWEGSRDPDEYNY

SEQ ID NO: 502 - protein, 127 aa, Artificial Sequence
  Description of Artificial Sequence Synthetic polypeptide
QVQLQASGGGLAQGGGSLRLSCKASGRTVSSDAMGWFRQAPGKEREFVAAINWNGASTYYADSVKGRFTISRDNAKNTVYLQMNSLKSEDTAVYYCAVSDWVVGGISLMNGAEYKYWGQGTLVTVSS

SEQ ID NO: 503 - protein, 9 aa, Artificial Sequence
  Description of Artificial Sequence Synthetic peptide
RTVSSDAMG

SEQ ID NO: 504 - protein, 17 aa, Artificial Sequence
  Description of Artificial Sequence Synthetic peptide
AINWNGASTYYADSVKG

SEQ ID NO: 505 - protein, 18 aa, Artificial Sequence
  Description of Artificial Sequence Synthetic peptide
SDWVVGGISLMNGAEYKY

SEQ ID NO: 506 - protein, 128 aa, Artificial Sequence; identical to SEQ ID NO: 98
  Description of Artificial Sequence Synthetic polypeptide

SEQ ID NO: 507 - protein, 9 aa, Artificial Sequence
  Description of Artificial Sequence Synthetic peptide
GTFSSYAMG

SEQ ID NO: 508 - protein, 17 aa, Artificial Sequence
  Description of Artificial Sequence Synthetic peptide
AIGGSGDSTYYADSVKG

SEQ ID NO: 509 - protein, 19 aa, Artificial Sequence
  Description of Artificial Sequence Synthetic peptide
DPTMFHKLYYGINPNEYDY

SEQ ID NO: 510 - protein, 124 aa, Artificial Sequence
  Description of Artificial Sequence Synthetic polypeptide
QVQLQESGGGLVQAGGSLRLSCAASGRTFSTHAMGWFRQAPGKEREFVAAIAWSGGSTYYADSVKGRFTISRDISKNTIYLQMNSLKPEDTALYYCAATNGGAWHYHERYFGSWGQGTQVTVSS

SEQ ID NO: 511 - protein, 9 aa, Artificial Sequence
  Description of Artificial Sequence Synthetic peptide
RTFSTHAMG

SEQ ID NO: 512 - protein, 17 aa, Artificial Sequence
  Description of Artificial Sequence Synthetic peptide
AIAWSGGSTYYADSVKG

SEQ ID NO: 513 - protein, 15 aa, Artificial Sequence
  Description of Artificial Sequence Synthetic peptide
TNGGAWHYHERYFGS

SEQ ID NO: 514 - protein, 124 aa, Artificial Sequence; identical to SEQ ID NO: 154
  Description of Artificial Sequence Synthetic polypeptide

SEQ ID NO: 515 - protein, 9 aa, Artificial Sequence
  Description of Artificial Sequence Synthetic peptide
RTFSTHAMG

SEQ ID NO: 516 - protein, 17 aa, Artificial Sequence
  Description of Artificial Sequence Synthetic peptide
AIAWSGGSTYYADSVKG

SEQ ID NO: 517 - protein, 15 aa, Artificial Sequence
  Description of Artificial Sequence Synthetic peptide
TNGGAWHYHERYFGS

SEQ ID NO: 518 - protein, 123 aa, Artificial Sequence; identical to SEQ ID NO: 6
  Description of Artificial Sequence Synthetic polypeptide

SEQ ID NO: 519 - protein, 9 aa, Artificial Sequence
  Description of Artificial Sequence Synthetic peptide
RTFSSYGMG

SEQ ID NO: 520 - protein, 17 aa, Artificial Sequence
  Description of Artificial Sequence Synthetic peptide
SISWSGGSTYYPDSVLG

SEQ ID NO: 521 - protein, 14 aa, Artificial Sequence
  Description of Artificial Sequence Synthetic peptide
SSTWNNNDPHEYDY

SEQ ID NO: 522 - protein, 127 aa, Artificial Sequence
  Description of Artificial Sequence Synthetic polypeptide
QVQLQASGGGLVQGGGSMRLSCAASGRTVSSDAMGWFRQAPGKEREFVAAINWNGASTYYADSVKGRFTISRDNAKNTVYLQMNSLKSEDTAVYYCAASDWVVGGISLMNAAEYKYWGQGTQVTVSS

SEQ ID NO: 523 - protein, 9 aa, Artificial Sequence
  Description of Artificial Sequence Synthetic peptide
RTVSSDAMG

SEQ ID NO: 524 - protein, 17 aa, Artificial Sequence
  Description of Artificial Sequence Synthetic peptide
AINWNGASTYYADSVKG

SEQ ID NO: 525 - protein, 18 aa, Artificial Sequence
  Description of Artificial Sequence Synthetic peptide
SDWVVGGISLMNAAEYKY

SEQ ID NO: 526 - DNA, 396 bp, Artificial Sequence
  Description of Artificial Sequence Synthetic polynucleotide
caggtgcagttgcaagagtctggtggcggtctggtgcaggctggtgggtccgtgaggctgagctgtgccgcatcaggtagcatctttagcatcaatgcaatgggctggtatcgccaggctccagggaagcagcgcgaactcgtcgccgttattacctccggcggtagcacgcactatgctgatagcgtgaagggccggtttactatttcccgtgacaatgccaaaaacacgctgtacctgcaaatgaacagcttgaagcccgaggacacagctgtctattactgtaacggtagacgcaagactaccggatggggctccctgcgtgagtgggaagggagtcgtgaccctgacgattacgactattggggccagggcactcaggtcaccgtgagtagt

SEQ ID NO: 527 - DNA, 369 bp, Artificial Sequence
  Description of Artificial Sequence Synthetic polynucleotide
caagtgcagctccaggagtttggtggaaccctggtccagccagggggaagtctgcgcctcagctgcgctgcctctggccgtacattttccgactatggaatgggctggttccgtcaggcacccggaaaggaacgtgagttcgtggcgtccagctcctggtctggacgtagcacatattacgcagctgccgtgcgcgggagatttacaatctcccgcgacaacgctaaatccaccgtgtacttgcagatgaactcactgaaagccgaggatacagctgtgtattactgcgcagcttccagctcctggaacaataacgacccatccgagtacgaatactggggccagggtacactggttaccgtatcttcc

SEQ ID NO: 528 - DNA, 396 bp, Artificial Sequence
  Description of Artificial Sequence Synthetic polynucleotide
caggtccagttgcaggaatcagggggcggactggtccaggcagggggctccctgcgccttagctgcgcggcttccggctctatcttcagcatcaacggtatgtcttggtatcgccaggctccaggaaagcagcgcgagctggtggccgtcatcacctcaggtggcagtacccactacgcagactctgtcaagggtcgcttcaccatctcccgcgacaacgcgaaaaacacactgtacctccagatgaactccctgaaacccgaagatacagccgtgtattactgtaacggtcgtcggaagacgaccggatggggcacgttgcgcgagtgggaaggctctcgcgacccggacgattatgactactggggccagggcacccaagtgacggtctcctcc

SEQ ID NO: 529 - DNA, 396 bp, Artificial Sequence
  Description of Artificial Sequence Synthetic polynucleotide
caggtgcagctccaggcatttggtggcgggctggtccaggctggaggttccttgcgcctctcatgcgcagcctctggcagcatctttagcatcaacgctatgggctggtatcgccaggcaccgggaaagcagagagagctggttgctgtcattacttccggtggcagcacccactacgccgatagcgtgaagggcaggtttaccatcagccgcgataacaccaagaacactctgtatctgcaaatgaactccctgaagcctgaggataccgccgtgtattactgcaatggcaggcgcaaaaccactggttggggcactctgagagaatgggagggctctagggaccctgatgactatgactactggggacagggcaccctcgtcacagtttcttct

SEQ ID NO: 530 - DNA, 378 bp, Artificial Sequence
  Description of Artificial Sequence Synthetic polynucleotide
caggttcaactgcaagaatctggaggcggtctggtccaggcgggcacatccctgcgcctttcctgtgctgcaagcgggcggacattctctagcttcatgggctggttccgtcaagctcctggcaaagagcgcgagttcgtcgcgacgattaaccgcagcggtggggcgacaggctacgccgattccgtgaaaggtagattcactattagccgcgataacgccaagaacactctgtatctccagatgaactccctcaaacctgaggataccgctgtgtactattgcgctgcggcacgtggcagctccgtatattacaccagctccgaccccgaagagtatggtcattggggccagggcacgctggtgacagtgtcctct

SEQ ID NO: 531 - DNA, 381 bp, Artificial Sequence
  Description of Artificial Sequence Synthetic polynucleotide
caggttcagttgcaggccagtggcgggggcttggtgcaggctggcggttctctgcgcctgagctgcaaagcgagcggtaggacggtctcttcagacgctatgggatggtttcgtcaggccccaggtaaggagcgtgagttcgtcgctgccataaattggaatggagcgtctacctactatgccgactccgtcaaagggcggtttactatttctcgcgataacgccaagaatacggtgtacctccagatgaacagcctgaaatctgaggacaccgccgtttattactgtgccgtttccgattgggtggtcggtggcattagtctgatgaatggtgccgagtacaagtattggggtcaaggtactcaagtcacagtgagttcc

SEQ ID NO: 532 - DNA, 348 bp, Artificial Sequence
  Description of Artificial Sequence Synthetic polynucleotide
caagtgcagctccaggcattcggcggaggcctggtgcaagcaggaggcagcctgggtctgagctgctctgccagcggcactatctttaaccttgactacatggggtggtatcgccaggcccttggcaagcagcgcgaactggttgccacaattacctctgatggccgcactaactacgccgactccgttaagggccgtttcaccatttttcgtgatggcgctaagaacgccattttgatgcagatgaatagtctgaagccagaggataccgctgtgtattactgcaaggcgtacctgtccgggcgcagcgattattggggtcagggaacccaggtcaccgtgagcagc

SEQ ID NO: 533 - DNA, 369 bp, Artificial Sequence
  Description of Artificial Sequence Synthetic polynucleotide
caggtccagttgcaagagttcggaggtggcttggttcaggccggtggctccttgcgcctgtcctgcgtggcttctggccgcactttcagttcatacggtatgggctggtttagacaagcgccgggtaaggagcgcgagttcgtggcatctatctcttggagcggaggttccacatattacccggatagtgttctggggcgcttcactatctctagggataacgcgaagaatgtgctttatcttcagatgaatagcctgaaaccagaagacacggcggtttattactgcgctgcaagcagtacctggaacaataacgatccccacgagtacgactattggggtcaaggcaccctcgtgactgtgtccagc

SEQ ID NO: 534 - DNA, 381 bp, Artificial Sequence
  Description of Artificial Sequence Synthetic polynucleotide
caggtgcagctccaggagagtggtggaggcctggttgagggaggcggatctttgcggctgtcatgcgccgcatctgggcgcacgttctccacctatgctatgggatggttcagacaagcgcctggtaaggagagggagttcgtggccgctattaactggaacggggccagcacttattacgctgactccgtcaagggacggttcaccatctcccgcgacaacgctaagaatactgtgtatctccagatgaacagcctcaagagcgaggatactgccgtttattactgtgcggcctcagattgggtcgtgggaggtatcagcttgatgaacggtgctgaatacgcctactggggtcagggaaccctggttactgtgtccagt

SEQ ID NO: 535 - DNA, 381 bp, Artificial Sequence
  Description of Artificial Sequence Synthetic polynucleotide
caggtccagctccaggaatttgggggtggcctggtgcaggccggaggttccctccggctgtcctgcgctgccagcggacgcaccgtcagctctgacgcaatgggctggttcagacaggcccccggtaaggagcgcgagttcgtcgccgctatcaactggaatggtgcctctacctattacgctgactctgttaagggccgtttcaccatctctcgtgataacgccaagaatactgtgtatctccagatgaatagtctcaagtccgaagacaccgccgtttattactgtgcggcttctgactgggtggtcggaggcatctccctgatgaatggagccgaatacgcttattgggggcagggcacacaggtaactgtctcttcc

SEQ ID NO: 536 - DNA, 381 bp, Artificial Sequence
  Description of Artificial Sequence Synthetic polynucleotide
caagtgcagctccaagagagtggcggaggcttggtgcaaggtgggggctccttgagattgtcttgtgcggcttccggtagaaccgtcagctccgatgctatgggctggttccgccaggctcctggaaaggaaagagagttcgtggcggctattaactggaacgggggaaacacctattacgcagactccgtaaaaggccgcttcacaatcagccgcgacaacgctaagaacactgtttatcttcagatgaactccctcaagtccgaagatacggcagtctattactgtgctgtctctgactgggttgtaggcgggatttccctgatgaacggtgcagagtacaagtactggggtcagggcaccctcgtgactgtctcaagc

SEQ ID NO: 537 - DNA, 381 bp, Artificial Sequence
  Description of Artificial Sequence Synthetic polynucleotide
caggtgcagctccaggaaagtggcggtgggctggtacaggctggcgggtctttgcgtctgagctgcgctgcgagtggtcgcaccgtctcttccgacgctatggggtggtttcgccaggctcccggtaaggagcgcgaatttgtggctgccattaactggaatggtgcaagcacctactatgcggactctgttaagggacgttttaccatcagtagagataacgccaaaaacacggtttacctccagatgaactctctcaaattcgaggatactgcggtgtactattgtgcggccagtgactgggttgtgggcggtatttccttgatgaacggtgccgagtacaagtactggggccagggtacacaagtgacagtttccagc

SEQ ID NO: 538 - DNA, 384 bp, Artificial Sequence
  Description of Artificial Sequence Synthetic polynucleotide
caggttcagctccaggagagcggcggtggcctggtgaaaggtggaggctccctccggttgagctgcgtggcttccgggggcacattcagctcttacgctatgggctggtttcgtcaggcccccggtaaggagcgcgagttcgtggctgccatcggaggctcaggcgactctacttattacgctgactcagtgaagggtaggttcaccatctcccgcgataacgccaagaactctgtgtaccttcagatgaactctctgaaacccgaggacacggcagtctattactgccaggcagatcccacaatgttccacaagctgtattacggcatcaatccaaacgaatacgactattggggacagggtacgctggtgacggtctcctct

SEQ ID NO: 539 - DNA, 396 bp, Artificial Sequence
  Description of Artificial Sequence Synthetic polynucleotide
caggtgcagctccaggaatccggtgggggcctggtacaggcaggcgggtcactcagactctcttgcgcggccagcggctccatctttagcatcaacggcatgggctggtacagacaggctcccggcaagcagcgcgagttggtggctgtcatcacttctggtggcagtacccactacgcggactccgtgaagggtcgcttcaccatctctcgcgacaatgccaagaacactctctacctgcaaatgaactccctgaagccagaagacaccgccgtttattactgcaacgggagacgcaagacaacgggatggggcaccctccgtgagtgggagggcagccgcgacccagatgactatgactactgggggcagggaacccaggtgacagtatcatcc

SEQ ID NO: 540 - DNA, 357 bp, Artificial Sequence
  Description of Artificial Sequence Synthetic polynucleotide
caggtgcaacttcaggccagcggcggtcgcctggtgaggccaggcgagagtctcaccctgagctgcgtcgtttcaggccgcacgtcaggtacgaccgcgatgggatggtttcgccaggctcgtggcaaggaaaggcagttcctcgcgcaaatctcctactccgacggctccacttattacgccgcgagcgttaagggccgcttcaacattagccgtgacaatgcgggcaacactgtgtatttgcagatggactccctgaccagtgaggacactgccacctactattgcgctcccactcgcggagaaggtagtcgcaacgtcaactggggacaggggacacaggtcacagtatccagc

SEQ ID NO: 541 - DNA, 345 bp, Artificial Sequence
  Description of Artificial Sequence Synthetic polynucleotide
caggtccagctccaggcttttggcgggggttccgtccaggccggaggtagcatgaagctcagctgtaaggttgacggggtgagtatctcaggaaacgccatgcgttggtatcgccagcttccaggtaaggaacgtacttgggccgctatcgtgctctccaatggtaatgagcactacgccaacagcgtgaagggccggtttgtcatctcccgcgatgacgctaagaacaccgtggacctccagatgaacaatctgaagcctgaagatacaggaacttactattgcaacctccagtcccctcaggggcaatactggggccagggcacccaggtgacagtttcttct

SEQ ID NO: 542 - DNA, 348 bp, Artificial Sequence
  Description of Artificial Sequence Synthetic polynucleotide
caggttcagctccaggccagtgggggcggactggtccaagccggtggctcactgggactgtcctgttccgcgtcagggaccattttcaatctggactacatgggctggtataggcaggccctgggaaagcagcgcgagctggtcgccaccattacttccgatggccgcactaattacgctgacagcgtgaagggcaggttcaccatcttcagagacggagccaaaaacgccattctgatgcagatgaacagcctgaagccagaggatactgccgtctattactgtaaagcctacctttctggccgctctgactactgggggcagggcaccctcgtgaccgtatccagt

SEQ ID NO: 543 - DNA, 381 bp, Artificial Sequence
  Description of Artificial Sequence Synthetic polynucleotide
caagtgcagctccaggagagcggcgggggactggtgcaggcgggcgggagcctgcgcttgtcctgcaaggcctctggccgcaccgtaagctccgatgctatgggatggtttcgtcaggctccgggtaaggagcgggagtttgttgctgcgatcaactggaatggggccagcacttattacgctgattctgtgaagggaagattcacaatcagcagagataacgctaagaacaccgtgtaccttcagatgaactctctgaagagcgaggacaccgccgtgtactattgtgccgtctctgattgggtagttggaggcatcagcctgatgaatggtgccgagtacaaatattgggggcagggcacccaagtgaccgtgtcatcc

SEQ ID NO: 544 - DNA, 348 bp, Artificial Sequence
  Description of Artificial Sequence Synthetic polynucleotide
caagtgcaactgcaagagagcggcggtggctctgtgcaggccggtggctccctcggactcttctgctccgcctccgggactatcttcaatattgatgtgatgggctggtatcgccaggcccctgggaagcaacgcgagctggttgcgaccattacttccgacggacgcaccaactacgccgactcagttaaggggcgctttactatctctcgcgacggggctaaaaatgcagtccacgtccagatgaatagtctgaagccggaggacacggctgtgtattactgtaaggcttacctgagcgggcgtagcgactattgggggcagggaacccaggttacagtatccagc

SEQ ID NO: 545 - DNA, 381 bp, Artificial Sequence
  Description of Artificial Sequence Synthetic polynucleotide
caggtccaactgcaagagtctgggggaggcctcgtgcaggctgggggttctctgcggctgtcctgcgcggctagtggccgcacagtgtctagcgacgcgatgggatggttccgccaggctccagggaaggagcgcgagtttgtggctgccatcaactggaacggtgccagtacctattacgcagacagcgtgaagggccgttttaccatttcccgcgataacgccaagaacaccgtgtacttgcagatgaactccctgaagtccgaggacactgctgtctattactgtgcagcgagtgattgggtggtcggtggcatctccctgatgaacggtgctgaatacgcttactggggccagggcacccaggtgaccgtgtctagc

SEQ ID NO: 546 - DNA, 396 bp, Artificial Sequence
  Description of Artificial Sequence Synthetic polynucleotide
caggtccagttgcaggagtttggcgggggcctggtgcaggcgggcggatctctgcgcctcagctgtgccgcgtccggctccatcttctctatcaatggcatgggctggtatcggcaggcccctggaaagcagcgcgaactcgtggctgttatcaccagtggaggctccacatattacgcagattccgtgaaagggcgcttcacgatctcccgcgacaacgccaagaacaccctgtacctccagatgaagagcttgaagccggaggacaccgccgtgtactattgcaacggtagacggaaaaccacaggttggggtactctccgcgagtgggagggcagcagagacccagatgactacgattattggggccaggggacgctggtcaccgtctctagc

SEQ ID NO: 547 - DNA, 384 bp, Artificial Sequence
  Description of Artificial Sequence Synthetic polynucleotide
caggtccagctccaggaatctgggggcggtctggtcaagggcggtgggtccctccggctctcatgtgtggccagcggaggcaccttctcctcatacgctatgggctggtttaggcaggcacctggcaaggagagggagctggtggctgccattggaggctccggcgactctacatactatgccgacagcgtcaaggggcgctttacgattagccgcgacaacgccaaaaacagcgtgtacctgcaaatgaacagcctgaagcctgaggataccgctgtgtattactgccaggccgacccgaccatgtttcacaaactgtattacggaattaacccgaacgaatatgactactggggccaagggacccaggtaacagtgtcctcc

SEQ ID NO: 548 - DNA, 369 bp, Artificial Sequence
  Description of Artificial Sequence Synthetic polynucleotide
caggttcaactccaggagagcggcggtggcctggtccagccaggtggcagtctgcgcctctcatgcgctgcatcaggccgtacattctctagttacgggatgggctggtttaggcaggctcctggcaaggaaagggaatttgtcagttcaatctcttggagtggacgcagcacctactataccaacgccgtgagaggcaggtttactatctcccgcgacaatgccaagaacactgtttacctccagatgaactctctgaaacctgaggacaccgcagtgtattactgcgccgcttctagcacctggaacaataacgaccccaccgagtacgattattggggccaaggtacacttgtgactgtgtccagt

SEQ ID NO: 549 - DNA, 372 bp, Artificial Sequence
  Description of Artificial Sequence Synthetic polynucleotide
caggttcagttgcaggagtccggtggcggtctggtgcaggcgggagggagcctgcgcctgtcatgtgctgccagcgaactcacgttctccacccacgctatggggtggtttcgccaggcccccggcaaagagcgcgagttcgtggctgcaatcgcctggagcggaggcagcacatattacgccgactctgtgaagggacgtttcactatcagtcgtgacatctccaagaataccatctatcttcagatgaacagcctcaaaccggaggataccgcgctgtattactgcgctgccaccaacggtggagcctggcactatcacgaacgctattttggctcatggggtcagggcacccaggtgaccgtcagcagt

SEQ ID NO: 550 - DNA, 396 bp, Artificial Sequence
  Description of Artificial Sequence Synthetic polynucleotide
caggtgcagctccaggagtccgggggtggactcgtccaggccggtggctccctgcgcctgtcttgcacagccagtggcagcatctccagtatcaacgcaatgggctggtatcgtcaggcccctggaaagcagcgcgagctggtggcggccatcacatccggcggatctactcactatgcggattccgttaagggtagattcaccatcagccgtgataacgcgaagaacacaatgtacttgcagatgaacagtctcaagccggaagacaccgccgtgtattactgcaacgctaggcgcatcacaaccggatggggcacaatgcgcgagtgggagggctcccgcgacccctacgaatatgattattggggacagggcacccttgtgaccgtgtccagc

SEQ ID NO: 551 - DNA, 369 bp, Artificial Sequence
  Description of Artificial Sequence Synthetic polynucleotide
caggtgcaactccaagagtctggtggcggactggtgcagcccggcggaagcctgcgtctgtcatgtgcagcttccggcggtactttttccgattacggcatgggatggttccgccaggcccccggtaaggaacgcgagtttgtgagcagtatctcctggagcggcaattccgtgtactatgccggagcggttcggggccgctttactattagccgcgataatgctaaaaatactgtctatctccagatgaactctctccagccagaagacacagctgtctattactgcgcggcttccagcacctggaataacaatgatcctactgaatatgagtattgggggcaagggacgctggttactgtcagttct

SEQ ID NO: 552 - DNA, 369 bp, Artificial Sequence
  Description of Artificial Sequence Synthetic polynucleotide
caagtgcagcttcaggaatttggcgggggcctcgtgcagcctggtggcagtctgcgcctgagttgcgctgcctccggcaggaccttctccgactatgcaatgggttggttccgtcaggccccaggcaaagaacgtgagtttgtggccagcatctcctggtcaggcggatctctttattacgcaggcgcggtgaggggccgctttacaatctcccgtgataatgctaagaacaccgtgtacctgcaaatgaactccctcaagcccgaagataccgccgtgtattactgtgccgcgtccagcacgtggaacaataacgatccgcatgaatacgactactggggccagggcacccaggttaccgtctcctcc

SEQ ID NO: 553 - DNA, 378 bp, Artificial Sequence
  Description of Artificial Sequence Synthetic polynucleotide
caggttcagttgcaggagttcggtggaggcctggtgcaggctggtgcgtccctccgcctgagctgcgccgcatctggtcgcactctgtcccgcttcatgggttggttcaggcaggcccccggcaaggagaggcgctttgttgccactattaactggaatggcggtgccaccggatatgccgatagtgtgaagggtcggttcacaatcagccgcgacaacgccaagaacacactgtacctccagatgaactctctgaagcccgatgacacagctgtgtattactgtgcagccgctcgcactagcagtatctattacaccagctctgacgcggaggaatatgactattggggccagggcacccaggtcaccgtgagcagt

SEQ ID NO: 554 - DNA, 381 bp, Artificial Sequence
  Description of Artificial Sequence Synthetic polynucleotide
caggtgcagctgcaacagtttggcggaggcctggtacaggcaggcgggtcccttcgcttgtcttgcgcggcatccggtcgtactgtgtccagcgacgcaatggggtggtttcgccaggcacccggcaaggaaagggagtttgttgcagctataaattggaacggcgcgtccacatactatgccgacagcgtgaagggccgtttcacaatctcacgtgacaacgctaagaacacagtgtatctgcaaatgaactctctgaagtccgaggacactgccgtgtattactgcgctgcgtccgactgggtagtgggcggaatctccctgatgaacggggccgaatatgcttactggggccagggaacccaggtaaccgtctcaagt

SEQ ID NO: 555 - DNA, 381 bp, Artificial Sequence
  Description of Artificial Sequence Synthetic polynucleotide
caagtgcagctccagcctagcggcgggggcctggtccaggctggtggatctctccggctgtcttgtgccgcgtccgggaggaccgtctcttccgacgctatgggatggttccgtcaggctcccggcaaagagcgcgagttcgtggctgccattaactggaacggagcctccacctattacgctgattccgtgaaaggccgctttaccattagcagagacaacgctaaaaacactgtgtacttgcagatgaactccttgaagagcgaggacacagccgtgtactattgcgccgcaagtgattgggtcgtgggtggaatcagcctgatgaacggagccgaatacgcttactggggccaaggaacacaggtgaccgtctctagc

SEQ ID NO: 556 - DNA, 381 bp, Artificial Sequence
  Description of Artificial Sequence Synthetic polynucleotide
caggtgcagctccaggccagcggcggaggtctcgtccaggctgggggcagcctgcgcctgtcctgtgcagcttctggccgcaccgtgtccagcgacgcgatggggtggttcagacaggcccctggaaaggagcgcgagttcgtggccgcaatcaactggaatggcgcttccacctattacggtgacagtgctcagggtcgttttaccatcagccgcgacaacgcgaagaacacagtttaccttcagatgaacagcctgaagagtgaggacactgccgtgtattactgcgctgcatccgactgggtcgtgggcgggatttcccttatgaacggcgcagaatataagtattggggacagggcacactggtgacagttagttcc

SEQ ID NO: 557 - DNA, 381 bp, Artificial Sequence
  Description of Artificial Sequence Synthetic polynucleotide
caggtgcagttgcaggcttttggcggaggcctcgtccaggctggcggttctctccgtctgtcttgcaaggctagtgggcgtactgtgtcctcagatgctatgggctggtttcgccaagcacccggaaaagagagggagttcgtggctgccatcaactggaacggggcctcaacttattacgccgacagcgtgaagggtcggttcaccatctctcgcgataacgctaagaacacggtgtatctccagatgaacagtttgaagagtgaggacacagctgtctattactgtgctgtgtccgactgggtggtcggcggtatctctctgatgaacggggcagaatacaagtactggggtcagggaacccaggtcaccgtcagctca

SEQ ID NO: 558 - DNA, 381 bp, Artificial Sequence
  Description of Artificial Sequence Synthetic polynucleotide
caggtccagctggccgaaagcggcggtggcctggtccaggccgggggcagcctgcggctctcttgcaaggcgtctggccgcaccgtcagctcagacgcgatgggctggtttcgccaggctccgggtaaggaaagggagttcgtggcagcgataaattggaacggagcgtccacatattacgctgattctgtgaagggccgcttcactatctccagagacaacgctaagaacaccgtgtacctccagatgaactccttgaagtctgaggacaccgccgtgtattactgtgcggtttccgattgggtggtcggcggtatctccctgatgaacggagccgaatatgagtactggggacagggcaccctcgtcactgtgtcctcc

SEQ ID NO: 559 - DNA, 384 bp, Artificial Sequence
  Description of Artificial Sequence Synthetic polynucleotide
caggtgcagctccaggagtccggcggtggcctcgtgcgcccaggcggaagcctgagactgtcctgcgctgccagcggtggcacctttagctcctacgcgatgggctggttccgccaggctcccggcaaggagagagagtttgtagcagctatcggagggagcggagattccacttattacgcagactccgtcaagggccgcttcaccatctctcgcgacaatgctaaaaacaccgcttatttgcagatgaactccttgaagcctgaagatacggcggtgtactattgccaggcagatcctactatgttccataagttgtattacggcattcatcctaacgaatatgagtactggggacaaggcaccctggtcactgtgtcctct

SEQ ID NO: 560 - DNA, 381 bp, Artificial Sequence
  Description of Artificial Sequence Synthetic polynucleotide
caagtccagctccaggagtttggcggaggcctggtccaagggggtggcagcctgagactgtcctgcgcggcctctgggcgcactgtgtcctctgacgcgatgggttggttccgtcaggcacccggcaaggagcgcgagttcgttgccgctatcaactggaatggtgcctccacctactatgctgacagcgtgaagggccggttcacaatcagtagggacaacgccaagaataccgtgtacttgcaaatgaactccctgaagtccgaggatactgcggtttattactgtgcggcctctgactgggtcgtagggggtatttctctgatgaacggggctgagtacgccttctggggacagggcacccaggtgacagtgtcaagt

SEQ ID NO: 561 - DNA, 381 bp, Artificial Sequence
  Description of Artificial Sequence Synthetic polynucleotide
caggtacagctcgcggagtctggtggaggcttggtgcaagcaggcgggtctctgcgcctgagttgtaaagctagtgggaggaccgtgtctagtgacgcgatgggttggttccggcaggcccccggcaaggagcgcgagtttgtcgctgcaatcaactggaacggcgcttctacctactatgccgacagcgtcaaaggccgctttactatcagcagggacaatgctaaaaacactgtgtacctgcaaatgaactccctgaagagcgaggataccgccgtatattactgcgccgtgtccgactgggtagtgggcggaattagcctgatgaacggggcggaatacaagtactggggccagggcacccaggtcacagttagctcc

SEQ ID NO: 562 - DNA, 369 bp, Artificial Sequence
  Description of Artificial Sequence Synthetic polynucleotide
caggtacagctgcaacagagcgggggcgatctggtgcagcctgggggctcccttaggttgagctgcgccgcttctggccggaccttctccgactacgggatgggttggttcagacaagcacctggcaaggaacgcgagttcgtggcgtccagctcctggtccggcaggtctacctattacgctgccgctgttcgcgggaggttcaccatcagtcgtgataacgcgaaaagtacagtttatctccagatgaactctctgaaggccgaagatacagccgtgtattactgtgctgcctcttcctcatggaacaataacgatccttctgagtacgaatattggggtcagggcacacaggtcacggtgtcctca

SEQ ID NO: 563 - DNA, 381 bp, Artificial Sequence
  Description of Artificial Sequence Synthetic polynucleotide
caagtacagctccaggagttcggcggtggcttggtccaggccgggggttctctgagactctcctgcaaggcttccggtaggactgtgtcatctgacgctatgggctggttccggcaggccccagggaaggagagagagttcgtggctgcgatcaactggaatggagcgagcacctactatgcagacagcgtcaaaggacgctttactatcagtagagacaacgccaagaacacggtctacttgcagatgaactccttgaagagcgaggacactgccgtctactattgtgctgtgagtgactgggtggtagggggaatttccctgatgaacggtgccgagtacaagtactggggtcagggcactcaggtcaccgtgagcagc

SEQ ID NO: 564 - DNA, 396 bp, Artificial Sequence
  Description of Artificial Sequence Synthetic polynucleotide
caggtgcagctccaggagtctggcggtggcctcgttcaggcgggcggttccctcaggctgagctgcgcagcctccggcagtatcttctccatcaacgcaatgggctggtatcgtcaagcgcccggcaagcagcgcgaactggtcgcagtcatcacctccggcgggtccactcattacgctgacagcgtgaagggtcgcttcaccatctccagagataacgctaagaatacgctgtacctccagatgaacagccttaaacccgaggataccgcagtttattactgcaatggccgcagaaaaacaactggctggggtacgctgcgcgaatgggagggatctcgcgatccagatgactacgattactggggccaggggaccctggtaaccgtgtcttca

SEQ ID NO: 565 - DNA, 381 bp, Artificial Sequence
  Description of Artificial Sequence Synthetic polynucleotide
caggtgcagctgcaacagtccggcggaggtctggtgcaagccggaggttccatgcgcctgtcctgtgccgcttcagggcgcactgtgtcctctgacgcgatgggatggttccggcaggcccccggaaaggaacgcgagttcgtggccgcgatcaactggaacggtgcaaacacttactatgcagacagcgtaaagggcagattcactattagtcgcgataacgcgaagaacaccgtttacctgcaaatgaactctctgaaatctgaggacacagctgtttattactgtgctgtaagcgattgggtcgtgggcgggatttcattgatgaacggtgctgagtacaagtattggggccagggaacccaagtaaccgtcagctcc

SEQ ID NO: 566 - DNA, 378 bp, Artificial Sequence
  Description of Artificial Sequence Synthetic polynucleotide
caggtgcaactccaagagagcggcggaggcttggtgcaggctggtacgtccctgcggctcagctgcgcggccagtggccgtaccttctcttccttcatggggtggttcagacaggcacccggaaaggaaagagagttcgtggccaccatcaacaggtcaggaggtgccaccgggtatgccgatagcgtgaagggcaggttcacaattagccgtgacaacgcgaaaaacacgctctatcttcagatgaatagcctcaaacctgaggatactgccgtctactattgtgcagctgcaagaggctctagcgtgtattacacatcaaccgacccagaagagtacggccactgggggcaggggacccaggttaccgtaagctca

SEQ ID NO: 567 - DNA, 378 bp, Artificial Sequence
  Description of Artificial Sequence Synthetic polynucleotide
caagtccagttgcaggagtccggtggcggtttggtccaggccggagccagcctcaggctctcctgccaggcgtccggtcgtacctttagctccttcatggggtggttccgccaggctccagggaaagagcggaagttcgttgccacgatcaatagaagcggtggagcgactggttatgccgatagcgtgaaaggaagatttacaatttcccgcgacaacgccaaaaacactctctacttgcaaatgaactctttgcgtcccgaagatacggcggtctactattgcgccgctgcacgcaccagttcaagctattacacatcttccgacccggaagagtacaattactggggccagggtacacaagtgaccgtttcctct

SEQ ID NO: 568 - DNA, 369 bp, Artificial Sequence
  Description of Artificial Sequence Synthetic polynucleotide
caggttcagctccaggccttcggaggcggaaccgtccaggctgggggctctctgcgtctgtcctgtaccgccagcggtggcaccttttctgactatggtatgggatggttccgccaggcccctggtaaggagcgcgagttcgtcagctccatttcttggtccggtaattccgtttactatgctggcgctgtgcgcggcagattcaccatctcacgtgacaacgccaagaacaccgtgtacttgcagatgaactctcttcagcctgaggacaccgcagtctattactgtgccgcttccagtacctggaacaataacgacccaaccgaatacgaatactggggccaaggcactcaggtgaccgtctccagc

SEQ ID NO: 569 - DNA, 369 bp, Artificial Sequence
  Description of Artificial Sequence Synthetic polynucleotide
caggtccagctccaacagtttggtgggggcctggttcaacctgggggctccctgcgtctgtcctgcgcagcttctggccgtaccttttccgactacggaatgggctggttccggcaagcgcccggcaaagaacgcgagttcgtggccagttcttcctggagtggccgctccacatattacgctgccgctgtccgtggccgcttcactatcagcagggacaatgctaagagcacagtatacttgcagatgaactctctcaaggccgaggataccgctgtgtattactgtgcggcatccagttcctggaacaataacgacccatcagagtatgagtattgggggcagggcacattggtgactgtatcctcc

SEQ ID NO: 570 - DNA, 381 bp, Artificial Sequence
  Description of Artificial Sequence Synthetic polynucleotide
caggtgcagctccaggagtcaggcggtggcctggtccaggctggtggatctctgcgcctcagttgtaaggcatccgggcgcacggtctcttccgacgctatgggctggtttagacaggcccctgggaaggagagagagtttgttgctgccattaactggaacggtgcctctacatattacgcagactcagtgaagggcagatttactattagccgggacaacgccaagaacaccgtgtaccttcagatgaatagtctgaagtccgaggatacagcagtgtactattgtgccgtgagcgattgggtcgtgggtggcttctcactgatgaacggtgctgagtacaagtattggggccagggcactcaggtgacggtgtctagc

SEQ ID NO: 571 - DNA, 372 bp, Artificial Sequence
  Description of Artificial Sequence Synthetic polynucleotide
caggtgcagctccaggagtcaggaggcggtttggtgcaggccggtggcagcctgcgcctgagctgtgccgctagtggccgcacattcagtacccacagtatgggcagattccgtcaggccccaggcaaggagcgcgagttcgtcgctgccattgcgtggtcaggaggctcaacatattacgccgattcagtgaagtgccgcttcaccatctcccgtgacatcagcaaaaatacaatctatcttcagatgaactccgtcaaacccgaggacaccgccctgtactattgtgccgctactaacgggggtgcttgtcattaccacgagcgctactatggctcatggggccaggggactcaggtgaccgtgtccagc

SEQ ID NO: 572 - DNA, 381 bp, Artificial Sequence
  Description of Artificial Sequence Synthetic polynucleotide
caggtgcagctccaggagttcggtggaggcctggtgcaagcgggtggctctctccgtctgagctgcgctgccagcggtagaacagcgagcagtgacgctatgggctggttcaggcagacccccggtaaggagcgggagttcgtcgccgctattaactggaacggcgcttctacatactatgctgactccgttaaaggccgttttacaatctcccgcgacaacgccaaaaataccgtgtatctccagatgaactcactgaagtctgaagataccgccgtgtactattgtgctgcgtcaaattggattgttggcgggatcagcctgatgaacggggccgaatacaacttctggggacagggaacgcaggttacggtcagcagc

SEQ ID NO: 573 - DNA, 381 bp, Artificial Sequence
  Description of Artificial Sequence Synthetic polynucleotide
caagttcaacttcaggaaagtggaggcggtctggtgcagggtggaggctctctgaggctgtcttgcgctgccagcggtcgcactgtcagctcagacgcgatgggatggttccgccaagcaccgggaaaggaacgcgagttcgtcgctgccataaattggaacggtgccaacacatattacgcagattctgtgaagggacgcttcactatcagccgcgataacgcgaagaacactgtgtacttgcagatgaactccttgaaaagtgaggacaccgccgtgtattactgtgcggtgtctgactgggtcgtgggtggcatctctttgatgaacggagctgaatataagtactggggacagggcacccaggtgaccgtatctagc

SEQ ID NO: 574 - DNA, 381 bp, Artificial Sequence
  Description of Artificial Sequence Synthetic polynucleotide
caggtgcagttgcaggcctttggcggtggccttgtccaggcaggagggagcctccgcctgagctgcgcagcgtctggtagaacggttagctcagacgctatgggctggttccgtcaggcacccggcaaggaacgggagtttgtggccgcaataaattggaacggtgccagcacctattacgccgactccgtgaaaggtcgtttcactatctcacgcgacaacgccaaaaacaccgtctacttgcagatgaatagcctgaagagcgaggacacggcggtgtattactgcgccgcttccgactgggtcgtgggaggtatcagcctgatgaacggtgtagagtacaagtattggggccaaggcacccaagtgaccgtgtcaagt

SEQ ID NO: 575 - DNA, 378 bp, Artificial Sequence
  Description of Artificial Sequence Synthetic polynucleotide
caggtgcagcttcaggagtttggcgggggcctggtccaggccggtgcctccctccgcctgagctgcgccgcttcaggcggaacattctcctattttatgggttggttccgccaggccccaggtaaggagagagagtttgtggccaccatcaataggaatggaggcgcaaccgggtacgcagattccgtcaagggtagatttacgatctcccgtgacaatgctaagaacaccctgtacctccagatggactccgtaacacccgaggacactgccgtctattactgcgctgccgcacgcgaaagctccgtttactatacctcaaccgaccctgctgagtacggctactgggggcaagggacccaggtcacggtcagctca

SEQ ID NO: 576 - DNA, 369 bp, Artificial Sequence
  Description of Artificial Sequence Synthetic polynucleotide
caggtccagcttcaggagttcgggggtggccttgtccagcctgggggtagcctgcggctgtcatgcgctgcaagcggacggactttctctgactatggcatggggtggttccgccaggctccgggcaaggaacgcgagttcgtggccagctcctcttggtccggtagaagcacatattacgctgccgctgtgcgtggccgcttcacaattagccgcgacaacgctaaatccactgtgtatctccagatgaactccctgaaggcggaagacacagccgtgtattactgtgctgcctccagtagctggaacaataacgatcccagcgaatacgagtattggggccagggcacccaggttactgtctcttct

SEQ ID NO: 577 - DNA, 369 bp, Artificial Sequence
  Description of Artificial Sequence Synthetic polynucleotide
caggtgcaactccaggccttcggcgggggcctcgtacagcctggtggcagtctgcggttgtcctgcgctgcctctggaggcaccttcagcgattacggtatgggatggttccgccaggctccggggaaagagcgtgagttcgttgcctcttcctcttggtctggccgcagtacatattacgccgcagccgtgagaggccgttttacaatctcccgcgataacgccaagtctctggtgtacttgcagatgaacaatctcaagccggaggacactgcggtctattactgcgctgcctcttccacctggaataacaatgatccttccgagtacgaatactggggtcagggcacccaggtgaccgttagctca

SEQ ID NO: 578 - DNA, 369 bp, Artificial Sequence
  Description of Artificial Sequence Synthetic polynucleotide
caggtgcaacttcaggccagcggcggaggcctggtgcaggccggtggcagcctccgcctctcctgcgttgctagtggccggacattcagctcttacggcatgggctggtttaggcaagcgccagggaaggaaagggagttcgttgccagtatctcttggtccggcggtagcacctattacccggacagcgtgctgggcaggttcaccatctccagagacaacgcaaaaaatgttttgtacctccagatgaactccctcaagcctgaagacacagcggtctactattgcgcagccagctccacttggaacaataacgacccccatgagtatgactactggggccagggcacgctggtgaccgtatcaagt

SEQ ID NO: 579 - DNA, 369 bp, Artificial Sequence
  Description of Artificial Sequence Synthetic polynucleotide
caggtgcagcttcaggagagcggcggaggcttggtcgaggctggcggttctctgcgcctgtcctgcgctgccagcggcgggaccttcagtgactacggtatgggttggtttcgtcaggcacctgggaaggagcgcgagttcgtgtccagtatcagttggtctggtaactcagtctattacgcaggagccgtccggggccgcttcaccatctcccgcgataacgccaagaacacagtatacctccagatgaactccctgcaacctgaggacacagccgtgtattactgcgctgccagctccacctggaacaataacgaccctaccgagtatgagtactggggccagggcacccaggtgaccgtgtccagc

SEQ ID NO: 580 - DNA, 378 bp, Artificial Sequence
  Description of Artificial Sequence Synthetic polynucleotide
caggtgcagcttcaggagtcaggtggaggcttggtccaggcaggagcttctctccgcctgtcctgtgctgccagcggaaggacattcagttctttcatggcttggtttcgccaggcacctggaaaggaacgtaaatttgtcgctaccatcaactggaacggtggcgctaccggatacgccgaccctgtcaagggtaggttcactatctctcgcgacaacgctaagaacaccctgtaccttcagatgaacagcctcaagcccgaagacaccgctgtgtattactgtgccgctgcgcggccctcttccgcctattacaccagcactgatgctgaggaatataactattggggccagggcaccctcgtgactgtctcttct

SEQ ID NO: 581 - DNA, 378 bp, Artificial Sequence
  Description of Artificial Sequence Synthetic polynucleotide
caggtccagcttcaggagagtggcggtggcctggtgcaggcgggggcctctctccgtctctcatgtgctgccagtggccgcaccttctcctcatttatgggctggttccgtcaggccccaggcaaggagaggaagtttgtggccaccatcaactggaacggcggtgccactggctacgcagactccgtcaagggccggtttaccatctcaagggacaacgccaaaaacaccctttacctccagatgaactctctgaaaccagaggataccgctgtttactattgcgcagccgctcgcactggtagcgcctattacacttcaagtgatgcgggtgaatacaattactggggacagggcacccaggtgaccgtctcctct

SEQ ID NO: 582 - DNA, 378 bp, Artificial Sequence
  Description of Artificial Sequence Synthetic polynucleotide
caggtgcaactccaggagagtgggggtggcctggtgcaggcgggcgcttccctccgcctgtcttgtgcggcctctggtcgcaccttctcttcatttatgggctggttccgccaagcgcccggtaaggagcgggagttcgtggccactatcaaccgtaacgggggcgcgaccggctactccgatagcgtgaagggccgcttcaccatctcacgtgacaacgctaggtctacactgtatctccagatgaacagcctgaaacctgaggacaccgccgtgtattactgtgctgccgctcgtggttcaagctcctactatacgagtacagaccccgaggaatatggccattggggacaaggaaccctggtgaccgtgagttcc

SEQ ID NO: 583 - DNA, 378 bp, Artificial Sequence
  Description of Artificial Sequence Synthetic polynucleotide
caagtgcaactgcaagagtctggcggtggcctggttcaggctggagccagcctgcgcctgagctgtgccgcttctggacgcacatttagtagcttcatggcttggttcagacaggctcctggaaaggagcgcaagttcgtagctactataaattggaacggaggtgccaccggctatgccgatagcgtgaaaggtaggtttacaatcagccgggacaatgctaagaacaccctgtacctccagatgaactccctgaagcccgaggatacagcagtgtattactgtgcggcagcgcgtccttcatctagctattacacctctaccgacgccgaagagtataactattgggggcagggcacgcaggtgactgtgtcttca

SEQ ID NO: 584 - DNA, 369 bp, Artificial Sequence
  Description of Artificial Sequence Synthetic polynucleotide
caggttcagctccaggaatctggcgggggcctggtccagcctggtggcagtctccgcctgtcctgcgctgcctctggccgtaccttctccgactacggtatgggctggttccgtcaagcccccggcaaggaacgcgagttcgtcgccagtagctcctggagcggtcgttctacttactatgccgctgccgttcgcggaagattcactatttctagggataacgccaagagcaccgtttatctgcaaatgaactccctgaaagccgaggatactgccgtctattactgtgctgccagctcctcttggaacaataacgacccgtccgagtatgagtactggggccagggcacgctcgtgacagttagtagt

SEQ ID NO: 585 - DNA, 369 bp, Artificial Sequence
  Description of Artificial Sequence Synthetic polynucleotide
caggtgcagcttcaggccagtgggggaggcctggtgcagcccggtggctctctccgcctgtcctgtgctgccagcggtcgcactttttccagttacgggatgggctggttccggcaggctccgggtaaagagcgcgagttcgtcgcaagcatcagctggagtggccgcagcacctactataccaacgctgtacgcggacgcttcacaatttctcgggacaacgcgaagaataccgtgtacttgcagatgaactccctgaagcctgaggacacagccgtgtactattgcgcagccagctccacctggaacaataacgacccgaccgagtatgagtactggggccagggcacccaggtaaccgtttcttct

SEQ ID NO: 586 - DNA, 369 bp, Artificial Sequence
  Description of Artificial Sequence Synthetic polynucleotide
caggtgcagctgcaagcatccggcgggggcctggtgcagccaggagggtcccttcgtcttagttgtgctgcctctggacgtaccttcagctcctacggcatgggctggttccgccaagcgcccggcaaggagcgtgagtttgtggcgtccattagctggagtggccggtccacttattacactaatgcagtacgtggccgcttcaccatctccagagacaacgccaaaaacaccgtctatctccagatgaacagcctgaagcctgaggataccgccgtgtattactgcgcagcctccagtacttggaacaataacgatcccaccgagtacgagttctggggccagggaacacaggtcaccgtgtccagt

SEQ ID NO: 587 - DNA, 396 bp, Artificial Sequence
  Description of Artificial Sequence Synthetic polynucleotide
caggttcagctccaggaaagcggcggaggtctggtccaggccggaggcagtctgagactgagttgtgctgcatccggctccatcttctccatcaatgctatgggatggtataggcaggctccgggcaagcaacgggagctggtagccgctatcacatcagggggaaacacctactatgtggacagtgtgaagggaagattttccatcagccgggacaacgccatgaacaccgtggatctgcaaatgaacagtctgaaacccgaagacaccgctgtgtactattgcaacgctcggcgcatcacaactggctgggggaccccacgcgagtgggagggtagtcgtgatcctaacgacaacgtgttttggggccagggcactcaggttactgtgtccagc

SEQ ID NO: 588 - DNA, 381 bp, Artificial Sequence
  Description of Artificial Sequence Synthetic polynucleotide
caagtgcagttgcaggcatccggtggcggtctggtgcagggcggtggaagcctgagactgagttgcaaagcctccggcagaactgtgagttccgatgctatgggctggttccgtcaggctccaggcaaggagcgtgagttcgtggcggccataaattggaacggagccagcacctactatgctgattccgtgaagggccgcttcaccatcagccgggataacgcgaagaacaccgtgtacctccagatgaactctctgaagtccgaggatacagctgtctattactgcgccgtatctgattgggttgtgggcgggattagccttatgaacggggcggaatataagtactgggggcaggggaccctggtgaccgtgagtagc

SEQ ID NO: 589 - DNA, 369 bp, Artificial Sequence
  Description of Artificial Sequence Synthetic polynucleotide
caggtgcagcttcaggaatccggcgggggattggtgcagcctggcgggtccctccgtctgtcttgtgcggcctcaggccgcaccttttctagttatgggatgggttggttccgccaggccccaggcaaggaacgtgagtttgtctcaagcatcagttggtcaggccgtagtacatattacacgaacgctgtgagaggtaggttcactatctccagagataacgcaaaaaacaccgtttacctgcaaatgaactctctgaagcccgaggatactgccgtatactattgtgcagcttctagcacctggaataacaatgacccaactgaatatgagtactggggccagggcacccaagtcaccgtttccagc

SEQ ID NO: 590 - DNA, 378 bp, Artificial Sequence
  Description of Artificial Sequence Synthetic polynucleotide
caggtgcagttgcaggagtctgggggcggacttgtgcaggccggggcctccctgcgcctgagctgtgctgcctctggccggaccttctccagcttcatggcttggttccgccaggcccccggcaaggagcgcgagttggtcgccaatatcaactggaatggcggggctacgggttacgctgattccgtgaaaggacggttcaccatctctcgtgacaacgcaaagaacaccctgtatatccagatgaactctctgaagcctgaggacactgccgtgtactattgtgccgcagcccgcccctccagccgctattacacttctaacgacgctgaagagtataactactgggggcagggcacgcaggttaccgtgtcttcc

SEQ ID NO: 591 - DNA, 378 bp, Artificial Sequence
  Description of Artificial Sequence Synthetic polynucleotide
caggtgcaactccaggagtctggaggcggattggtgcaggccggggccagcctgagactcagttgtgccgcgagcggtcggacatctagttccttcatgggctggttcaggcaggcaccaggcaaggagagaaagttcgtggccactatcaactggaacggaggcgctactggttacgctgactctgtgaagggcaggtttaccattagcagggacaacgcgaagaataccctttatctccagatgaacagtctcaagcctgaggacaccgctgtctattactgtgcagcggctcgcacctctagctattactatacaagcacagacgccgaggaatacaactactggggccagggaacactggtcaccgtgtccagc

SEQ ID NO: 592 - DNA, 381 bp, Artificial Sequence
  Description of Artificial Sequence Synthetic polynucleotide
caggtccagcttcaggcttacggcggaggtctggtacaggctggcggatctctgagactctcctgtaaggcctccggcaggaccgtgtcaagcgatgcgatgggatggttcaggcaggctccggggaaagagcgcgagttcgtcgccgctatcaactggaacggtgcctccacatattacgccgatagcgtgaaaggccgcttcactatctcccgcgataacgctaagaacacagtgtacctgcaaatgaactccctcaagtatgaagacaccgctgtgtattactgtgctgtttccgattgggtcgtgggcggtatcagcctgatgaacggagccgagtataagtactggggtcagggcacactcgtgacggtgtcctcc

SEQ ID NO: 593 - DNA, 396 bp, Artificial Sequence
  Description of Artificial Sequence Synthetic polynucleotide
caggtgcagttgcaggagtctggaggcggactcgtgcaggctggcggttccctgcgcttgagctgtgcggcctcaggttctatcttttccatcaatggtatgggctggtatcgccaagccccaggtaagcaacgtgaactcgtggcgctcatcaccagcggtggcagcactcactacgccgacagcgtgaagggccgtttcaccatctcccgcgataacattaaaaacaccctgtacttgcagatgaactctctgaagccggaagatacagctgtttattactgtaatggccgtcgcaaaaccacgggatgggggaccctgagggaatgggagggtagccgcgaccccgatgaccacgattattggggacagggaacccaggtgaccgtatcctcc

SEQ ID NO: 594 - DNA, 381 bp, Artificial Sequence
  Description of Artificial Sequence Synthetic polynucleotide
caggtgcagttgcaggccagcggcgggggcctggtgcagggcggaggctccatgcgtctgtcttgcaaggcatctggtaggacggtttccagtgatgcgatgggatggttccgccaagcgcctggcaaggaacgggagttcgtggctgccattaactggaacggagcgagcacgtattacgctgatagcgtcaagggacggtttaccatcagtcgggacaacgctaaaaataccgtgtaccttcagatgaacagcctgaaaagcgaagatacagccgtgtactattgtgctgtttctgattgggtcgtaggtggaatctctctgatgaatggggccgagtacaagtattggggccagggaactctggtcactgtctcctct

SEQ ID NO: 595 - DNA, 381 bp, Artificial Sequence
  Description of Artificial Sequence Synthetic polynucleotide
caggtgcagttgcaggaatctggtggcgggctcgtgaagggtggaggtagcttgcgcctcagctgtgtggcgtccgggggcactttttcctcttatgcgatgggttggtttcgccaggctcctgggaaggaaagggagtttgtagcagccatcggaggtagcggagattccacctattacgccgacagcgttaagggccgctttaccattagccgcgacaacgctaagaacagcgtctatttgcagatgaataaccttaaaccagaggatactgccatctattactgtgccgcagcccgtgaatctagcgtctactatacctctaccgaccctgaagagtatggatattggggccagggcaccttggtgacagtgtctagc

SEQ ID NO: 596 - DNA, 381 bp, Artificial Sequence
  Description of Artificial Sequence Synthetic polynucleotide
caggtccagcttcaggagtcaggaggcgatctggtccagcgtgggggctccctgcgcctgagctgcaaggcaagcggtcgcacggtttcctctgatgctatgggttggttcagacaggctcccggcaaggaaagggagtttgtggcggccatcaactggaacggtgccagtacctactatgcggacagtgtcaagggacgcttcacaatttctcgggacaacgccaagaataccgtttaccttcagatgaacagcctgaagtctgaggacaccgccgtatattactgcgccgtgtccgattgggtcgtgggaggtatcagtctgatgaatggcgcagaatacaagtactggggccagggaacacaagttaccgtgagttct

SEQ ID NO: 597 - DNA, 369 bp, Artificial Sequence
  Description of Artificial Sequence Synthetic polynucleotide
caggtccaacttcaggaatccggcggaggcctggtcgagactggtggcagtctttccctgagctgcgctgcctctggacgcactttctccgactacggcatgggctggttccgtcaggcaccagggaaggagcgcgagttcgttgcatccagtagctggtctggtcggtccacttattacgccgcagccgtgcgtggacgcttcaccgtcagtagggacaacgcaaaaagcaccgtgtaccttcagatgaactccctgaagccagaggacacggctgtgtactattgcgcagccagttccacttggaacaataacgacccttctgaatacgaatactggggacaggggacccaggtgaccgtgagtagt

SEQ ID NO: 598 - DNA, 384 bp, Artificial Sequence
  Description of Artificial Sequence Synthetic polynucleotide
caagtccagttgcaggagagtggcggtgggctggtaaagggtggcgggtccctgcgcctgagctgtgtggcgtcaggtggcacgttctcatcttacgcgatggggtggtttcgccaagccccagggaaggaacgcgagttcgtggccgcaatcgggggctctggtgactccacatattacgcggactctgtgaaagggcgtttcacaatctcacgcgacaatgctaagaactccgtgtacttgcagatgaactccctcaagcccgaggatacagccgtatattactgccaggcggacccgacaatgtttcacaagctgtattacggcatcaatcctaacgaatatgactattgggggcaaggaacccaggtgaccgtgagcagc

SEQ ID NO: 599 - DNA, 381 bp, Artificial Sequence
  Description of Artificial Sequence Synthetic polynucleotide
caggtgcagctgcaacagtttggtggcggtttggtccaagcaggagggtctctcagacttagctgtgcagccagtgggcgcacattttctgggtacgcgatgggctggttccgccaggcacccggaaaagaacgcgagttcgtcgctgccattaactggtctgggggcagcacttattacgccgattctgtgaagggccgctacactatctcccgcgacaacgccaagaacacgggatacctccagatgaacagcctgaaatccgaagacactgcggtgtattactgtgccgcttctgactgggtggttggcgggatcagcttgatgaatggcgctgagtataagtattggggtcagggtactctggtcaccgtcagtagc

SEQ ID NO: 600 - DNA, 348 bp, Artificial Sequence
  Description of Artificial Sequence Synthetic polynucleotide
caggtgcagcttcaggagtccggtggcggtctggtgcaggcgggtgggtccctcggcctgtcctgctccgcctcaggaaccatcttcaacctggactacatgggttggtatcgccaggccctgggcaagcagcgtgaactcgttgccaccattacctcagatggcaggactaactacgccgactccgtgaaggggcgcttcaccatcttcagagacggcgcaaagaacgccattttgatgcagatgaactccctgaagcccgaggatacagcggtctattactgcaaggcgtacctgtctggccgtagcgactactggggccagggaacgcaggtgactgtttcctct

SEQ ID NO: 601 - DNA, 396 bp, Artificial Sequence
  Description of Artificial Sequence Synthetic polynucleotide
caagtgcagctccaggagttcggcgggggactggtgcaggccggagggtcccttcgtctgtcctgtgctgcgtctggctctattagccgcattaacgctatgggatggtatcgccaggctcctggcaagcagcgtgaactggtggccgtcattacaagcggaggtagcacgcattatgccgactccgtgaagggccgctttaccatcagccgggacaacgcaaagaacacactgtacctgcaaatgaactccctgaaacctgaggacactgcggtatattactgcaatggccgtcgcaagacgaccggctggggttcattgcgtgagtgggagggttcccgcgatcccgacgagtacaactattggggccaaggcactctggtcaccgtgtccagc

SEQ ID NO: 602 - DNA, 381 bp, Artificial Sequence
  Description of Artificial Sequence Synthetic polynucleotide
caggttcagctccaggccagcggaggcggactggcccagggcggagggtctctgcgcctgagttgcaaggcttctggccgcaccgtgtcttccgacgctatgggatggtttcgccaggcccctgggaaggagagggagtttgtcgccgctatcaactggaatggagccagcacctattacgcagactctgtcaaggggcgcttcaccatcagccgggacaacgccaagaacaccgtctacctccagatgaactctctgaagtcagaggacacggcggtgtattactgcgccgtcagcgactgggtagtgggcggtatcagcctgatgaatggagcggagtataagtattggggccagggaaccctggtgactgtctcttca

SEQ ID NO: 603 - DNA, 384 bp, Artificial Sequence
  Description of Artificial Sequence Synthetic polynucleotide
caggtgcaacttcaggaatccgggggtggcctggttcagaccggcggttccctgacactgagttgtgtggcaagcggcgggacattcagttcatacgctatgggctggttccgccaggctcctgggaaagagagagaatttgtcgcggccatcggaggcagtggtgattccacttactatgctgattcagtgaaggggagattcacaatttcacgtgataatgctaaaaacagcgtttacctccagatgaactcactcaagccagaggataccgccgtgtactattgtcaggctgatcccaccatgttccataagctgtattacgggatcaacccgaacgagtatgactactggggccagggaaccctggttactgtctctagt

SEQ ID NO: 604 - DNA, 372 bp, Artificial Sequence
  Description of Artificial Sequence Synthetic polynucleotide
caagtccagttgcaggaaagcgggggcgggctggtgcaggcagggggaagcctccggctgagctgcgccgcttctggccgcacctttagcactcacgctatgggctggttccggcaagcgccgggaaaggaacgcgagtttgtggccgctattgcgtggtcagggggaagtacctactatgccgactccgtgaagggtcgttttacaatctcacgtgacattagtaagaacaccatctacttgcagatgaactccctgaagcctgaagacacggcgttgtattactgtgctgccaccaacggcggtgcatggcactaccacgagcgctactttggttcctggggccaagggacccaggtgactgtctcctct

SEQ ID NO: 605 - DNA, 372 bp, Artificial Sequence
  Description of Artificial Sequence Synthetic polynucleotide
caagttcagctccaggagtccgggggaggcctggtgcaggcgggcggtagcctgcgcttgtcttgcgcggccagtggacgcactttcagcactcacgctatgggctggttccggcaggcccctggcaaggagcgcgagtttgtggcggcaatcgcatggtccggtggctccacctactatgccgactccgtgaagggccgcttcaccatttcccgtgacatctccaagaacaccatctatctccagatgaatagcctgaagccggaagatacggctctctactattgtgccgcgaccaacggcggagcctggcactatcacgaacgctacttcgggtcctgggggcaggggaccctggttaccgttagctct

SEQ ID NO: 606 - DNA, 369 bp, Artificial Sequence
  Description of Artificial Sequence Synthetic polynucleotide
caagtgcagctccaggagagtggcggtggactggtacaggctggtggctccctgcgtctcagctgtgtggcctctggccgcacatttagttcctacggcatgggatggttccgtcaggcccctggcaaggaacgtgagttcgttgccagcatttcctggagtgggggttccacctactatcctgacagcgtcctgggccgcttcaccatttctcgtgacaacgcaaaaaacgtgttgtatttgcagatgaacagcctgaagcctgaagataccgctgtctactattgtgcggcctctagcacctggaataacaatgatccccatgagtacgactactggggccagggcaccctggtgaccgtgtctagt

SEQ ID NO: 607 - DNA, 381 bp, Artificial Sequence
  Description of Artificial Sequence Synthetic polynucleotide
caggtgcagctgcaagcgagcggaggtgggctggtccagggtggaggttccatgcgcctcagctgcgctgcatccggtcgcaccgtgtccagcgatgctatgggctggttccggcaggcccctgggaaggagcgtgagttcgtcgctgccatcaactggaacggcgcaagcacctactatgcagattccgtgaaaggtcgtttcacaatttctcgcgacaacgctaagaataccgtctacttgcagatgaactcccttaaatcagaggacaccgccgtgtattactgcgcagccagcgattgggttgtgggaggcatcagccttatgaacgcagctgaatacaaatattggggtcagggcactcaggtgaccgtctcctct

SEQ ID NO: 608 - protein, 10 aa, Unknown
  Description of Unknown c-Myc sequence
EQKLISEEDL

SEQ ID NO: 609 - protein, 6 aa, Artificial Sequence
  Description of Artificial Sequence Synthetic 6xHis tag
HHHHHH

SEQ ID NO: 610 - protein, 8 aa, Artificial Sequence
  Description of Artificial Sequence Synthetic 8xHis tag
HHHHHHHH

SEQ ID NO: 611 - protein, 6 aa, Artificial Sequence
  Description of Artificial Sequence Synthetic His tag
  SITE (1)..(6): This sequence may encompass 3-6 residues
HHHHHH